(19) United States
(12) Patent Application Publication
(10) Pub. No.: US 2015/0295179 A1
(43) Pub. Date: Oct. 15, 2015

(54) MONOMERIC, OLIGOMERIC AND POLYMERIC SEMICONDUCTORS CONTAINING FUSED RINGS AND THEIR DEVICES

(71) Applicant: Yuning LI, (US)

(72) Inventor: Yuning Li, Ontario (CA)

(21) Appl. No.: 14/441,614

(22) PCT Filed: Nov. 7, 2013
(86) PCT No.: PCT/CA2013/050854

§ 371 Date: May 8, 2015

Related U.S. Application Data
(60) Provisional application No. 61/724,461, filed on Nov. 9, 2012.

(51) Int. Cl. H01L 51/00; C09K 11/06; C08G 73/06; C07D 493/04; C08G 75/00

(52) U.S. Cl. CPC H01L 51/0036; C07D 493/04; H01L 51/0072; H01L 51/0073; C08G 75/00; C08G 73/0672; H01L 51/0035; C09K 11/06; C09K 2211/1029; C09K 2211/1088; C09K 2211/1011; C09K 2211/1483; C09K 2211/1475; C09K 2211/1416

(57) ABSTRACT

The present invention describes the monomeric, oligomeric and polymeric materials comprising a fused-ring moiety, which can be used as organic semiconductors for use in electronic, optical, or optoelectronic devices such as organic thin film transistors and organic photovoltaics.

### BACKGROUND

Organic electronics can be manufactured at lower costs as compared to conventional silicon-based electronics and are suitable for widespread applications including displays, radio-frequency identification (RFID) tags, chemo-/biosensors, memory devices, solar cells, photodiodes, etc. In addition, organic semiconductors can be processed at low temperatures and deposited on plastic substrates to enable light weight, flexible, and ultra-thin electronic devices. However, organic semiconductors, especially solution-processed organic semiconductors, have shown insufficient electronic performance as compared with inorganic semiconductors. For example, the charge carrier mobility of solution-processed organic semiconductors is typically lower than 1 cm²V⁻¹s⁻¹, which is inadequate as channel semiconductor materials in organic thin film transistors (OTFTs) for many target applications. Therefore there is a need to develop solution-processable organic semiconductors, including monomers, oligomers and polymers, with mobility greater than 0.5 cm²V⁻¹s⁻¹.

### SUMMARY

The present invention discloses semiconducting organic compounds comprising a fused-ring moiety, which can be used as high performance organic semiconductors for OTFTs, organic photovoltaics (OPVs), sensors, and other electronic devices.

One objective of the present invention is to develop monomeric, oligomeric or polymeric semiconductor materials comprising said fused-ring moiety for electronic devices such as OTFTs, OPVs, and sensors.

Another objective is to develop OTFTs, OPVs, sensors, and other electronic devices comprising said organic semiconductors comprising such fused-ring moieties.

The present application also provides for a mixture or blend comprising one or more of said organic semiconducting compound and one or more compounds or polymers having semiconducting, charge transport, hole transport, electron transport, hole blocking, electron blocking, electrically conducting, photoconducting or light emitting properties.

Further, the present application provides for a formulation comprising said organic semiconducting compound and an organic solvent.

Furthermore, the present application provides for the use of said organic semiconducting compound as charge transport, semiconducting, electrically conducting, photoconducting or light emitting material in optical, electrooptical, electronic, electroluminescent or photoluminescent components or devices.

Additionally the present application provides for charge transport, semiconducting, electrically conducting, photoconducting or light emitting materials comprising said organic semiconducting compound.

The present application also provides for a component or device comprising such organic semiconducting compound, said component or device being selected from the group consisting of organic field effect transistors (OFET), thin film transistors (TFT), integrated circuits (IC), logic circuits, capacitors, radio frequency identification (RFID) tags, devices or components, organic light emitting diodes (OLED), organic light emitting transistors (OLET), flat panel displays, backlights of displays, organic photovoltaic devices (OPV), organic solar cells (OSC), photodiodes, laser diodes, photoconductors, organic photodetectors (OPD), electrophotographic devices, electrophotographic recording devices, organic memory devices, sensor devices, charge injection layers, charge transport layers or interlayers in polymer light emitting diodes (PLEDs), Schottky diodes, planarising layers, antistatic films, polymer electrolyte membranes (PEM), conducting substrates, conducting patterns, electrode materials in batteries, alignment layers, biosensors, biochips, security markings, security devices, and components or devices for detecting and discriminating DNA sequences.

The present application further relates to conjugated polymers comprising one or more repeating units which comprise said fused-ring moiety and/or one or more groups selected from aryl and heteroaryl groups.

The invention further relates to monomers comprising said fused-ring moiety and further comprising one or more reactive groups which can be reacted to form a conjugated polymer as described herein.

The invention also relates to small molecules comprising said fused-ring moiety and one or more inert groups.

The invention further relates to the use of a polymer, formulation, mixture or polymer blend of the present invention as charge transport, semiconducting, electrically conducting, photoconducting or light emitting material, or in an optical, electrooptical, electronic, electroluminescent or photoluminescent device, or in a component of such a device or in an assembly comprising such a device or component.

The optical, electrooptical, electronic, electroluminescent and photoluminescent devices include, without limitation, organic field effect transistors (OFET), organic thin film transistors (OTFT), organic light emitting diodes (OLED), organic light emitting transistors (OLET), organic photovoltaic devices (OPV), organic photodetectors (OPD), organic solar cells, laser diodes, Schottky diodes, photoconductors and photodetectors.

The components of the above devices include, without limitation, charge injection layers, charge transport layers, interlayers, planarizing layers, antistatic films, polymer electrolyte membranes (PEM), conducting substrates and conducting patterns.

The assemblies comprising such devices or components include, without limitation, integrated circuits (IC), radio frequency identification (RFID) tags or security markings or security devices containing them, flat panel displays or backlights thereof, electrophotographic devices, electrophotographic recording devices, organic memory devices, sensor devices, biosensors and biochips.

Other objectives and advantages of the invention will become readily apparent from the following discussion.

### BRIEF DESCRIPTION OF DRAWINGS

FIG. 1 depicts a typical bottom gate/top contact OTFT structure.

FIG. 2 depicts a typical bottom gate/bottom contact OTFT structure.

FIG. 3 depicts a typical top gate/bottom contact OTFT structure.

FIG. 4 depicts a typical top gate/top contact OTFT structure.

FIG. 5 shows the output (left) and transfer (right) characteristics of an OTFT device with PIBDFT-24 (annealed at 200° C. for 15 minutes) in the electron accumulation regime. L=30 μm, W=1 mm.

FIG. 6 shows the output (left) and transfer (right) characteristics of an OTFT device with PIBDFV-24 (annealed at 200° C. for 15 minutes) in the electron accumulation regime. L=30 μm, W=1 mm.

FIG. 7 shows the output (left) and transfer (right) characteristics of an OTFT device with PIBDFV-26 (annealed at 200° C. for 15 minutes) in the electron accumulation regime. L=30 μm, W=1 mm.

FIG. 8 shows the output (left) and transfer (right) characteristics of an OTFT device with PIBDFV-40 (annealed at 200° C. for 15 minutes) in the electron accumulation regime. L=30 μm, W=1 mm.

FIG. 9 shows the output (left) and transfer (right) characteristics of an OTFT device with PIBDFBT-40 (annealed at 200° C. for 15 minutes) in the electron accumulation regime. L=30 μm, W=1 mm.

FIG. 10 shows the output (left) and transfer (right) characteristics of an OTFT device with PIBDFBT-40 (annealed at 200° C. for 15 minutes) in the hole accumulation regime. L=30 μm, W=1 mm.

### DETAILED DESCRIPTION

For the purposes of the present application the term “substituted” is used to denote substitution, i.e. replacement of a hydrogen, by a substituent R^{S} selected from the group consisting of halogen atoms, alkyl having from 1 to 60, preferably from 1 to 50, more preferably from 1 to 30 and most preferably from 1 to 20 carbon atoms, alkyl having from 1 to 60, preferably from 1 to 50, more preferably from 1 to 30 and most preferably from 1 to 20 carbon atoms wherein at least one of the hydrogen atoms is replaced by a halogen atom, alkyl having from 1 to 60, preferably from 1 to 50, more preferably from 1 to 30 and most preferably from 1 to 20 carbon atoms wherein at least one of the methylene moieties (CH₂) is replaced by an oxygen atom, aryl having from 5 to 20 ring atoms with the ring atoms being independently of each other selected from the group consisting of carbon and heteroatoms as defined below, and aryl having from 5 to 20 ring atoms with the ring atoms being independently of each other selected from the group consisting of carbon and heteroatoms as defined below and at least one hydrogen is replaced by a halogen atom.

For the purposes of the present application the term “polymer” will be understood to mean a molecule of high relative molecular mass, the structure of which essentially comprises the multiple repetition of units derived, actually or conceptually, from molecules of low relative molecular mass (Pure Appl. Chem., 1996, 68, 2291). The term “oligomer” will be understood to mean a molecule of intermediate relative molecular mass, the structure of which essentially comprises a small plurality of units derived, actually or conceptually, from molecules of lower relative molecular mass (Pure Appl. Chem., 1996, 68, 2291). In a preferred meaning as used herein a polymer will be understood to mean a compound having >1, i.e. at least 2 repeat units, preferably ≧5 repeat units, and an oligomer will be understood to mean a compound with >1 and <10, preferably <5, repeat units.

Further, as used herein, the term “polymer” will be understood to mean a molecule that encompasses a backbone (also referred to as “main chain”) of one or more distinct types of repeat units (the smallest constitutional unit of the molecule) and is inclusive of the commonly known terms “oligomer”, “copolymer”, “homopolymer” and the like. Further, it will be understood that the term polymer is inclusive of, in addition to the polymer itself, residues from initiators, catalysts and other elements attendant to the synthesis of such a polymer, where such residues are understood as not being covalently incorporated thereto. Further, such residues and other elements, while normally removed during post polymerization purification processes, are typically mixed or co-mingled with the polymer such that they generally remain with the polymer when it is transferred between vessels or between solvents or dispersion media.

As used herein, the terms “repeat unit”, “repeating unit” and “monomeric unit” are used interchangeably and will be understood to mean the constitutional repeating unit (CRU), which is the smallest constitutional unit the repetition of which constitutes a regular macromolecule, a regular oligomer molecule, a regular block or a regular chain (Pure Appl. Chem., 1996, 68, 2291). As further used herein, the term “unit” will be understood to mean a structural unit which can be a repeating unit on its own, or can together with other units form a constitutional repeating unit.

As used herein, a “terminal group” will be understood to mean a group that terminates a polymer backbone. The expression “in terminal position in the backbone” will be understood to mean a divalent unit or repeat unit that is linked at one side to such a terminal group and at the other side to another repeat unit. Such terminal groups include endcap groups or reactive groups that are attached to a monomer forming the polymer backbone which did not participate in the polymerisation reaction, like for example a group having the meaning of R^{e} or R^{f} as defined below.

As used herein, the term “endcap group” will be understood to mean a group that is attached to, or replacing, a terminal group of the polymer backbone. The endcap group can be introduced into the polymer by an endcapping process. Endcapping can be carried out for example by reacting the terminal groups of the polymer backbone with a monofunctional compound (“endcapper”) like for example an alkyl- or arylhalide, an alkyl- or arylstannane or an alkyl- or arylboronate. The endcapper can be added for example after the polymerisation reaction. Alternatively the endcapper can be added in situ to the reaction mixture before or during the polymerisation reaction. In situ addition of an endcapper can also be used to terminate the polymerisation reaction and thus control the molecular weight of the forming polymer.

As used herein, the terms “donor” or “donating” and “acceptor” or “accepting” will be understood to mean an electron donor and electron acceptor, respectively. “Electron donor” will be understood to mean a chemical entity that donates electrons to another compound or another group of atoms of a compound. “Electron acceptor” will be understood to mean a chemical entity that accepts electrons transferred to it from another compound or another group of atoms of a compound. See also International Union of Pure and Applied Chemistry, Compendium of Chemical Technology, Gold Book, Version 2.3.2, 19. August 2012, pages 477 and 480.

As used herein, the term “n-type” or “n-type semiconductor” will be understood to mean an extrinsic semiconductor in which the conduction electron density is in excess of the mobile hole density, and the term “p-type” or “p-type semiconductor” will be understood to mean an extrinsic semiconductor in which mobile hole density is in excess of the conduction electron density (see also, J. Thewlis, Concise Dictionary of Physics, Pergamon Press, Oxford, 1973).

As used herein, the term “leaving group” will be understood to mean an atom or group (which may be charged or uncharged) that becomes detached from an atom in what is considered to be the residual or main part of the molecule taking part in a specified reaction (see also Pure Appl. Chem., 1994, 66, 1134).

As used herein, the term “conjugated” will be understood to mean a compound (for example a polymer) that contains mainly C atoms with sp²-hybridisation (or optionally also sp-hybridization), and wherein these C atoms may also be replaced by hetero atoms. In the simplest case this is for example a compound with alternating C—C single and double (or triple) bonds, but is also inclusive of compounds with aromatic units like for example 1,4-phenylene. The term “mainly” in this connection will be understood to mean that a compound with naturally (spontaneously) occurring defects, or with defects included by design, which may lead to interruption of the conjugation, is still regarded as a conjugated compound. See also International Union of Pure and Applied Chemistry, Compendium of Chemical Technology, Gold Book, Version 2.3.2, 19. August 2012, pages 322-323.

As used herein, unless stated otherwise the molecular weight is given as the number average molecular weight Mₙ or weight average molecular weight M_{w}, which is determined by gel permeation chromatography (GPC) against polystyrene standards in eluent solvents such as tetrahydrofuran, trichloromethane (TCM, chloroform), chlorobenzene or 1,2,4-trichlorobenzene. Unless stated otherwise, 1,2,4-trichlorobenzene is used as solvent. The molecular weight distribution (“MWD”), which may also be referred to as polydispersity index (“PDI”), of a polymer is defined as the ratio M_{w}/Mₙ. The degree of polymerization, also referred to as total number of repeat units, m (or n), will be understood to mean the number average degree of polymerization given as m (or n)=Mₙ/Mᵤ, wherein Mₙ is the number average molecular weight and Mᵤ is the molecular weight of the single repeat unit, see J. M. G. Cowie, Polymers: Chemistry & Physics of Modern Materials, Blackie, Glasgow, 1991.

The present invention relates to the development and applications of monomeric, oligomeric and polymeric semiconductor materials comprising a fused-ring moiety (I):

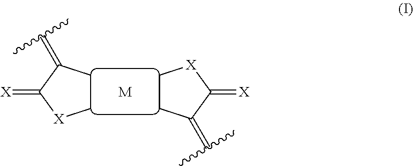

X is independently (i.e., the four X's in (I) can have different structures) oxygen (O), sulphur (S), or NR (R is independently hydrogen or optionally substituted hydrocarbon, cyclic and/or acyclic, with 1 to 60, in a first preferred aspect with 1 to 40 and in a second preferred aspect with 41 (or 45) to 60 carbon atoms such as, for example, alkyl, substituted alkyl, alkenyl, substituted alkenyl, alkynyl, substituted alkynyl, aryl, and substituted aryl, or any other suitable group);

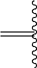

indicates the linkage with another moiety (or moieties);

M is a suitable conjugated moiety selected from, but not restricted to, the following structures and a combination of them:

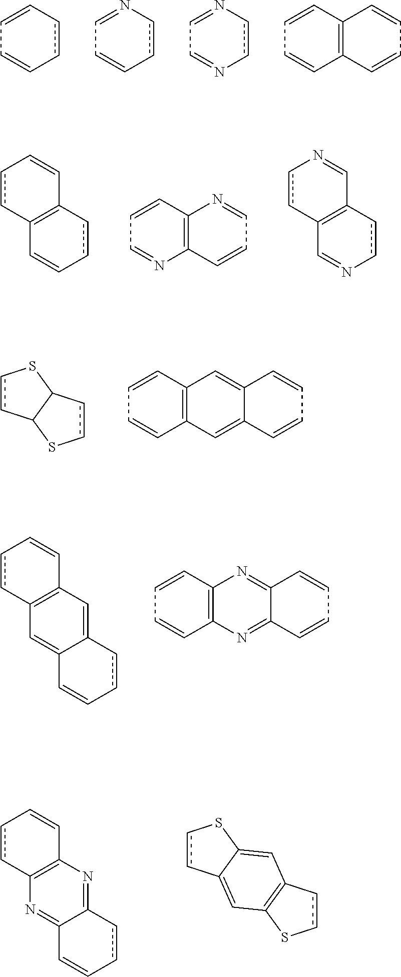

Wherein:

--- indicates the fusion of M with the adjacent five-membered rings and the fusion can be in any suitable direction;

each structure can be substituted, where is applicable, with one or more suitable groups independently selected from optionally substituted hydrocarbon with about 1 to about 60, in a first preferred aspect with 1 to 40 and in a second preferred aspect with 41 (or 45) to 60 carbon (C) atoms (such as, for example, alkyl, substituted alkyl, alkenyl, substituted alkenyl, alkynyl, substituted alkynyl, aryl, substituted aryl, alkoxy, and substituted alkoxy), cyano (CN), nitro, and halogen, or any other suitable group.

An alkyl or alkoxy radical, i.e. where the terminal CH₂ group is replaced by —O—, can be straight-chain or branched. Suitable examples of such alkyl and alkoxy radical are methyl, ethyl, propyl, butyl, pentyl, hexyl, heptyl, octyl, nonyl, decyl, undecyl, dodecyl, tridecyl, tetradecyl, pentadecyl, methoxy, ethoxy, propoxy, butoxy, pentoxy, hexoxy, heptoxy, octoxy, nonoxy, decoxy, undecoxy, dodecoxy, tridecoxy or tetradecoxy. Preferred alkyl and alkoxy radicals have from 1 to 60, preferably from 1 to 50, more preferably from 1 to 40, even more preferably from 1 to 30 and most preferably from 1 to 20 carbon atoms. Suitable examples of such preferred alkyl and alkoxy radicals may be selected from the group consisting of methyl, ethyl, propyl, butyl, pentyl, hexyl, heptyl, octyl, nonyl, decyl, undecyl, dodecyl, 2-ethylhexyl, 2-butyloctyl, 2-hexyldecyl, 2-octyldodecyl, 2-decyltetradecyl, 4-decylhexadecyl, 7-decylnonadecyl, 4-octadecyldocosyl, methoxy, ethoxy, propoxy, butoxy, pentoxy, hexoxy, heptoxy, octoxy, nonoxy, decoxy, undecoxy, dodecoxy, 2-ethyl-hexoxy, 2-butyloctoxy, 2-hexyldecoxy, and 2-octyldodecoxy.

An alkenyl group, wherein one or more CH₂ groups are replaced by —CH═CH— can be straight-chain or branched. It preferably has 2 to 40, more preferably 2 to 30, even more preferably 2 to 20 and most preferably 2 to 10 C atoms. Preferred examples of alkenyl groups may be selected from the group consisting of vinyl, prop-1-enyl, or prop-2-enyl, but-1-enyl, but-2-enyl or but-3-enyl, pent-1-enyl, pent-2-enyl, pent-3-enyl or pent-4-enyl, hex-1-enyl, hex-2-enyl, hex-3-enyl, hex-4-enyl or hex-5-enyl, hept-1-enyl, hept-2-enyl, hept-3-enyl, hept-4-enyl, hept-5-enyl or hept-6-enyl, oct-1-enyl, oct-2-enyl, oct-3-enyl, oct-4-enyl, oct-5-enyl, oct-6-enyl or oct-7-enyl, non-1-enyl, non-2-enyl, non-3-enyl, non-4-enyl, non-5-enyl, non-6-enyl, non-7-enyl or non-8-enyl, dec-1-enyl, dec-2-enyl, dec-3-enyl, dec-4-enyl, dec-5-enyl, dec-6-enyl, dec-7-enyl, dec-8-enyl or dec-9-enyl.

Especially preferred alkenyl groups are C₂-C₂₀-1E-alkenyl, C₄-C₂₀-3E-alkenyl, C₅-C₂₀-4-alkenyl, and C₆-C₂₀-5-alkenyl, in particular C₂-C₇-1E-alkenyl, C₄-C₇-3E-alkenyl and C₅-C₇-4-alkenyl. Examples for particularly preferred alkenyl groups are vinyl, 1E-propenyl, 1E-butenyl, 1E-pentenyl, 1E-hexenyl, 1E-heptenyl, 3-butenyl, 3E-pentenyl, 3E-hexenyl, 3E-heptenyl, 4-pentenyl, 4Z-hexenyl, 4E-hexenyl, 4Z-heptenyl, 5-hexenyl, 6-heptenyl and the like. Alkenyl groups having up to 12 C atoms are generally preferred.

The terms “aryl” and “heteroaryl” as used herein preferably mean a mono-, bi- or tricyclic aromatic or heteroaromatic group with 4 to 30 ring C atoms that may also comprise condensed rings and is optionally substituted with one or more groups L, wherein L is selected from halogen, —CN, —NC, —NCO, —NCS, —OCN, —SCN, —C(═O)NR⁰R⁰⁰, —C(═O)X⁰, —C(═O)R⁰, —NH₂, —NR⁰R⁰⁰, —SH, —SR⁰, —SO₃H, —SO₂R⁰, —OH, —NO₂, —CF₃, —SF₅, optionally substituted silyl, or carbyl or hydrocarbyl with 1 to 60, preferably with 1 to 50, more preferably with 1 to 40, even more preferably with 1 to 30 and most preferably with 1 to 20 C atoms that is optionally substituted and optionally comprises one or more hetero atoms, and is preferably alkyl, alkoxy, thiaalkyl, alkylcarbonyl, alkoxycarbonyl or alkoxycarbonyloxy with 1 to 40, preferably with 1 to 30 and more preferably with 1 to 20 C atoms that is optionally fluorinated, and R⁰, R⁰⁰ and X⁰ have the meanings given above and below.

Very preferred substituents L are selected from halogen, most preferably F, or alkyl, alkoxy, oxaalkyl, thioalkyl, fluoroalkyl and fluoroalkoxy with 1 to 24 or with 1 to 12 C atoms or alkenyl, and alkynyl with 2 to 24 or with 2 to 12 C atoms.

Especially preferred aryl and heteroaryl groups are phenyl, phenyl wherein one or more CH groups are replaced by N, naphthalene, thiophene, selenophene, thienothiophene, dithienothiophene, fluorene and oxazole, all of which can be unsubstituted, mono- or polysubstituted with L as defined above. Very preferred rings are selected from pyrrole, preferably N-pyrrole, furan, pyridine, preferably 2- or 3-pyridine, pyrimidine, pyridazine, pyrazine, triazole, tetrazole, pyrazole, imidazole, isothiazole, thiazole, thiadiazole, isoxazole, oxazole, oxadiazole, thiophene, preferably 2-thiophene, selenophene, preferably 2-selenophene, thieno[3,2-b]thiophene, thieno[2,3-b]thiophene, furo[3,2-b]furan, furo[2,3-b]furan, seleno[3,2-b]selenophene, seleno[2,3-b]selenophene, thieno[3,2-b]selenophene, thieno[3,2-b]furan, indole, isoindole, benzo[b]furan, benzo[b]thiophene, benzo[1,2-b;4,5-b]dithiophene, benzo[2,1-b;3,4-b′]dithiophene, quinole, 2-methylquinole, isoquinole, quinoxaline, quinazoline, benzotriazole, benzimidazole, benzothiazole, benzisothiazole, benzisoxazole, benzoxadiazole, benzoxazole, benzothiadiazole, all of which can be unsubstituted, mono- or polysubstituted with L as defined above. Further examples of aryl and heteroaryl groups are those selected from the groups shown hereinafter.

Specifically, (I) is one of the following exemplary structures:

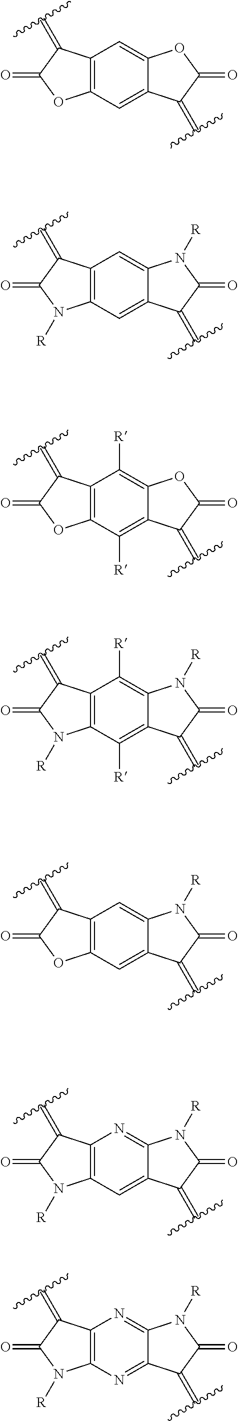

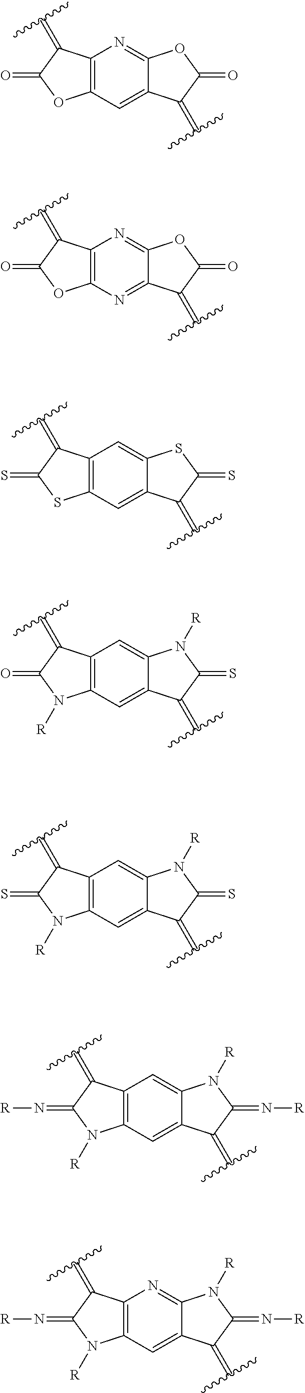

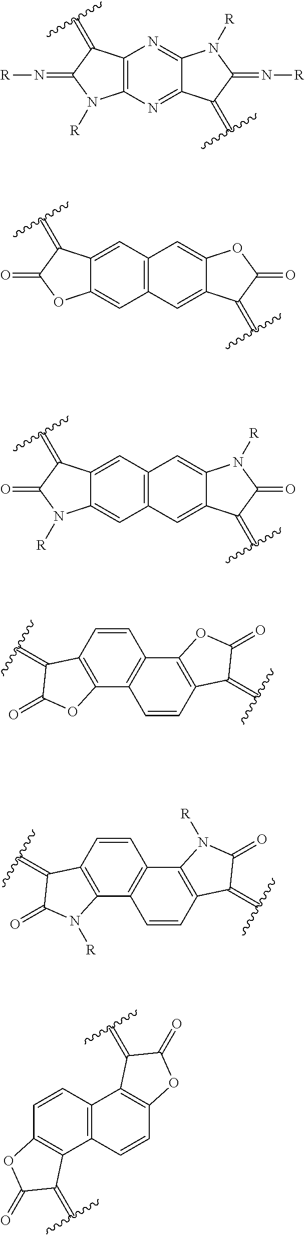

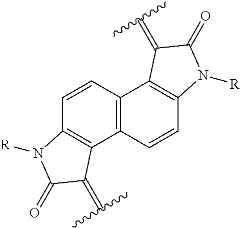

wherein R is independently hydrogen or optionally substituted hydrocarbon with 1 to 60, in a first preferred aspect with 1 to 40 and in a second preferred aspect with 41 (or 45) to 60 carbon atoms such as, for example, alkyl, substituted alkyl, alkenyl, substituted alkenyl, alkynyl, substituted alkynyl, aryl, and substituted aryl, or any other suitable group;

R′ is independently hydrogen or optionally substituted hydrocarbon with 1 to 60, in a first preferred aspect with 1 to 40 and in a second preferred aspect with 41 (or 45) to 60 carbon atoms such as, for example, alkyl, substituted alkyl, alkenyl, substituted alkenyl, alkynyl, substituted alkynyl, aryl, and substituted aryl, or any other suitable group;

each structure can be further substituted, where is applicable, with one or more suitable groups independently selected from optionally substituted hydrocarbon with 1 to 60, in a first preferred aspect with 1 to 40 and in a second preferred aspect with 41 (or 45) to 60 carbon atoms (such as, for example, alkyl, substituted alkyl, alkenyl, substituted alkenyl, alkynyl, substituted alkynyl, aryl, substituted aryl, alkoxy, and substituted alkoxy), cyano (CN), nitro, and halogen, or any other suitable group.

More specifically, (I) is one of the following structures;

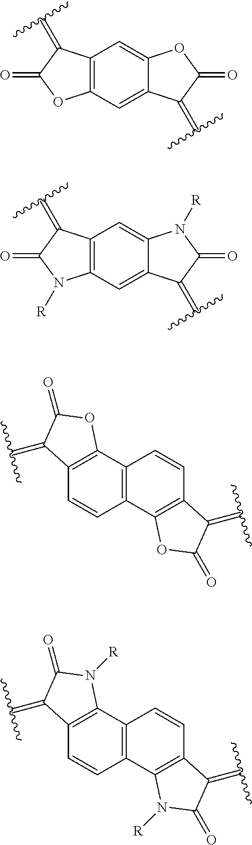

where R is independently hydrogen or optionally substituted hydrocarbon with 1 to 60, in a first preferred aspect with 1 to 40 and in a second preferred aspect with 41 (or 45) to 60 carbon atoms such as, for example, alkyl, substituted alkyl, alkenyl, substituted alkenyl, alkynyl, substituted alkynyl, aryl, and substituted aryl, or any other suitable group;

each structure can be further substituted, where is applicable, with one or more suitable groups independently selected from optionally substituted hydrocarbon with 1 to 60, in a first preferred aspect with 1 to 40 and in a second preferred aspect with 41 (or 45) to 60 carbon atoms (such as, for example, alkyl, substituted alkyl, alkenyl, substituted alkenyl, alkynyl, substituted alkynyl, aryl, substituted aryl, alkoxy, and substituted alkoxy), cyano (CN), nitro, and halogen, or any other suitable group;

More specifically, this invention relates to the development of monomeric, oligomeric and polymeric semiconductor materials comprising a moiety (I) with the following general structure (PI) and (PII):

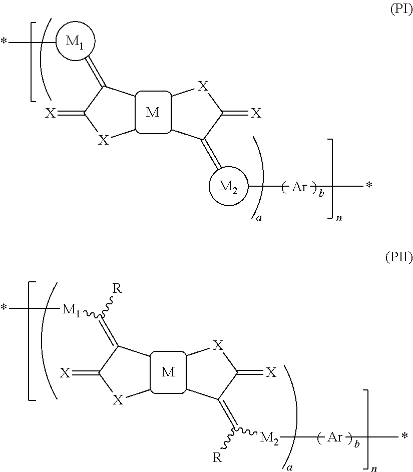

wherein

X, M and R are defined as above;

a is an integer from 1 to 20;

b or c is an integer from 0 (zero) to 20;

the unit Ar and the unit M₁-(I)-M₂ can be connected in a random or alternating manner, e.g., (PI) or (PII) can be a random copolymer, an alternating copolymer, or a block copolymer;

n is a number from about 1 to 1,000,000;

[symbol] indicates the linkage can be a cis- or trans-structure;

the terminal “*” can be hydrogen or any other suitable group or moiety.

Other groups or moieties suitable as “*” may be as defined for R^{a} or R^{c} below.

Ar is independently (i.e. in the case of b>1, each Ar may have a different structure from the other) a π-conjugated moiety selected from, but not restricted to, the following structures and a combination of them:

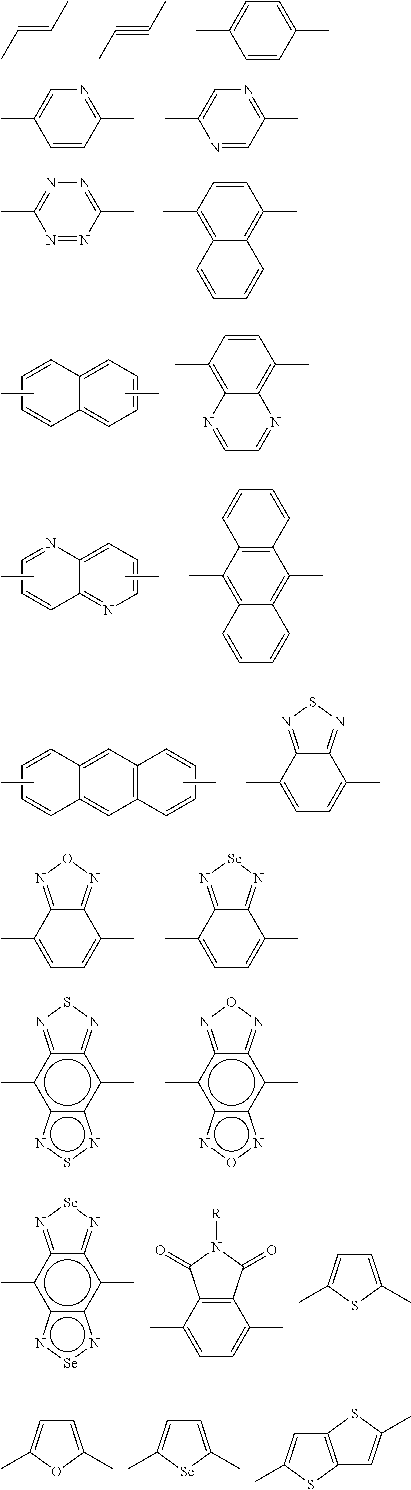

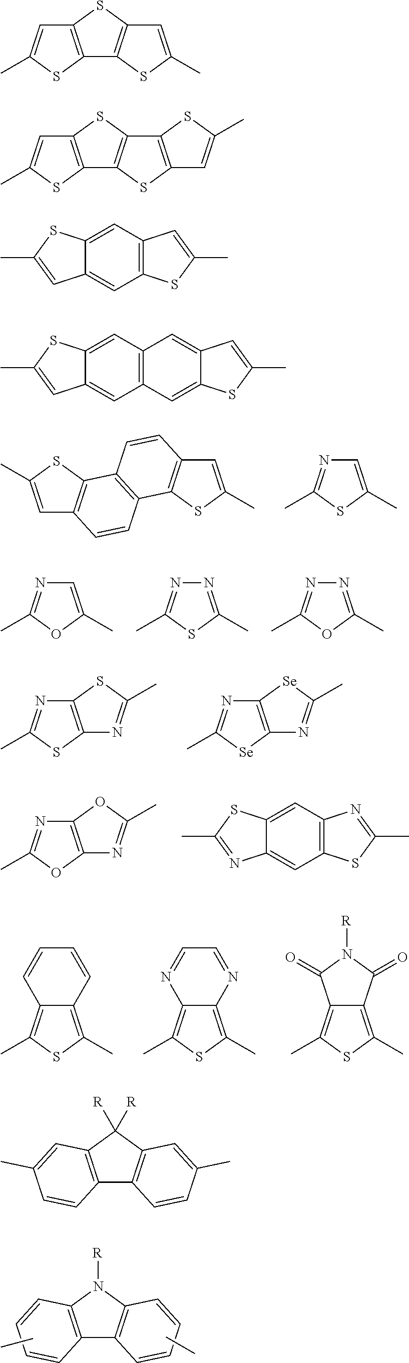

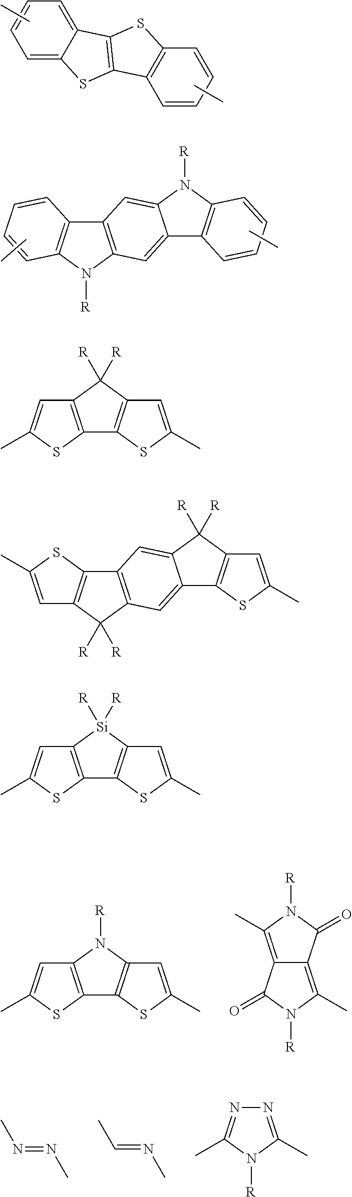

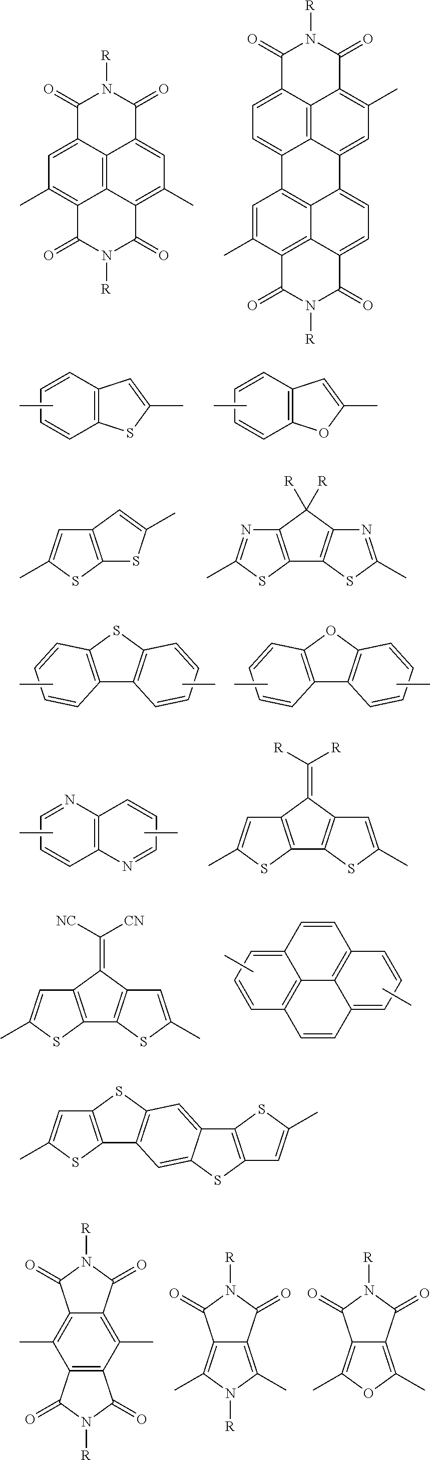

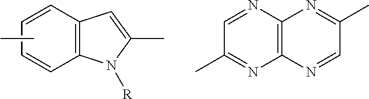

wherein each structure can be substituted, where is applicable, with one or more suitable groups independently selected from optionally substituted hydrocarbon with 1 to 60, in a first preferred aspect with 1 to 40 and in a second preferred aspect with 41 (or 45) to 60 carbon atoms (such as, for example, alkyl, substituted alkyl, alkenyl, substituted alkenyl, alkynyl, substituted alkynyl, aryl, substituted aryl, alkoxy, and substituted alkoxy), cyano (CN), nitro, and halogen, or any other suitable group;

R is independently hydrogen, optionally substituted hydrocarbon with 1 to 60, in a first preferred aspect with 1 to 40 and in a second preferred aspect with 41 (or 45) to 60 carbon atoms (such as, for example, alkyl, substituted alkyl, alkenyl, substituted alkenyl, alkynyl, substituted alkynyl, aryl, or substituted aryl), or any other suitable group;

M₁ and M₂ are independently a π-conjugated moiety selected from, but not restricted to, the following structures and a combination of them:

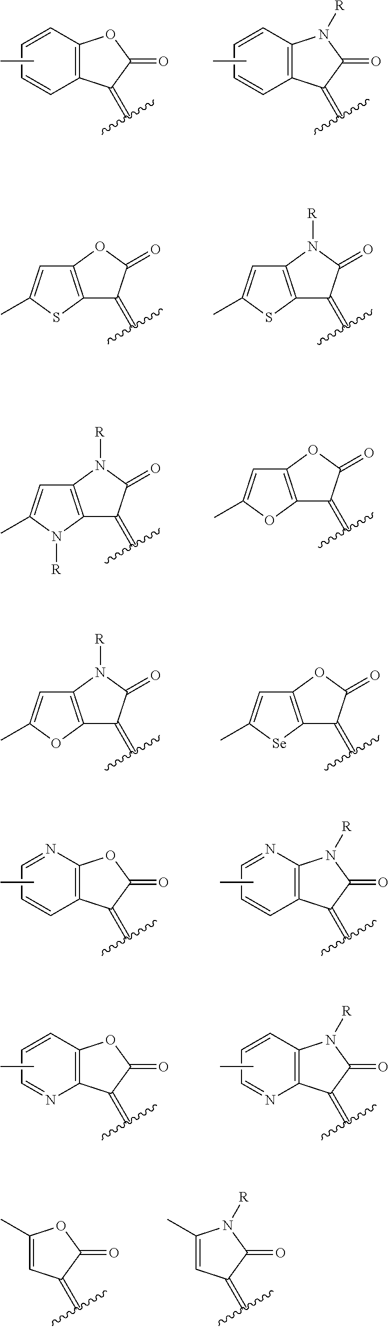

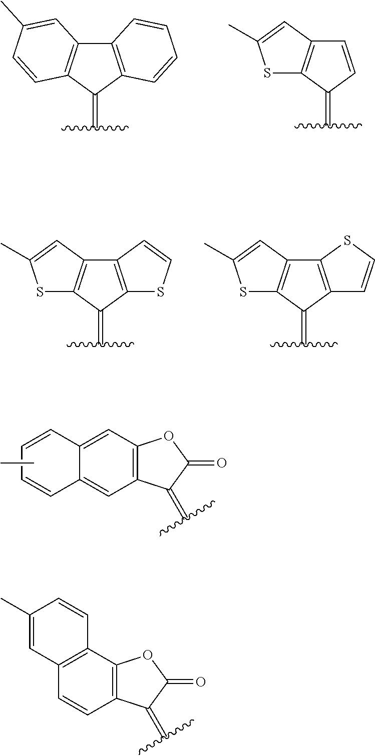

wherein each structure can be substituted, where is applicable, with one or more suitable groups independently selected from optionally substituted hydrocarbon with 1 to 60, in a first preferred aspect with 1 to 40 and in a second preferred aspect with 41 (or 45) to 60 carbon atoms (such as, for example, alkyl, substituted alkyl, alkenyl, substituted alkenyl, alkynyl, substituted alkynyl, aryl, substituted aryl, alkoxy, and substituted alkoxy), cyano (CN), nitro, and halogen, or any other suitable group; R is independently hydrogen, optionally substituted hydrocarbon with 1 to 60, in a first preferred aspect with 1 to 40 and in a second preferred aspect with 41 (or 45) to 60 carbon atoms (such as, for example, alkyl, substituted alkyl, alkenyl, substituted alkenyl, alkynyl, substituted alkynyl, aryl, or substituted aryl), or any other suitable group.

More specifically, the (PI) and (PII) are selected form the following structures:

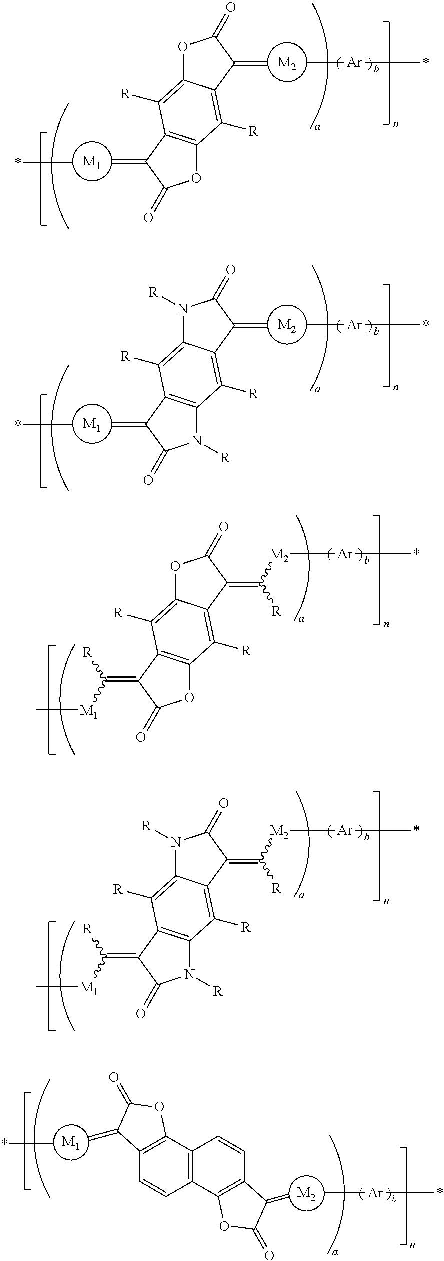

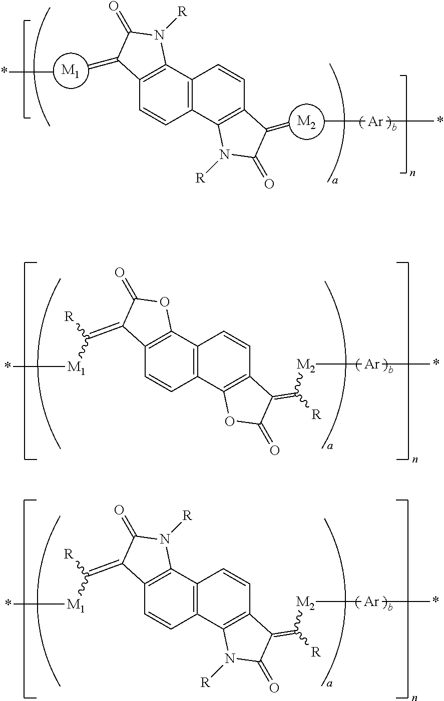

wherein:

M₁, M₂, Ar, R, a, b, and n are defined as above;

each structure can be substituted, where is applicable, with one or more suitable groups independently selected from optionally substituted hydrocarbon with 1 to 60, in a first preferred aspect with 1 to 40 and in a second preferred aspect with 41 (or 45) to 60 carbon atoms (such as, for example, alkyl, substituted alkyl, alkenyl, substituted alkenyl, alkynyl, substituted alkynyl, aryl, substituted aryl, alkoxy, and substituted alkoxy), cyano (CN), nitro, and halogen, or any other suitable group;

n is a number from 1 to 1,000,000;

[symbol] indicates the linkage can be a cis- or trans-structure;

the terminal “*” can be hydrogen or any other suitable group or moiety.

Other groups or moieties suitable as “*” may be as defined for R^{a} or R^{c} below.

Preferred embodiments of the present invention are illustrated in structures (1) through (230):

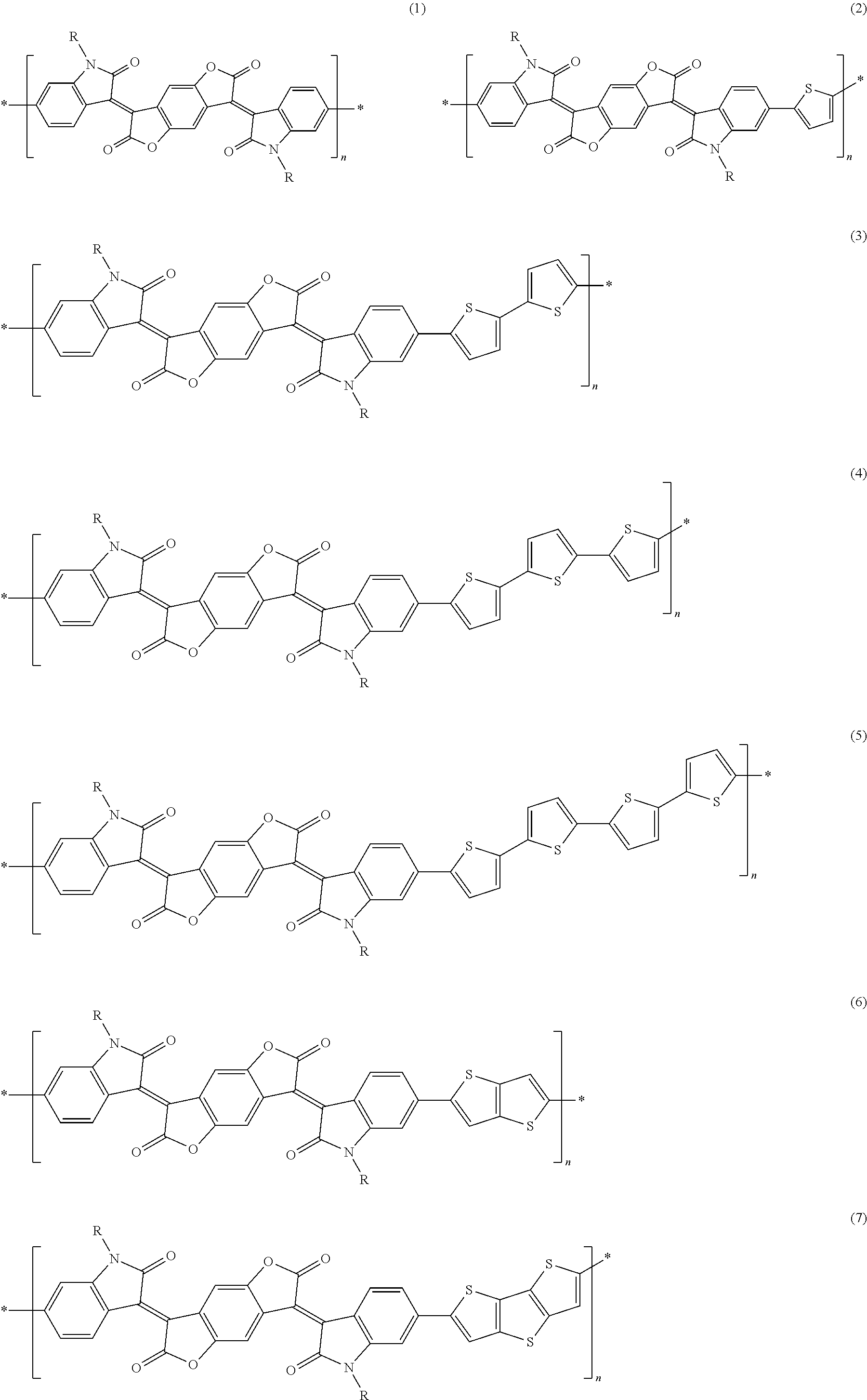

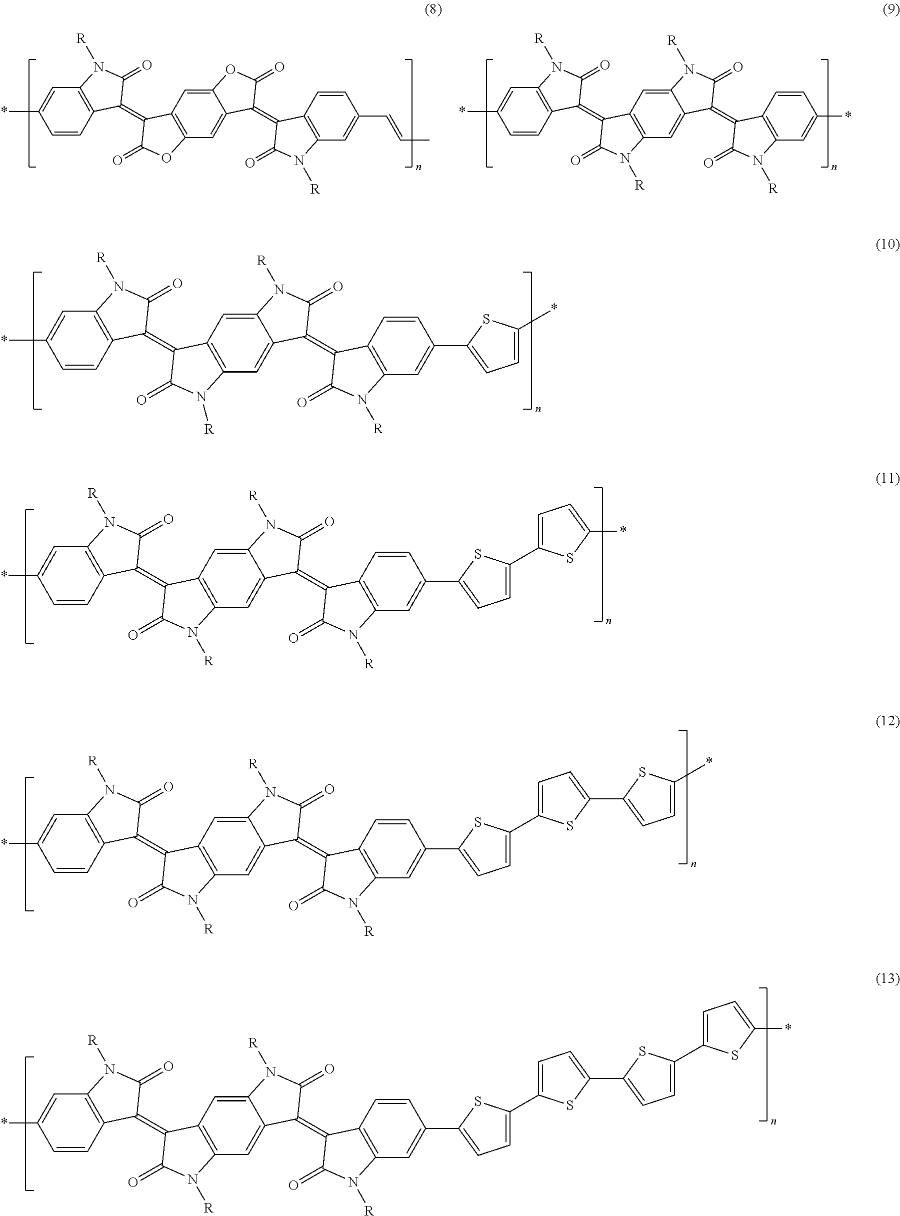

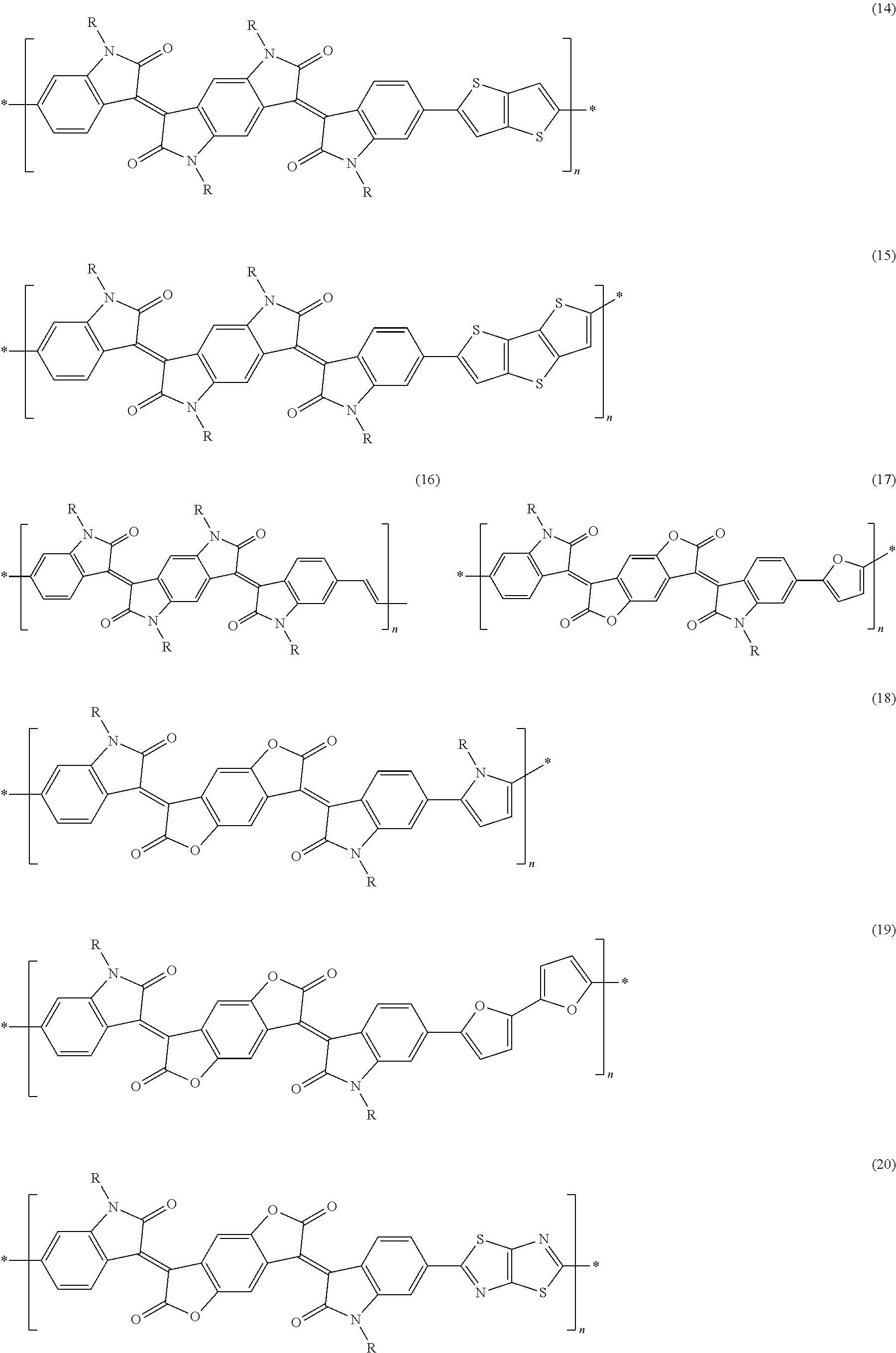

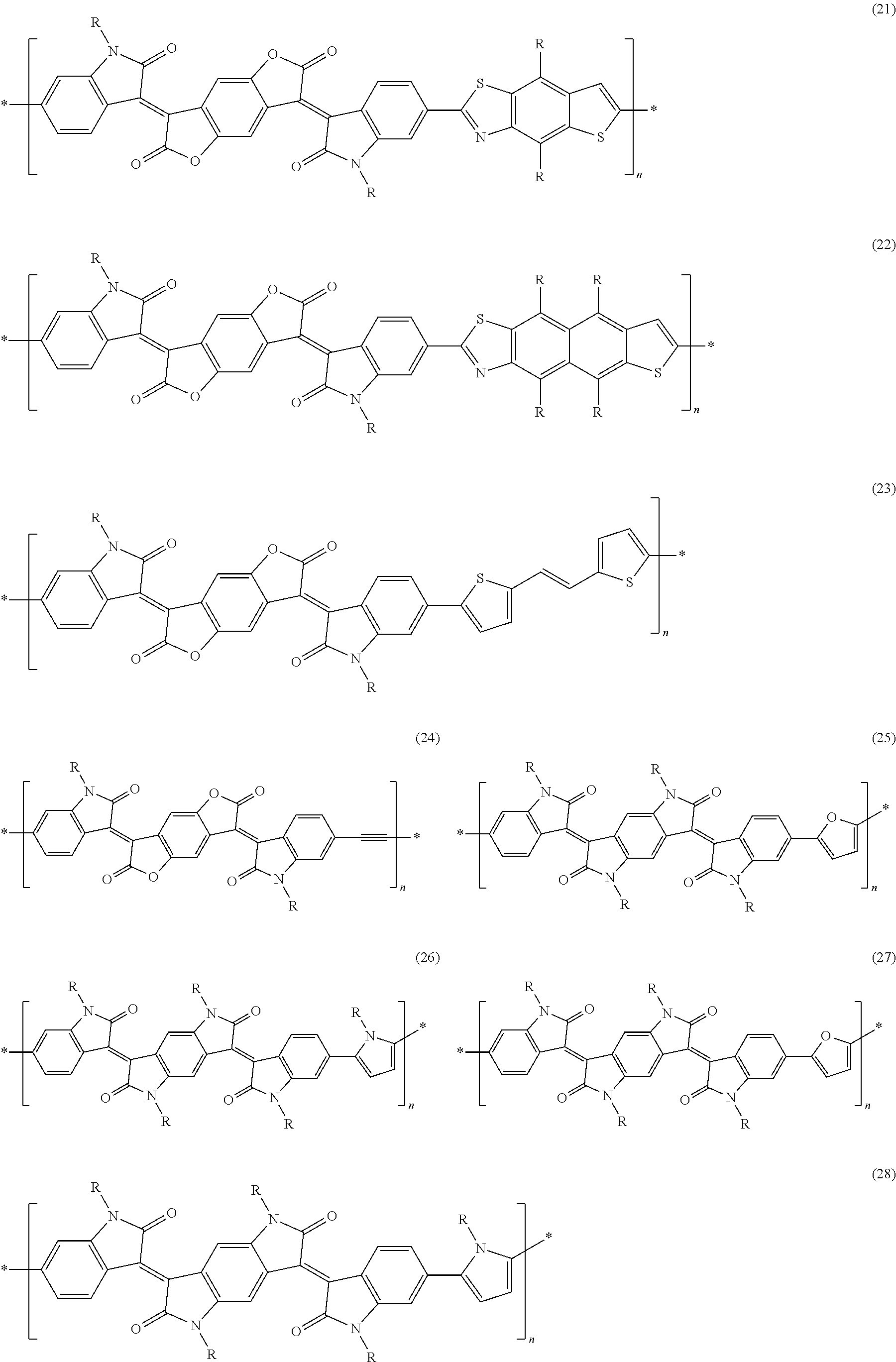

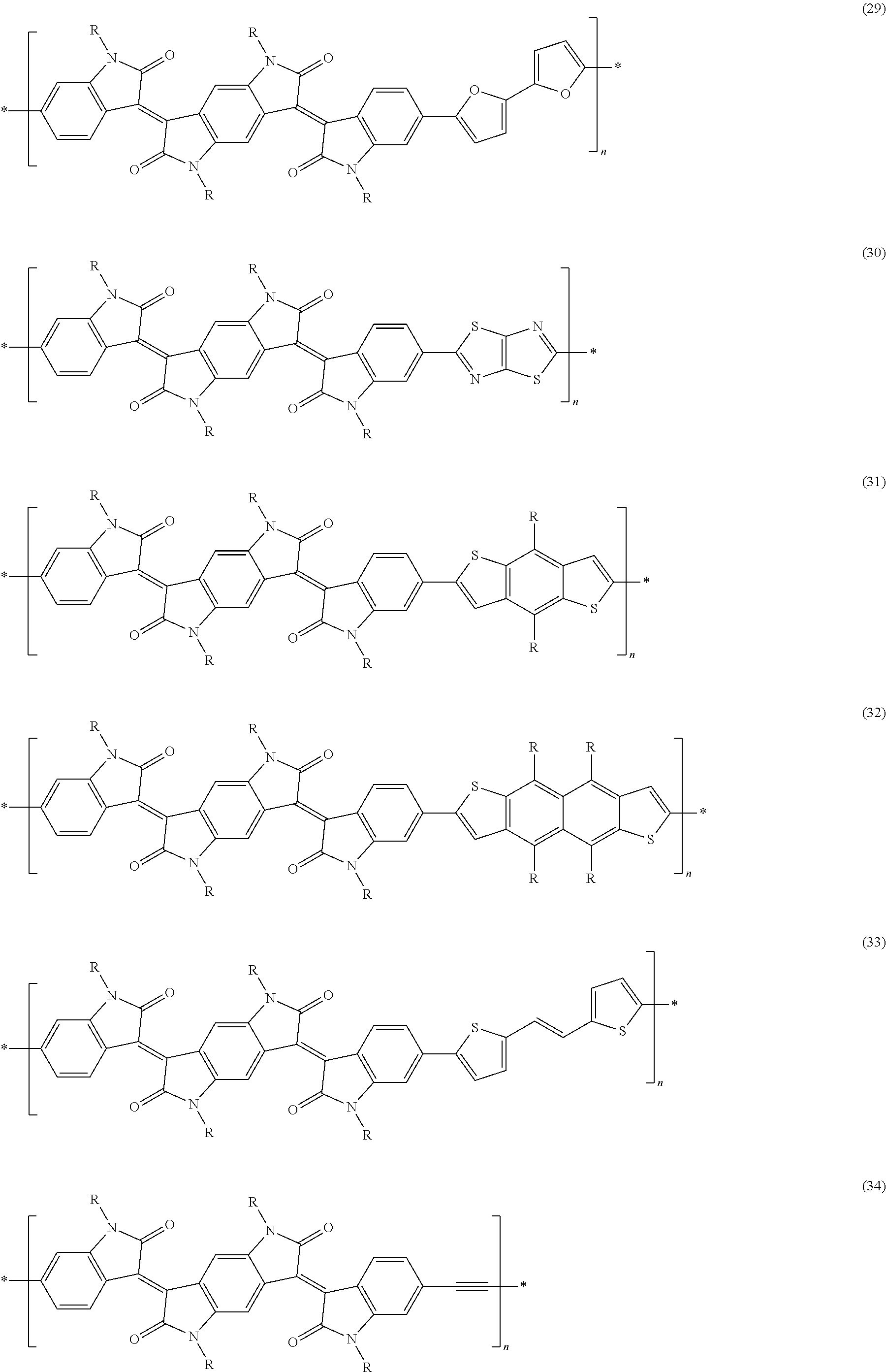

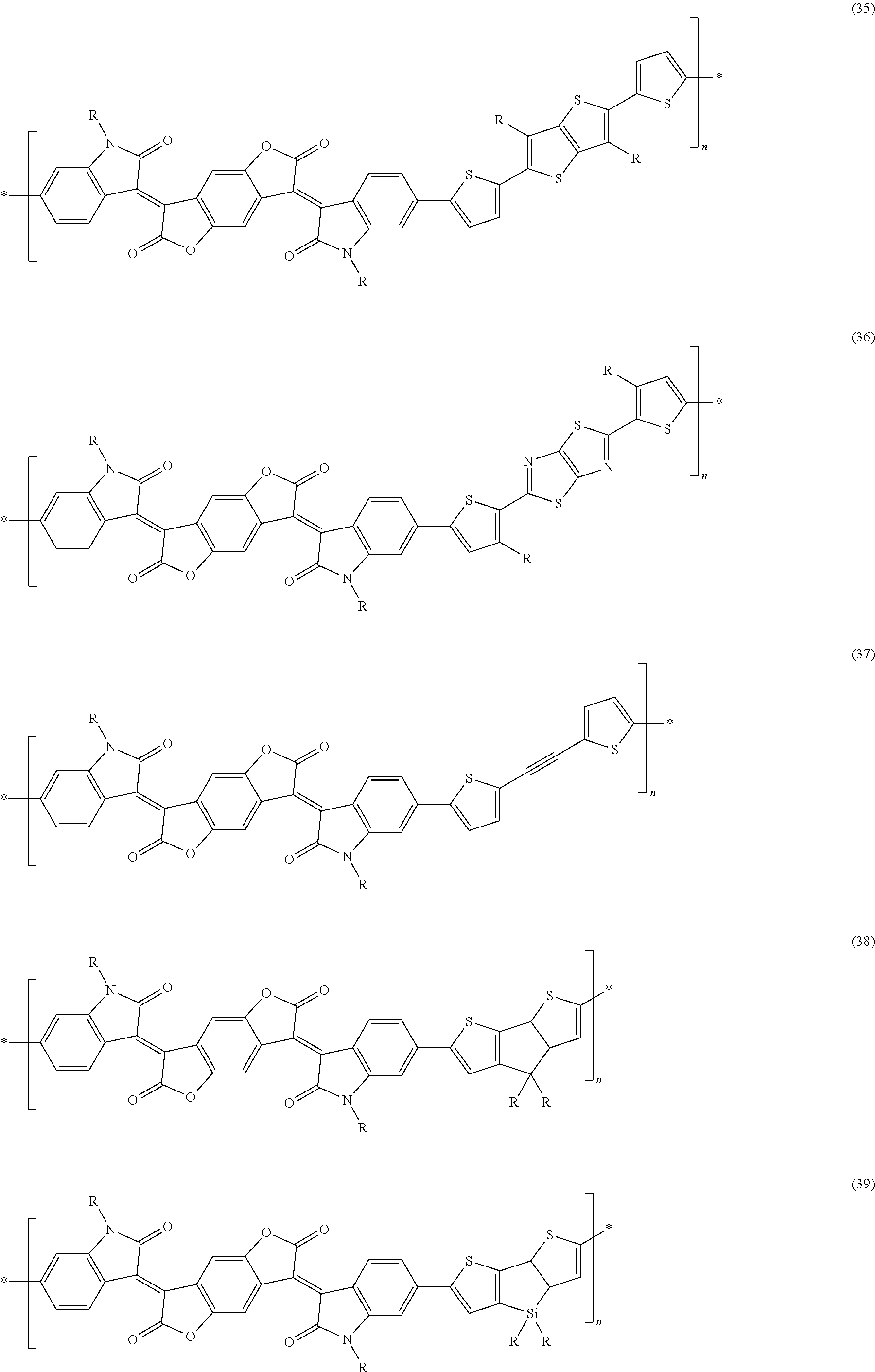

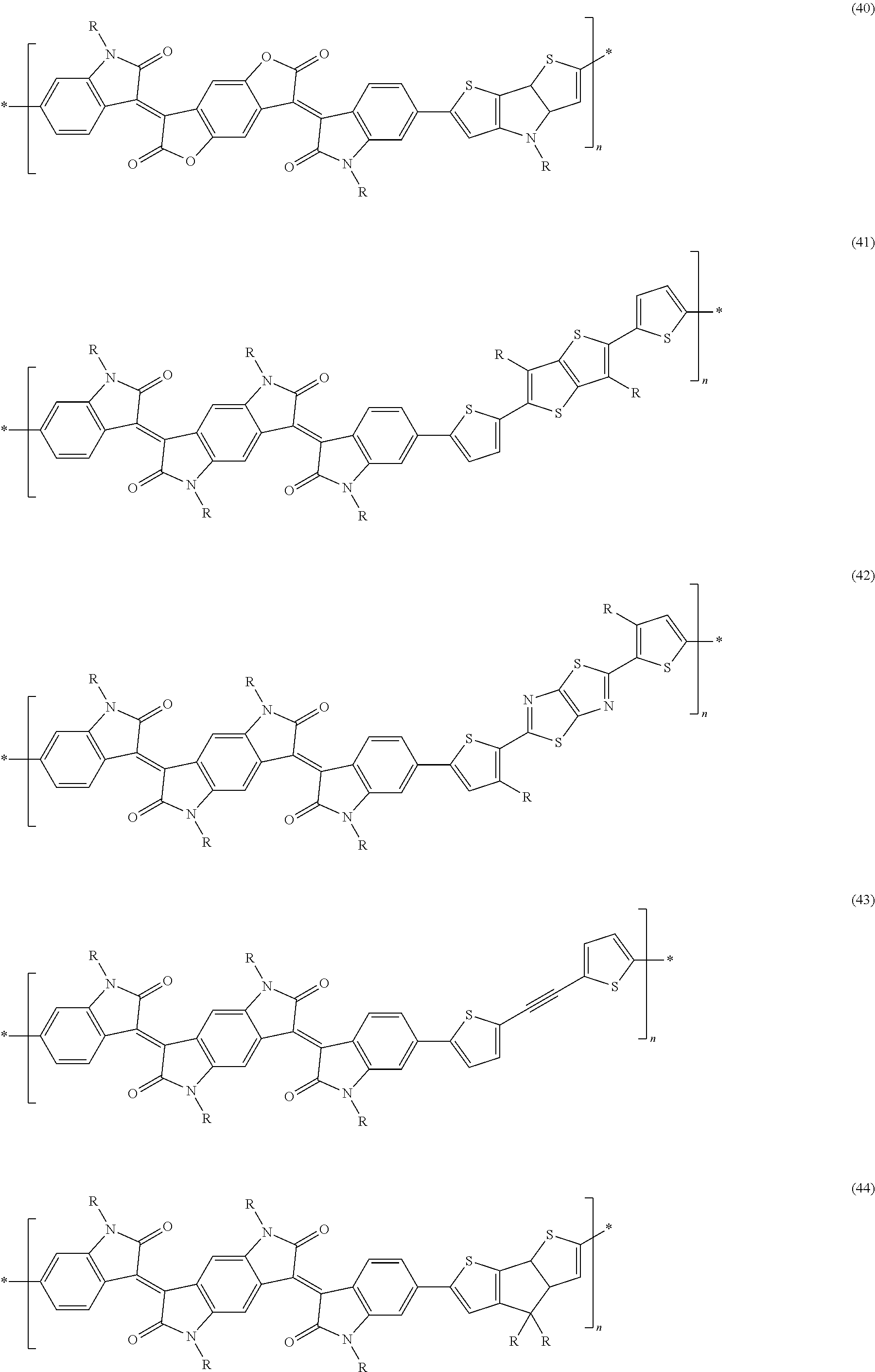

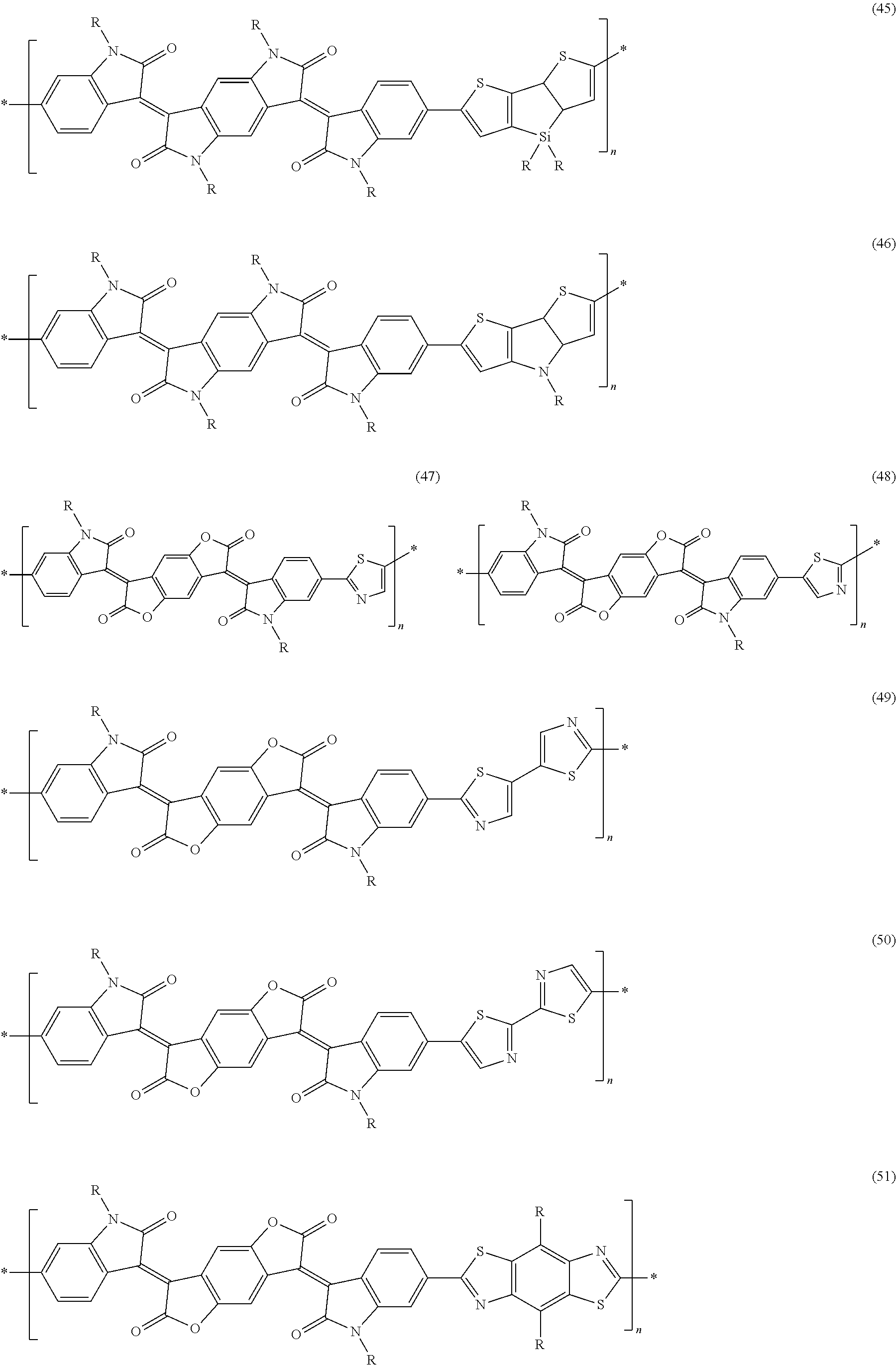

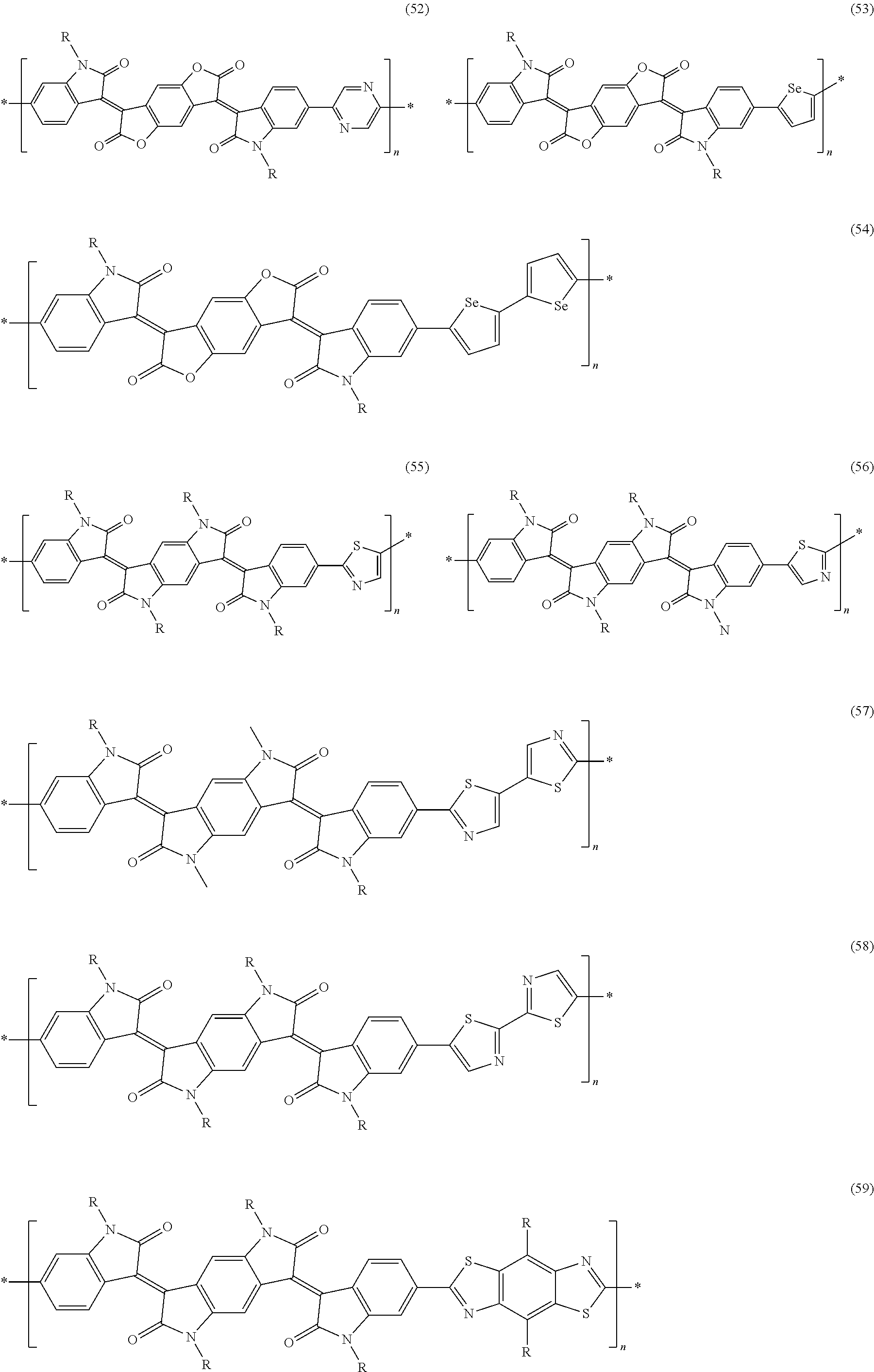

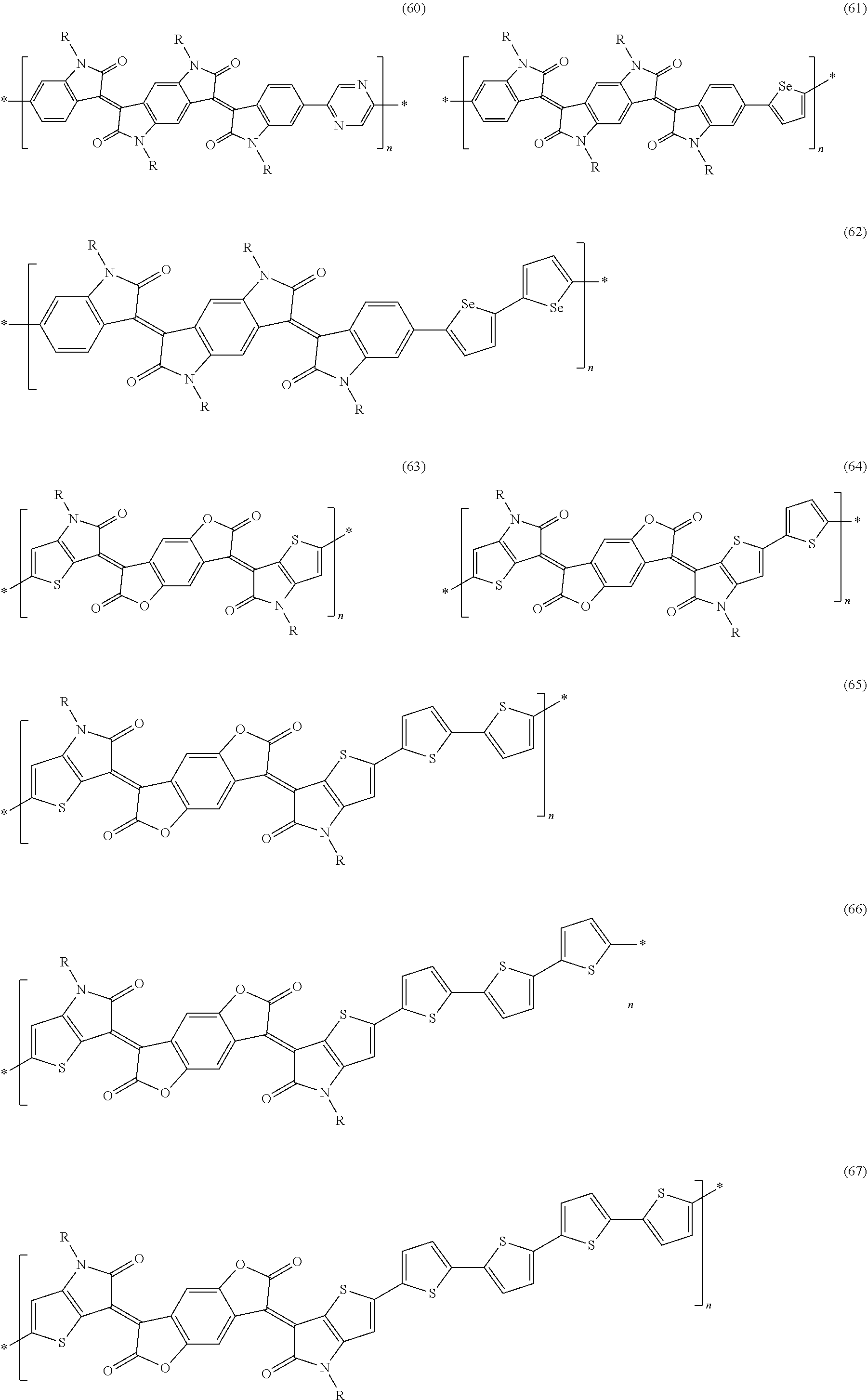

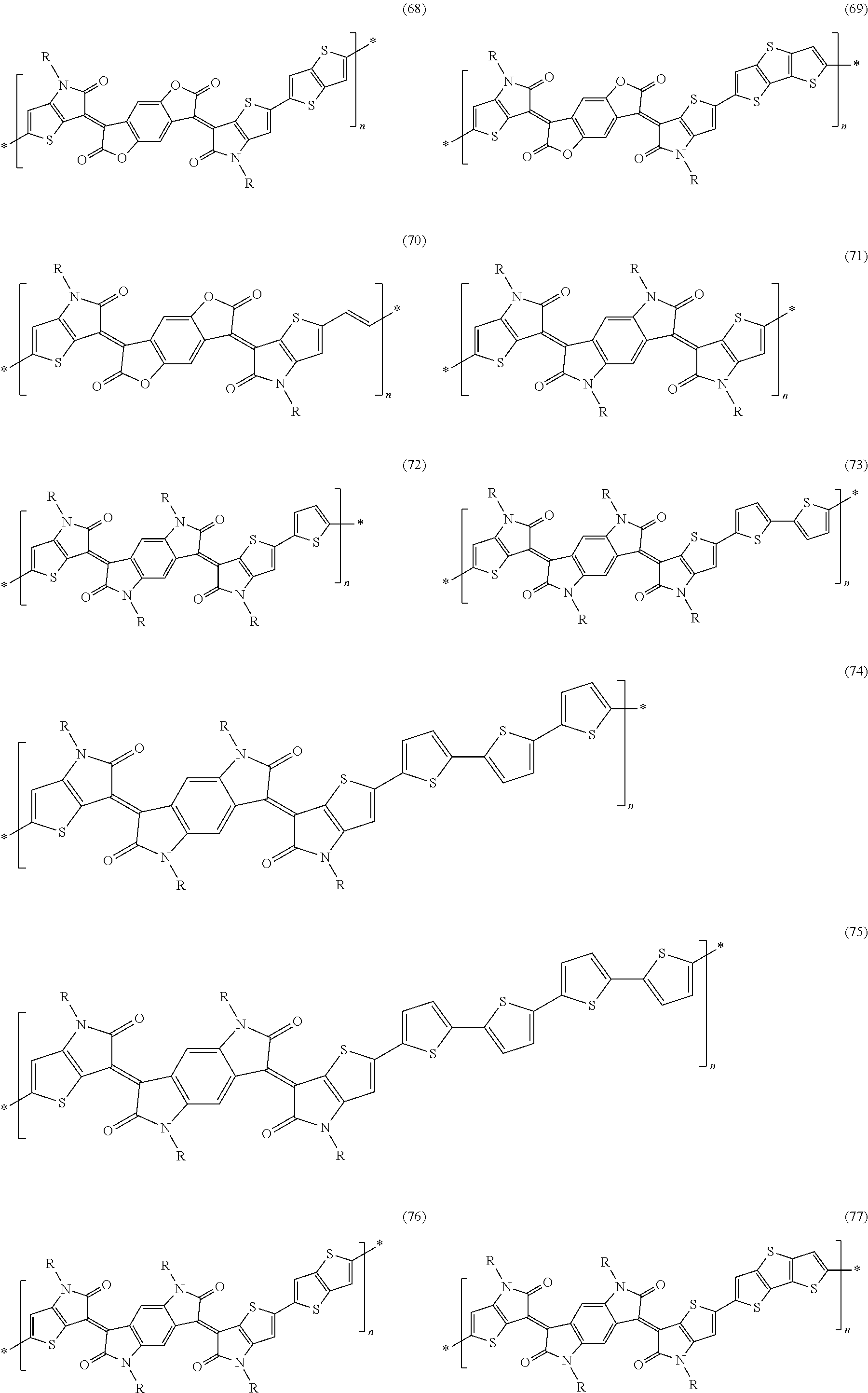

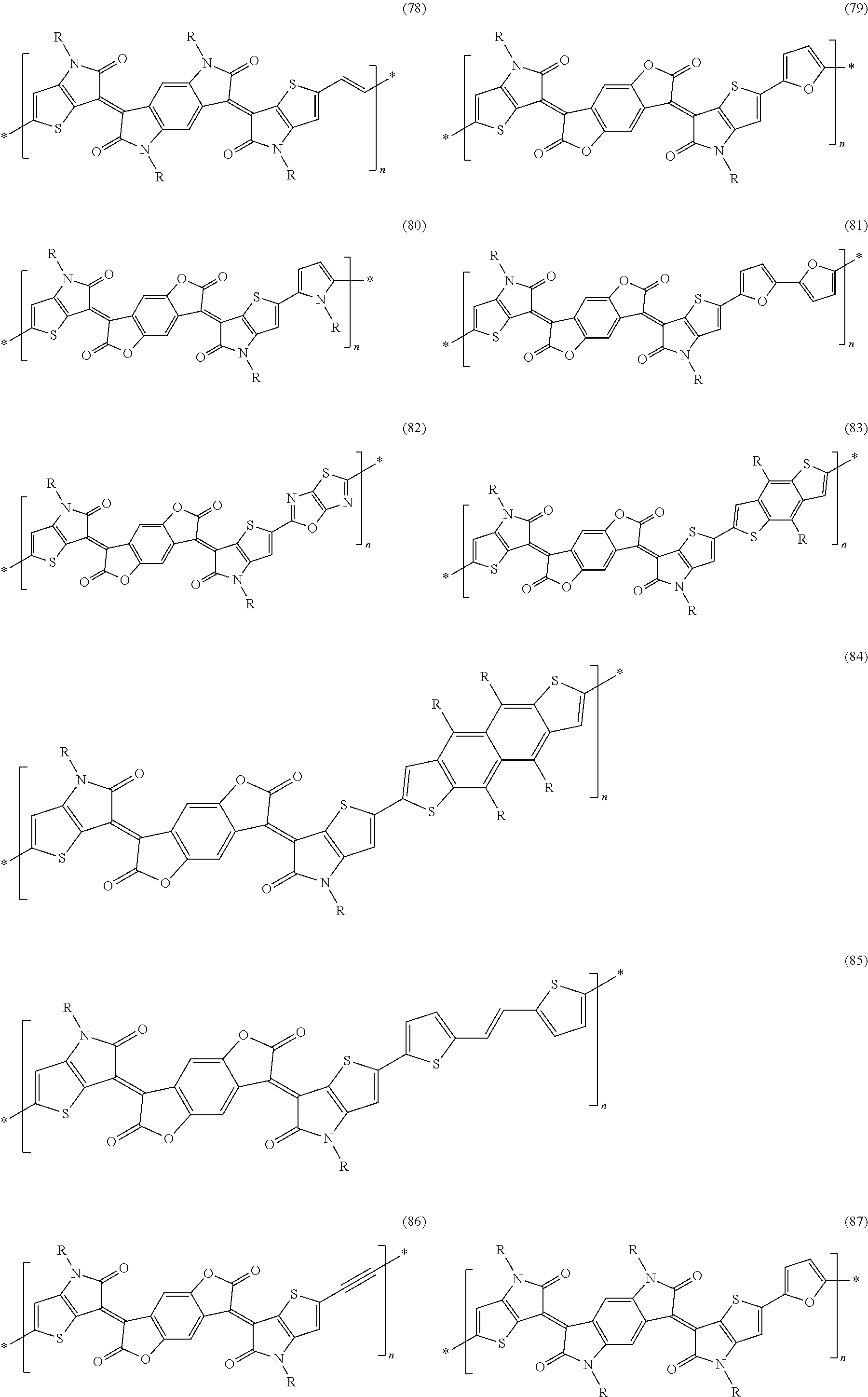

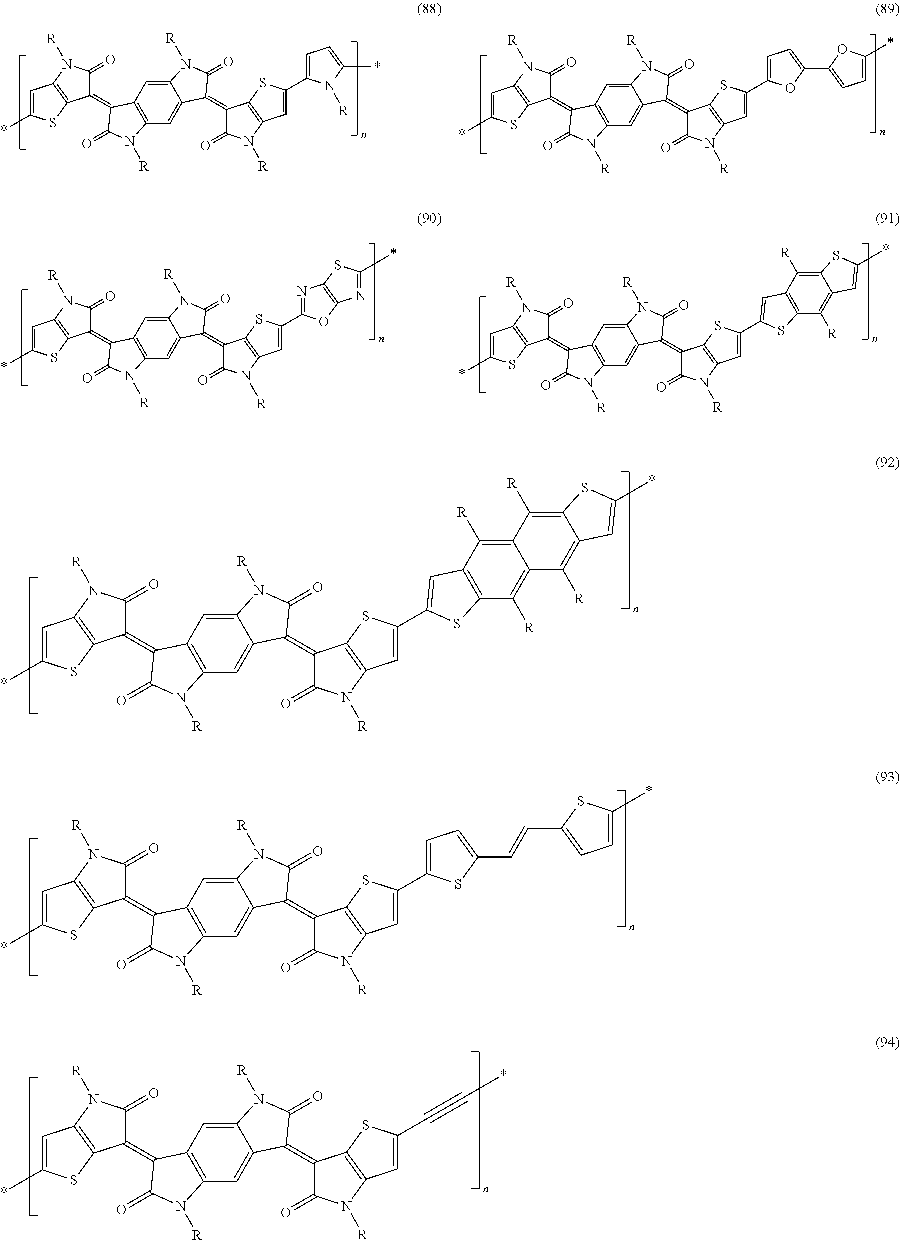

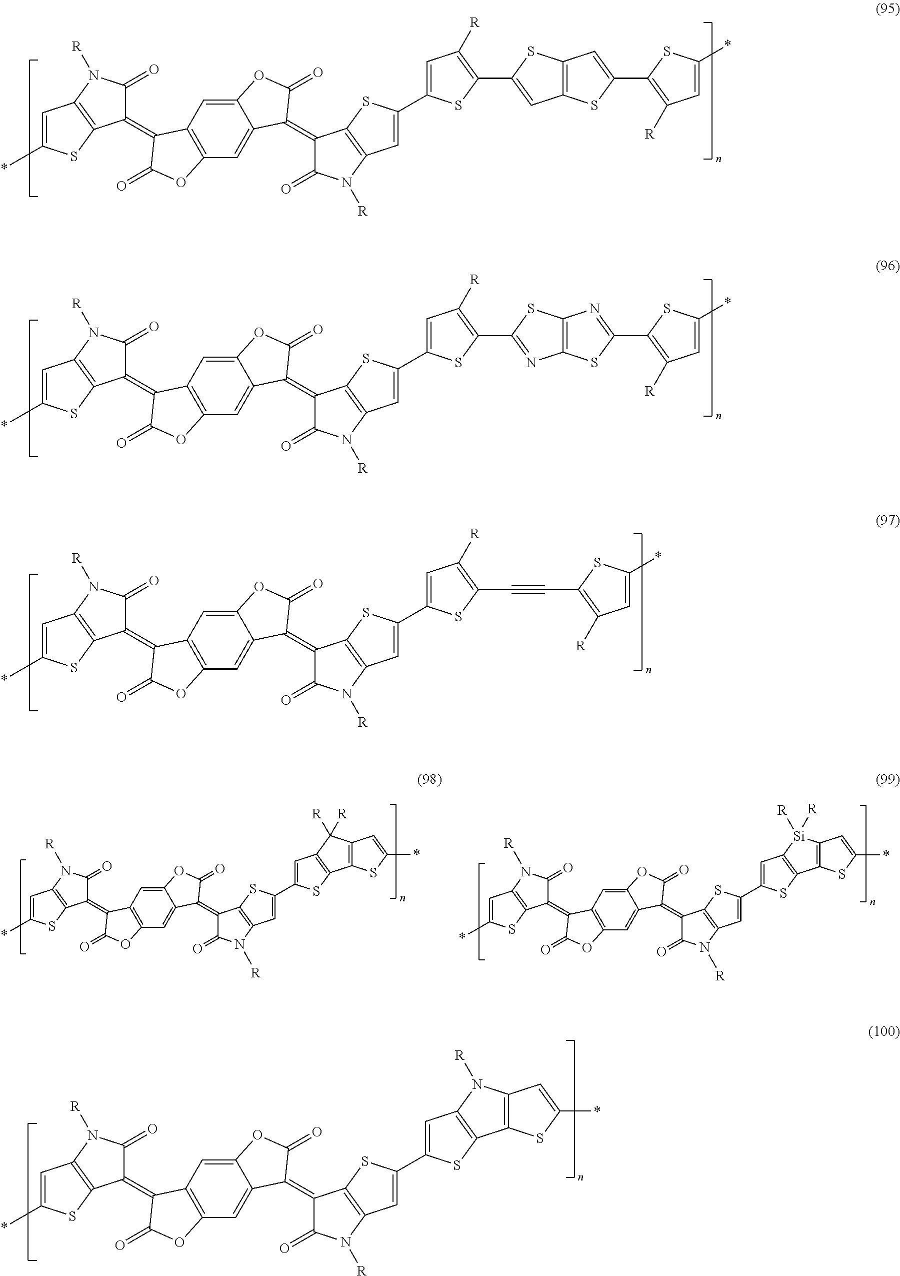

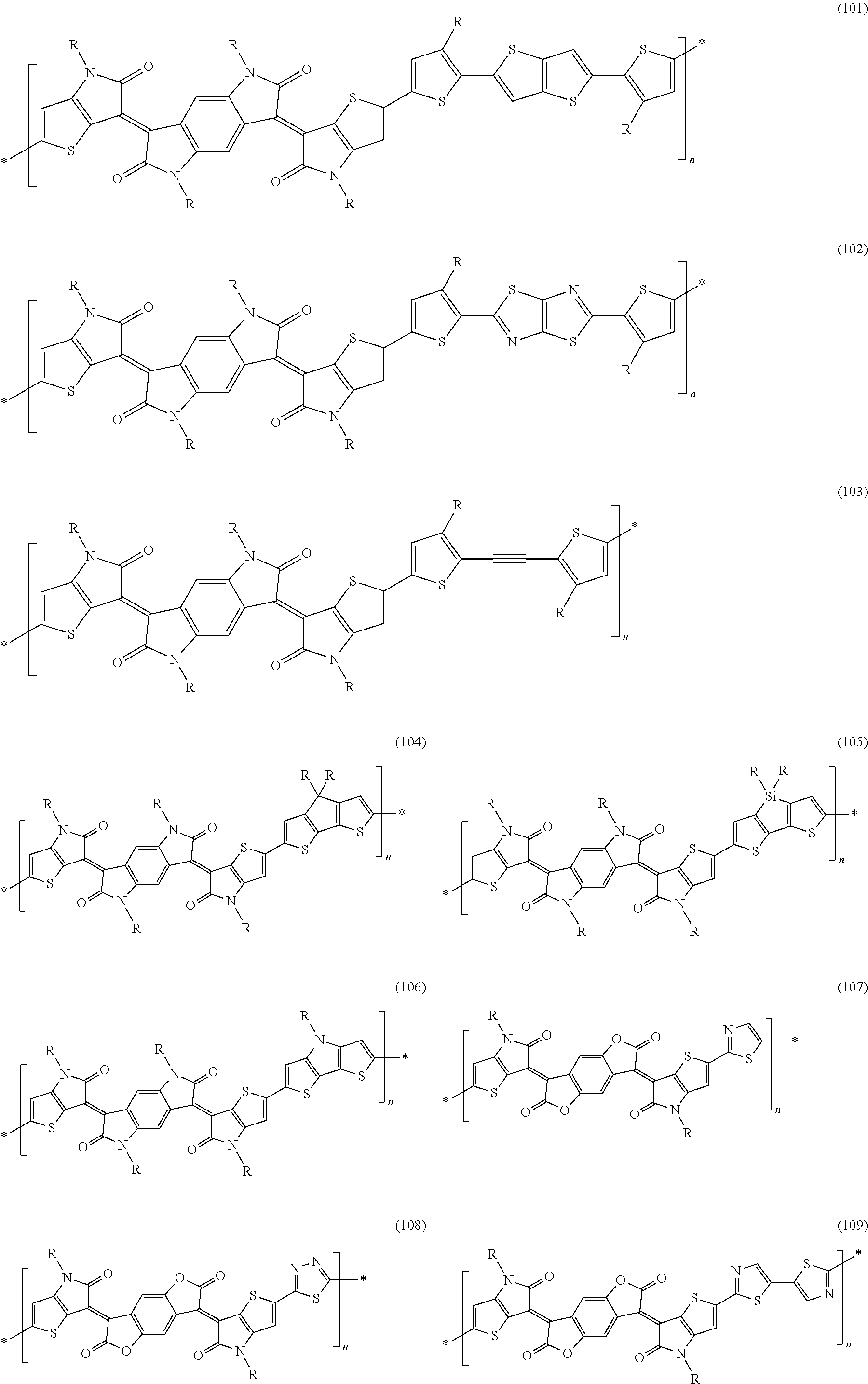

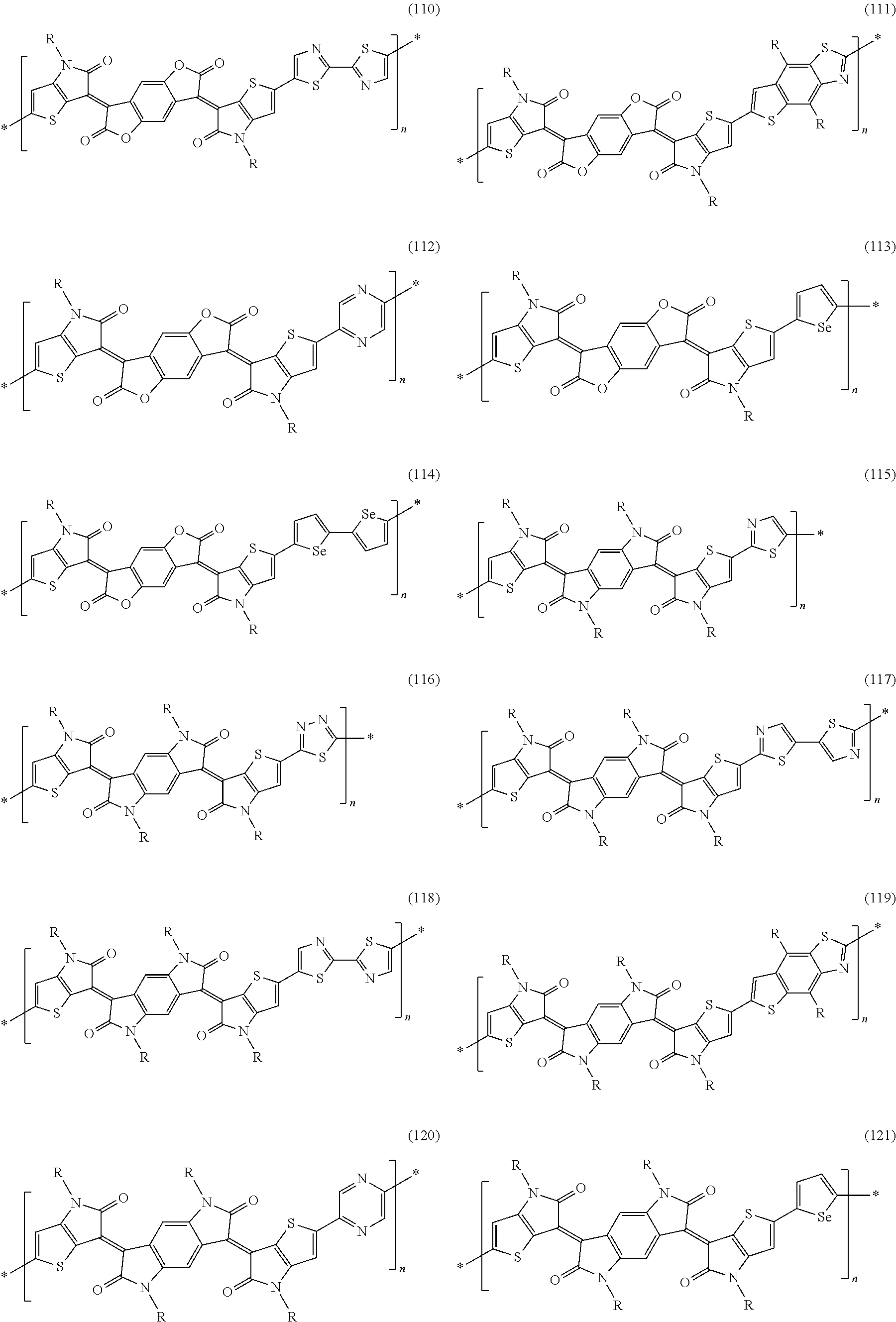

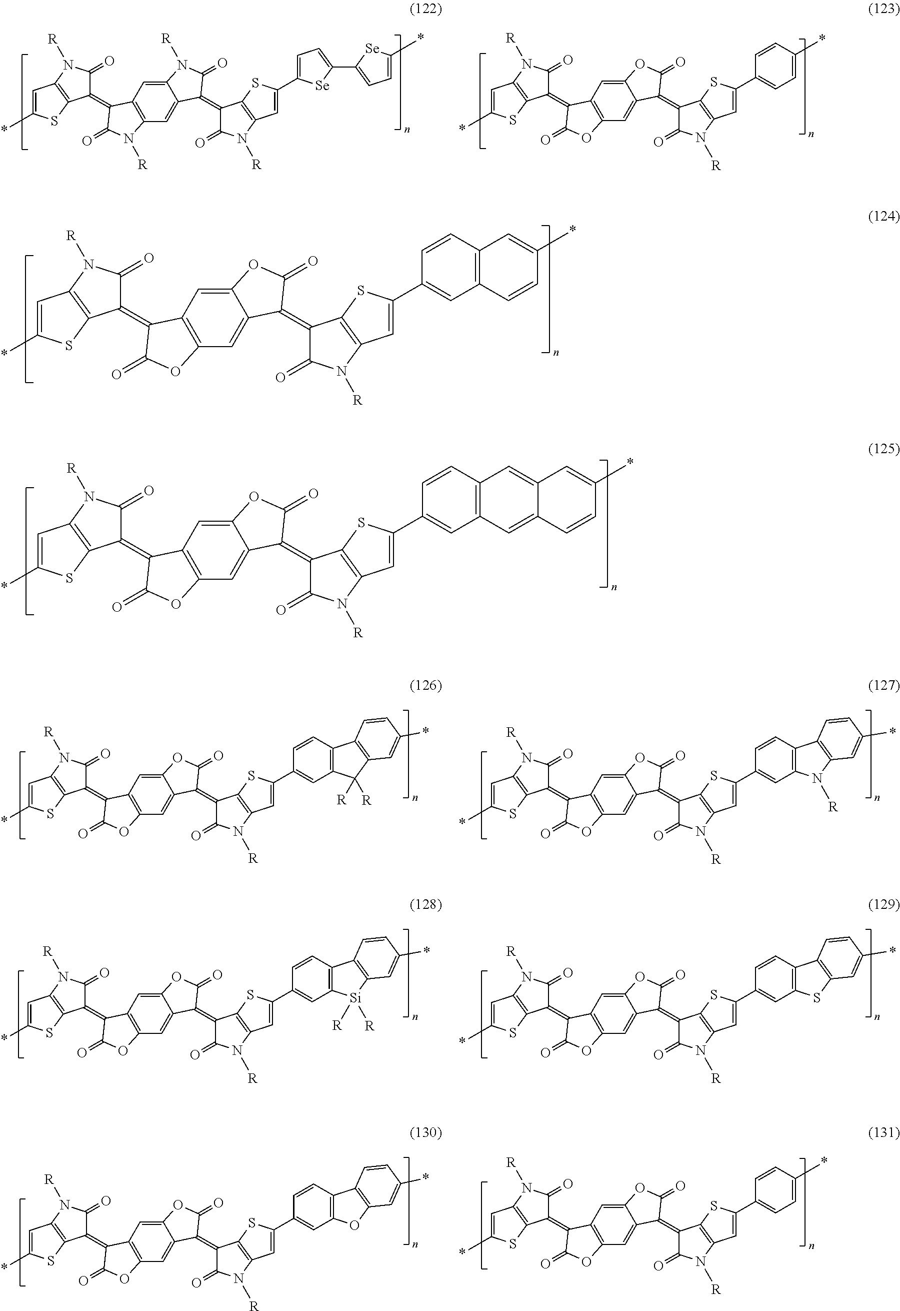

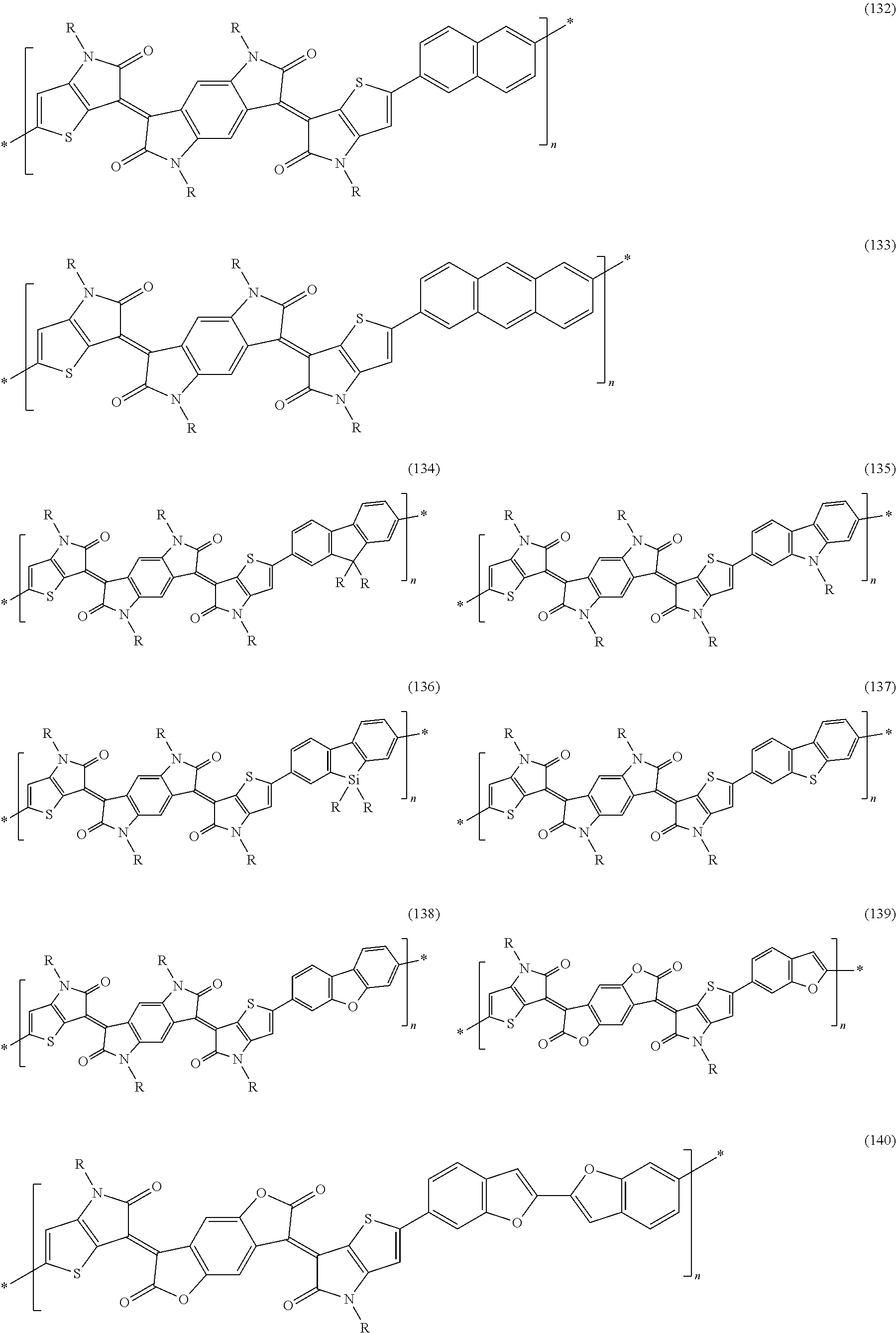

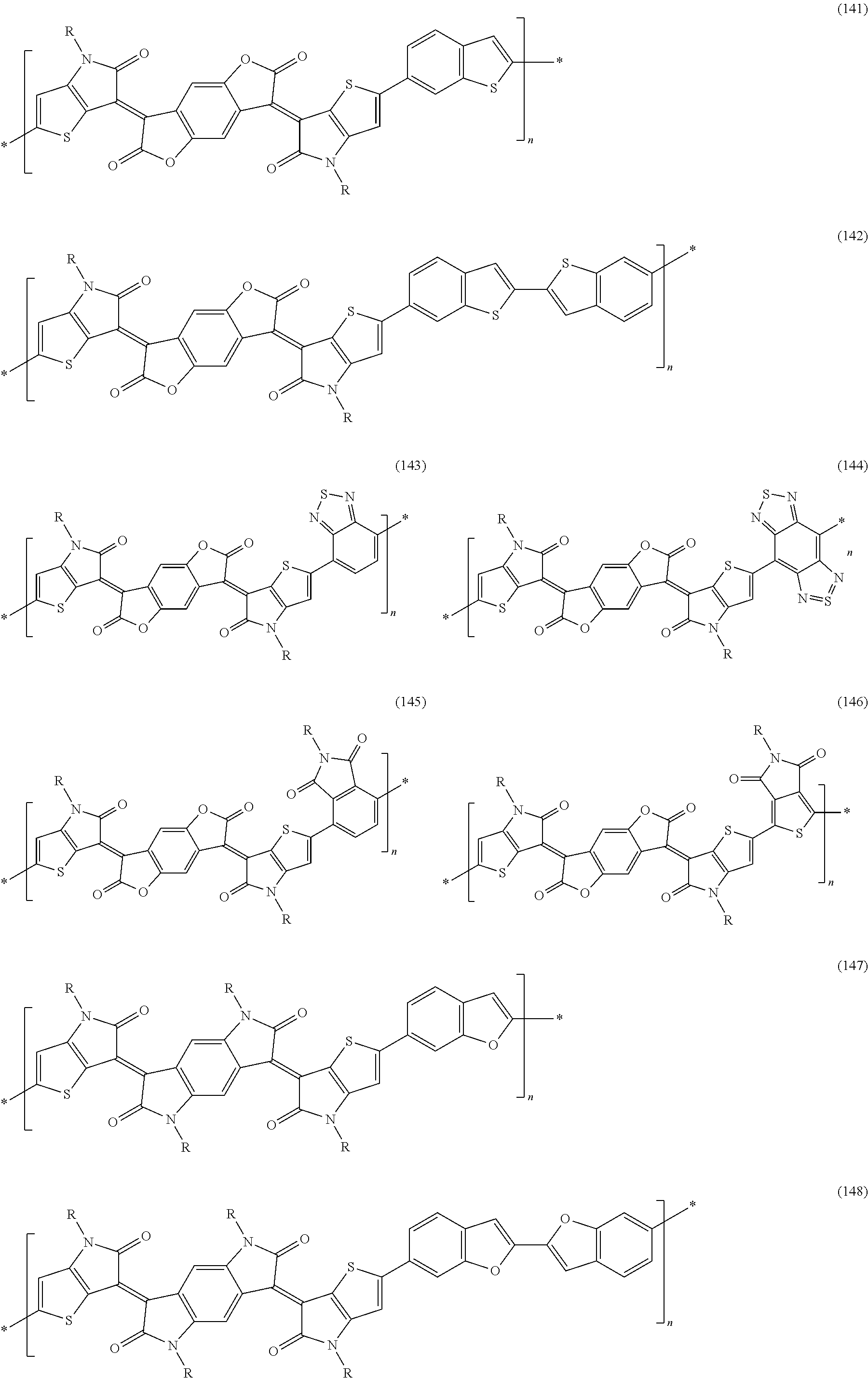

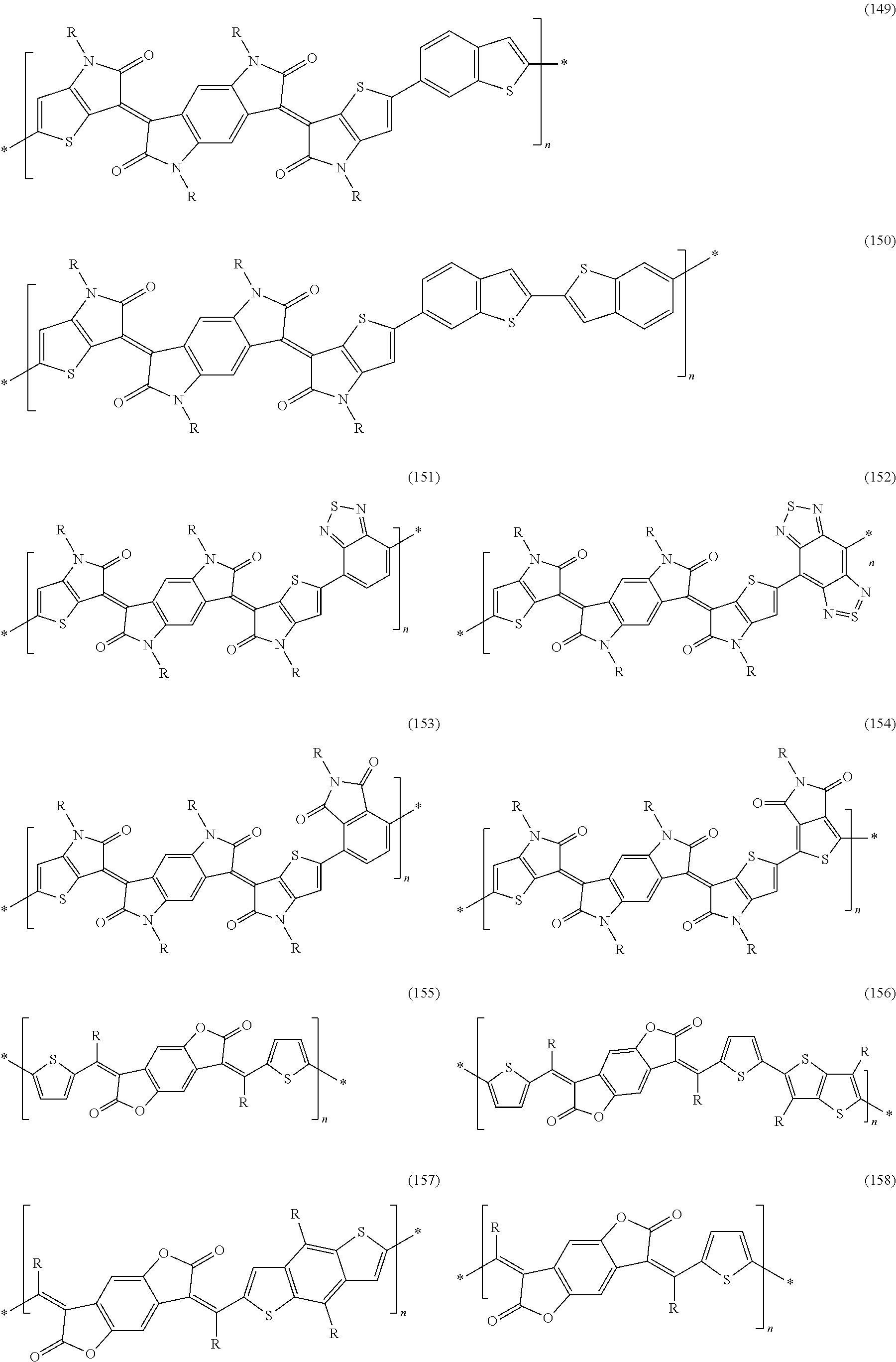

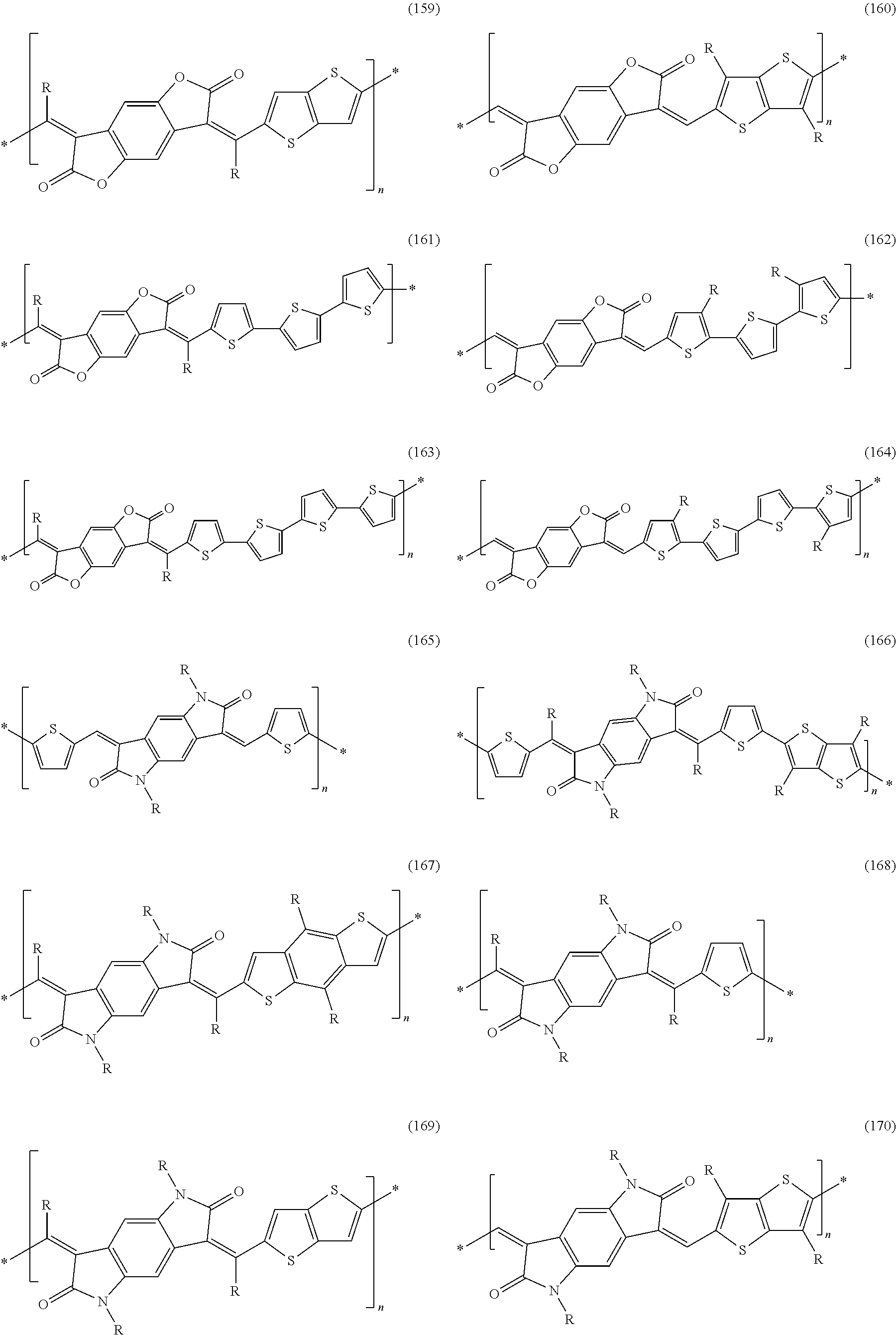

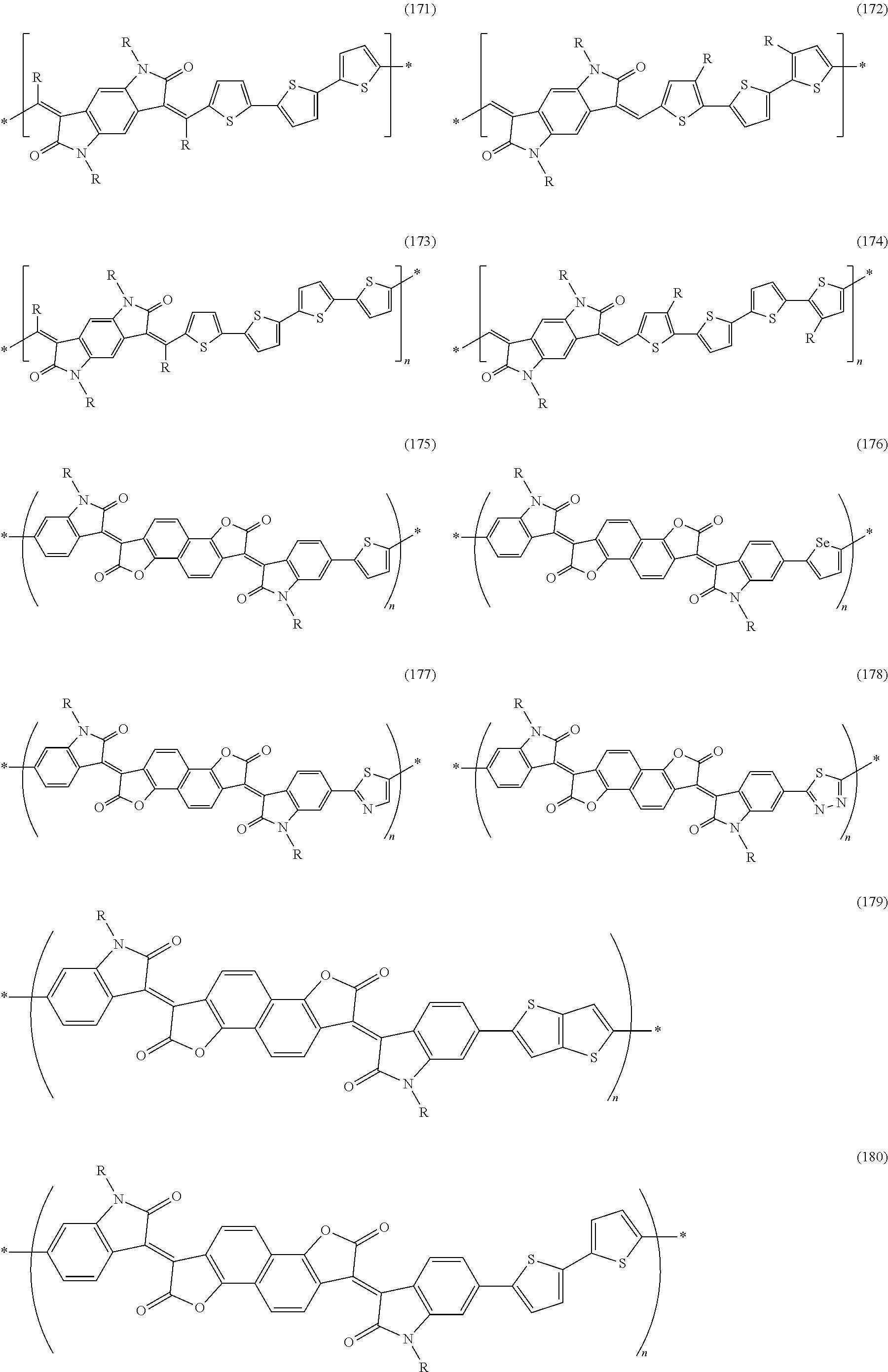

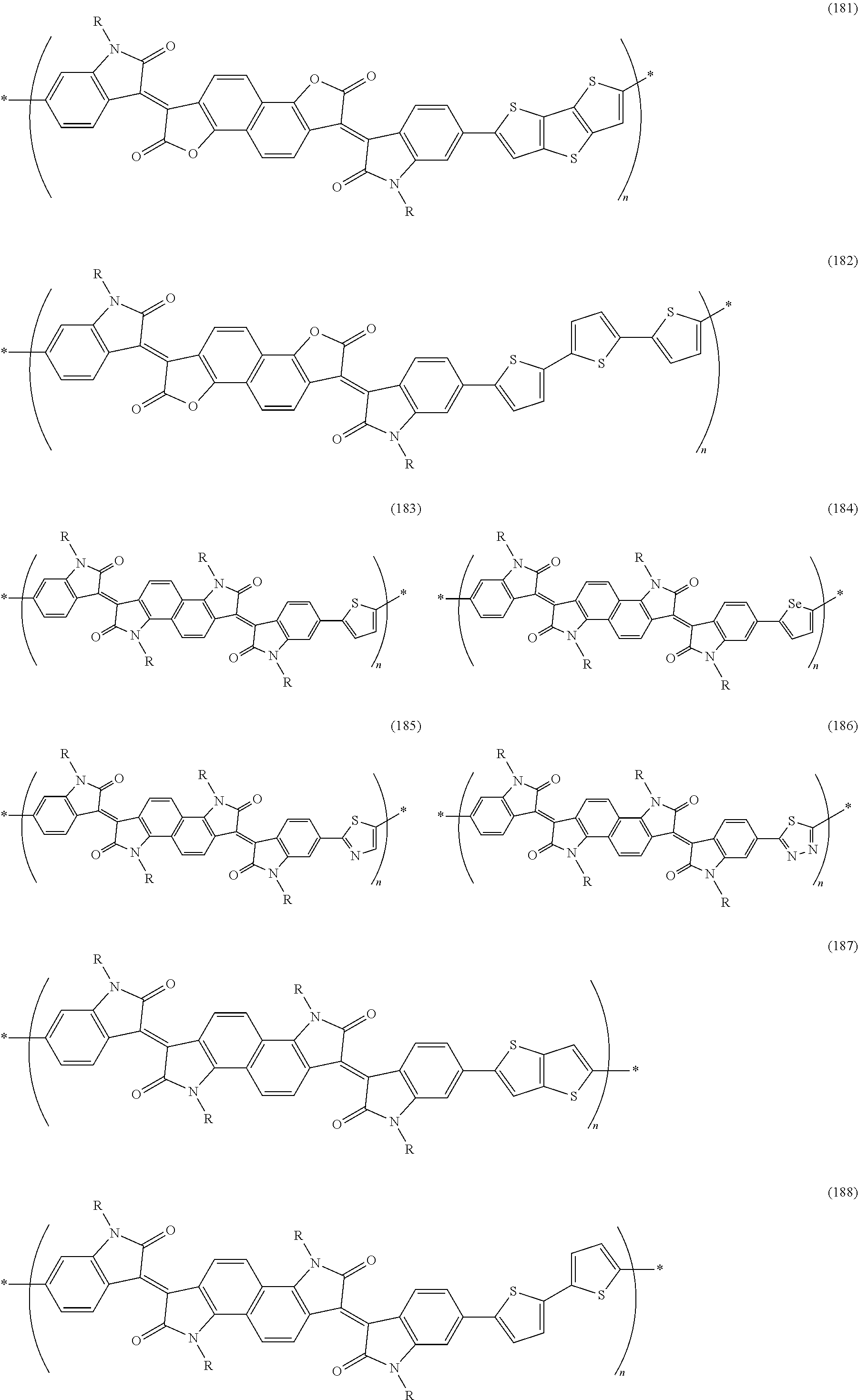

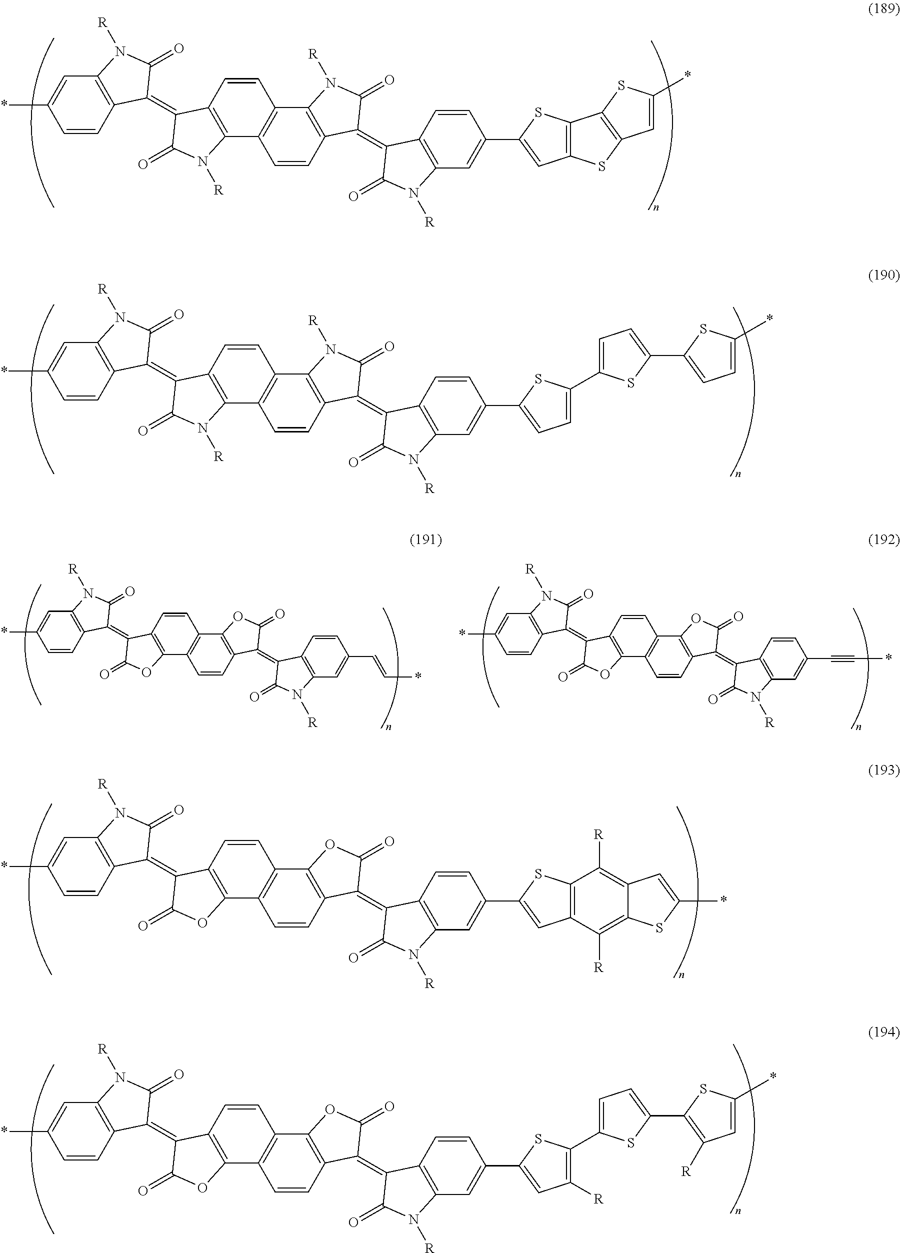

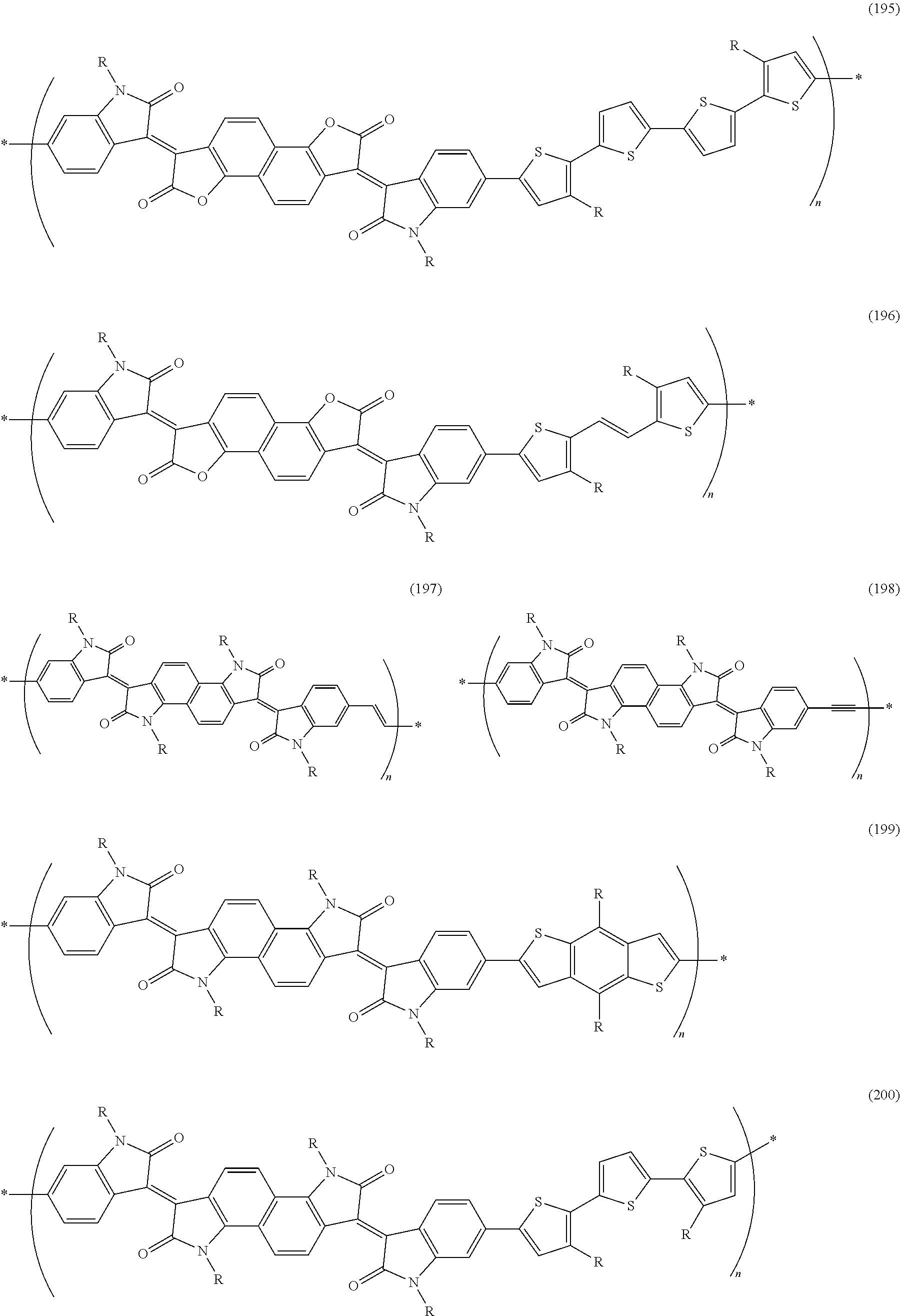

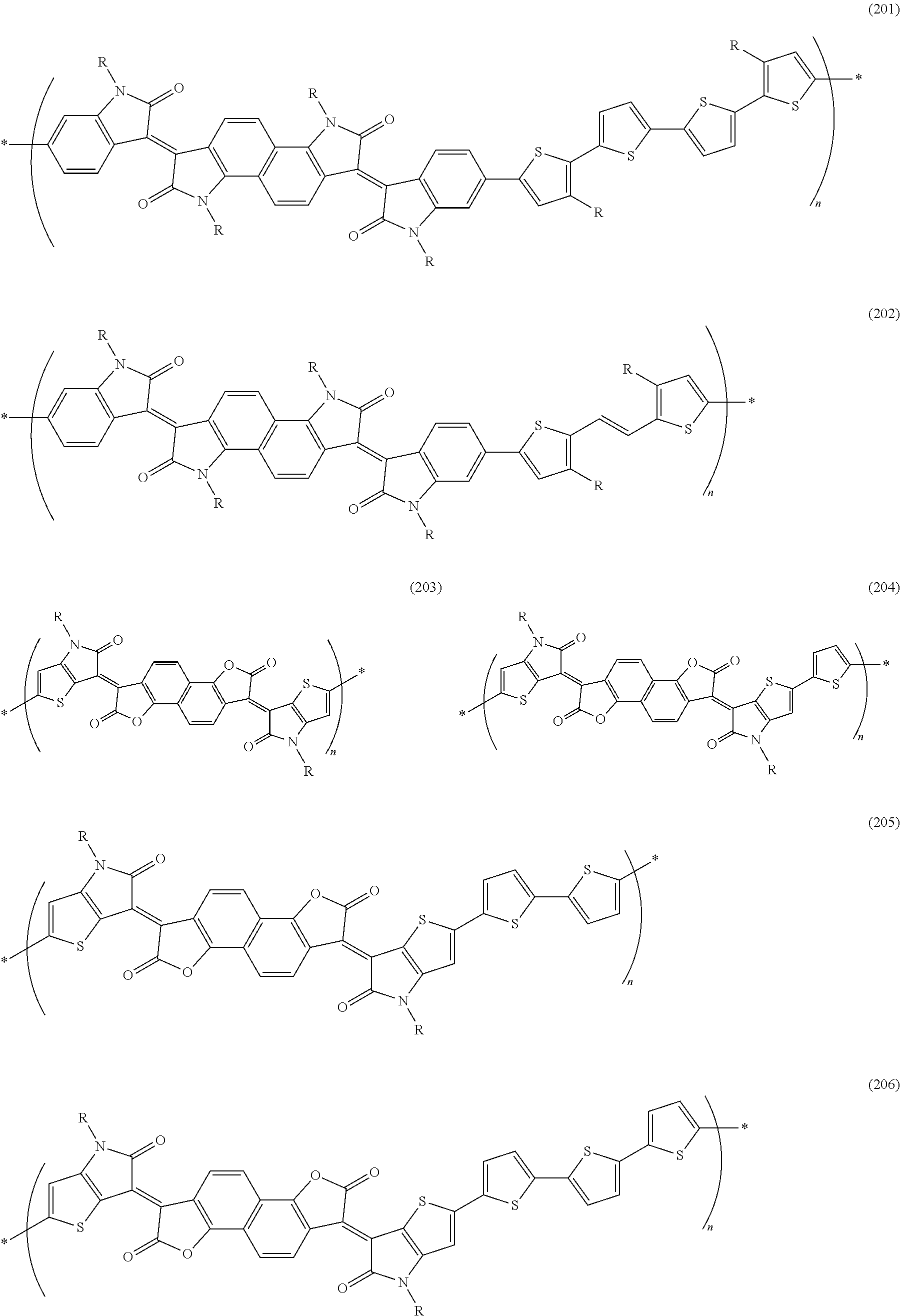

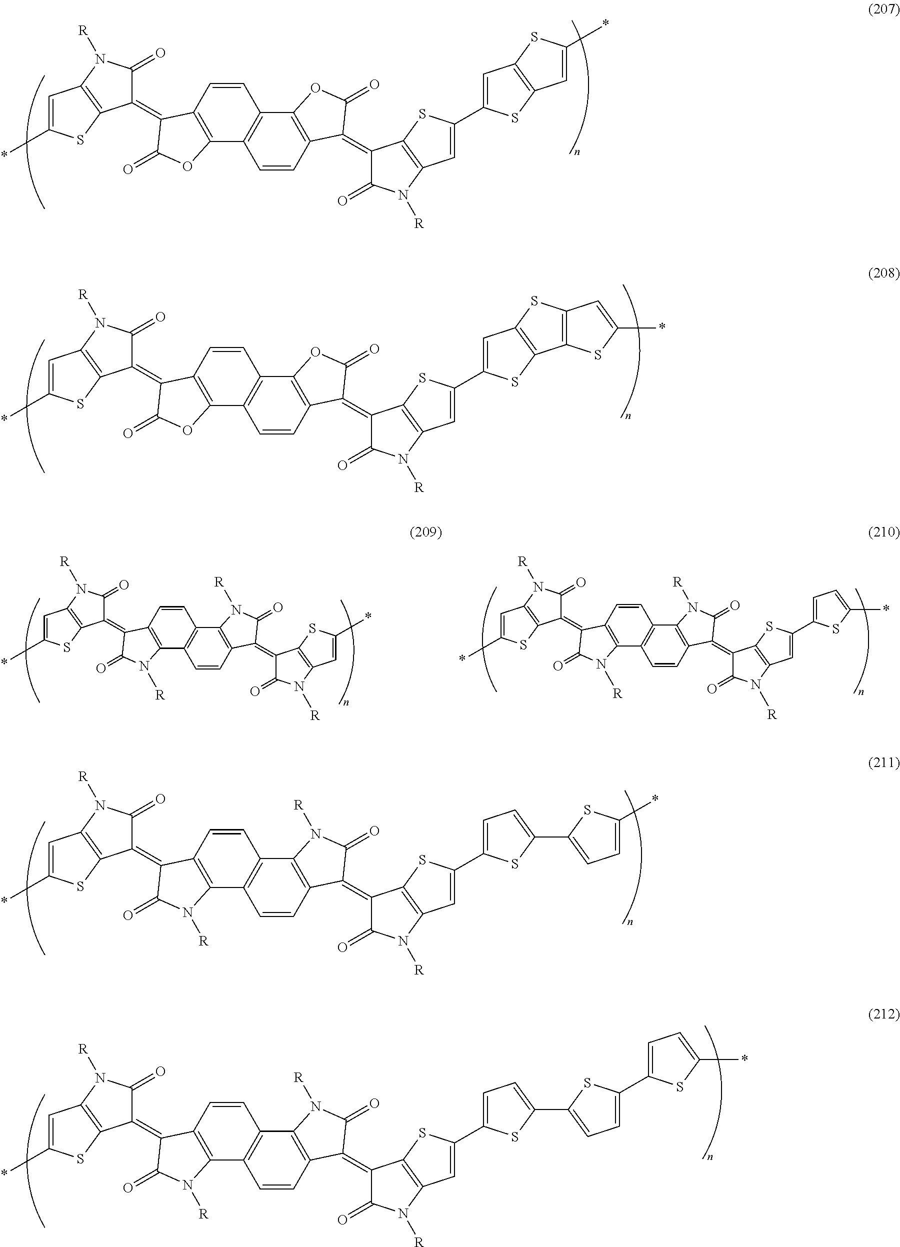

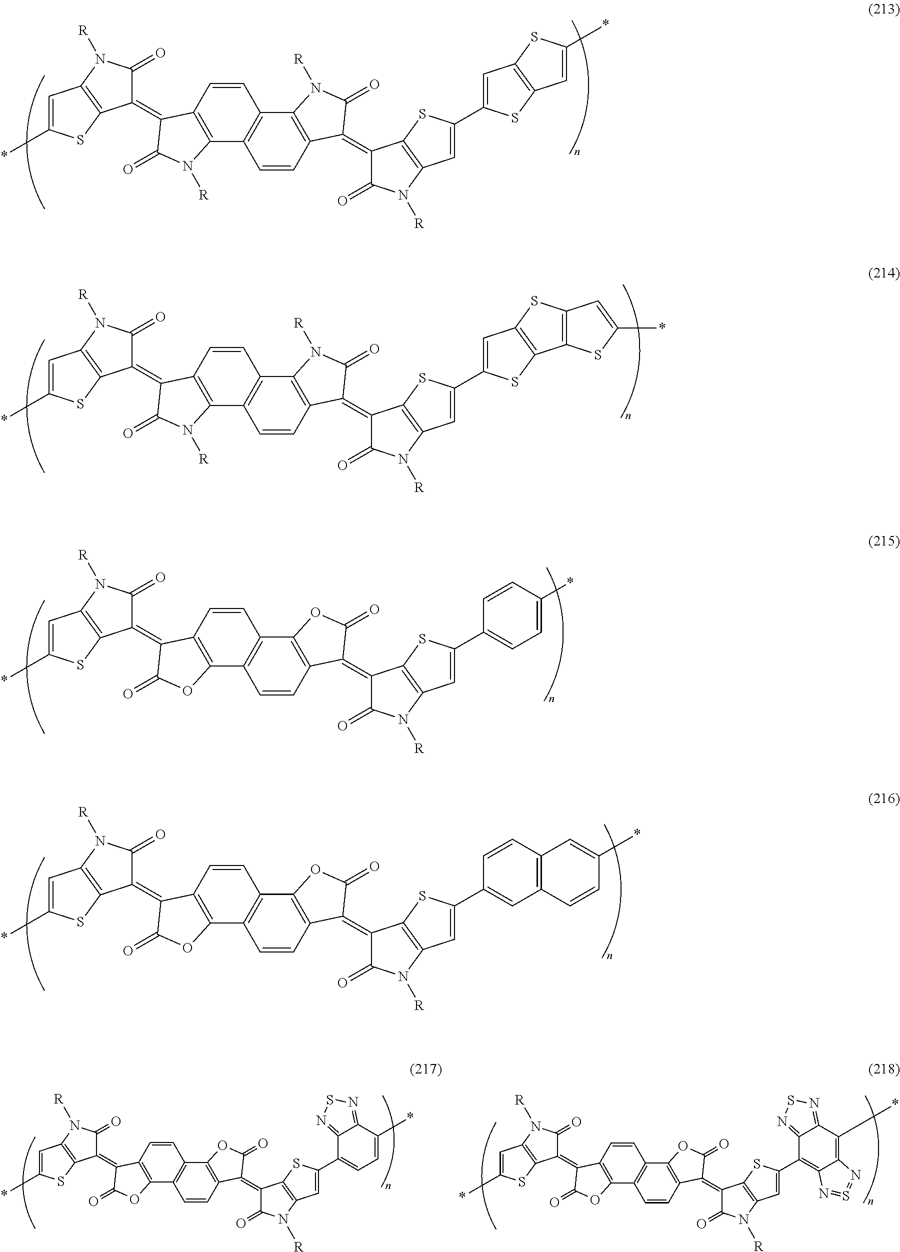

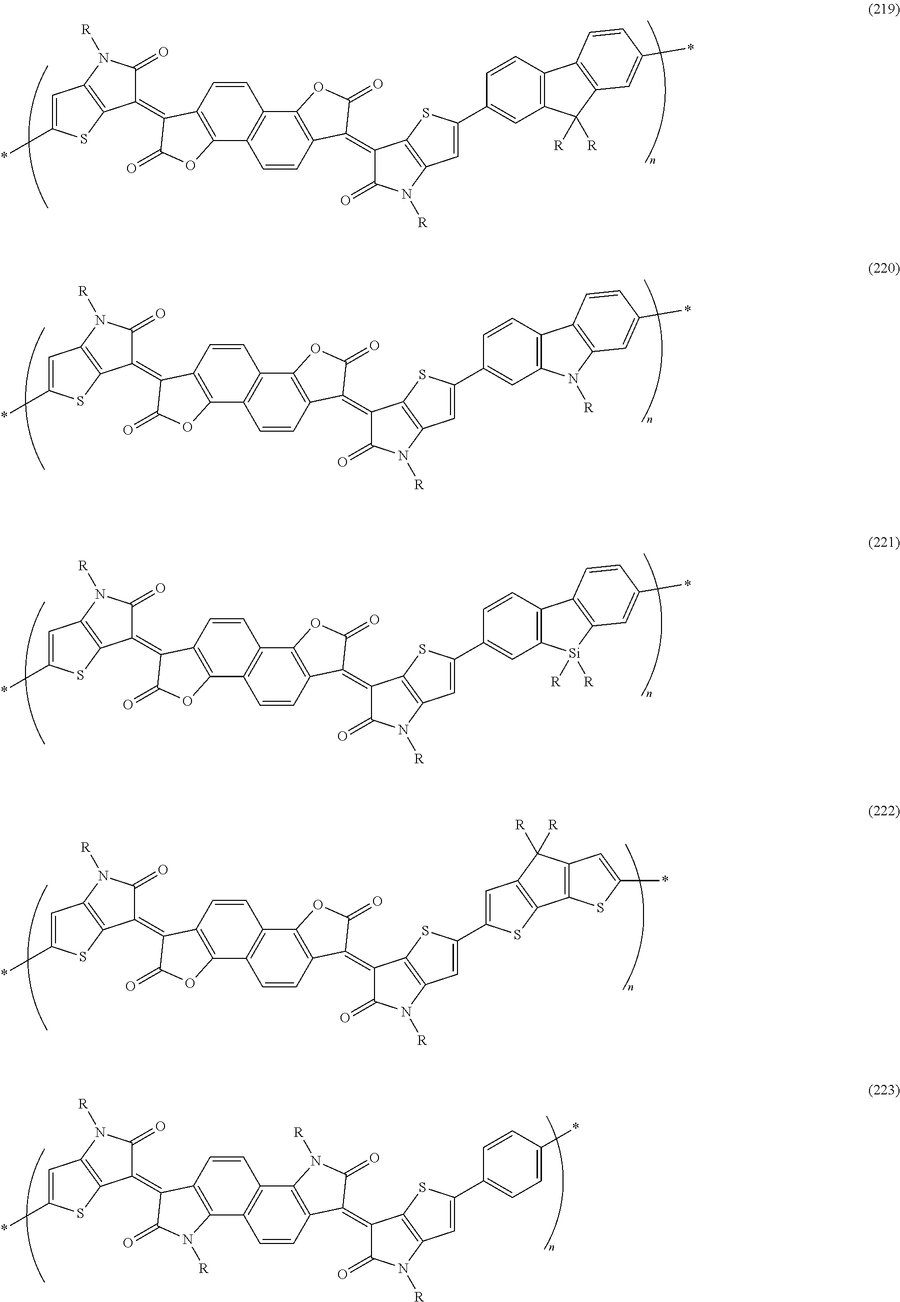

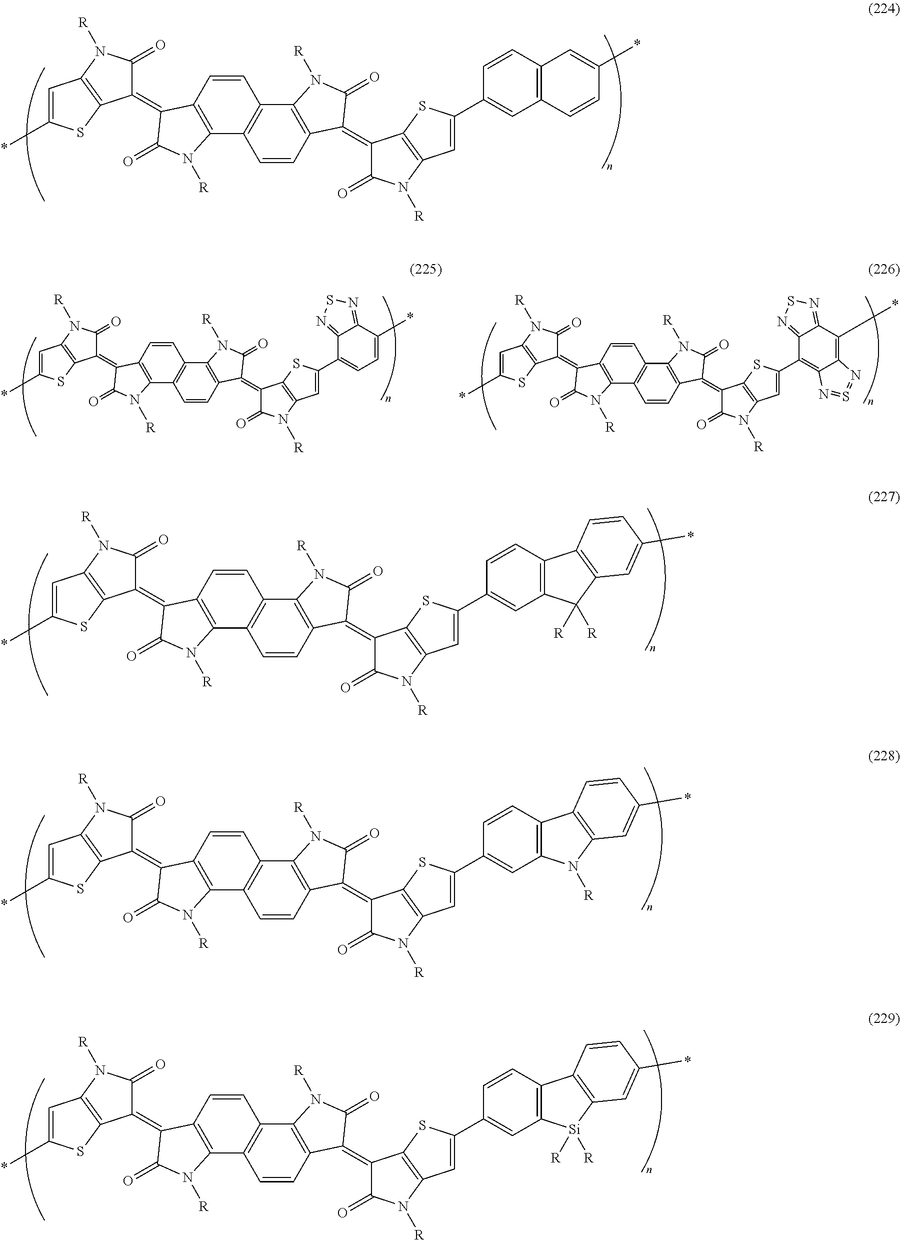

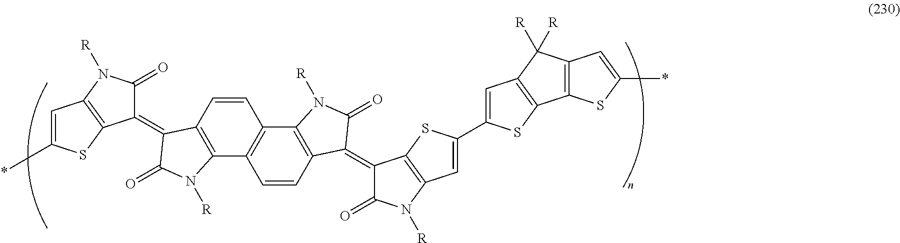

wherein:

R is independently selected from hydrogen, optionally substituted hydrocarbon with 1 to 60, in a first preferred aspect with 1 to 40 and in a second preferred aspect with 41 (or 45) to 60 carbon atoms (such as, for example, alkyl, substituted alkyl, alkenyl, substituted alkenyl, alkynyl, substituted alkynyl, aryl, and substituted aryl), or any other suitable group;

each structure can be substituted, where is applicable, with one or more suitable groups independently selected from optionally substituted hydrocarbon with 1 to 60, in a first preferred aspect with 1 to 40 and in a second preferred aspect with 41 (or 45) to 60 carbon atoms (such as, for example, alkyl, substituted alkyl, alkenyl, substituted alkenyl, alkynyl, substituted alkynyl, aryl, substituted aryl, alkoxy, and substituted alkoxy), cyano (CN), nitro, and halogen, or any other suitable group;

n is a number from 1 to 1,000,000;

the terminal “*” can be hydrogen or any other suitable group or moiety.

Other groups or moieties suitable as “*” may be as defined for R^{a} or R^{c} below.

Compounds comprising said fused-ring moiety as defined above may preferably be selected from the group consisting of small molecules, monomers and polymers. As used herein, the term “small molecule” will be used to denote a compound comprising said fused-ring moiety and two inert chemical groups, which are inert under use condition and thus inhibit such a small molecule from being polymerized. In contrast hereto, the term “monomer” is used to denote a compound comprising said fused-ring moiety and at least one reactive chemical group, which allows such monomer to be reacted so as to form part of a polymer.

### First Preferred Embodiment

In a first preferred embodiment the present application relates to the following aspects EA-1 to EA-8:

EA-1. A monomer, oligomer or polymer comprising a fused-ring moiety (I):

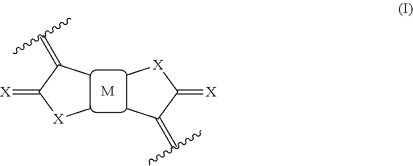

X is independently oxygen (O), sulphur (S), or NR (R is independently hydrogen or optionally substituted hydrocarbon with 1 to 40 carbon atoms such as, for example, alkyl, substituted alkyl, alkenyl, substituted alkenyl, alkynyl, substituted alkynyl, aryl, and substituted aryl, or any other suitable group);

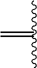

indicates the linkage with another moiety (or moieties);

M is a conjugated moiety.

EA-2. The monomer, oligomer or polymer of EA-1, wherein M is selected from a group of structures:

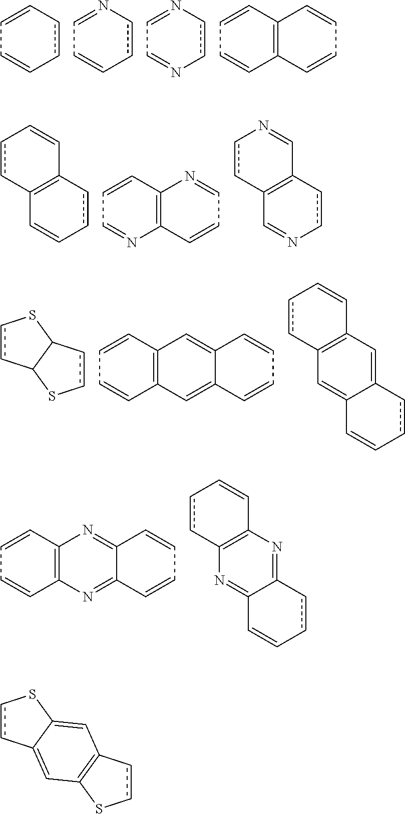

wherein:

--- indicates the fusion of M with the adjacent five-membered rings and the fusion can be in any suitable direction;

each structure can be substituted, where is applicable, with one or more suitable groups independently selected from optionally substituted hydrocarbon with about 1 to about 40 carbon atoms (such as, for example, alkyl, substituted alkyl, alkenyl, substituted alkenyl, alkynyl, substituted alkynyl, aryl, substituted aryl, alkoxy, and substituted alkoxy), cyano (CN), nitro, and halogen, or any other suitable group.

EA-3. The monomer, oligomer or polymer of EA-1, wherein (I) is selected from the following structures:

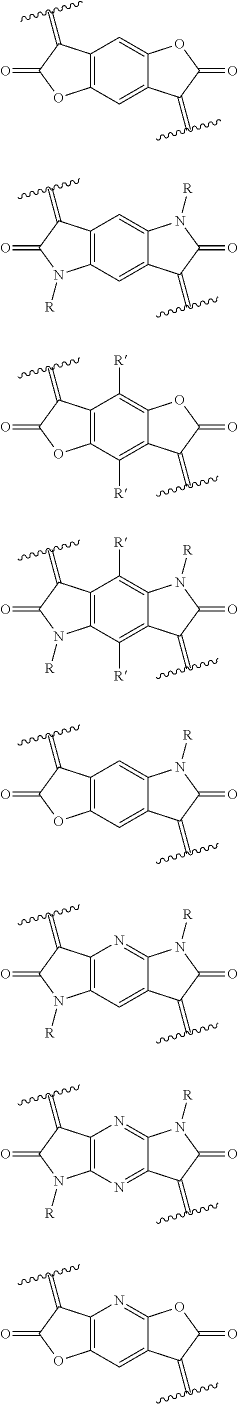

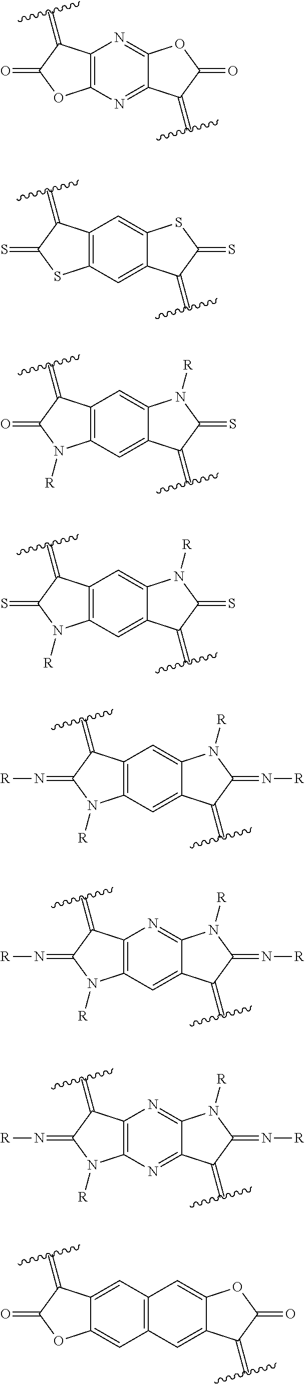

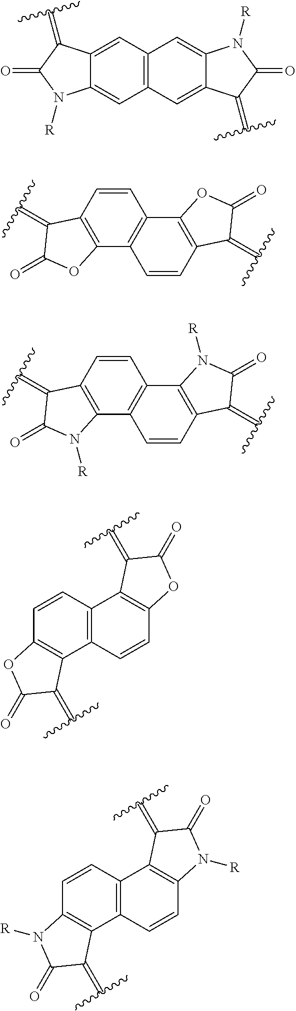

where R is independently hydrogen or optionally substituted hydrocarbon with 1 to 40 carbon atoms such as, for example, alkyl, substituted alkyl, alkenyl, substituted alkenyl, alkynyl, substituted alkynyl, aryl, and substituted aryl, or any other suitable group;

R′ is independently hydrogen or optionally substituted hydrocarbon with 1 to 40 carbon atoms such as, for example, alkyl, substituted alkyl, alkenyl, substituted alkenyl, alkynyl, substituted alkynyl, aryl, and substituted aryl, or any other suitable group;

each structure can be further substituted, where is applicable, with one or more suitable groups independently selected from optionally substituted hydrocarbon with 1 to 40 carbon atoms (such as, for example, alkyl, substituted alkyl, alkenyl, substituted alkenyl, alkynyl, substituted alkynyl, aryl, substituted aryl, alkoxy, and substituted alkoxy), cyano (CN), nitro, and halogen, or any other suitable group.

EA-4. A monomer, oligomer or polymer of EA-1 to EA-3 with the following structure PI and PII:

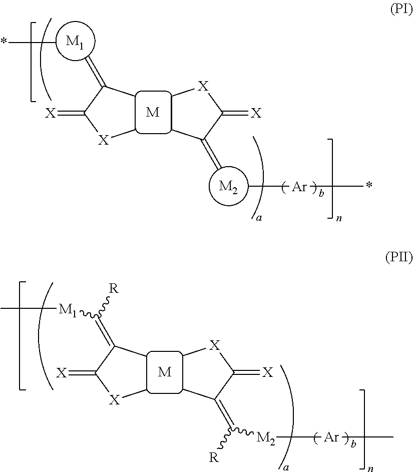

wherein X, M and R are defined as above; a is an integer from 1 to 20; b or c is an integer from 0 (zero) to 20; the unit Ar and the unit M₁-(I)-M₂ can be connected in a random or alternating manner, i.e., (PI) or (PII) can be a random copolymer, an alternating copolymer, or a block copolymer; n is a number from 1 to 1,000,000; [symbol] indicates the linkage can be a cis- or trans-structure; the terminal “*” can be hydrogen or any other suitable group or moiety; Ar is independently a n-conjugated moiety.

EA-5. The monomer, oligomer or polymer of EA-4, wherein Ar is independently selected from the following structures or a combination of the following structures:

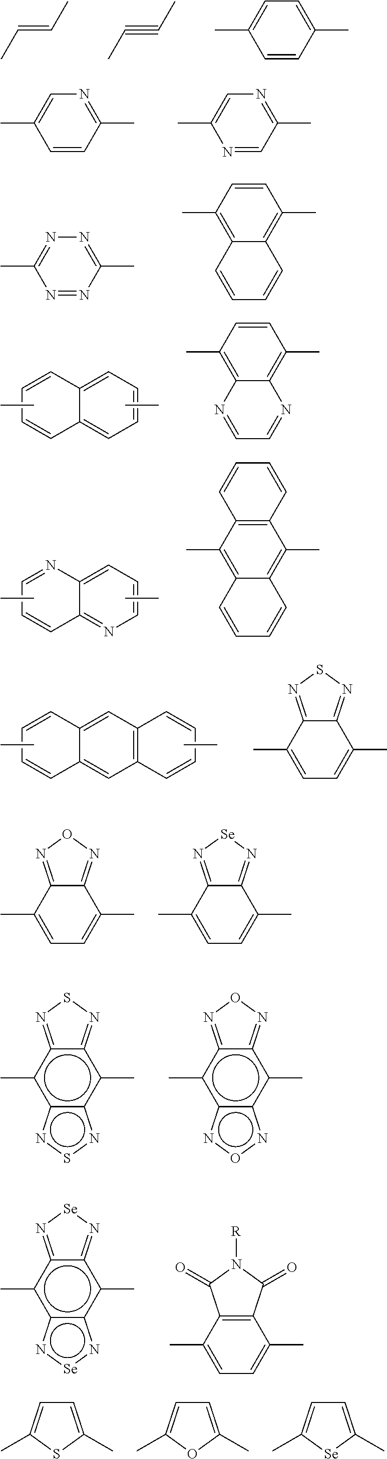

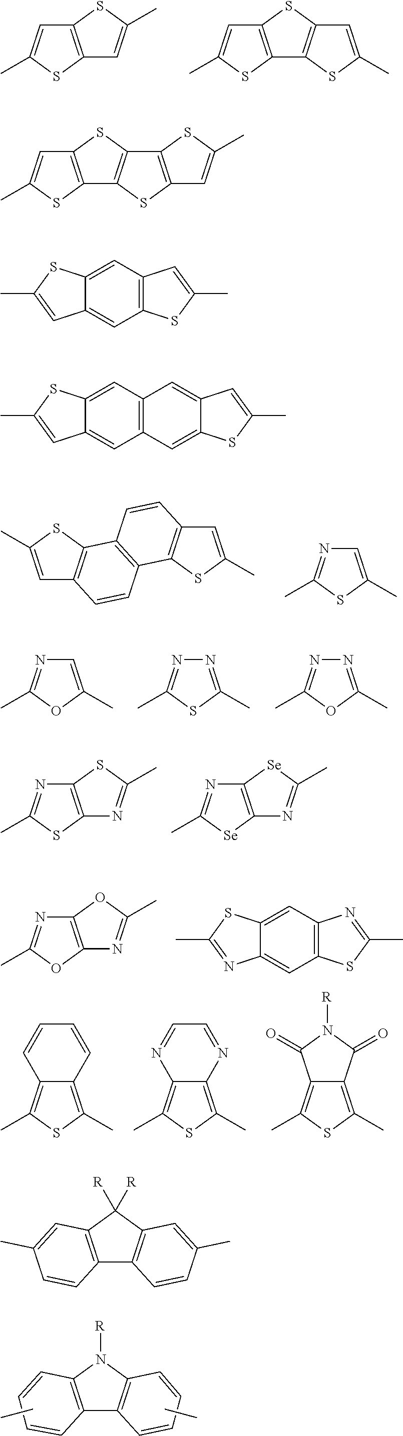

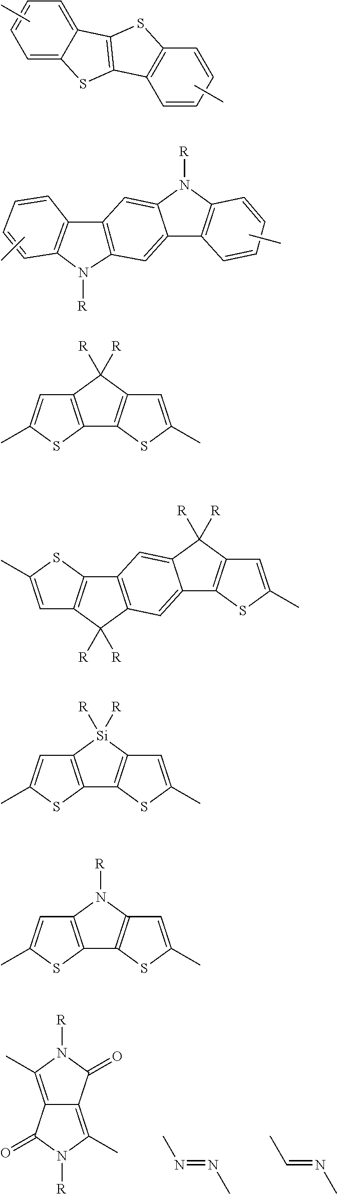

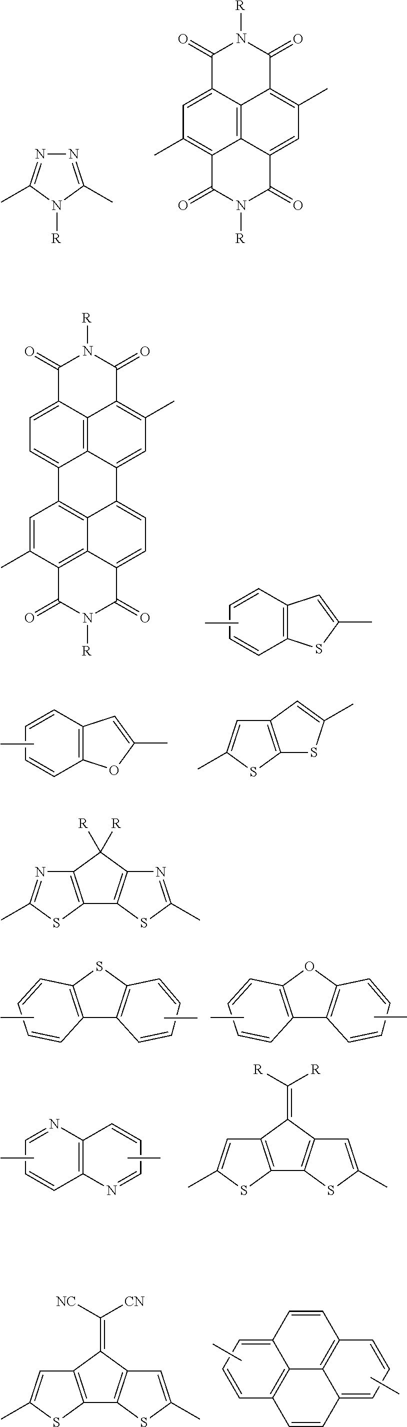

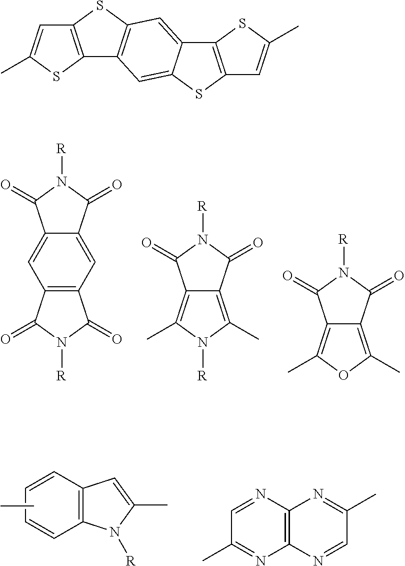

wherein R is independently hydrogen or optionally hydrocarbon of alkyl, substituted alkyl, alkenyl, substituted alkenyl, alkynyl, substituted alkynyl, aryl, or substituted aryl with 1 to 40 carbon atoms;

each structure can be substituted, where is applicable, with one or more suitable groups independently selected from optionally substituted hydrocarbon with 1 to 40 carbon atoms (such as, for example, alkyl, substituted alkyl, alkenyl, substituted alkenyl, alkynyl, substituted alkynyl, aryl, substituted aryl, alkoxy, and substituted alkoxy), cyano (CN), nitro, and halogen, or any other suitable group.

EA-6. The monomer, oligomer or polymer of EA-4 and EA-5, wherein Ar are optionally substituted with one or more suitable groups independently selected from optionally substituted hydrocarbon with 1 to 40 carbon atoms, nitro, and halogen.

EA-7. The monomer, oligomer or polymer of EA-4, which has one of the following structures.

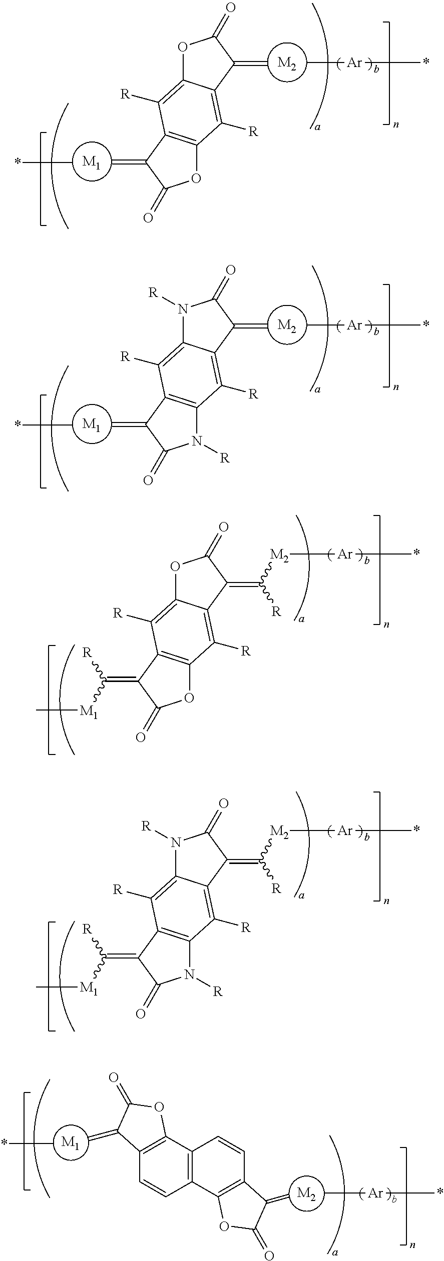

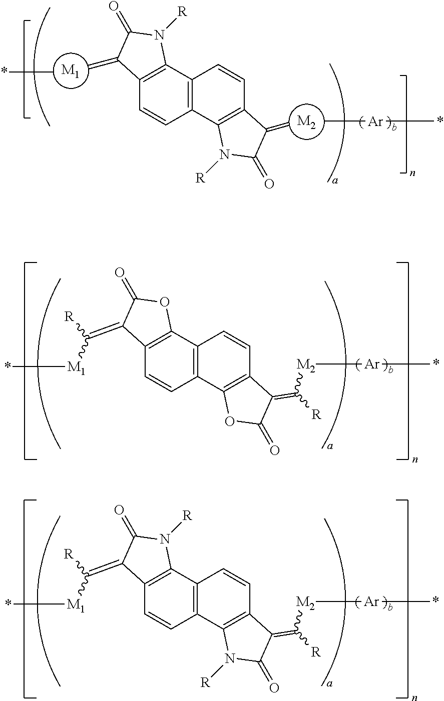

wherein

M₁, M₂, Ar, R, a, b, and n are defined as above;

each structure can be substituted, where is applicable, with one or more suitable groups independently selected from optionally substituted hydrocarbon with 1 to 40 carbon atoms (such as, for example, alkyl, substituted alkyl, alkenyl, substituted alkenyl, alkynyl, substituted alkynyl, aryl, substituted aryl, alkoxy, and substituted alkoxy), cyano (CN), nitro, and halogen, or any other suitable group;

n is a number from 1 to 1,000,000;

[symbol] indicates the linkage can be a cis- or trans-structure;

the terminal “*” can be hydrogen or any other suitable group or moiety.

EA-8. The monomer, oligomer or polymer EA-4 and EA-7 is selected from above structures (1) through (230) wherein:

R is independently selected from hydrogen, optionally substituted hydrocarbon with 1 to 40 carbon atoms (such as, for example, alkyl, substituted alkyl, alkenyl, substituted alkenyl, alkynyl, substituted alkynyl, aryl, and substituted aryl), or any other suitable group;

each structure can be substituted, where is applicable, with one or more suitable groups independently selected from optionally substituted hydrocarbon with 1 to 40 carbon atoms (such as, for example, alkyl, substituted alkyl, alkenyl, substituted alkenyl, alkynyl, substituted alkynyl, aryl, substituted aryl, alkoxy, and substituted alkoxy), cyano (CN), nitro, and halogen, or any other suitable group;

n is a number from 1 to 1,000,000;

the terminal “*” can be hydrogen or any other suitable group or moiety.

### Second Preferred Embodiment

In a first preferred embodiment the present application relates to the following aspects EB-1 to EB-8:

EB-1. A monomer, oligomer or polymer comprising a fused-ring moiety (I):

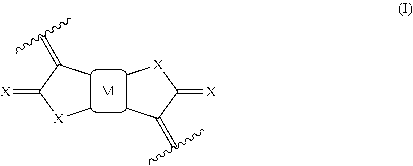

X is independently oxygen (O), sulphur (S), or NR (R is independently hydrogen or optionally substituted hydrocarbon with 41 (or 45) to 60 carbon atoms such as, for example, alkyl, substituted alkyl, alkenyl, substituted alkenyl, alkynyl, substituted alkynyl, aryl, and substituted aryl, or any other suitable group);

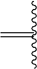

indicates the linkage with another moiety (or moieties);

M is a conjugated moiety.

EB-2. The monomer, oligomer or polymer of EB-1, wherein M is selected from a group of structures:

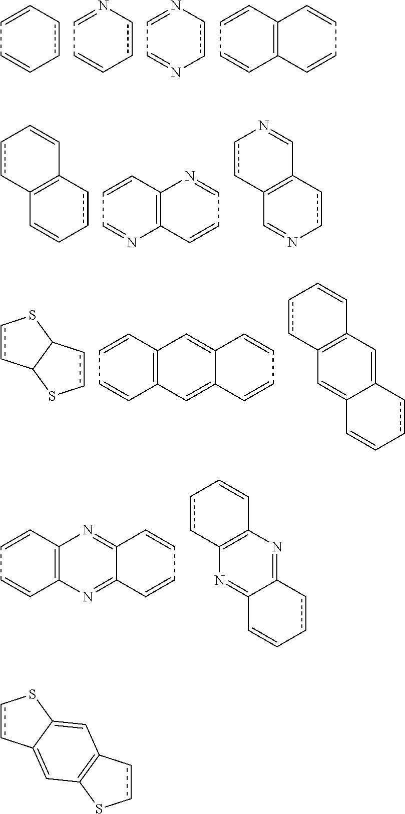

wherein:

--- indicates the fusion of M with the adjacent five-membered rings and the fusion can be in any suitable direction;

each structure can be substituted, where is applicable, with one or more suitable groups independently selected from optionally substituted hydrocarbon with about 41 (or 45) to about 60 carbon atoms (such as, for example, alkyl, substituted alkyl, alkenyl, substituted alkenyl, alkynyl, substituted alkynyl, aryl, substituted aryl, alkoxy, and substituted alkoxy), cyano (CN), nitro, and halogen, or any other suitable group.

EB-3. The monomer, oligomer or polymer of EB-1, wherein (I) is selected from the following structures:

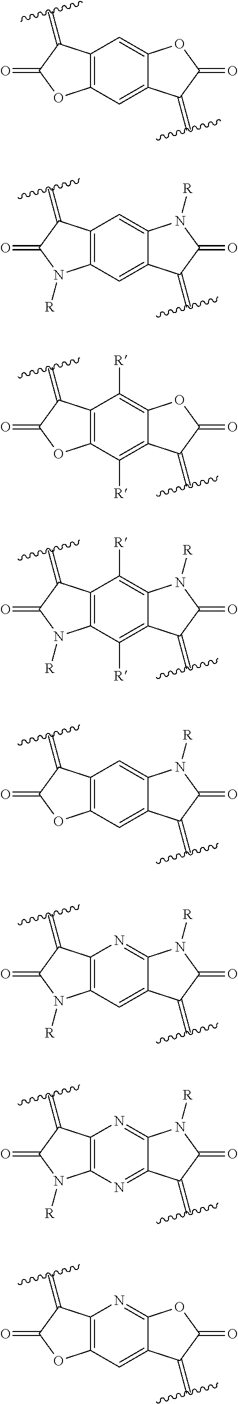

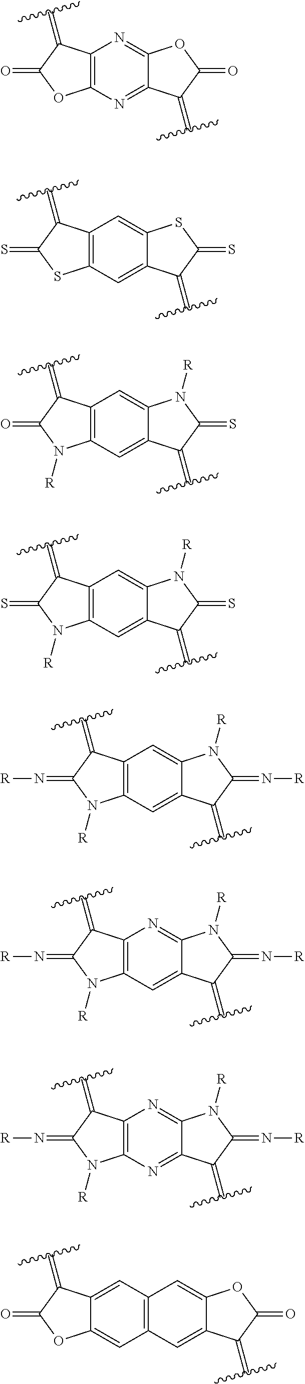

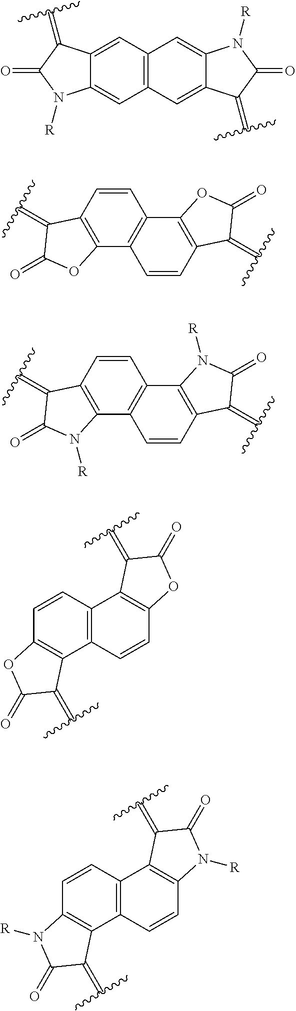

where R is independently hydrogen or optionally substituted hydrocarbon with 41 (or 45) to 60 carbon atoms such as, for example, alkyl, substituted alkyl, alkenyl, substituted alkenyl, alkynyl, substituted alkynyl, aryl, and substituted aryl, or any other suitable group;

R′ is independently hydrogen or optionally substituted hydrocarbon with 41 (or 45) to 60 carbon atoms such as, for example, alkyl, substituted alkyl, alkenyl, substituted alkenyl, alkynyl, substituted alkynyl, aryl, and substituted aryl, or any other suitable group;

each structure can be further substituted, where is applicable, with one or more suitable groups independently selected from optionally substituted hydrocarbon with 41 (or 45) to 60 carbon atoms (such as, for example, alkyl, substituted alkyl, alkenyl, substituted alkenyl, alkynyl, substituted alkynyl, aryl, substituted aryl, alkoxy, and substituted alkoxy), cyano (CN), nitro, and halogen, or any other suitable group.

EB-4. A monomer, oligomer or polymer of EB-1 to EB-3 with the following structure PI and PII:

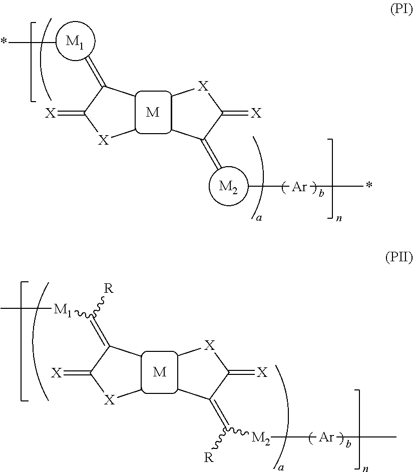

wherein X, M and R are defined as above; a is an integer from 1 to 20; b or c is an integer from 0 (zero) to 20; the unit Ar and the unit M₁-(I)-M₂ can be connected in a random or alternating manner, i.e., (PI) or (PII) can be a random copolymer, an alternating copolymer, or a block copolymer; n is a number from 1 to 1,000,000; [symbol] indicates the linkage can be a cis- or trans-structure; the terminal “*” can be hydrogen or any other suitable group or moiety; Ar is independently a π-conjugated moiety.

EB-5. The monomer, oligomer or polymer of EB-4, wherein Ar is independently selected from the following structures or a combination of the following structures:

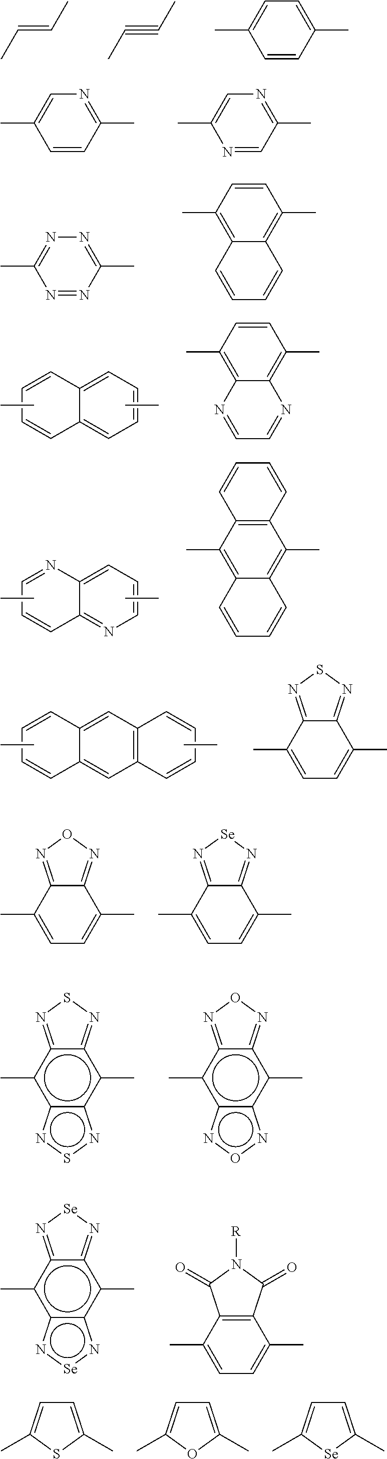

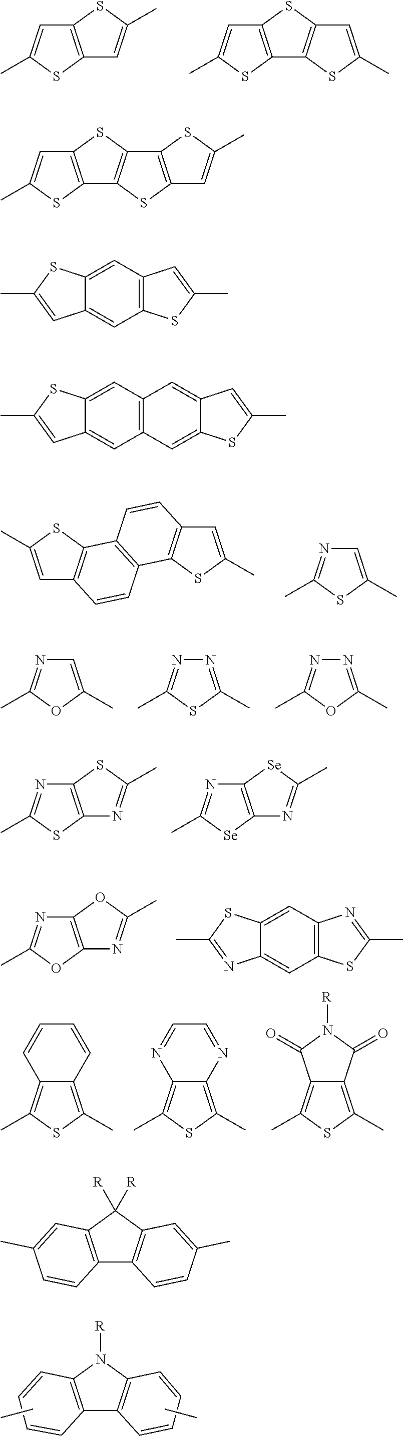

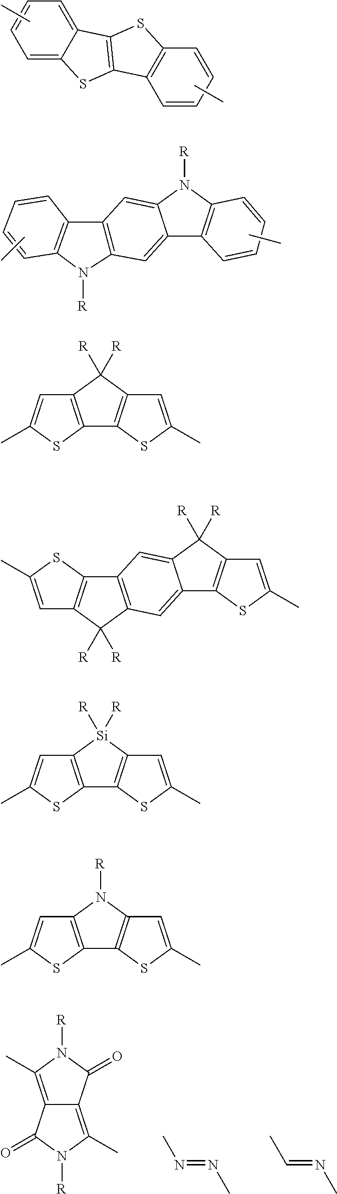

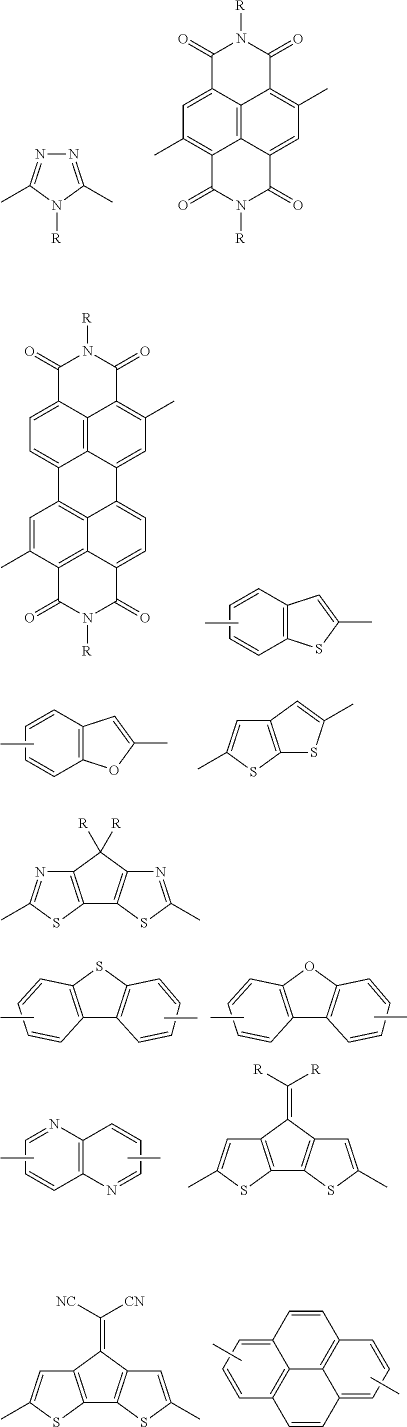

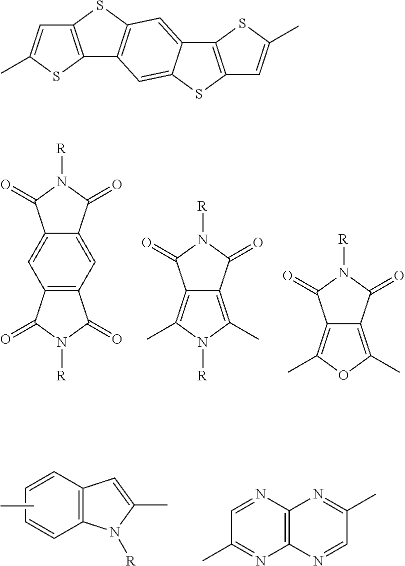

wherein R is independently hydrogen or optionally hydrocarbon of alkyl, substituted alkyl, alkenyl, substituted alkenyl, alkynyl, substituted alkynyl, aryl, or substituted aryl with 41 (or 45) to 60 carbon atoms;

each structure can be substituted, where is applicable, with one or more suitable groups independently selected from optionally substituted hydrocarbon with 41 (or 45) to 60 carbon atoms (such as, for example, alkyl, substituted alkyl, alkenyl, substituted alkenyl, alkynyl, substituted alkynyl, aryl, substituted aryl, alkoxy, and substituted alkoxy), cyano (CN), nitro, and halogen, or any other suitable group.

EB-6. The monomer, oligomer or polymer of EB-4 and EB-5, wherein Ar are optionally substituted with one or more suitable groups independently selected from optionally substituted hydrocarbon with 41 (or 45) to 60 carbon atoms, nitro, and halogen.

EB-7. The monomer, oligomer or polymer of EB-4, which has one of the following structures.

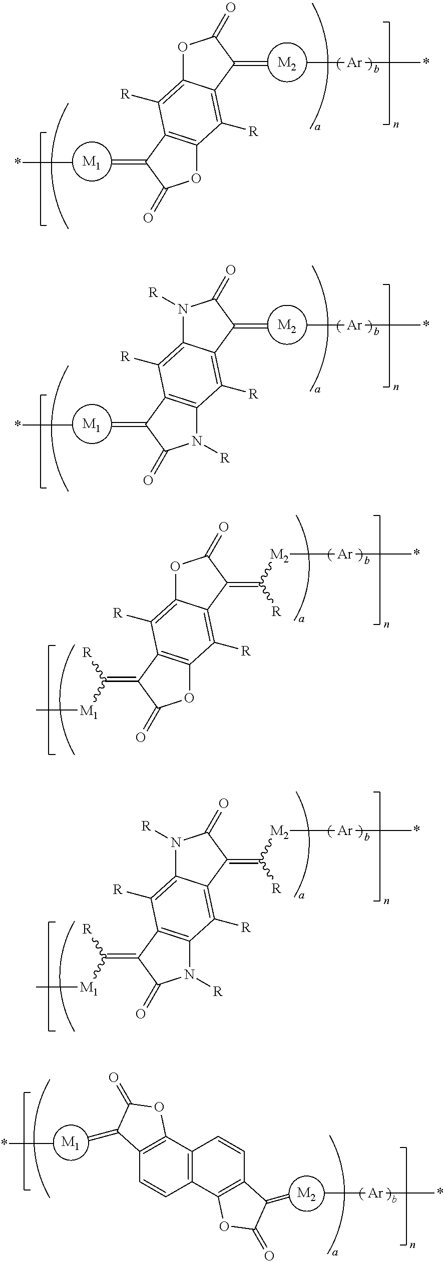

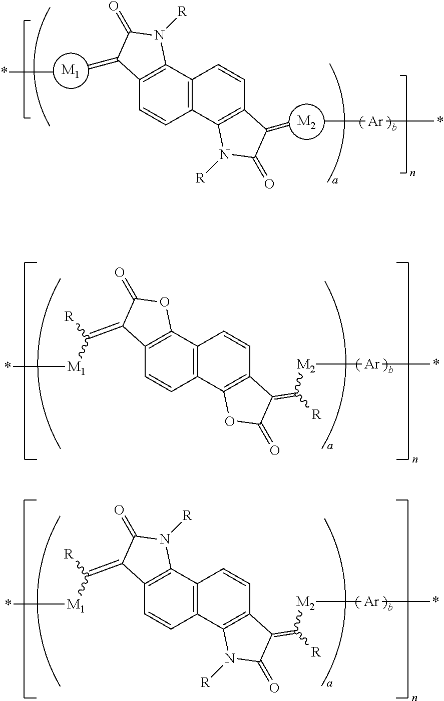

wherein

M₁, M₂, Ar, R, a, b, and n are defined as above;

each structure can be substituted, where is applicable, with one or more suitable groups independently selected from optionally substituted hydrocarbon with 41 (or 45) to 60 carbon atoms (such as, for example, alkyl, substituted alkyl, alkenyl, substituted alkenyl, alkynyl, substituted alkynyl, aryl, substituted aryl, alkoxy, and substituted alkoxy), cyano (CN), nitro, and halogen, or any other suitable group;

n is a number from 1 to 1,000,000;

[symbol] indicates the linkage can be a cis- or trans-structure;

the terminal “*” can be hydrogen or any other suitable group or moiety.

EB-8. The monomer, oligomer or polymer EB-4 and EB-7 is selected from above structures (1) through (230) wherein:

R is independently selected from hydrogen, optionally substituted hydrocarbon with 41 (or 45) to 60 carbon atoms (such as, for example, alkyl, substituted alkyl, alkenyl, substituted alkenyl, alkynyl, substituted alkynyl, aryl, and substituted aryl), or any other suitable group;

each structure can be substituted, where is applicable, with one or more suitable groups independently selected from optionally substituted hydrocarbon with 41 (or 45) to 60 carbon atoms (such as, for example, alkyl, substituted alkyl, alkenyl, substituted alkenyl, alkynyl, substituted alkynyl, aryl, substituted aryl, alkoxy, and substituted alkoxy), cyano (CN), nitro, and halogen, or any other suitable group;

n is a number from 1 to 1,000,000;

the terminal “*” can be hydrogen or any other suitable group or moiety.

Monomers containing (I) can be readily synthesized by known procedures in the literature such as, for example, Connor, et al. U.S. Pat. No. 6,492,533 B1 (Dec. 10, 2002) and Nesvadba, et al. U.S. Pat. No. 6,503,937 B1.

In embodiments, the monomeric, oligomeric or polymeric materials comprising moiety (I) in the present invention can be used in electronic devices such as thin film transistors, photovoltaics, and sensors. The use of the present monomer, oligomer or polymer as a semiconductor in electronic devices is illustrated herein using thin film transistors.

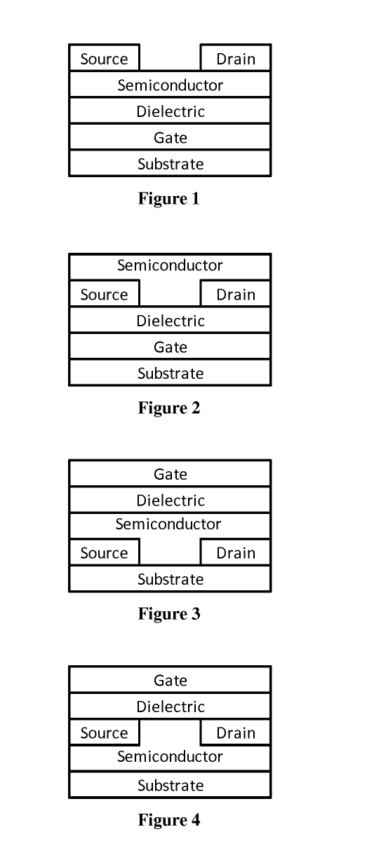

In FIG. 1, there is schematically illustrated a bottom-gate, top-contact OTFT configuration comprised of a substrate, in contact therewith a gate electrode and a layer of a gate dielectric. On top of the gate dielectric there is an organic semiconductor layer. Two conductive contacts, source electrode and drain electrode, are deposited on top of the organic semiconductor layer.

FIG. 2 schematically illustrates a bottom-gate, bottom-contact OTFT configuration comprised of a substrate, a gate electrode, a source electrode and a drain electrode, a gate dielectric layer, and an organic semiconductor layer.

FIG. 3 schematically illustrates a top-gate, bottom-contact OTFT configuration comprised of a substrate, a gate electrode, a source electrode and a drain electrode, a gate dielectric layer, and an organic semiconductor layer.

FIG. 4 schematically illustrates a top-gate, top-contact OTFT configuration comprised of a substrate, a gate electrode, a source electrode and a drain electrode, a gate dielectric layer, and an organic semiconductor layer.

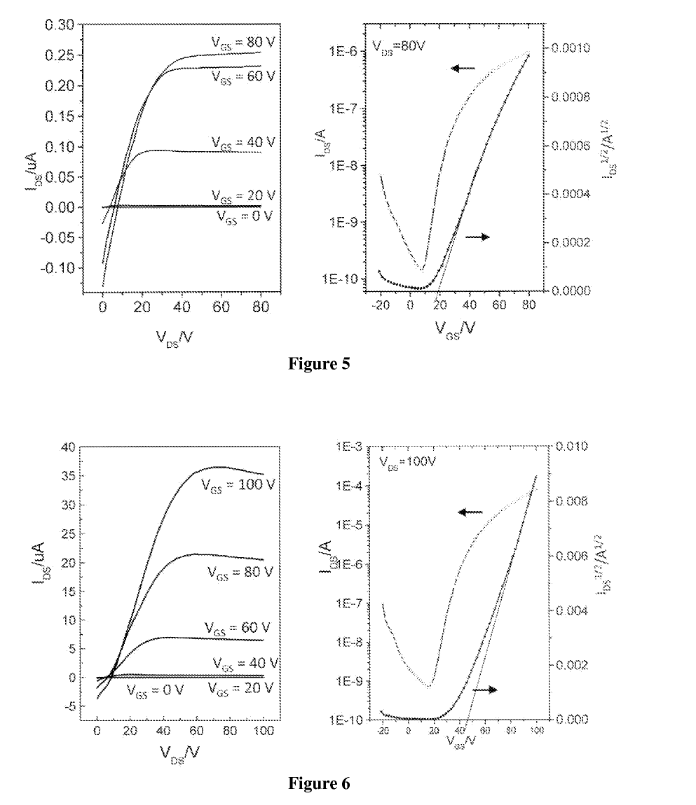

FIG. 5 shows the output (left) and transfer (right) characteristics of an OTFT device with PIBDFT-24 (annealed at 200° C. for 15 minutes) in the electron accumulation regime. L=30 μm, W=1 mm.

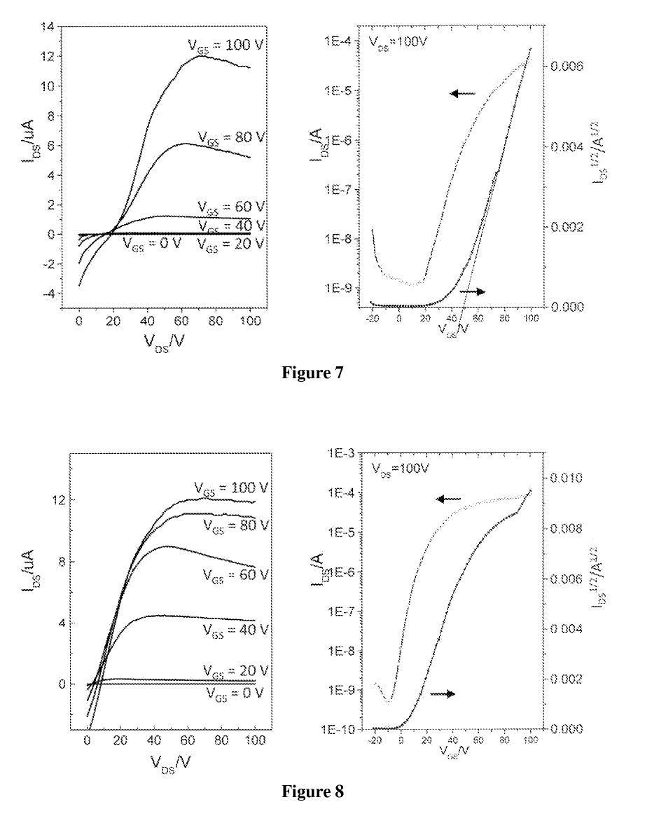

FIG. 6 shows the output (left) and transfer (right) characteristics of an OTFT device with PIBDFV-24 (annealed at 200° C. for 15 minutes) in the electron accumulation regime. L=30 μm, W=1 mm.

FIG. 7 shows the output (left) and transfer (right) characteristics of an OTFT device with PIBDFV-26 (annealed at 200° C. for 15 minutes) in the electron accumulation regime. L=30 μm, W=1 mm.

FIG. 8 shows the output (left) and transfer (right) characteristics of an OTFT device with PIBDFV-40 (annealed at 200° C. for 15 minutes) in the electron accumulation regime. L=30 μm, W=1 mm.

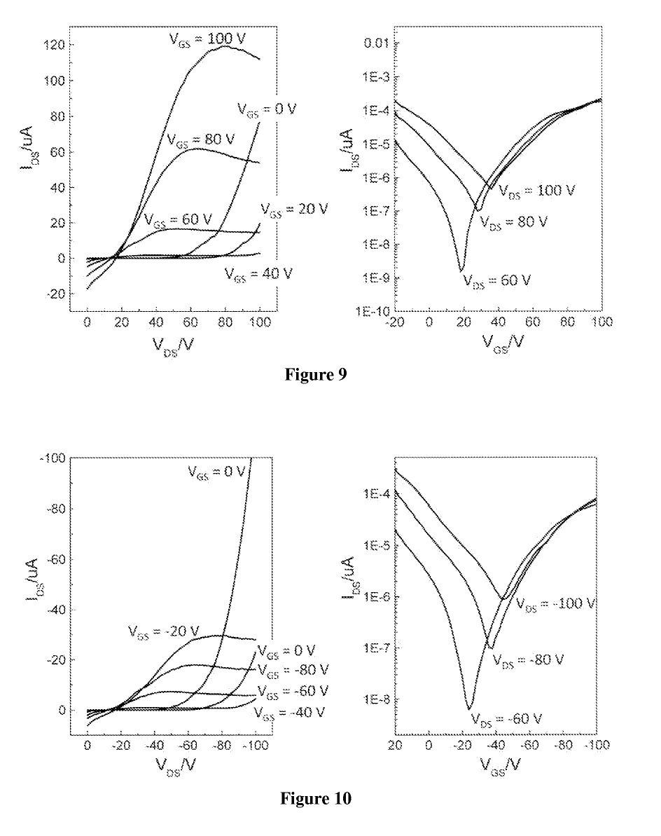

FIG. 9 shows the output (left) and transfer (right) characteristics of an OTFT device with PIBDFBT-40 (annealed at 200° C. for 15 minutes) in the electron accumulation regime. L=30 μm, W=1 mm.

FIG. 10 shows the output (left) and transfer (right) characteristics of an OTFT device with PIBDFBT-40 (annealed at 200° C. for 15 minutes) in the hole accumulation regime. L=30 μm, W=1 mm.

The semiconductor layer has a thickness ranging for example from about 10 nanometers to about 1 micrometer with a preferred thickness of from about 20 to about 200 nanometers. The OTFT devices contain a semiconductor channel with a width, W and length, L. The semiconductor channel width may be, for example, from about 1 micrometer to about 5 millimeters, with a specific channel width being about 5 micrometers to about 1 millimeter. The semiconductor channel length may be, for example, from about 10 nanometers to about 1 millimeter with a more specific channel length being from about 20 nanometers to about 500 micrometers.

Small Molecule and Monomer

In one aspect the present application provides for a small molecule, i.e. for a compound comprising said fused-ring moiety and two inert chemical groups R^{a} and R^{b}. Such a small molecule may for example be represented by formula (II-a)

R^{a}-Q-R^{b} (II-a)

wherein Q comprises said fused-ring moiety and R^{a} and R^{b} are inert chemical groups. Such inert chemical groups R^{a} and R^{b} may independently of each other for example be chosen from the group consisting of hydrogen, fluorine, alkyl having from 1 to 60, preferably from 1 to 50, more preferably from 1 to 30 and most preferably from 1 to 20 carbon atoms, fluoroalkyl having from 1 to 60, preferably from 1 to 50, more preferably from 1 to 30 and most preferably from 1 to 20 carbon atoms, aromatic ring systems of from 5 to 30 carbon atoms and aromatic ring systems of from 5 to 30 carbon atoms wherein one or more hydrogen atom may independently of each other be replaced by fluorine or alkyl having from 1 to 40, preferably from 1 to 30, more preferably from 1 to 20 and most preferably from 1 to 10 carbon atoms.

In another aspect the present application provides for a monomer, i.e. for a compound comprising said fused-ring moiety and at least one reactive chemical group R^{c} which may be selected from group consisting of Cl, Br, I, O-tosylate, O-triflate, O-mesylate, O-nonaflate, —SiMe₂F, —SiMeF₂, —O—SO₂Z¹, —B(OZ²)₂, —CZ³═C(Z³)₂, —C≡CH, —C≡CSi(Z¹)₃, —ZnX⁰ and —Sn(Z⁴)₃, wherein X⁰ is as defined above, and Z¹, Z², Z³ and Z⁴ are selected from the group consisting of alkyl and aryl, each being optionally substituted, and two groups Z² may also together form a cyclic group. Alternatively such a monomer may comprise two reactive chemical groups and is for example represented by formula (II-b)

R^{c}-Q-R^{d} (II-b)

wherein Q comprises said fused-ring moiety and R^{c} and R^{d} are reactive chemical groups as defined above for R^{c}.

Preferably, Q in formulae (II-a) and (II-b) may further comprise one or more (for example 2, 3, 4, 5, 6, 7, 8, 9 or 10) aryl or heteroaryl as defined above. Preferred examples of Q may be comprise, preferably consist of, the following

*—U^{a}ₘ₁—Ar^{a}ₘ₂—U^{b}ₘ₃—Ar^{b}ₘ₄—Ar^{c}ₘ₅—* (III)

wherein
U^{a} and U^{b} are independently of each other selected from said fused-ring moiety and any of its subformulae as defined above;
Ar^{a}, Ar^{b} and Ar^{c} are independently of each other aryl or heteroaryl different from U^{a} and U^{b};
m1, m2, m3 and m4 are independently of each other selected from the group consisting of 0, 1 and 2, with the provision that at least one of m1 and m3 is not 0; and
m5 is 0 or an integer from 1 to 10 (for example 2, 3, 4, 5, 6, 7, 8 or 9).

Preferably Ar^{a}, Ar^{b} and Ar^{c} are selected from aryl having from 5 to 30 ring atoms and heteroaryl having from 5 to 30 ring atoms. Said aryl and heteroaryl may optionally be substituted with at least one substituent L as defined earlier. Preferred substituents L are selected from alkyl having from 1 to 60 carbon atoms, more preferably from alkyl having from 1 to 40 carbon atoms, even more preferably from alkyl having from 1 to 30 carbon atoms and most preferably from alkyl having from 1 to 20 carbon atoms. Optionally, said alkyl may be partially or completely fluorinated.

Preferred small molecules and monomers are those with Q selected from one of the following formula (III-a-1) and (III-a-2)

*—Ar^{a}ₘ₂—U^{b}—Ar^{b}ₘ₄—* (III-a-1)

*—U^{a}ₘ₁—Ar^{a}ₘ₂—U^{b}ₘ₃—* (III-a-2)

with Ar^{a}, Ar^{b}, U^{a}, U^{b}, m1, m2, m3 and m4 as defined above.

Especially preferred small molecules and monomers are those with Q selected from one of the following formulae (III-b-1) to (III-b-5)

*—Ar^{a}—U^{a}—Ar^{b}—* (III-b-1)

*—U^{a}—* (III-b-2)

*—Ar^{a}—U^{a}—* (III-b-3)

*—U^{a}—Ar^{b}—* (III-b-4)

*—U^{a}—Ar^{a}—U^{b}—* (III-b-5)

with Ar^{a}, Ar^{b}, U^{a} and U^{b} as defined above.

Particularly preferred examples of Q of formulae (III), (III-a-1), (III-a-2) and (III-b-1) to (III-b-5) are those wherein one or more of Ar^{a}, Ar^{b} and Ar^{c} denote aryl or heteroaryl, preferably having electron donor properties or electron acceptor properties.

Suitable examples of aryl and heteroaryl with electron donor properties may be selected from the group consisting of the following formulae (D1) to (D126)

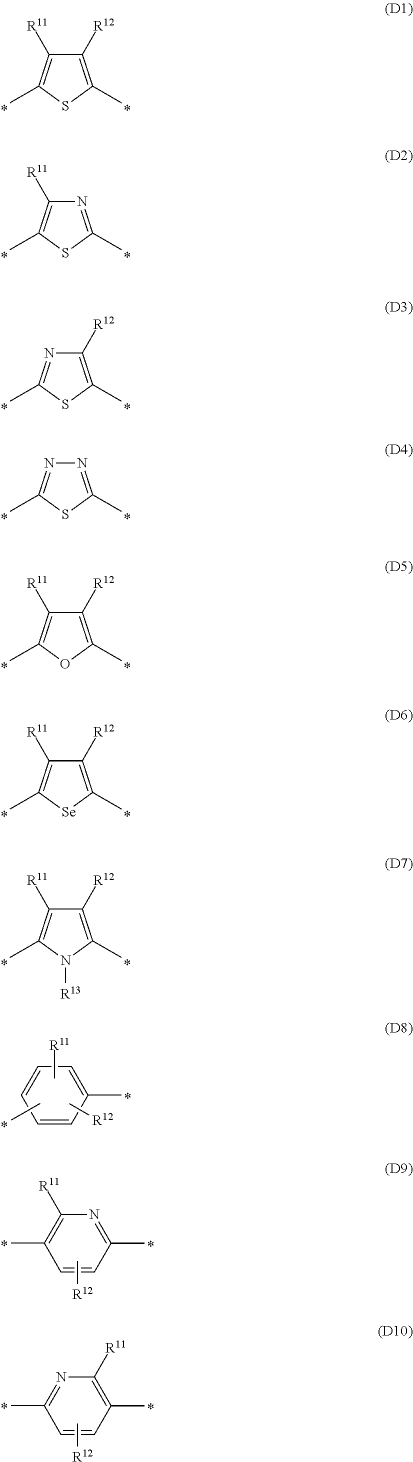

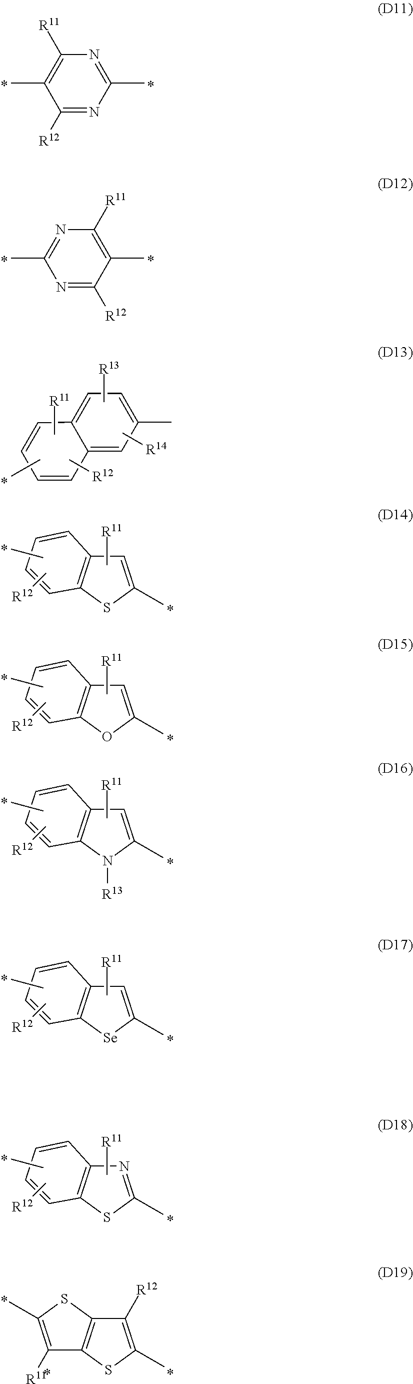

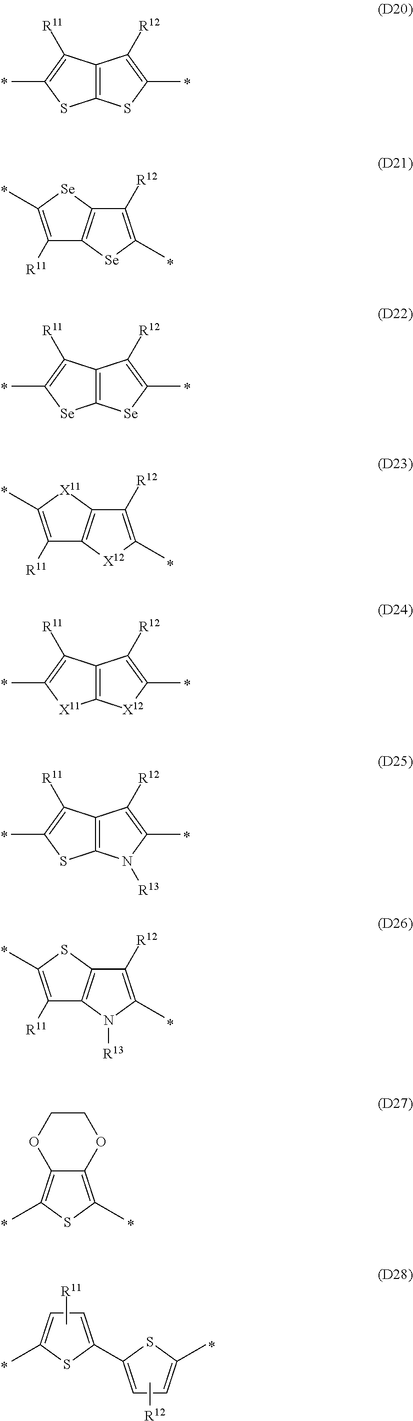

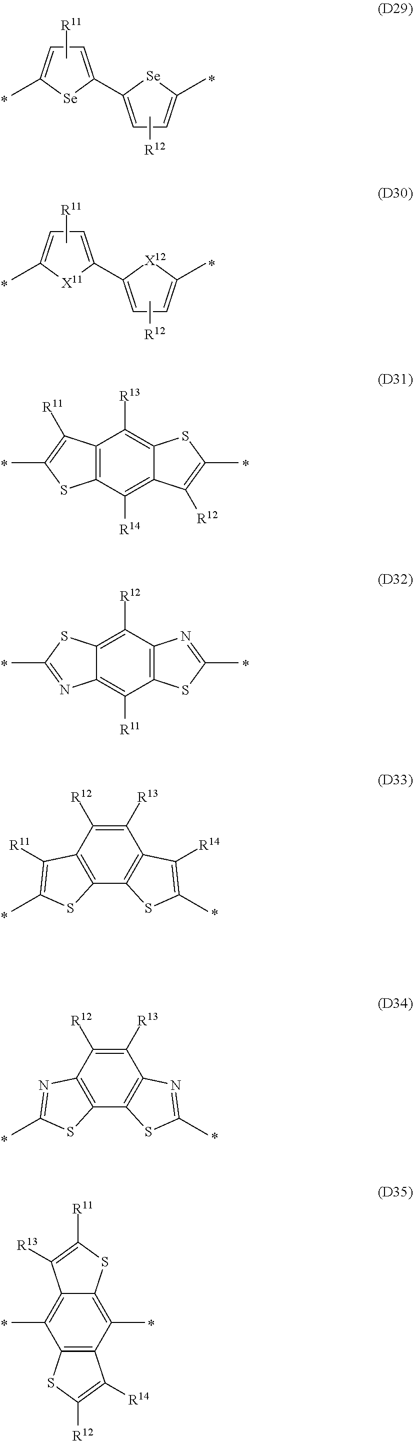

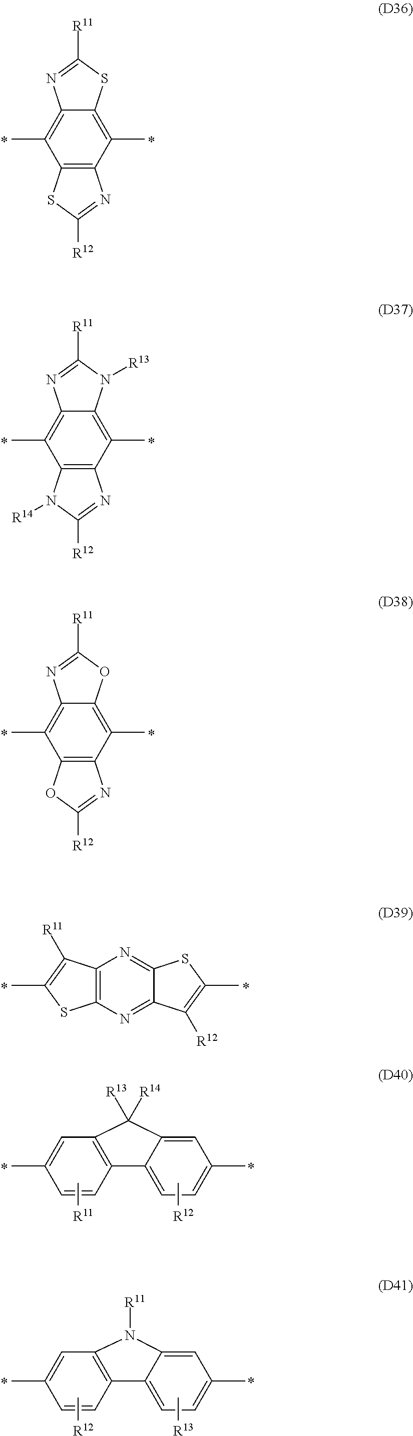

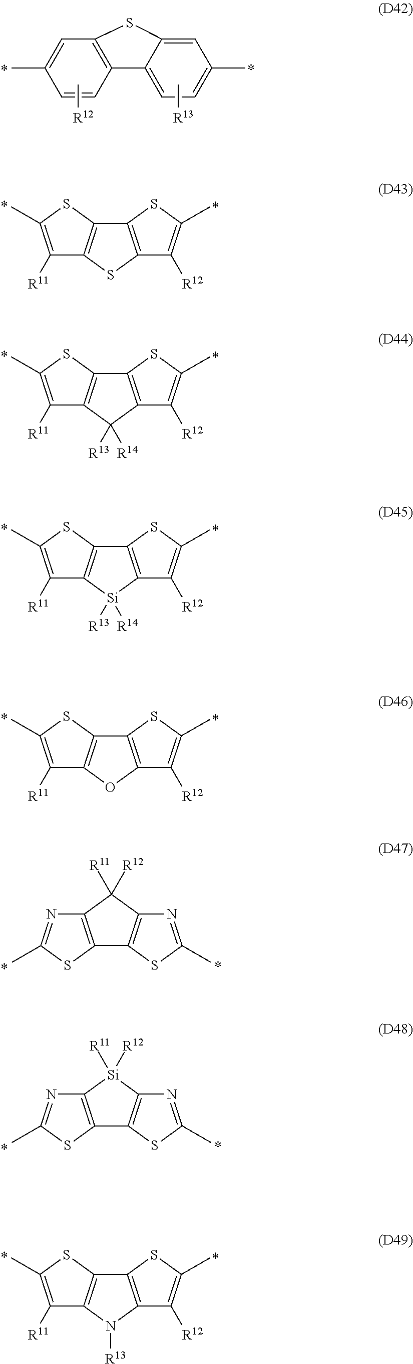

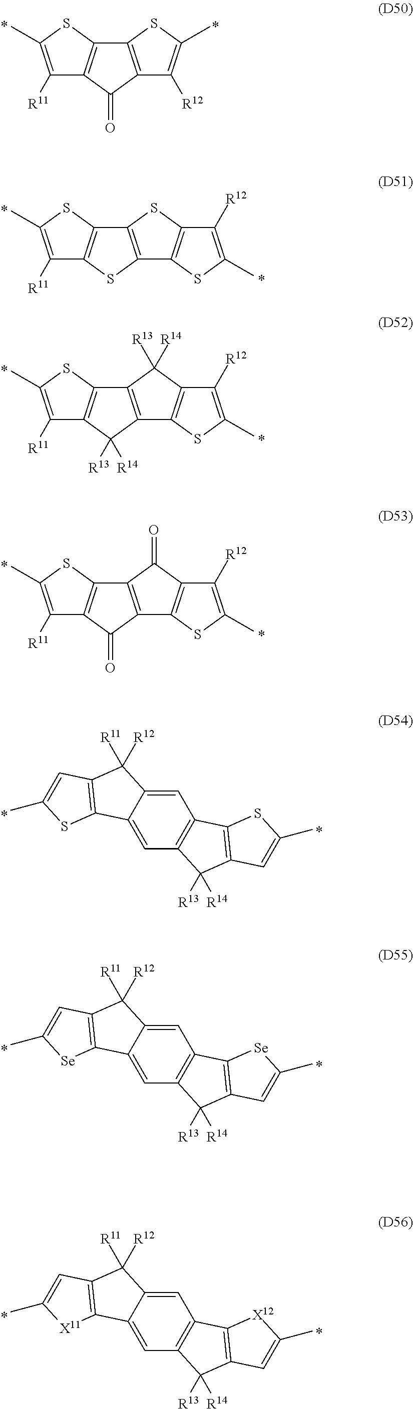

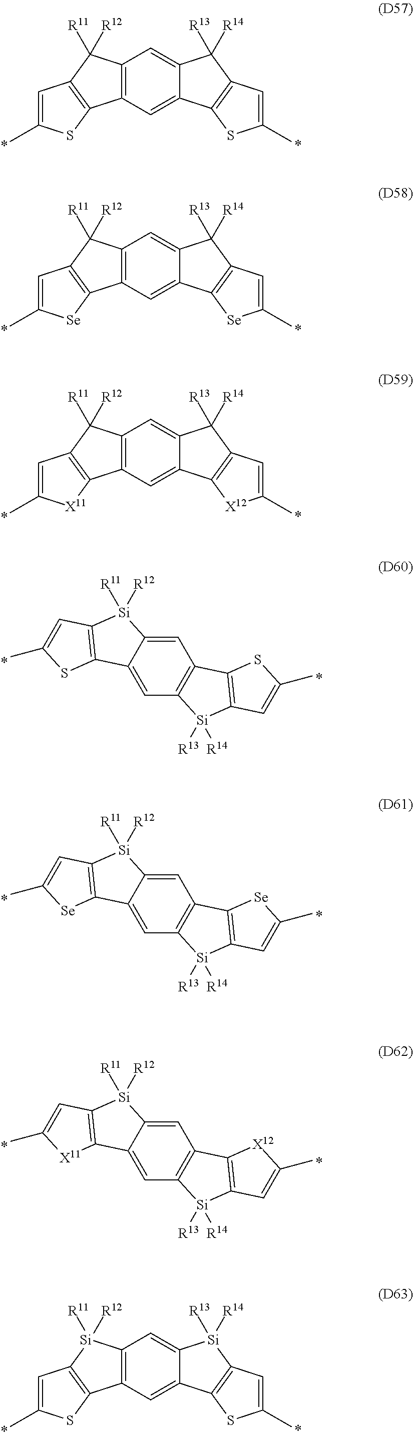

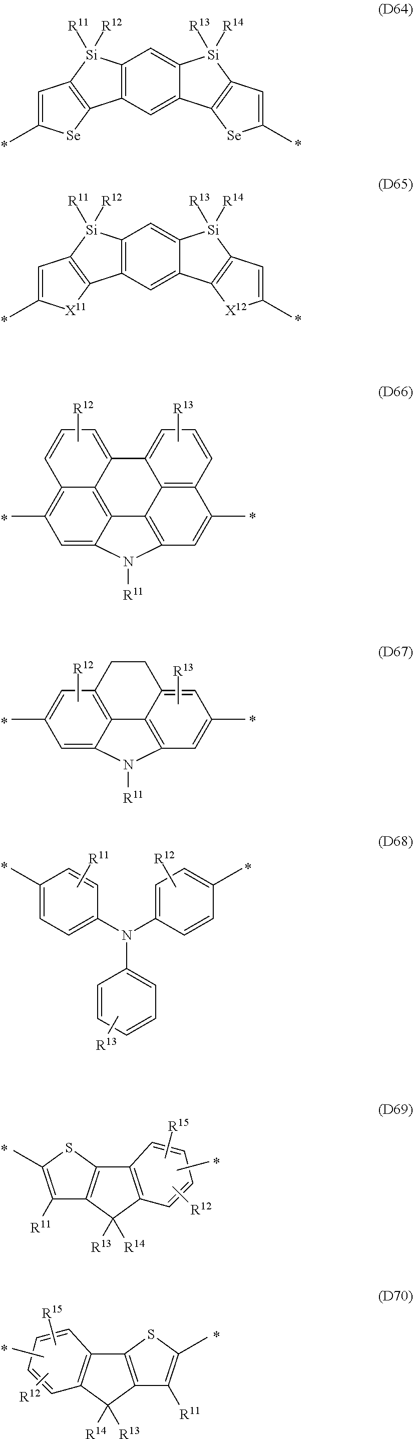

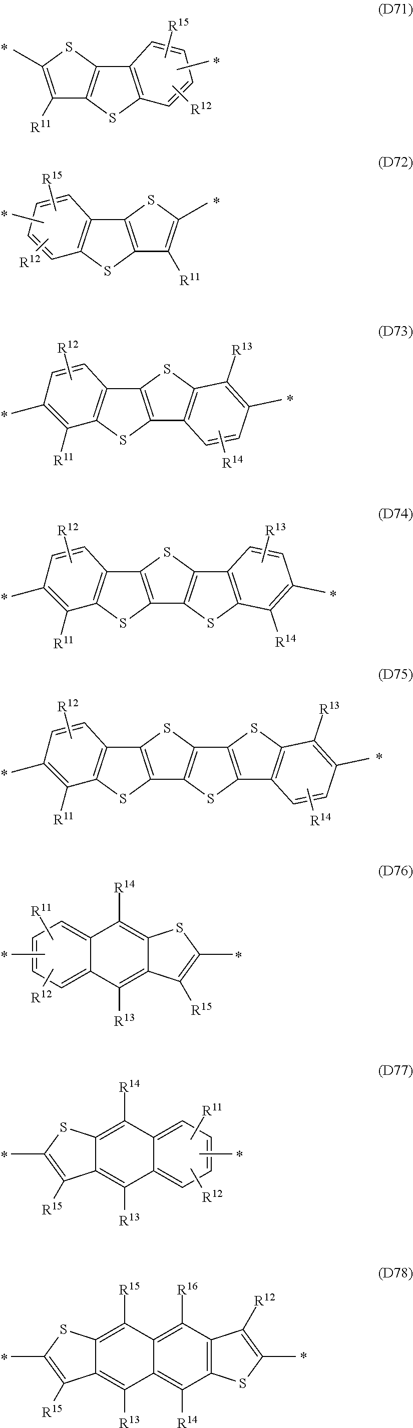

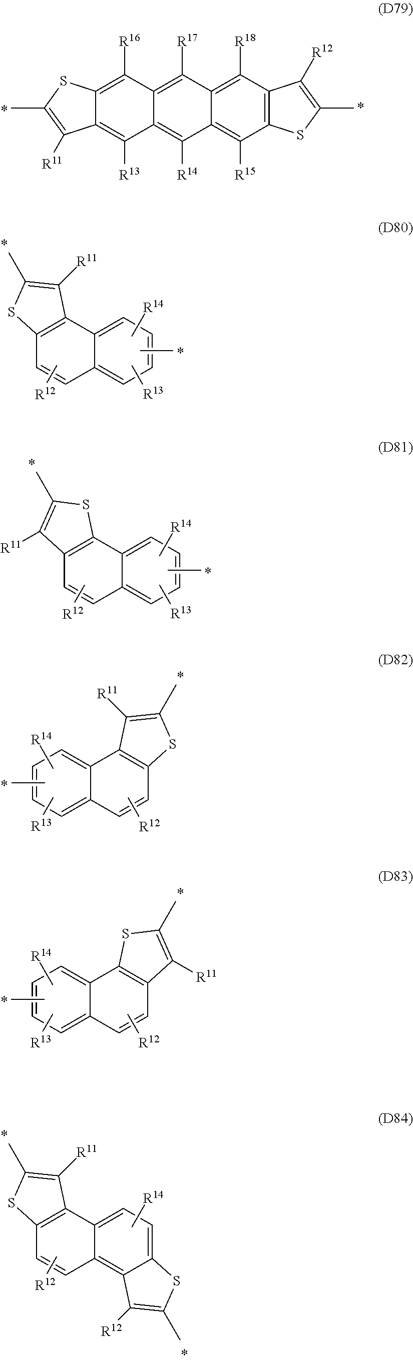

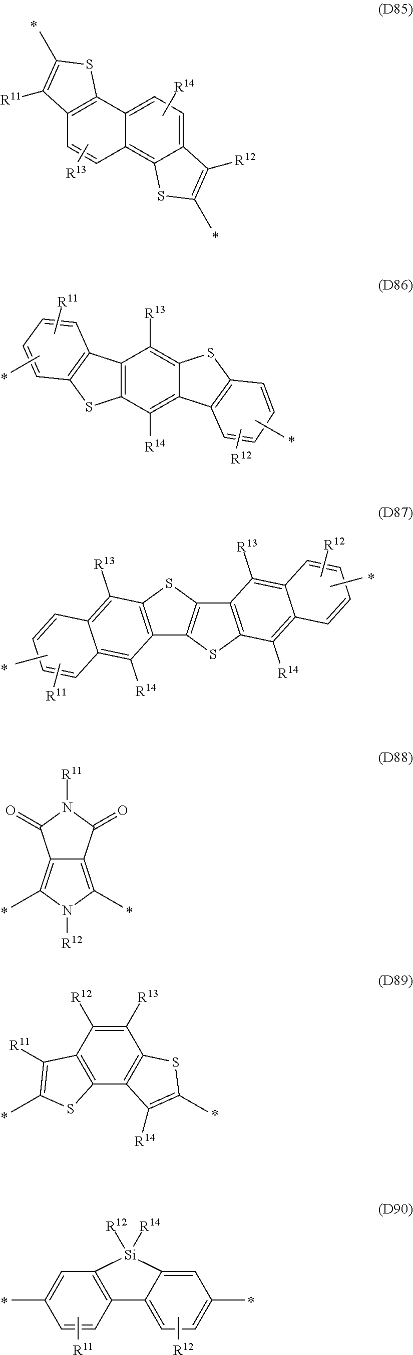

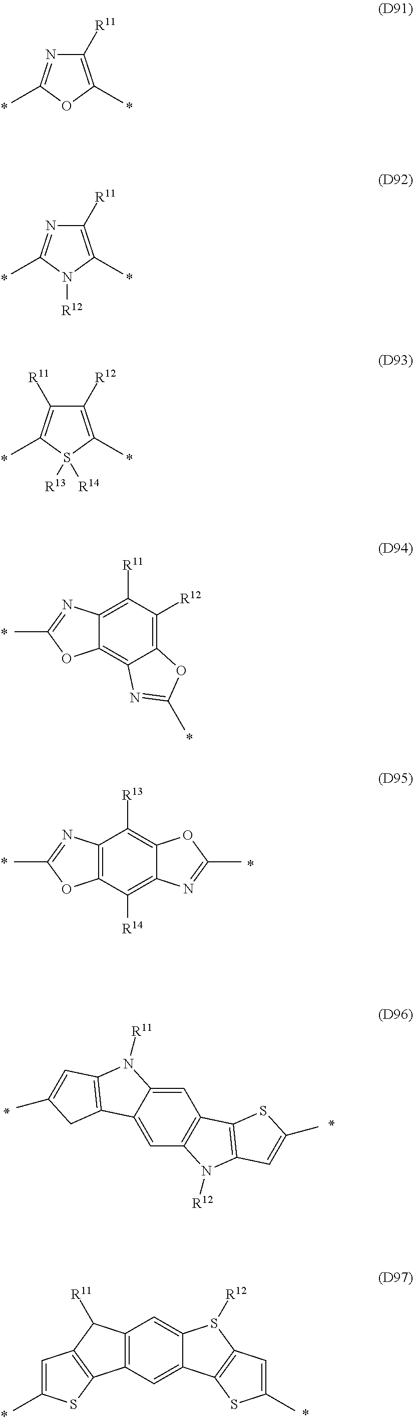

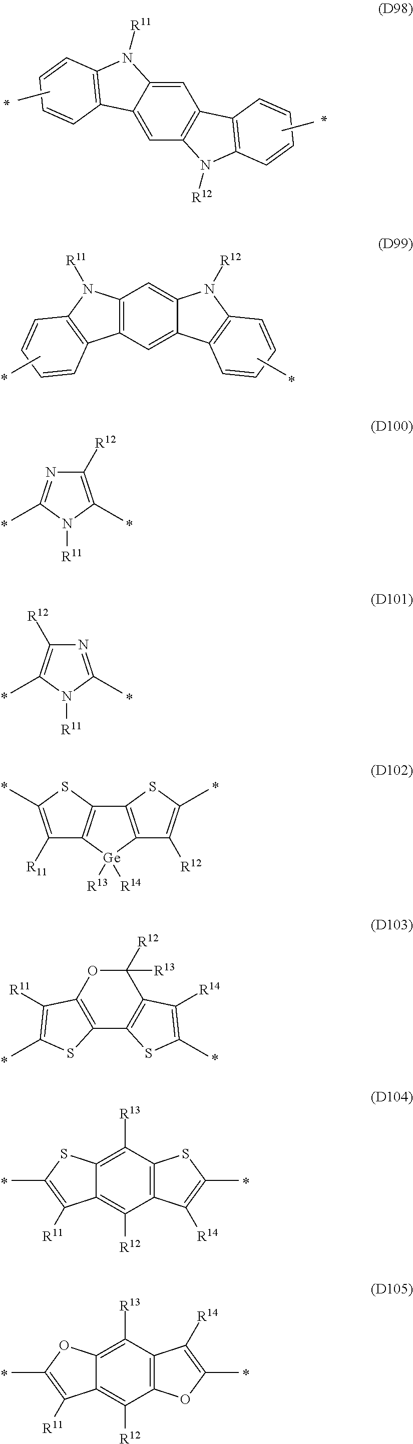

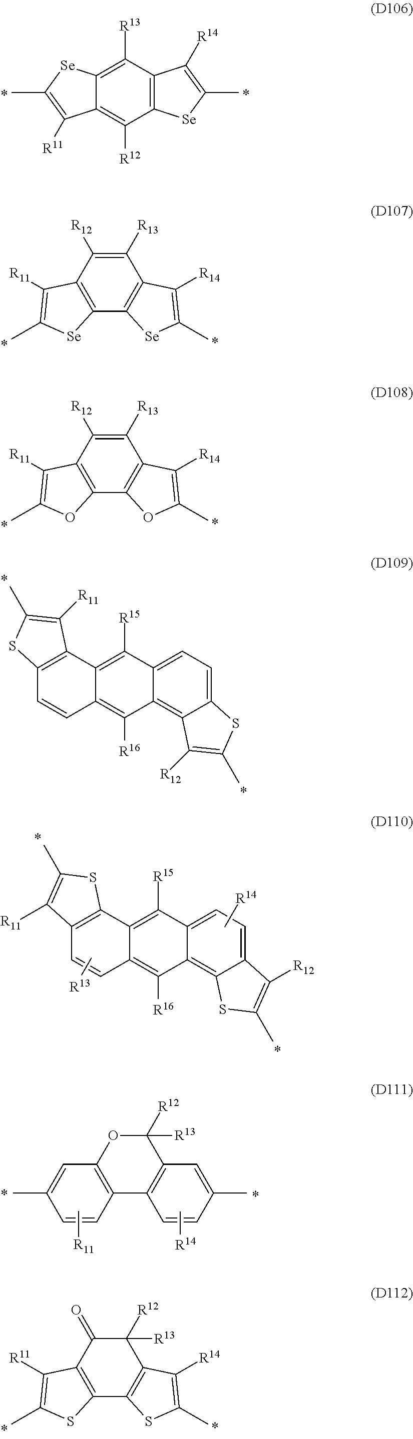

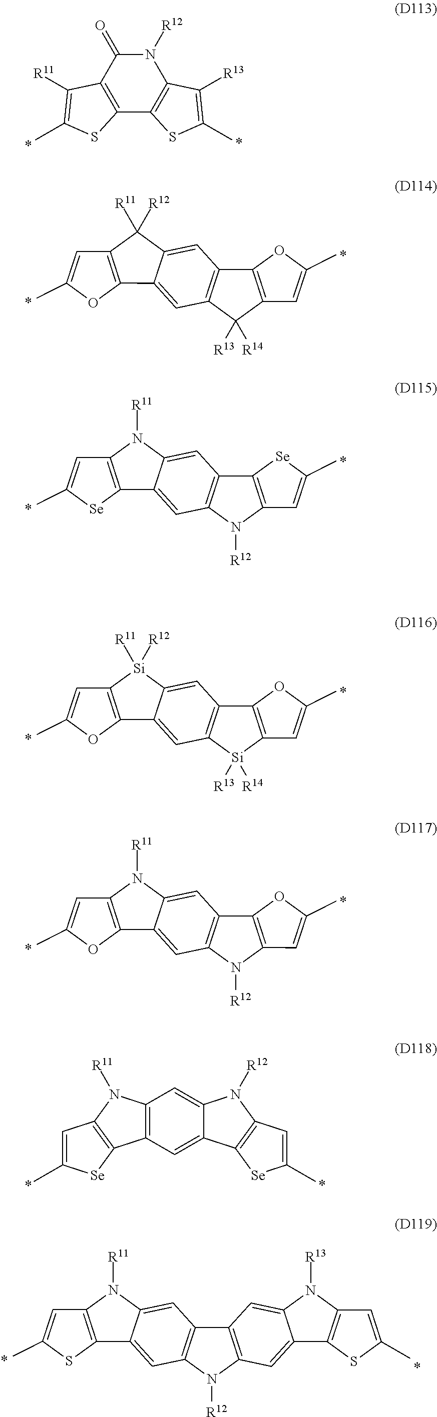

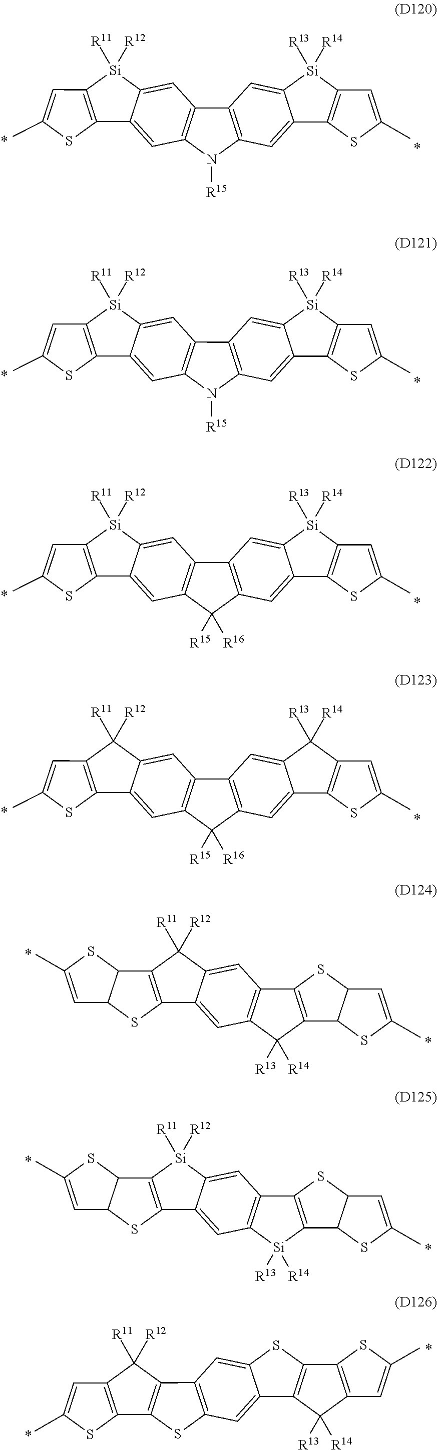

wherein one of X¹¹ and X¹² is S and the other is Se, and R¹¹, R¹², R¹³, R¹⁴, R¹⁵, R¹⁶, R¹⁷ and R¹⁸ being independently of each other selected from the group consisting of hydrogen, F, Br, Cl, —CN, —NC, —NCO, —NCS, —OCN, —SCN, —C(O)NR⁰R⁰⁰, —C(O)X⁰, —C(O)R⁰, —NH₂, —NR⁰R⁰⁰, —SH, —SR⁰, —SO₃H, —SO₂R⁰, —OH, —NO₂, —CF₃, —SF₅, optionally substituted silyl or hydrocarbyl with 1 to 60, preferably with 1 to 50 and more preferably with 1 to 40 C atoms that is optionally substituted and optionally comprises one or more hetero atoms, with X°, R⁰ and R⁰⁰ as defined earlier.

Suitable examples of aryl and heteroaryl with electron acceptor properties may be selected from the group consisting of the following formula (A-1) to (A-91)

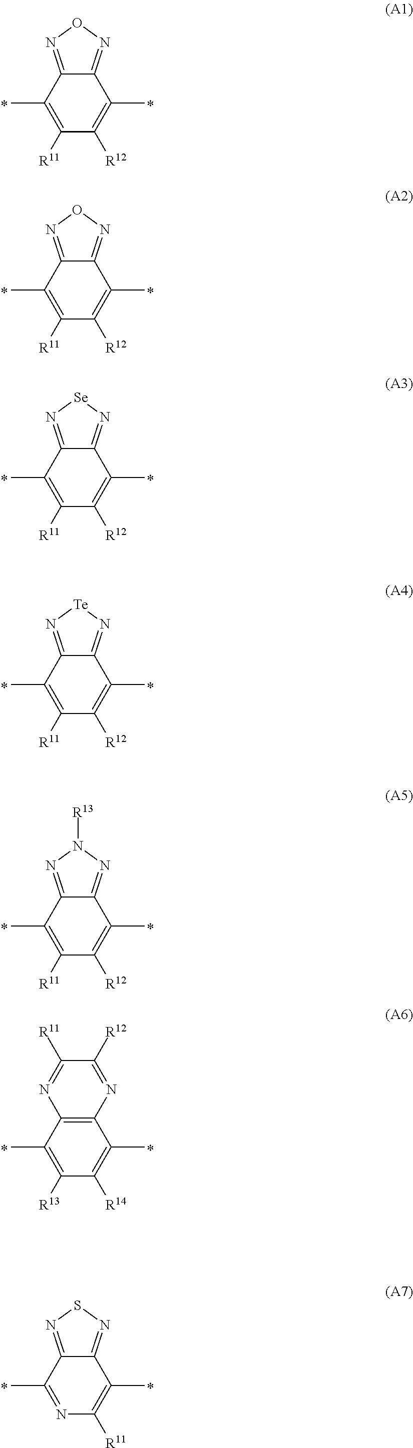

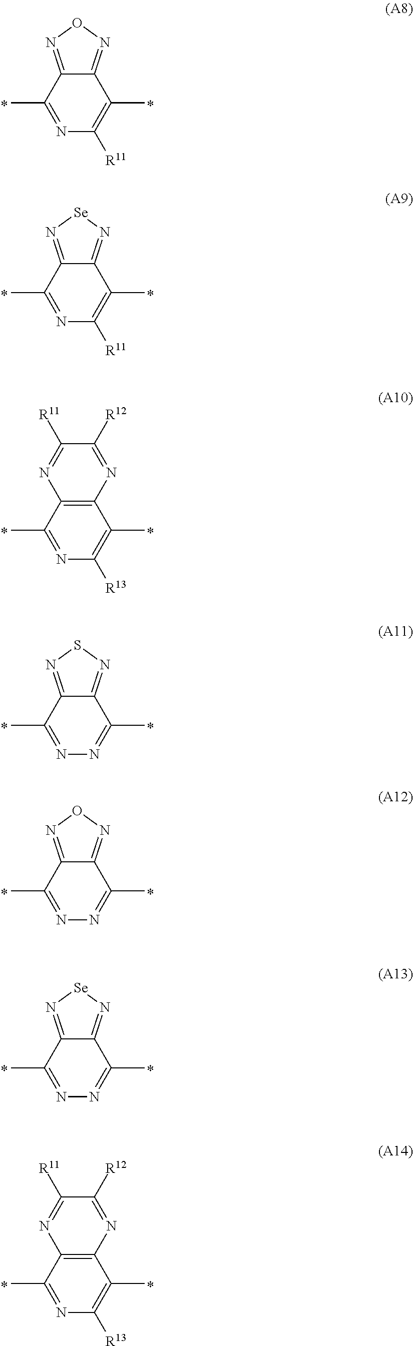

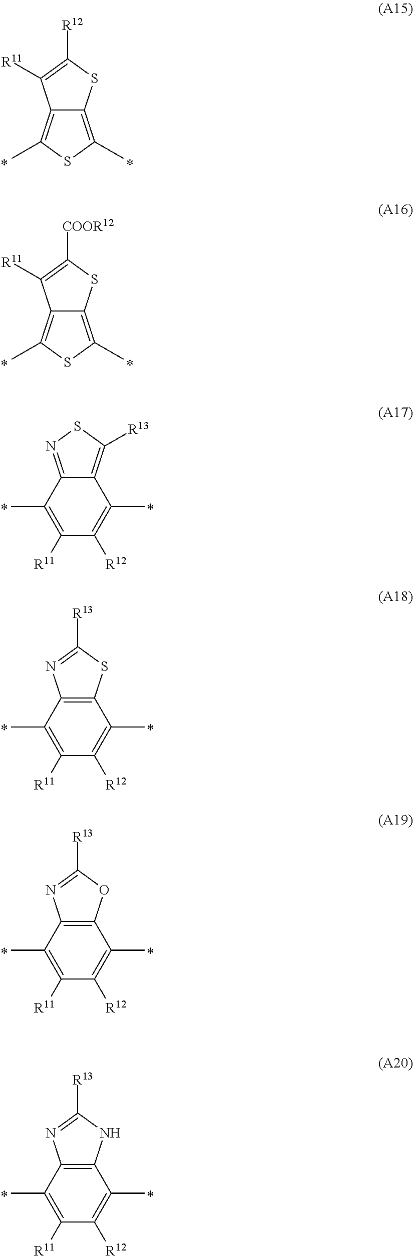

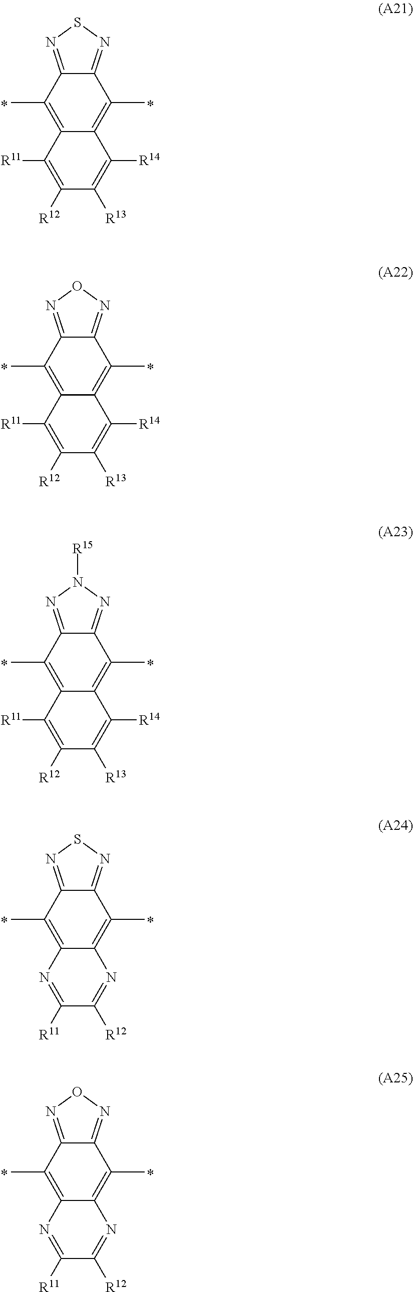

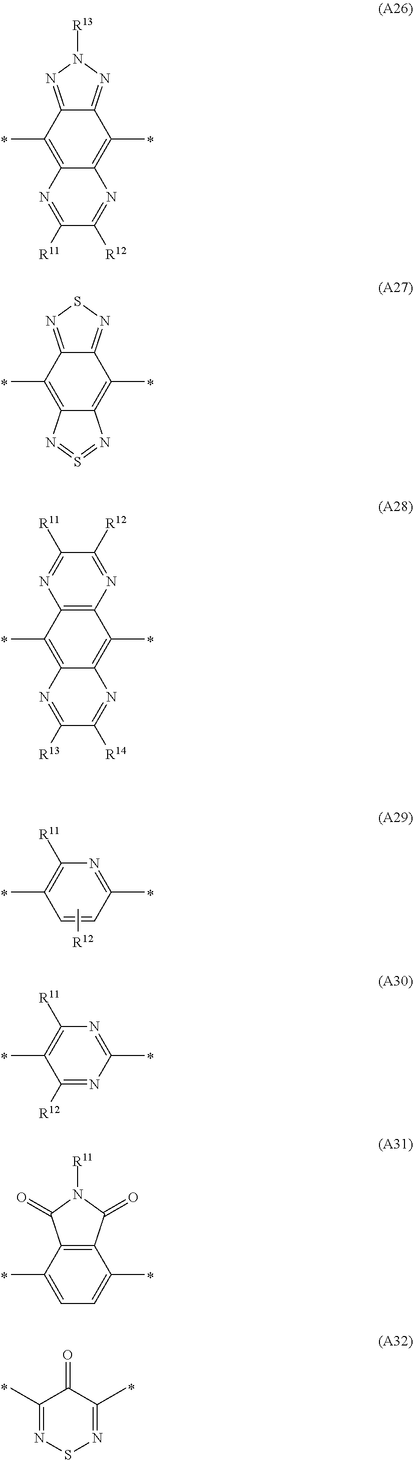

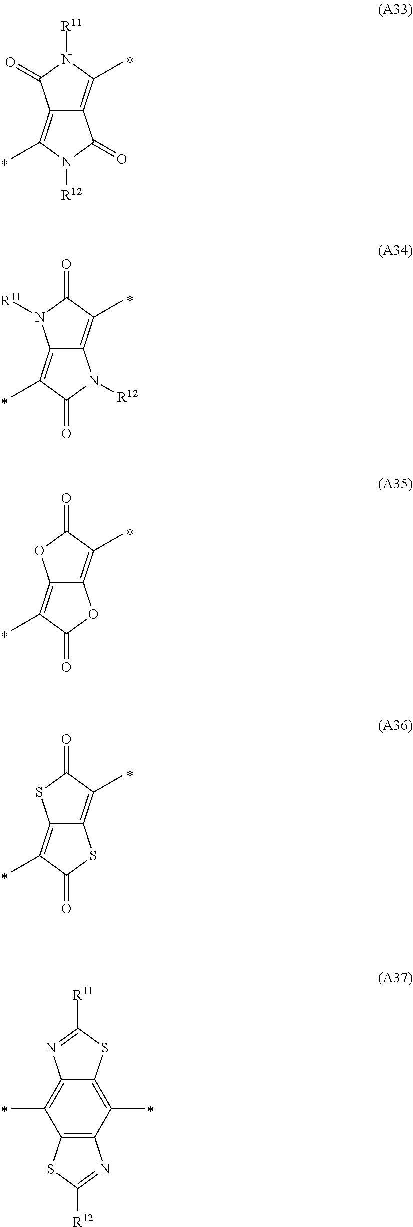

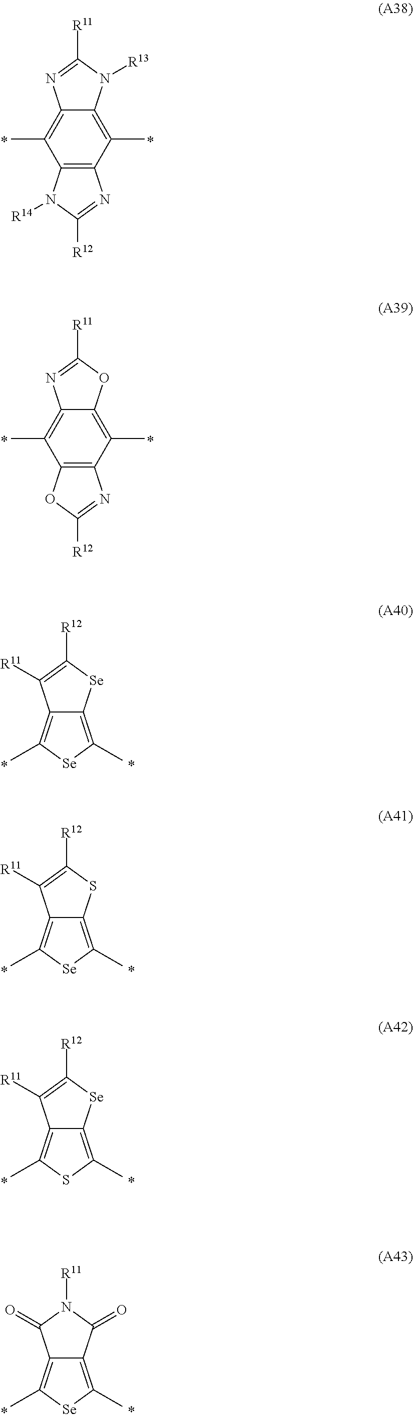

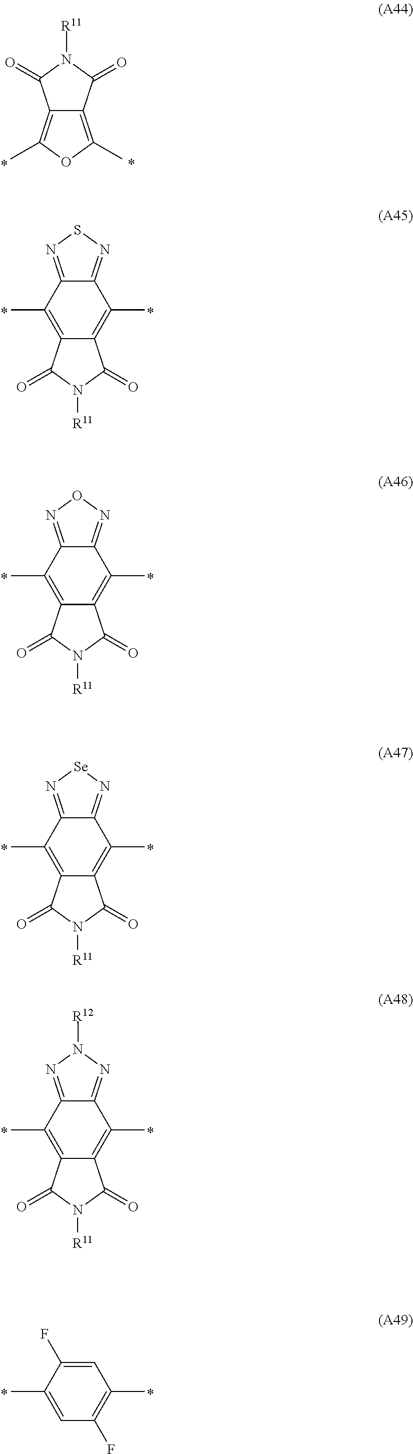

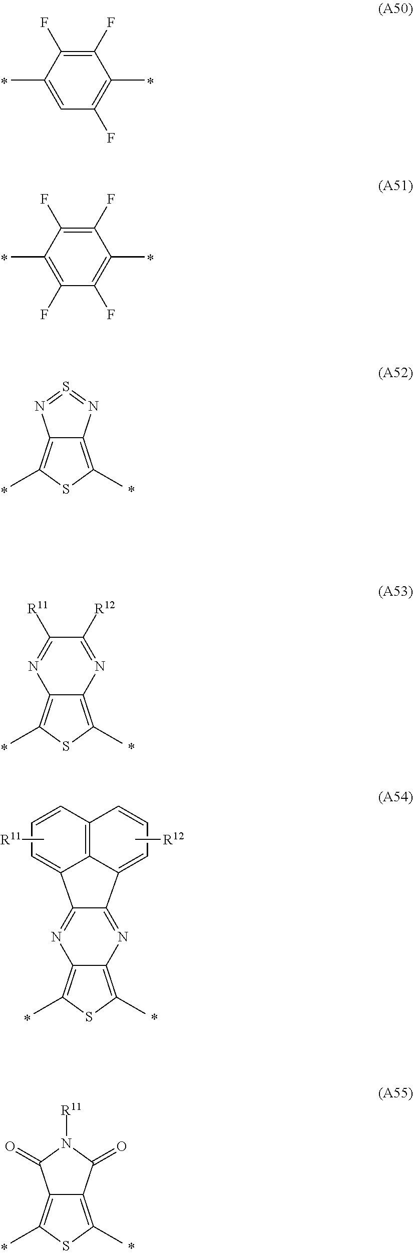

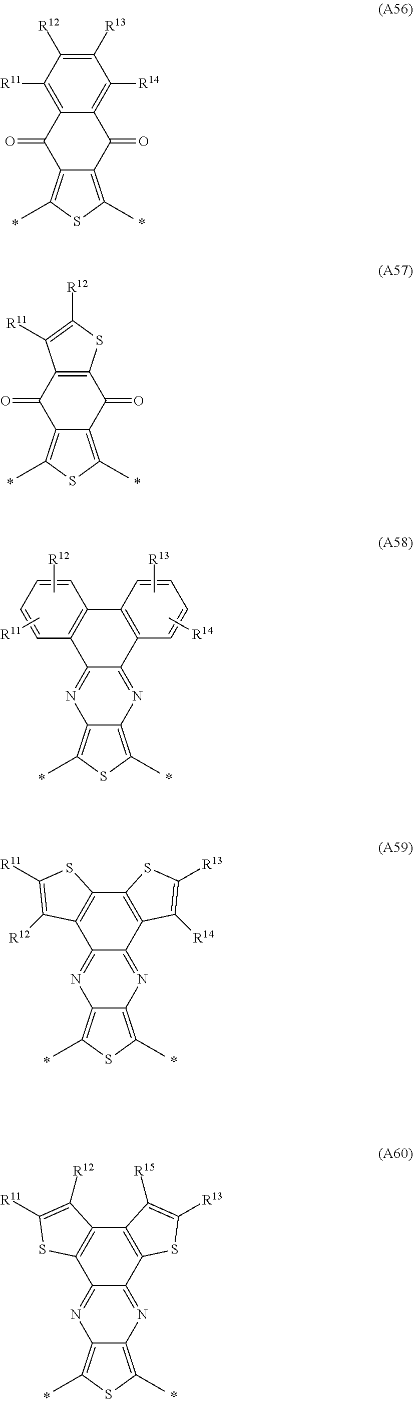

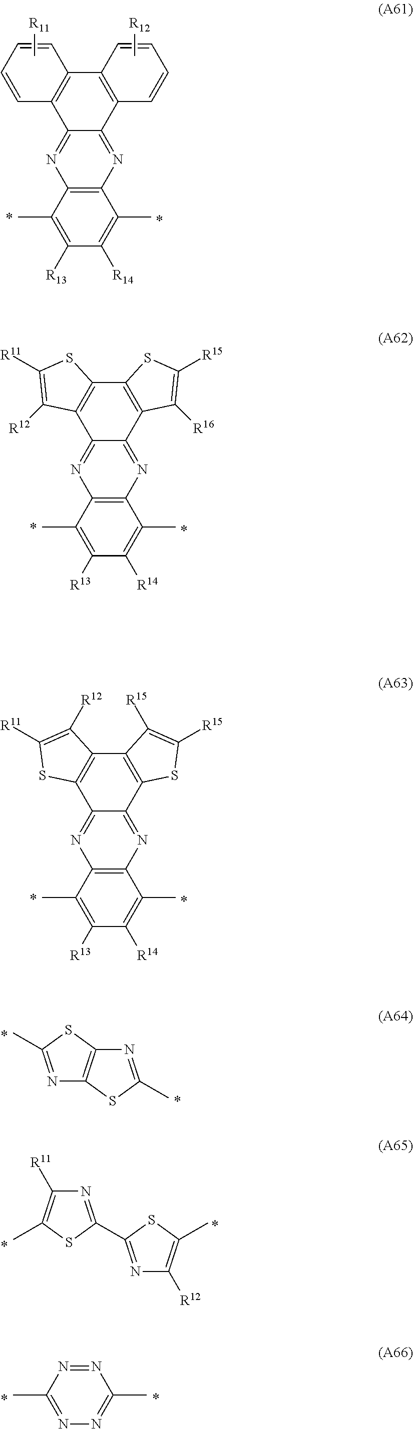

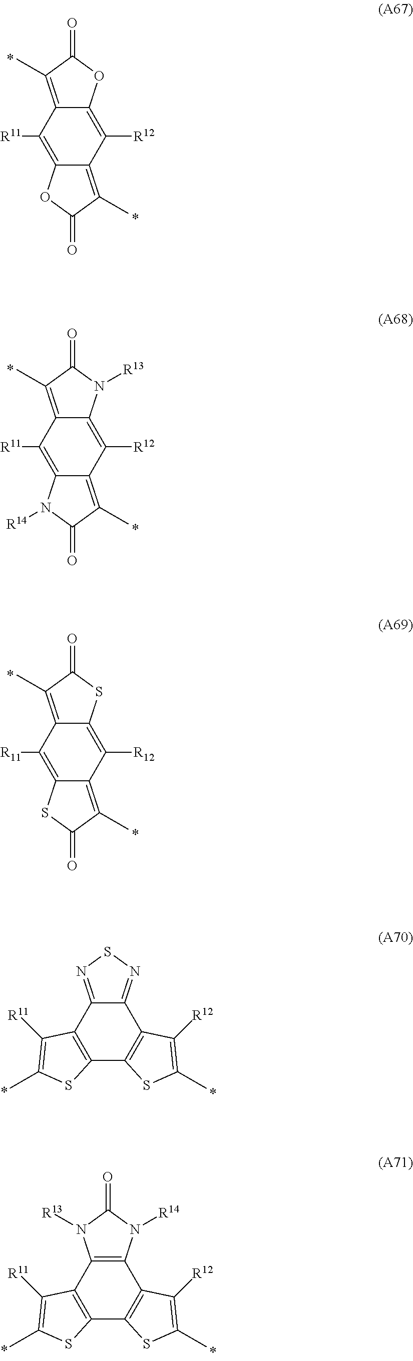

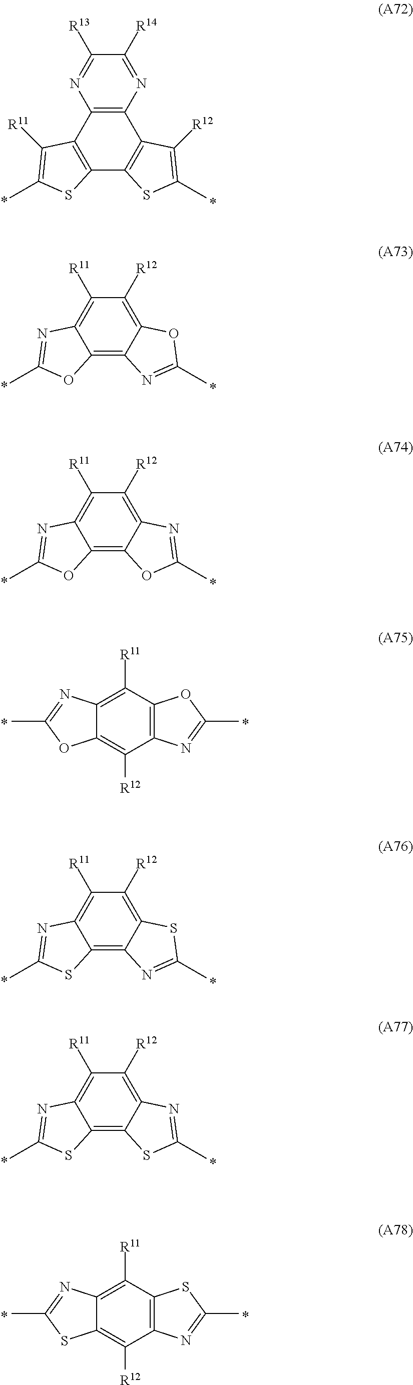

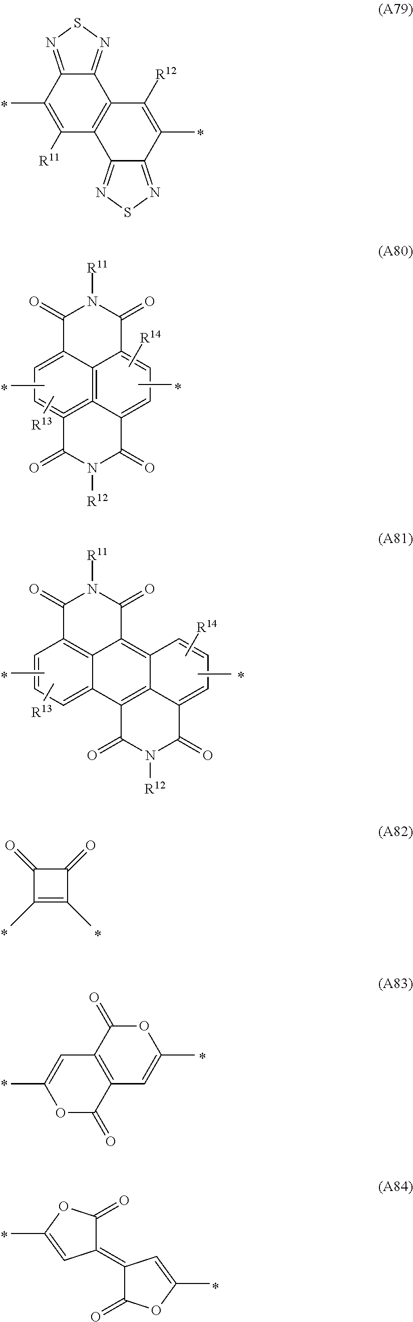

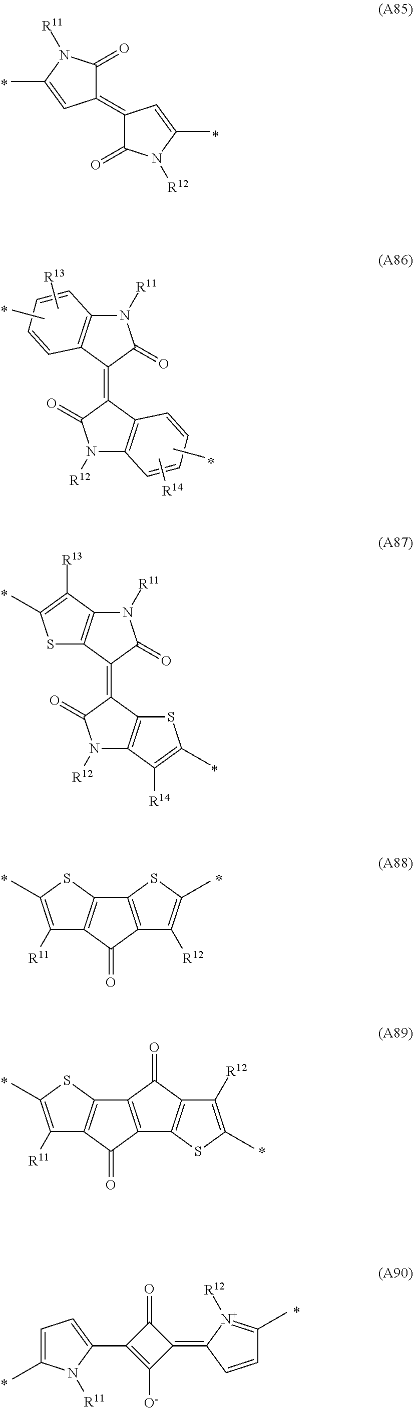

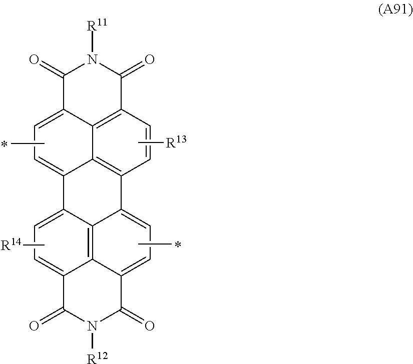

wherein one of X¹¹ and X¹² is S and the other is Se, and R¹¹, R¹², R¹³, R¹⁴ and R¹⁵ being independently of each other selected from the group consisting of hydrogen, F, Br, Cl, —CN, —NC, —NCO, —NCS, —OCN, —SCN, —C(O)NR⁰R⁰⁰, —C(O)X°, —C(O)R⁰, —NH₂, —NR⁰R⁰⁰, —SH, —SR⁰, —SO₃H, —SO₂R⁰, —OH, —NO₂, —CF₃, —SF₅, optionally substituted silyl or hydrocarbyl with 1 to 60, preferably with 1 to 50 and more preferably with 1 to 40 C atoms that is optionally substituted and optionally comprises one or more hetero atoms, with X⁰, R⁰ and R⁰⁰ as defined earlier.

Polymer

In a further aspect the present application provides for an oligomer or polymer, i.e. for a compound comprising more than one said fused-ring moiety. Preferably such oligomer or polymer comprises more than one group Q as defined in any one of formulae (III), (III-a-1), (III-a-2) and (III-b-1) to (III-b-5). At each occurrence Q may be the same or different.

Optionally, such oligomer or polymer may further comprise a repeating unit comprising a group selected from monocyclic or polycyclic aryl or heteroaryl groups that are optionally substituted. Preferably such further repeating units are selected from one of the following

*—[—Ar^{d}ₘ₆—Ar^{a}ₘ₂—Ar^{e}ₘ₇—Ar^{b}ₘ₄—Ar^{c}ₘ₅]—* (IV)

wherein
Ar^{a}, Ar^{b}, Ar^{c}, m2, m4 and m5 are as defined above;
Ar^{d} and Ar^{e} are independently of each aryl or heteroaryl with electron donor properties or electron acceptor properties, preferably independently of each other chosen from the group consisting of formulae (D-1) to (D-126) and (A-1) to (A-91); and
m6 and m7 are independently of each other 0, 1 or 2, provided that at least one of m6 and m7 is not 0 (for example m6 is 0 and m7 is 1, or m6 is 1 and m7 is 0, or m6 is 1 and m7 is 1).

Preferred oligomers and polymers may for example comprise a polymer chain of formula (V)

*(Q¹)ₘₓ-(Q²)_{my}-(Q³)_{mz}ₘ* (V)

wherein
m is an integer >1;
Q¹, Q² and Q³ are independently of each other monomeric units as defined below, provided that at least one of Q¹, Q² and Q³ comprises said fused-ring moiety;
mx is >0 and ≦1;
my is ≧0 and <1; and
mz is ≧0 and <1,

with the provision that mx+my+mz=1.

Preferably Q¹, Q² and Q³ are independently of each other selected from the group consisting of Q as defined in and for above formulae (III), (III-a-1), (III-a-2) and (III-b-1) to (III-b-5).

Examples of suitable polymer chains of formula (IV) may be selected from the following formulae (V-1) to (V-10)

*—[(Ar^{a}—U^{a}—Ar^{b})ₘₓ—(Ar^{c})_{my}]ₘ—* (V-1)

*—[(Ar^{a}—U^{a}—Ar^{b})ₘₓ—(Ar^{c}—Ar^{c})_{my}]ₘ—* (V-2)

*—[(Ar^{a}—U^{a}—Ar^{b})ₘₓ—(Ar^{c}—Ar^{c}—Ar^{c})_{my}]ₘ—* (V-3)

*—[(Ar^{a})ₘ₂—(U^{a})ₘ₁—(Ar^{b})ₘ₄—(Ar^{c})ₘ₅]ₘ—* (V-4)

*—([(Ar^{a})ₘ₂—(U^{a})ₘ₁—(Ar^{b})ₘ₄—(Ar^{c})ₘ₅]ₘₓ-[(Ar^{a})ₘ₂—(Ar^{d})ₘ₆—(Ar^{b})ₘ₄—(Ar^{c})ₘ₅- ]_{my})ₘ—* (V-5)

*—[(U^{a}—Ar^{a}—U^{b})ₘₓ—(Ar^{b}—Ar^{c})_{my}]ₘ—* (V-6)

*—[(U^{a}—Ar^{a}—U^{b})ₘₓ—(Ar^{b}—Ar^{c}—Ar^{b})_{my}]ₘ—* (V-7)

*—[(U^{a})ₘ₁—(Ar^{a})ₘ₂—(U^{b})ₘ₃—(Ar^{b})ₘ₄]ₘ—* (V-8)

*—([(U^{a})ₘ₁—(Ar^{a})ₘ₂—(U^{b})ₘ₃—(Ar^{b})ₘ₄-]ₘₓ-[(Ar^{d})ₘ₆—(Ar^{a})ₘ₂—(Ar^{e})ₘ₇—(Ar^{b})ₘ₄]_{my})ₘ—*(V-9)

*—[(U^{a}—Ar^{a})ₘₓ—(U^{b}—Ar^{b})_{my}—(U^{c}—Ar^{c})_{mz}]ₘ—* (V-10)

wherein Ar^{a}, Ar^{b}, Ar^{c}, Ar^{d}, Ar^{e} U^{a}, U^{b}, m1, m2, m3, m4, m5, m6, m7, m, mx, my and mz are as defined above, and U^{c} is as defined above for U^{a} and U^{b}.

Such polymers can be alternating or random copolymers. With respect to formulae (V-4) and (V-6) it is preferred that in at least one of the repeating units [(Ar^{a})ₘ₂—(U^{a})ₘ₁—(Ar^{b})ₘ₄—(Ar^{c})ₘ₅], and—if present—in at least one of the repeating units [(Ar^{a})ₘ₂—(Ar^{d})ₘ₆—(Ar^{b})ₘ₄—(Ar^{c})ₘ₅] m1 is at least 1 and m4 is at least 1. With respect to formulae (V-8) and (V-9) it is preferred that in at least one of the repeating units [(U^{a})ₘ₁—(Ar^{a})ₘ₂—(U^{b})ₘ₃—(Ar^{b})ₘ₄], and—if present—in at least one of the repeating units [(Ar^{d})ₘ₆—(Ar^{a})ₘ₂—(Ar^{e})ₘ₇—(Ar^{b})ₘ₄] m1 is at least 1 and m6 is at least 1.

For the present oligomers and polymers the total number m of repeating units is preferably from 2 to 10000. For a polymer the total number m of repeating units is preferably at least 10 and most preferably at least 50.

The present oligomers and polymers include homopolymers and copolymers, such as for example statistical or random copolymers, alternating copolymers and block copolymers as well as any combination of these.

Particularly preferred are polymers selected from the following groups
a) Group 1 consisting of homopolymers of the unit U^{a} or (Ar^{a}—U^{a}) or (Ar^{a}—U^{a}—Ar^{b}) or (Ar^{a}—U^{a}—Ar^{c}) or (U^{a}—Ar^{b}—Ar^{c}) or (Ar^{a}—U^{a}—Ar^{b}—Ar^{c}) or (U^{a}—Ar^{a}—U^{a}), i.e. where all repeating units are identical,
b) Group 2 consisting of random or alternating copolymers formed by identical units (Ar^{a}—U^{a}—Ar^{b}) or (U^{a}—Ar^{a}—U^{a}) and identical units (Ar^{c}),
c) Group 3 consisting of random or alternating copolymers formed by identical units (Ar^{a}—U^{a}—Ar^{b}) or (U^{a}—Ar^{a}—U^{b}) and identical units (Ar^{a}),
d) Group 4 consisting of random or alternating copolymers formed by identical units (Ar^{a}—U^{a}—Ar^{b}) or (U^{a}—Ar^{a}—U^{b}) and identical units (Ar^{a}—Ar^{d}—Ar^{b}) or (Ar^{d}—Ar^{a}—Ar^{e}),

wherein in all these groups Ar^{a}, Ar^{b}, Ar^{c}, Ar^{d}, Ar^{e}, U^{a} and U^{b} are as defined above and below, in groups 1, 2 and 3 Ar^{a}, Ar^{b} and Ar^{c} are different from a single bond, and in group 4 one of Ar^{a} and Ar^{b} may also denote a single bond.

Preferred polymers of formulae (V) and (V-1) to (V-10) may be those of formula (VI)

R^{e}-chain-R^{f} (VI)

wherein “chain” denotes a polymer chain of any one of formulae (V) or (V-1) to (V-10), and R^{e} and R^{f} have independently of each other one of the meanings of R^{S} as defined above, or denote, independently of each other, H, F, Br, Cl, I, —CH₂Cl, —CHO, —CR′═CR″₂, —SiR′R″R′″, —SiR′X″X′″, —SiR′R″X″, —SnR′R″R′″, —BR′R″, —B (OR′)(OR″), —B(OH)₂, —O—SO₂—R′, —C≡CH, —C≡C—SiR′₃, —ZnX″ or an endcap group, X″ and X′″ denote halogen, R′, R″ and R″ have independently of each other one of the meanings of R⁰ as defined earlier, and two of R′, R″ and R′″ may also form a ring together with the atom to which they are attached.

Preferred endcap groups R^{e} and R^{f} are H, C₁₋₆₀ alkyl, or optionally substituted C₆₋₁₂ aryl or C₂₋₁₀ heteroaryl. More preferred endcap groups R^{e} and R^{f} are H, alkyl having from 1 to 50 carbon atoms or phenyl. Even more preferred endcap groups R^{e} and R^{f} are H, alkyl having from 1 to 40 carbon atoms or phenyl. Still even more preferred endcap groups R^{e} and R^{f} are H, alkyl having from 1 to 30 or from 1 to 20 carbon atoms or phenyl. Most preferred endcap groups R^{e} and R^{f} are H, alkyl having from 1 to 10 carbon atoms or phenyl.

In the polymer chains of formulae (V) and (V-1) to (V-10) mx, my and mz denote the mole fraction of units Q¹, Q² and Q³, respectively, and m denotes the degree of polymerisation. These formulae are intended to include block copolymers, random or statistical copolymers and alternating copolymers of Q¹, Q² and Q³, as well as homopolymers of M¹ for the case when mx>0 and my=mz=0.

Further preferred are repeating units, monomers, oligomers and polymers of formulae (II-a), (II-b), (III), (III-a-1), (III-a-2), (III-b-1) to (III-b-5), (IV), (V), (V-1) to (V-10) and (VI) characterised by one or more of the following preferred or alternative aspects provided that such aspects are not mutually exclusive:
0<my<1 and mz=0;
0<my<1 and 0<mz<1;
M_{w} is at least 5,000, preferably at least 8,000, more preferably at least 10,000;
R¹ and R² are phenyl which is mono- or polysubstituted, preferably mono-substituted in the 4-position (i.e. the para-position), with substituents selected from linear or branched alkyl having from 1 to 60, preferably from 1 to 50, more preferably from 1 to 40, even more preferably from 1 to 30 and most preferably from 1 to 20 carbon atoms, wherein one or more H may optionally be replaced by F;
all groups R^{S} denote H;
at least one group R^{S} is different from H;
R^{S} may be selected independently at each occurrence from the group consisting of primary alkyl having from 1 to 60, preferably from 1 to 50, more preferably from 1 to 40 and most preferably from 1 to 30 carbon atoms, secondary alkyl having from 3 to 60, preferably from 1 to 50, more preferably from 1 to 40 and most preferably from 1 to 30 carbon atoms, and tertiary alkyl having from 4 to 60, preferably from 1 to 50, more preferably from 1 to 40 and most preferably from 1 to 30 carbon atoms, wherein one or more H may optionally be replaced by F;
R^{S} may be selected independently at each occurrence from the group consisting of aryl and heteroaryl, each of which may optionally be fluorinated, alkylated or alkoxylated and has from 4 to 30 ring atoms;
R^{S} is selected, on each occurrence identically or differently, from the group consisting of aryl and heteroaryl, each of which is optionally fluorinated, or alkylated and has 4 to 30 ring atoms,
R^{S} is selected, on each occurrence identically or differently, from the group consisting of primary alkoxy or sulfanylalkyl with 1 to 60, preferably from 1 to 50, more preferably from 1 to 40 and most preferably from 1 to 30 C atoms, secondary alkoxy or sulfanylalkyl with 3 to 60, preferably from 1 to 50, more preferably from 1 to 40 and most preferably from 1 to 30 C atoms, and tertiary alkoxy or sulfanylalkyl with 4 to 60, preferably from 1 to 50, more preferably from 1 to 40 and most preferably from 1 to 30 C atoms, wherein in all these groups one or more H atoms are optionally replaced by F,
R^{S} is selected, on each occurrence identically or differently, from the group consisting of aryloxy and heteroaryloxy, each of which is optionally alkylated or alkoxylated and has 4 to 30 ring atoms,
R^{S} is selected, on each occurrence identically or differently, from the group consisting of alkylcarbonyl, alkoxycarbonyl and alkylcarbonyloxy, all of which are straight-chain or branched, are optionally fluorinated, and have from 1 to 60, preferably from 1 to 50, more preferably from 1 to 40 and most preferably from 1 to 30 C atoms,
R^{S} denotes, on each occurrence identically or differently, F, Cl, Br, I, CN, R^{g}, —C(O)—R^{g}, —C(O)—O—R^{g}, or —O—C(O)—R^{g}, —SO₂—R^{g}, —SO₃—R^{g}, wherein R^{g} is straight-chain, branched or cyclic alkyl with 1 to 60, preferably from 1 to 50, more preferably from 1 to 40 and most preferably from 1 to 30 C atoms, in which one or more non-adjacent C atoms are optionally replaced by —O, —S—, —C(O)—, —C(O)—O—, —O—C(O)—, —O—C(O)—O—, —SO₂—, —SO₃—, —CR⁰═CR⁰⁰- or —C≡C— and in which one or more H atoms are optionally replaced by F, Cl, Br, I or CN, or R^{g} is aryl or heteroaryl having 4 to 30 ring atoms which is unsubstituted or which is substituted by one or more halogen atoms or by one or more groups R¹ as defined above,
R⁰ and R⁰⁰ are selected from H or C₁-C₆₀-alkyl, preferably from H or C₁-C₅₀-alkyl, more preferably from H or C₁-C₄₀-alkyl, even preferably from H or C₁-C₃₀-alkyl, still even preferably from H or C₁-C₂₀-alkyl and most preferably from H or C₁-C₁₀-alkyl,
R^{e} and R^{f} are independently of each other selected from H, halogen, —CH₂Cl, —CHO, —CH═CH₂—SiR′R″R′″, —SnR′R″R′″, —BR′R″, —B(OR′)(OR″), —B(OH)₂, C₁-C₆₀-alkyl, C₁-C₆₀-alkoxy, C₂-C₆₀-alkenyl, C₁-C₆₀-fluoroalkyl and optionally substituted aryl or heteroaryl, preferably phenyl,
R^{c} and R^{d} are independently of each other selected from the group consisting of Cl, Br, I, O-tosylate, O-triflate, O-mesylate, O-nonaflate, —SiMe₂F, —SiMeF₂, —O—SO₂Z¹, —B(OZ²)₂, —CZ³═C(Z⁴)₂, —C≡CH, C≡CSi(Z¹)₃, —ZnX⁰ and —Sn(Z⁴)₃, wherein X⁰ is halogen, and Z¹, Z², Z³ and Z⁴ are selected from the group consisting of alkyl and aryl, each being optionally substituted, and two groups Z² may also form a cyclic group.

The compounds of the present invention can be synthesized according to or in analogy to methods that are known to the skilled person and are described in the literature. Other methods of preparation can be taken from the examples. For example, the polymers can be suitably prepared by aryl-aryl coupling reactions, such as Yamamoto coupling, Suzuki coupling, Stille coupling, Sonogashira coupling, Heck coupling or Buchwald coupling. Suzuki coupling, Stille coupling and Yamamoto coupling are especially preferred. The monomers which are polymerized to form the repeat units of the polymers can be prepared according to methods which are known to the person skilled in the art.

Thus, the process for preparing the present polymers comprises the step of coupling monomers, therein comprised a monomer comprising said fused-ring moiety, said monomers comprising at least one or alternatively two functional monovalent group selected from the group consisting of Cl, Br, I, O-tosylate, O-triflate, O-mesylate, O-nonaflate, —SiMe₂F, —SiMeF₂, —O—SO₂Z¹, —B(OZ²)₂, —CZ³═C(Z³)₂, —C≡CH, —C≡CSi(Z¹)₃, —ZnX⁰ and —Sn(Z⁴)₃, wherein X⁰ is halogen, and Z¹, Z², Z³ and Z⁴ are independently of each other selected from the group consisting of alkyl and aryl, each being optionally substituted, and two groups Z² may also together form a cyclic group.

Preferably the polymers are prepared from monomers of general formula (II-b) or their preferred subformulae as described above and below.

Another aspect of the invention is a process for preparing a polymer by coupling one or more identical or different monomeric units comprising said fused-ring moiety or monomers of general formula (II-a) with each other and/or with one or more comonomers in a polymerisation reaction, preferably in an aryl-aryl, aryl-alkenyl, or aryl-alkynyl coupling reaction.

Suitable and preferred comonomers may be selected from the following formulae

R^{c}-(Ar^{a})ₘ₂—Ar^{d}-(Ar^{b})ₘ₄—R^{d} (VII-1)

R^{c}—Ar^{a}-R^{d} (VII-2)

R^{c}—Ar^{d}-R^{d} (VII-3)

wherein Ar^{a}, Ar^{b}, Ar^{d}, m2, m4, R^{c} and R^{d} are as defined herein.

Very preferred is a process for preparing a polymer by coupling one or more monomers selected from formula (III-a-1) or (III-a-2) with one or more monomers of formula (VII-1), and optionally with one or more monomers selected from formula (VII-2) and (VII-3), in an aryl-aryl coupling reaction, wherein preferably R^{c} and R^{d} are selected from Cl, Br, I, —B(OZ²)₂ and —Sn(Z⁴)₃.

For example, preferred embodiments of the present invention relate to

a) a process of preparing a polymer by coupling a monomer of formula (VII-1)

R^{c}—Ar^{a}—U^{a}—Ar^{b}-R^{d}

with a monomer of formula (VII-2)

R^{c}—Ar^{a}-R^{d} (VII-2)

in an aryl-aryl coupling reaction; or

b) a process of preparing a polymer by coupling a monomer of formula

R^{c}—U^{a}—R^{d}

with a monomer of formula (VII-1)

R^{c}—Ar^{a}—Ar^{d}—Ar^{b}-R^{d} (VII-1)

in an aryl-aryl coupling reaction; or

c) a process of preparing a polymer by coupling a monomer of formula

R^{c}—U^{a}—R^{d}

with a monomer of formula (VII-3)

R^{c}—Ar^{d}-R^{d} (VII-3)

in an aryl-aryl coupling reaction; or

d) a process of preparing a polymer by coupling a monomer of formula

R^{c}—U^{a}—R^{d}

with a monomer of formula (VII-3)

R^{c}—Ar^{d}-R^{d} (VII-3)

and a monomer of formula (VII-2)

R^{c}—Ar^{a}-R^{d} (VII-2)

in an aryl-aryl coupling reaction; or

e) a process of preparing a polymer by coupling a monomer of formula

R^{c}—U^{a}—Ar^{a}—U^{b}—R^{d}

with a monomer of formula (VII-2)

R^{c}—Ar^{a}-R^{d} (VII-2)

in an aryl-aryl coupling reaction; or

a process of preparing a polymer by coupling a monomer of formula

R^{c}—U^{a}—R^{d}

with a monomer of formula (VII-2)

R^{c}—Ar^{a}-R^{d} (VII-2)

and a monomer of formula (VII-3)

R^{c}—Ar^{d}-R^{d} (VII-3)

in an aryl-aryl coupling reaction,

wherein Ar^{a}, Ar^{b}, Ar^{d}, U^{a}, U^{b}, R^{c} and R^{d} are as defined herein, with R^{c} and R^{d} preferably selected from Cl, Br, I, —B(OZ²)₂ and —Sn(Z⁴)₃ as defined in respect to formulae (II-a) and (II-b).

Preferred aryl-aryl coupling and polymerisation methods used in the processes described above and below are Yamamoto coupling, Kumada coupling, Negishi coupling, Suzuki coupling, Stille coupling, Sonogashira coupling, Heck coupling, C—H activation coupling, Ullmann coupling or Buchwald coupling. Especially preferred are Suzuki coupling, Negishi coupling, Stille coupling and Yamamoto coupling. Suzuki coupling is described for example in WO 00/53656 A1. Negishi coupling is described for example in J. Chem. Soc., Chem. Commun., 1977, 683-684. Yamamoto coupling is described for example in T. Yamamoto et al., Prog. Polym. Sci., 1993, 17, 1153-1205, or WO 2004/022626 A1, and Stille coupling is described for example in Z. Bao et al., J. Am. Chem. Soc., 1995, 117, 12426-12435. For example, when using Yamamoto coupling, monomers having two reactive halide groups are preferably used. When using Suzuki coupling, compounds of formula (IV-b) having two reactive boronic acid or boronic acid ester groups or two reactive halide groups are preferably used. When using Stille coupling, monomers having two reactive stannane groups or two reactive halide groups are preferably used. When using Negishi coupling, monomers having two reactive organozinc groups or two reactive halide groups are preferably used.

Preferred catalysts, especially for Suzuki, Negishi or Stille coupling, are selected from Pd(0) complexes or Pd(II) salts. Preferred Pd(0) complexes are those bearing at least one phosphine ligand, for example Pd(Ph₃P)₄. Another preferred phosphine ligand is tris(ortho-tolyl)phosphine, for example Pd(o-Tol₃P)₄. Preferred Pd(II) salts include palladium acetate, for example Pd(OAc)₂. Alternatively the Pd(0) complex can be prepared by mixing a Pd(0) dibenzylideneacetone complex, for example tris(dibenzyl-ideneacetone)dipalladium(0), bis(dibenzylideneacetone)-palladium(0), or Pd(II) salts e.g. palladium acetate, with a phosphine ligand, for example triphenylphosphine, tris(ortho-tolyl)phosphine or tri(tert-butyl)phosphine. Suzuki polymerisation is performed in the presence of a base, for example sodium carbonate, potassium carbonate, lithium hydroxide, potassium phosphate or an organic base such as tetraethylammonium carbonate or tetraethylammonium hydroxide. Yamamoto polymerisation employs a Ni(0) complex, for example bis(1,5-cyclooctadienyl)nickel(0).

Suzuki and Stille polymerisation may be used to prepare homopolymers as well as statistical, alternating and block random copolymers. Statistical or block copolymers can be prepared for example from the above monomers of formula (VI) or its subformulae, wherein one of the reactive groups is halogen and the other reactive group is a boronic acid, boronic acid derivative group or and alkylstannane. The synthesis of statistical, alternating and block copolymers is described in detail for example in WO 03/048225 A2 or WO 2005/014688 A2.

As alternatives to halogens as described above, leaving groups of formula —O—SO₂Z¹ can be used wherein Z¹ is as described above. Particular examples of such leaving groups are tosylate, mesylate and triflate.

Blends, Formulations and Devices

The compounds and polymers according to the present invention can also be used in mixtures or polymer blends, for example together with small molecules or monomeric compounds or together with other polymers having charge-transport, semiconducting, electrically conducting, photoconducting and/or light emitting semiconducting properties, or for example with polymers having hole blocking or electron blocking properties for use as interlayers or charge blocking layers in OLED devices. Thus, another aspect of the invention relates to a polymer blend comprising one or more polymers according to the present invention and one or more further polymers having one or more of the above-mentioned properties. These blends can be prepared by conventional methods that are described in the prior art and are known to the skilled person. Typically the polymers are mixed with each other or dissolved in suitable solvents and the solutions combined.

Another aspect of the invention relates to a formulation comprising one or more small molecules, polymers, mixtures or polymer blends as described above and below and one or more organic solvents.

Preferred solvents are aliphatic hydrocarbons, chlorinated hydrocarbons, aromatic hydrocarbons, ketones, ethers and mixtures thereof. Additional solvents which can be used include 1,2,4-trimethylbenzene, 1,2,3,4-tetra-methyl benzene, pentylbenzene, mesitylene, cumene, cymene, cyclohexylbenzene, diethylbenzene, tetralin, decalin, 2,6-lutidine, 2-fluoro-m-xylene, 3-fluoro-o-xylene, 2-chlorobenzotrifluoride, N,N-dimethylformamide, 2-chloro-6-fluorotoluene, 2-fluoroanisole, anisole, 2,3-dimethylpyrazine, 4-fluoroanisole, 3-fluoroanisole, 3-trifluoro-methylanisole, 2-methylanisole, phenetol, 4-methylanisole, 3-methylanisole, 4-fluoro-3-methylanisole, 2-fluorobenzonitrile, 4-fluoroveratrol, 2,6-dimethylanisole, 3-fluorobenzo-nitrile, 2,5-dimethylanisole, 2,4-dimethylanisole, benzonitrile, 3,5-dimethyl-anisole, N,N-dimethylaniline, ethyl benzoate, 1-fluoro-3,5-dimethoxy-benzene, 1-methylnaphthalene, N-methylpyrrolidinone, 3-fluorobenzo-trifluoride, benzotrifluoride, dioxane, trifluoromethoxy-benzene, 4-fluorobenzotrifluoride, 3-fluoropyridine, toluene, 2-fluoro-toluene, 2-fluorobenzotrifluoride, 3-fluorotoluene, 4-isopropylbiphenyl, phenyl ether, pyridine, 4-fluorotoluene, 2,5-difluorotoluene, 1-chloro-2,4-difluorobenzene, 2-fluoropyridine, 3-chlorofluoro-benzene, 1-chloro-2,5-difluorobenzene, 4-chlorofluorobenzene, chloro-benzene, o-dichlorobenzene, 2-chlorofluorobenzene, p-xylene, m-xylene, o-xylene or mixture of o-, m-, and p-isomers. Solvents with relatively low polarity are generally preferred. For inkjet printing solvents and solvent mixtures with high boiling temperatures are preferred. For spin coating alkylated benzenes like xylene and toluene are preferred.

Examples of especially preferred solvents include, without limitation, dichloromethane, trichloromethane, chlorobenzene, o-dichlorobenzene, tetrahydrofuran, anisole, morpholine, toluene, o-xylene, m-xylene, p-xylene, 1,4-dioxane, acetone, methylethylketone, 1,2-dichloroethane, 1,1,1-trichloroethane, 1,1,2,2-tetrachloroethane, ethyl acetate, n-butyl acetate, N,N-dimethylformamide, dimethylacetamide, dimethylsulfoxide, tetraline, decaline, indane, methyl benzoate, ethyl benzoate, mesitylene and/or mixtures thereof.

The concentration of the compounds or polymers in the solution is preferably 0.1 to 10% by weight, more preferably 0.5 to 5% by weight, with % by weight given relative to the total weight of the solution. Optionally, the solution also comprises one or more binders to adjust the rheological properties, as described for example in WO 2005/055248 A1.

After appropriate mixing and ageing, solutions are evaluated as one of the following categories: complete solution, borderline solution or insoluble. The contour line is drawn to outline the solubility parameter-hydrogen bonding limits dividing solubility and insolubility. ‘Complete’ solvents falling within the solubility area can be chosen from literature values such as published in J. D. Crowley et al., Journal of Paint Technology, 1966, 38 (496), 296. Solvent blends may also be used and can be identified as described in Solvents, W. H. Ellis, Federation of Societies for Coatings Technology, p. 9-10, 1986. Such a procedure may lead to a blend of ‘non’-solvents that will dissolve both the polymers of the present invention, although it is desirable to have at least one true solvent in a blend.

The compounds and polymers according to the present invention can also be used in patterned OSC layers in the devices as described above and below. For applications in modern microelectronics it is generally desirable to generate small structures or patterns to reduce cost (more devices/unit area), and power consumption. Patterning of thin layers comprising a polymer according to the present invention can be carried out for example by photolithography, electron beam lithography or laser patterning.

For use as thin layers in electronic or electrooptical devices the compounds, polymers, polymer blends or formulations of the present invention may be deposited by any suitable method. Liquid coating of devices is more desirable than vacuum deposition techniques. Solution deposition methods are especially preferred. The formulations of the present invention enable the use of a number of liquid coating techniques. Preferred deposition techniques include, without limitation, dip coating, spin coating, ink jet printing, nozzle printing, letter-press printing, screen printing, gravure printing, doctor blade coating, roller printing, reverse-roller printing, offset lithography printing, dry offset lithography printing, flexographic printing, web printing, spray coating, curtain coating, brush coating, slot dye coating or pad printing.

Ink jet printing is particularly preferred when high resolution layers and devices need to be prepared. Selected formulations of the present invention may be applied to prefabricated device substrates by ink jet printing or microdispensing. Preferably industrial piezoelectric print heads such as but not limited to those supplied by Aprion, Hitachi-Koki, InkJet Technology, On Target Technology, Picojet, Spectra, Trident, Xaar may be used to apply the organic semiconductor layer to a substrate. Additionally semi-industrial heads such as those manufactured by Brother, Epson, Konica, Seiko Instruments Toshiba TEC or single nozzle microdispensers such as those produced by Microdrop and Microfab may be used.

In order to be applied by ink jet printing or microdispensing, the compounds or polymers should be first dissolved in a suitable solvent. Solvents must fulfill the requirements stated above and must not have any detrimental effect on the chosen print head. Additionally, solvents should have boiling points >100° C., preferably >140° C. and more preferably >150° C. in order to prevent operability problems caused by the solution drying out inside the print head. Apart from the solvents mentioned above, suitable solvents include substituted and non-substituted xylene derivatives, di-C₁₋₂-alkyl formamide, substituted and non-substituted anisoles and other phenol-ether derivatives, substituted heterocycles such as substituted pyridines, pyrazines, pyrimidines, pyrrolidinones, substituted and non-substituted N,N-di-C₁₋₂-alkylanilines and other fluorinated or chlorinated aromatics.

A preferred solvent for depositing a compound or polymer according to the present invention by ink jet printing comprises a benzene derivative which has a benzene ring substituted by one or more substituents wherein the total number of carbon atoms among the one or more substituents is at least three. For example, the benzene derivative may be substituted with a propyl group or three methyl groups, in either case there being at least three carbon atoms in total. Such a solvent enables an ink jet fluid to be formed comprising the solvent with the compound or polymer, which reduces or prevents clogging of the jets and separation of the components during spraying. The solvent(s) may include those selected from the following list of examples: dodecylbenzene, 1-methyl-4-tert-butylbenzene, terpineol, limonene, isodurene, terpinolene, cymene, diethylbenzene. The solvent may be a solvent mixture, that is a combination of two or more solvents, each solvent preferably having a boiling point >100° C., more preferably >140° C. Such solvent(s) also enhance film formation in the layer deposited and reduce defects in the layer.

The ink jet fluid (that is mixture of solvent, binder and semiconducting compound) preferably has a viscosity at 20° C. of 1-100 mPa·s, more preferably 1-50 mPa·s and most preferably 1-30 mPa·s.

The polymer blends and formulations according to the present invention can additionally comprise one or more further components or additives selected for example from surface-active compounds, lubricating agents, wetting agents, dispersing agents, hydrophobing agents, adhesive agents, flow improvers, defoaming agents, deaerators, diluents which may be reactive or non-reactive, auxiliaries, colourants, dyes or pigments, sensitizers, stabilizers, nanoparticles or inhibitors.

The compounds and polymers to the present invention are useful as charge transport, semiconducting, electrically conducting, photoconducting or light emitting materials in optical, electrooptical, electronic, electroluminescent or photoluminescent components or devices. In these devices, the polymers of the present invention are typically applied as thin layers or films.

Thus, the present invention also provides the use of the semiconducting compound, polymer, polymers blend, formulation or layer in an electronic device. The formulation may be used as a high mobility semiconducting material in various devices and apparatus. The formulation may be used, for example, in the form of a semiconducting layer or film. Accordingly, in another aspect, the present invention provides a semiconducting layer for use in an electronic device, the layer comprising a compound, polymer blend or formulation according to the invention. The layer or film may be less than about 30 microns. For various electronic device applications, the thickness may be less than about 1 micron thick. The layer may be deposited, for example on a part of an electronic device, by any of the aforementioned solution coating or printing techniques.

The invention additionally provides an electronic device comprising a compound, polymer, polymer blend, formulation or organic semiconducting layer according to the present invention. Preferred devices are OFETs, TFTs, ICs, logic circuits, capacitors, RFID tags, OLEDs, OLETs, OPEDs, OPVs, OPDs, solar cells, laser diodes, photoconductors, photodetectors, electrophotographic devices, electrophotographic recording devices, organic memory devices, sensor devices, charge injection layers, Schottky diodes, planarising layers, antistatic films, conducting substrates and conducting patterns. Particularly preferred devices are OLEDs.

Especially preferred electronic device are OFETs, OLEDs, OPV and OPD devices, in particular bulk heterojunction (BHJ) OPV devices. In an OFET, for example, the active semiconductor channel between the drain and source may comprise the layer of the invention. As another example, in an OLED device, the charge (hole or electron) injection or transport layer may comprise the layer of the invention.

For use in OPV or OPD devices the polymer according to the present invention is preferably used in a formulation that comprises or contains, more preferably consists essentially of, very preferably exclusively of, a p-type (electron donor) semiconductor and an n-type (electron acceptor) semiconductor. The n-type semiconductor is constituted by a polymer according to the present invention.

Preferably the polymer according to the present invention is blended with a p-type semiconductor, to form the active layer in an OPV or OPD device. The device preferably further comprises a first transparent or semi-transparent electrode on a transparent or semi-transparent substrate on one side of the active layer, and a second metallic or semi-transparent electrode on the other side of the active layer. Further preferably the OPV or OPD device comprises, between the active layer and the first or second electrode, one or more additional buffer layers acting as hole transporting layer and/or electron blocking layer, which comprise a material such as metal oxide, like for example, ZTO, MoOₓ, NiOₓ, a conjugated polymer electrolyte, like for example PEDOT:PSS, a conjugated polymer, like for example polytriarylamine (PTAA), an organic compound, like for example N,N′-diphenyl-N,N′-bis(1-naphthyl)(1,1′-biphenyl)-4,4′diamine (NPB), N,N′-diphenyl-N,N′-(3-methylphenyl)-1,1′-biphenyl-4,4′-diamine (TPD), or alternatively as hole blocking layer and/or electron transporting layer, which comprise a material such as metal oxide, like for example, ZnOₓ, TiOₓ, a salt, like for example LiF, NaF, CsF, a conjugated polymer electrolyte, like for example poly[3-(6-trimethylammoniumhexyl)thiophene], poly(9,9-bis(2-ethylhexyl)-fluorene]-b-poly[3-(6-trimethylammoniumhexyl)thiophene], or poly[(9,9-bis(3′-(N,N-dimethyl-amino)propyl)-2,7-fluorene)-alt-2,7-(9,9-dioctylfluorene)] or an organic compound, like for example tris(8-quinolinolato)-aluminium(III) (Alq₃), 4,7-diphenyl-1,10-phenanthroline.

To produce thin layers in BHJ OPV devices the compounds, polymers, polymer blends or formulations of the present invention may be deposited by any suitable method. Liquid coating of devices is more desirable than vacuum deposition techniques. Solution deposition methods are especially preferred. The formulations of the present invention enable the use of a number of liquid coating techniques. Preferred deposition techniques include, without limitation, dip coating, spin coating, ink jet printing, nozzle printing, letter-press printing, screen printing, gravure printing, doctor blade coating, roller printing, reverse-roller printing, offset lithography printing, dry offset lithography printing, flexographic printing, web printing, spray coating, curtain coating, brush coating, slot dye coating or pad printing. For the fabrication of OPV devices and modules area printing method compatible with flexible substrates are preferred, for example slot dye coating, spray coating and the like.

In the preparation of formulations, suitable solvent must be selected to ensure full dissolution of both component, p-type and n-type and take into account the boundary conditions (for example rheological properties) introduced by the chosen printing method.

Organic solvent are generally used for this purpose. Typical solvents can be aromatic solvents, halogenated solvents or chlorinated solvents, including chlorinated aromatic solvents. Examples include, but are not limited to chlorobenzene, 1,2-dichlorobenzene, chloroform, 1,2-dichloroethane, dichloromethane, carbon tetrachloride, toluene, cyclohexanone, ethylacetate, tetrahydrofuran, anisole, morpholine, o-xylene, m-xylene, p-xylene, 1,4-dioxane, acetone, methylethylketone, 1,2-dichloroethane, 1,1,1-trichloroethane, 1,1,2,2-tetrachloroethane, ethyl acetate, n-butyl acetate, dimethylformamide, dimethylacetamide, dimethylsulfoxide, tetraline, decaline, indane, methyl benzoate, ethyl benzoate, mesitylene and combinations thereof.

For use in OPV or OPD devices that comprise or contain, more preferably consists essentially of, very preferably exclusively of, a p-type (electron donor) semiconductor and an n-type (electron acceptor) semiconductor, whereas the n-type semiconductor is constituted by a polymer according to the present invention. The p-type semiconductor in the said OPV or OPD devices can be an organic or inorganic material. The p-type organic semiconductor material used in the said OPV or OPD devices can be either small molecules or oligomers such as for example copper phthalocyanine, zinc phthalocyanine, pentacene, sexithiophenes, and any other p-type small molecule or oligomeric semiconductor (see e.g. Mishra, A. and Bäuerle, P. Angew. Chem. Int. Ed. 2012, 51, 2020-2067; Huang, Q. L. and Li, H. X. Chinese Science Bulletin 2013, 58, 2677-2686; Sun, Y., et al. Nat Mater. 2011, 11, 44-8; Kyaw, A. K. K., et al. Adv. Mater. 2013, 25, 2397-2402), and a polymer semiconductor such as for example poly(3-hexyl)thiophene and any other suitable polymer (see e.g. Thompson, B. C. and Fréchet, J. M. J. Angew. Chem. Int. Ed. 2008, 47, 58-77; Dennler, G., et al. Adv. Mater. 2009, 21, 1323-1338; Cheng, Y. J., et al. Chem. Rev. 2009, 109, 5868-5923; Facchetti, A. Materials Today 2013, 16, 123-132). The p-type inorganic semiconductor material use in the said OPV or OPD devices can be p-type silicon, copper(I) sulfide (Cu₂S), copper(I) oxide (Cu₂O), cooper(II) oxide (CuO), copper indium gallium selenide (CIGS), and any other suitable inorganic semiconductor. The solvents and formulation procedures that are suitable for processing OPV or OPD devices described above can also be used for the n-type semiconductor that is constituted by a polymer according to the present invention.

The OPV device can for example be of any type known from the literature (see e.g. Waldauf et al., Appl. Phys. Lett., 2006, 89, 233517) including very preferably an OPV device where the photoactive layer comprises a p-type polymer semiconductor and an n-type polymer semiconductor (see e.g. Halls, J. J. et al. Nature, 1995, 376, 498-500), where the n-type polymer semiconductor is constituted by a polymer according to the present invention.

A first preferred OPV device according to the invention comprises the following layers (in the sequence from bottom to top):
optionally a substrate,
a high work function electrode, preferably comprising a metal oxide, like for example ITO, serving as anode,
an optional conducting polymer layer or hole transport layer, preferably comprising an organic poymer or polymer blend, for example of PEDOT:PSS (poly(3,4-ethylenedioxythiophene): poly(styrene-sulfonate), or TBD (N,N′-dyphenyl-N—N′-bis(3-methylphenyl)-1,1′biphenyl-4,4′-diamine) or NBD (N,N′-dyphenyl-N—N′-bis(1-napthylphenyl)-1,1′biphenyl-4,4′-diamine),
a layer, also referred to as “active layer”, comprising a p-type and an n-type organic semiconductor, which can exist for example as a p-type/n-type bilayer or as distinct p-type and n-type layers, or as blend or p-type and n-type semiconductor, forming a BHJ,
optionally a layer having electron transport properties, for example comprising LiF,
a low work function electrode, preferably comprising a metal like for example aluminum, serving as cathode,

wherein at least one of the electrodes, preferably the anode, is transparent to visible light, and

wherein the n-type semiconductor is a polymer according to the present invention.

A second preferred OPV device according to the invention is an inverted OPV device and comprises the following layers (in the sequence from bottom to top):
optionally a substrate,
a high work function metal or metal oxide electrode, comprising for example ITO, serving as cathode,
a layer having hole blocking properties, preferably comprising a metal oxide like TiOₓ or Znₓ,
an active layer comprising a p-type and an n-type organic semiconductor, situated between the electrodes, which can exist for example as a p-type/n-type bilayer or as distinct p-type and n-type layers, or as blend or p-type and n-type semiconductor, forming a BHJ,
an optional conducting polymer layer or hole transport layer, preferably comprising an organic poymer or polymer blend, for example of PEDOT:PSS or TBD or NBD,
an electrode comprising a high work function metal like for example silver, serving as anode,

wherein at least one of the electrodes, preferably the cathode, is transparent to visible light, and

wherein the n-type semiconductor is a polymer according to the present invention.

When the active layer is deposited on the substrate, it forms a BHJ that phase separates at nanoscale level. For discussion on nanoscale phase separation see Dennler et al, Proceedings of the IEEE, 2005, 93 (8), 1429 or Hoppe et al, Adv. Func. Mater, 2004, 14(10), 1005. An optional annealing step may be then necessary to optimize blend morpohology and consequently OPV device performance.

Another method to optimize device performance is to prepare formulations for the fabrication of OPV (BHJ) devices that may include high boiling point additives to promote phase separation in the right way. 1,8-Octanedithiol, 1,8-diiodooctane, nitrobenzene, chloronaphthalene, and other additives have been used to obtain high-efficiency solar cells. Examples are disclosed in J. Peet, et al, Nat. Mater., 2007, 6, 497 or Fréchet et al. J. Am. Chem. Soc., 2010, 132, 7595-7597.

The compounds, polymers, formulations and layers of the present invention are also suitable for use in an OFET as the semiconducting channel. Accordingly, the invention also provides an OFET comprising a gate electrode, an insulating (or gate insulator) layer, a source electrode, a drain electrode and an organic semiconducting channel connecting the source and drain electrodes, wherein the organic semiconducting channel comprises a compound, polymer, polymer blend, formulation or organic semiconducting layer according to the present invention. Other features of the OFET are well known to those skilled in the art.

OFETs where an OSC material is arranged as a thin film between a gate dielectric and a drain and a source electrode, are generally known, and are described for example in U.S. Pat. No. 5,892,244, U.S. Pat. No. 5,998,804, U.S. Pat. No. 6,723,394 and in the references cited in the background section. Due to the advantages, like low cost production using the solubility properties of the compounds according to the invention and thus the processibility of large surfaces, preferred applications of these FETs are such as integrated circuitry, TFT displays and security applications.

The gate, source and drain electrodes and the insulating and semiconducting layer in the OFET device may be arranged in any sequence, provided that the source and drain electrode are separated from the gate electrode by the insulating layer, the gate electrode and the semiconductor layer both contact the insulating layer, and the source electrode and the drain electrode both contact the semiconducting layer.

An OFET device according to the present invention preferably comprises:
a source electrode,
a drain electrode,
a gate electrode,
a semiconducting layer,
one or more gate insulator layers, and
optionally a substrate,

wherein the semiconductor layer preferably comprises a compound, polymer, polymer blend or formulation as described above and below.

The OFET device can be a top gate device or a bottom gate device. Suitable structures and manufacturing methods of an OFET device are known to the skilled in the art and are described in the literature, for example in US 2007/0102696 A1.

The gate insulator layer preferably comprises a fluoropolymer, like e.g. the commercially available Cytop 809M® or Cytop 107M® (from Asahi Glass). Preferably the gate insulator layer is deposited, e.g. by spin-coating, doctor blading, wire bar coating, spray or dip coating or other known methods, from a formulation comprising an insulator material and one or more solvents with one or more fluoro atoms (fluorosolvents), preferably a perfluorosolvent. A suitable perfluorosolvent is e.g. FC75® (available from Acros, catalogue number 12380). Other suitable fluoropolymers and fluorosolvents are known in prior art, like for example the perfluoropolymers Teflon AF® 1600 or 2400 (from DuPont) or Fluoropel® (from Cytonix) or the perfluorosolvent FC 43® (Acros, No. 12377). Especially preferred are organic dielectric materials having a low permittivity (or dielectric contant) from 1.0 to 5.0, very preferably from 1.8 to 4.0 (“low k materials”), as disclosed for example in US 2007/0102696 A1 or U.S. Pat. No. 7,095,044.

In security applications, OFETs and other devices with semiconducting materials according to the present invention, like transistors or diodes, can be used for RFID tags or security markings to authenticate and prevent counterfeiting of documents of value like banknotes, credit cards or ID cards, national ID documents, licenses or any product with monetary value, like stamps, tickets, shares, cheques etc.

Alternatively, the materials according to the invention can be used in OLEDs, e.g. as the active display material in a flat panel display applications, or as backlight of a flat panel display like e.g. a liquid crystal display. Common OLEDs are realized using multilayer structures. An emission layer is generally sandwiched between one or more electron-transport and/or hole-transport layers. By applying an electric voltage electrons and holes as charge carriers move towards the emission layer where their recombination leads to the excitation and hence luminescence of the lumophor units contained in the emission layer. The inventive compounds, materials and films may be employed in one or more of the charge transport layers and/or in the emission layer, corresponding to their electrical and/or optical properties. Furthermore their use within the emission layer is especially advantageous, if the compounds, materials and films according to the invention show electroluminescent properties themselves or comprise electroluminescent groups or compounds. The selection, characterization as well as the processing of suitable monomeric, oligomeric and polymeric compounds or materials for the use in OLEDs is generally known by a person skilled in the art, see, e.g., Müller et al, Synth. Metals, 2000, 111-112, 31-34, Alcala, J. Appl. Phys., 2000, 88, 7124-7128 and the literature cited therein.

According to another use, the materials according to this invention, especially those showing photoluminescent properties, may be employed as materials of light sources, e.g. in display devices, as described in EP 0 889 350 A1 or by C. Weder et al., Science, 1998, 279, 835-837.

A further aspect of the invention relates to both the oxidised and reduced form of the compounds according to this invention. Either loss or gain of electrons results in formation of a highly delocalised ionic form, which is of high conductivity. This can occur on exposure to common dopants. Suitable dopants and methods of doping are known to those skilled in the art, e.g. from EP 0 528 662, U.S. Pat. No. 5,198,153 or WO 96/21659.

The doping process typically implies treatment of the semiconductor material with an oxidating or reducing agent in a redox reaction to form delocalised ionic centres in the material, with the corresponding counterions derived from the applied dopants. Suitable doping methods comprise for example exposure to a doping vapor in the atmospheric pressure or at a reduced pressure, electrochemical doping in a solution containing a dopant, bringing a dopant into contact with the semiconductor material to be thermally diffused, and ion-implantation of the dopant into the semiconductor material.

When electrons are used as carriers, suitable dopants are for example halogens (e.g., I₂, Cl₂, Br₂, ICl, ICl₃, Mr and IF), Lewis acids (e.g., PF₅, AsF₅, SbF₅, BF₃, BCl₃, SbCl₅, BBr₃ and SO₃), protonic acids, organic acids, or amino acids (e.g., HF, HCl, HNO₃, H₂SO₄, HClO₄, FSO₃H and ClSO₃H), transition metal compounds (e.g., FeCl₃, FeOCl, Fe(ClO₄)₃, Fe(4-CH₃C₆H₄SO₃)₃, TiCl₄, ZrCl₄, HfCl₄, NbF₅, NbCl₅, TaCl₅, MoF₅, MoCl₅, WF₅, WCl₆, UF₆ and LnCl₃ (wherein Ln is a lanthanoid), anions (e.g., Cl⁻, Br⁻, I⁻, I₃⁻, HSO₄⁻, SO₄²⁻, NO₃⁻, ClO₄⁻, BF₄⁻, PF₆⁻, AsF₆⁻, SbF₆⁻, FeCl₄⁻, Fe(CN)₆³⁻, and anions of various sulfonic acids, such as aryl-SO₃⁻). When holes are used as carriers, examples of dopants are cations (e.g., H⁺, Li⁺, Na⁺, K⁺, Rb⁺and Cs⁺), alkali metals (e.g., Li, Na, K, Rb, and Cs), alkaline-earth metals (e.g., Ca, Sr, and Ba), O₂, XeOF₄, (NO₂⁺) (SbF₆⁻), (NO₂⁺) (SbCl₆⁻), (NO₂⁺) (BF₄⁻), AgClO₄, H₂IrCl₆, La(NO₃)₃.6H₂O, FSO₂OOSO₂F, europium acetylcholine, R₄N⁺, (R is an alkyl group), R₄P⁺ (R is an alkyl group), R₆As⁺ (R is an alkyl group), and R₃S⁺ (R is an alkyl group).

The conducting form of the compounds of the present invention can be used as an organic “metal” in applications including, but not limited to, charge injection layers and ITO planarising layers in OLED applications, films for flat panel displays and touch screens, antistatic films, printed conductive substrates, patterns or tracts in electronic applications such as printed circuit boards and condensers.

The compounds and formulations according to the present invention may also be suitable for use in organic plasmon-emitting diodes (OPEDs), as described for example in Koller et al., Nat. Photonics, 2008, 2, 684.

According to another use, the materials according to the present invention can be used alone or together with other materials in or as alignment layers in LCD or OLED devices, as described for example in US 2003/0021913. The use of charge transport compounds according to the present invention can increase the electrical conductivity of the alignment layer. When used in an LCD, this increased electrical conductivity can reduce adverse residual dc effects in the switchable LCD cell and suppress image sticking or, for example in ferroelectric LCDs, reduce the residual charge produced by the switching of the spontaneous polarisation charge of the ferroelectric LCs. When used in an OLED device comprising a light emitting material provided onto the alignment layer, this increased electrical conductivity can enhance the electroluminescence of the light emitting material. The compounds or materials according to the present invention having mesogenic or liquid crystalline properties can form oriented anisotropic films as described above, which are especially useful as alignment layers to induce or enhance alignment in a liquid crystal medium provided onto said anisotropic film. The materials according to the present invention may also be combined with photoisomerisable compounds and/or chromophores for use in or as photoalignment layers, as described in US 2003/0021913 A1.

According to another use the materials according to the present invention, especially their water-soluble derivatives (for example with polar or ionic side groups) or ionically doped forms, can be employed as chemical sensors or materials for detecting and discriminating DNA sequences. Such uses are described for example in L. Chen, D. W. McBranch, H. Wang, R. Helgeson, F. Wudl and D. G. Whitten, Proc. Natl. Acad. Sci. U.S.A., 1999, 96, 12287; D. Wang, X. Gong, P. S. Heeger, F. Rininsland, G. C. Bazan and A. J. Heeger, Proc. Natl. Acad. Sci. U.S.A., 2002, 99, 49; N. DiCesare, M. R. Pinot, K. S. Schanze and J. R. Lakowicz, Langmuir, 2002, 18, 7785; D. T. McQuade, A. E. Pullen, T. M. Swager, Chem. Rev., 2000, 100, 2537.

Unless the context clearly indicates otherwise, as used herein plural forms of the terms herein are to be construed as including the singular form and vice versa.

Throughout the description and claims of this specification, the words “comprise” and “contain” and variations of the words, for example “comprising” and “comprises”, mean “including but not limited to”, and are not intended to (and do not) exclude other components.

It will be appreciated that variations to the foregoing embodiments of the invention can be made while still falling within the scope of the invention. Each feature disclosed in this specification, unless stated otherwise, may be replaced by alternative features serving the same, equivalent or similar purpose. Thus, unless stated otherwise, each feature disclosed is one example only of a generic series of equivalent or similar features.

All of the features disclosed in this specification may be combined in any combination, except combinations where at least some of such features and/or steps are mutually exclusive. In particular, the preferred features of the invention are applicable to all aspects of the invention and may be used in any combination. Likewise, features described in non-essential combinations may be used separately (not in combination).

Above and below, unless stated otherwise percentages are percent by weight and temperatures are given in degrees Celsius. The values of the dielectric constant c (“permittivity”) refer to values taken at 20° C. and 1,000 Hz.

The invention will now be described in detail with respect to specific representative embodiments thereof, it being understood that these examples are intended to be illustrative only and the invention is not intended to be limited to the materials, conditions, or process parameters recited herein.

### EXEMPLARY EMBODIMENTS

Exemplary embodiments of methods according to the invention as well as reactants and further processes that may be used are described below.

The synthesis of exemplary polymers PIBDFT-24, PIBDFBT-24, PIBDFV-24, PIBDFV-26, PIBDFV-40, and PIBDFBT-40 is outlined in Scheme 1, Scheme 2, and Scheme 3.

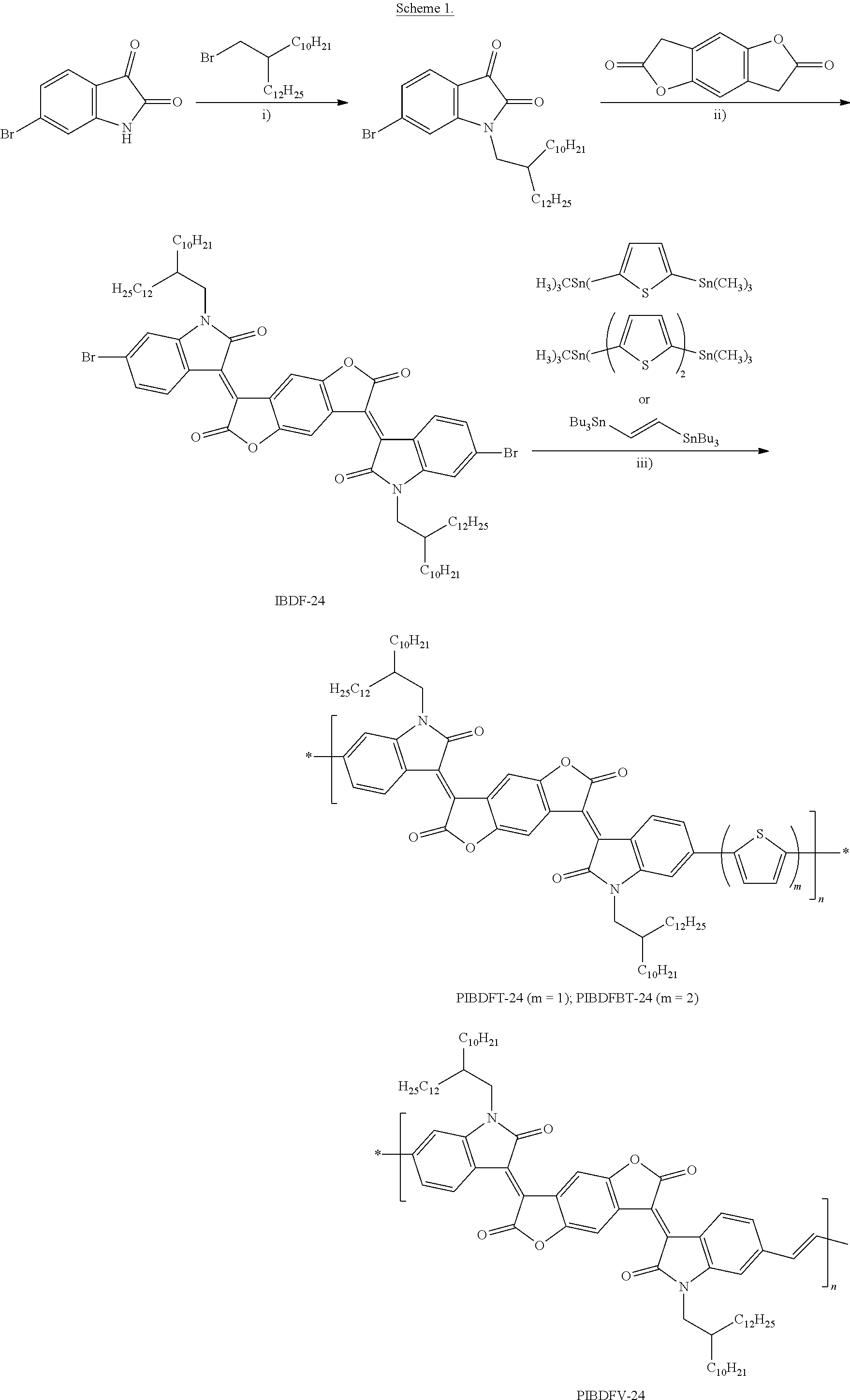

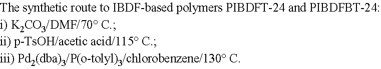

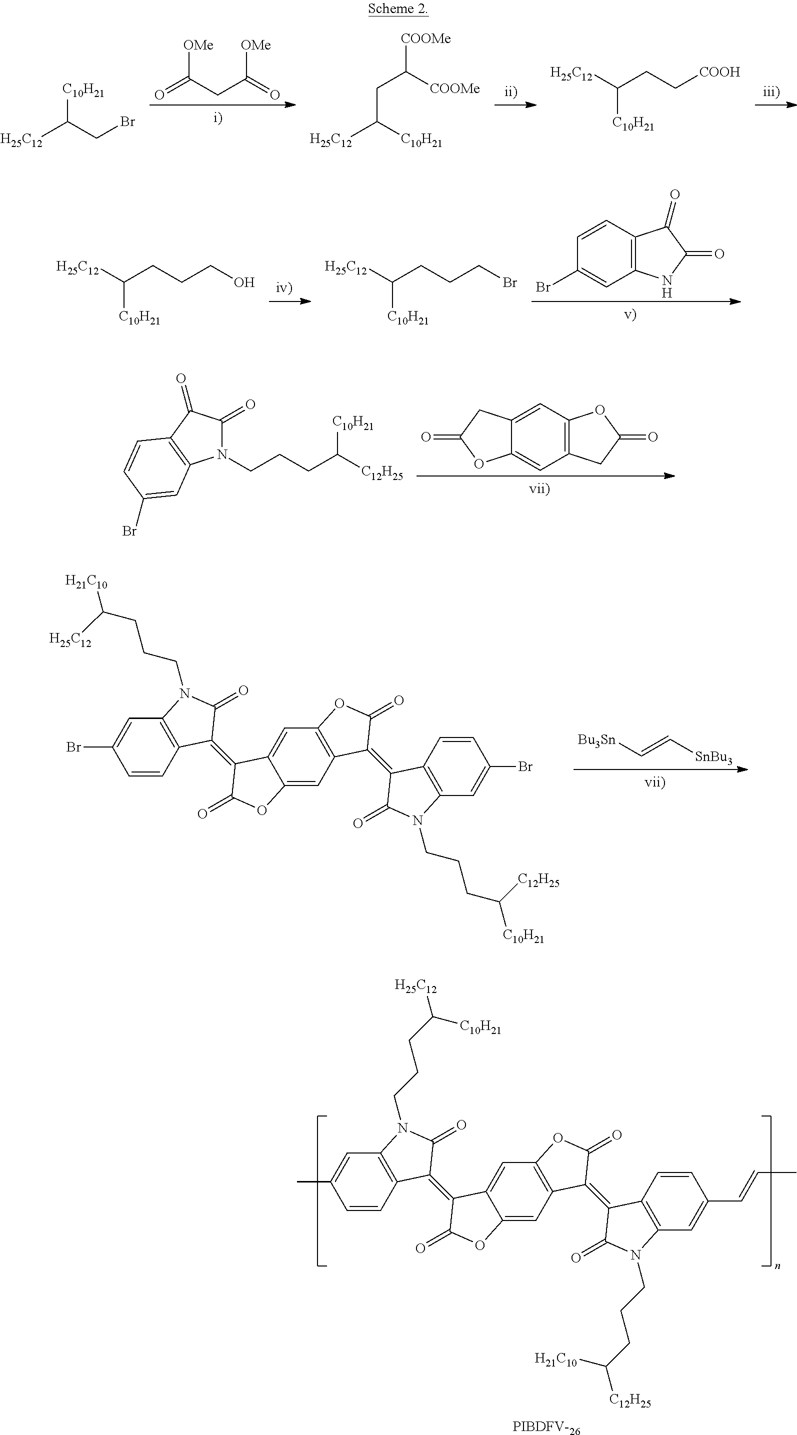

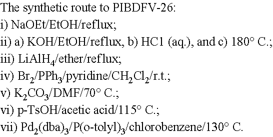

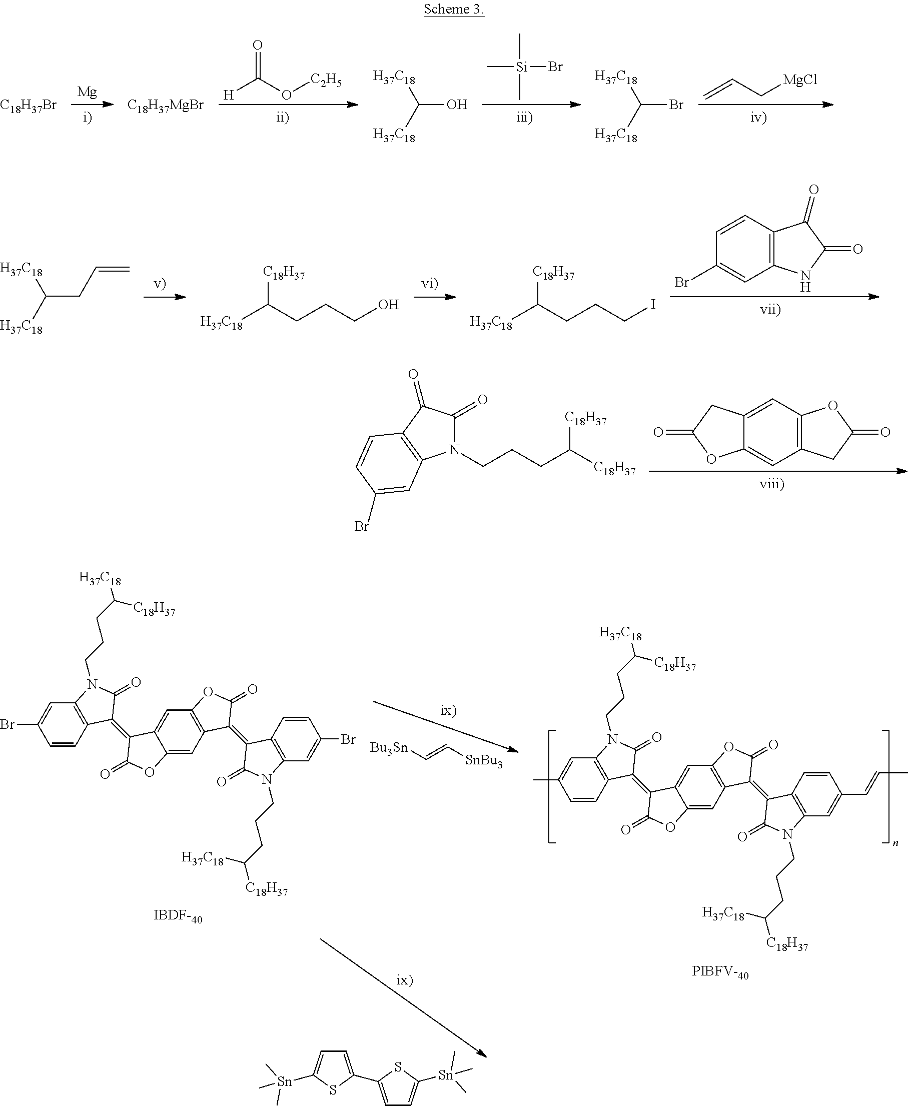

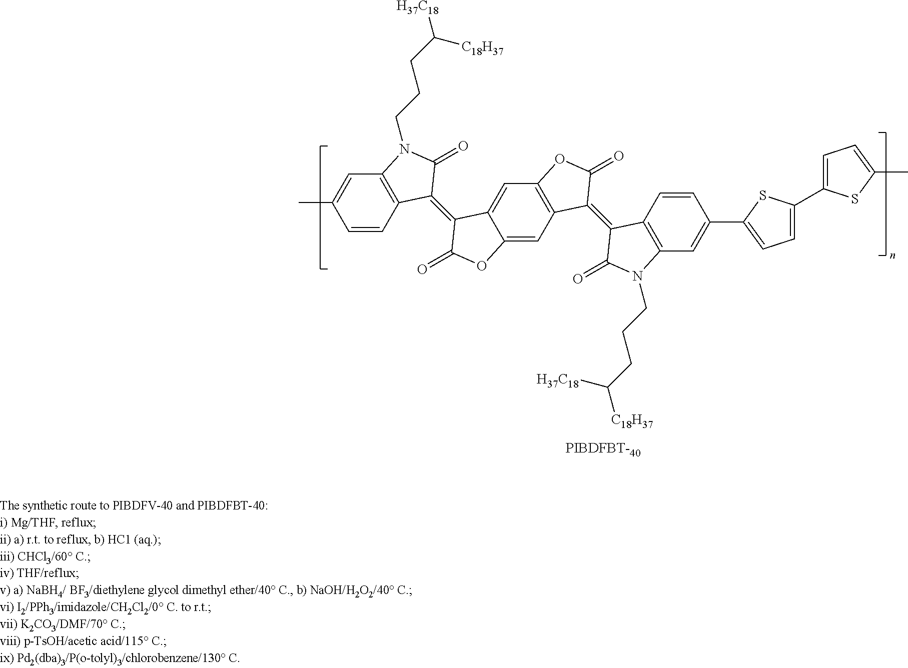

### EXAMPLES

### Example 1

### Synthesis of 6-bromo-1-(2-decyltetradecyl)indoline-2,3-dione

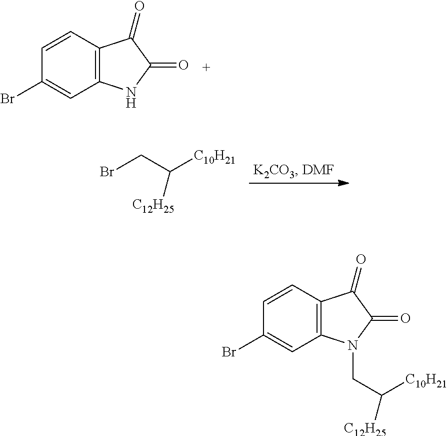

To a mixture of 6-bromoindoline-2,3-dione (1.5 g, 6.64 mmol), K₂CO₃ (3.6 g, 26.05 mmol) and anhydrous DMF (18 cm³) in a 50 cm³ two-necked round bottom flask was added 11-(bromomethyl)tricosane (4.16 g, 9.95 mmol). The reaction mixture was heated with stirring at 70° C. under argon for 20 hours. Solvent was then evaporated under reduced pressure and 50 cm³ de-ionized (DI) water was added. The mixture was extracted with dichloromethane (3×50 cm³) and the combined organic phase was dried over Na₂SO₄, filtered, and concentrated under a reduced pressure. The residue were purified through column chromatography on silica gel with dichloromethane:hexane (2:1, v:v) as the eluent to yield 2.46 g (66%) of 6-bromo-1-(2-decyltetradecyl)indoline-2,3-dione as an orange liquid. ¹H NMR (300 MHz, CDCl₃): δ 7.46 (d, J=6.0 Hz, 1H), 7.26 (d, J=6.0 Hz, 1H), 7.01 (s, 1H), 3.56 (d, J=6.0 Hz, 2H), 1.81 (br, 1H), 1.15-1.40 (m, 40H), 0.84-0.89 (m, 6H).

### Example 2

### Synthesis of (3E,7E)-3,7-bis(6-bromo-1-(2-decyltetradecyl)-2-oxoindolin-3-ylidene)benzo[1,2-b:4,5-b′]difuran-2,6(3H,7H)-dione (IBDF-24)

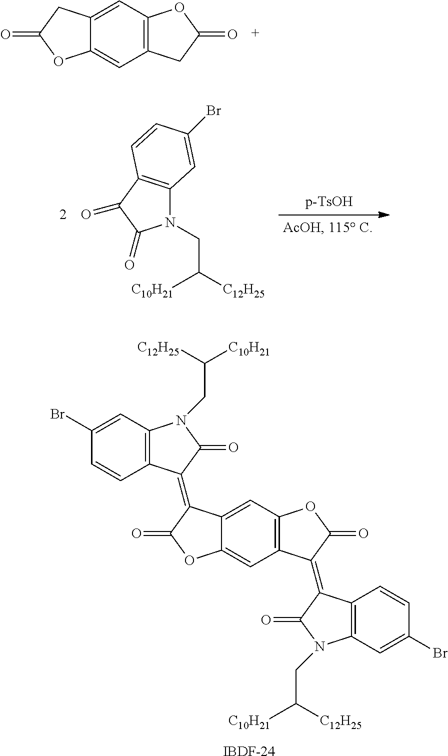

Benzo[1,2-b:4,5-b′]difuran-2,6(3H,7H)-dione was synthesized using a similar procedure described in J. H. Wood, C. S. Colburn, L. Cox and H. C. Garland, J. Am. Chem. Soc., 1944, 66, 1540.

Benzo[1,2-b:4,5-b′]difuran-2,6(3H,7H)-dione (0.285 g, 1.50 mmol), 6-bromo-1-(2-decyltetradecyl)indoline-2,3-dione (3.00 mmol), and p-toluenesulfonic acid (p-TsOH) (0.08 g, 0.42 mmol) were stirred in acetic acid (15 cm³) at 115° C. under argon for 17 hours. The reaction mixture was then cooled to room temperature and filtered. The solid was washed with acetic acid (7 cm³) and methanol (5 cm³) and dried under vacuum. The solid was then purified through column chromatography on silica gel using dichloromethane:hexane (1:1, v:v) as the eluent to yield a dark purple solid (1.6 g). The solid was recrystallized from isopropanol to give the target product. Yield: 1.33 g (69.0%). ¹H NMR (300 MHz, CDCl₃): δ 9.06 (s, 2H), 8.91 (d, J=6.0 Hz, 2H), 7.18 (d, J=6.0 Hz, 2H), 6.88 (s, 2H), 3.61 (d, J=6.0 Hz, 4H), 1.87 (m, 2H), 1.14-1.45 (m, 80H), 0.82-0.88 (m, 12H). UV-Vis (in chloroform): 449 (max), 427 (shoulder) and 584 nm. UV-Vis (in solid state): 426 (max), 625, and 673 nm. Found: C, 69.5; H, 8.4; N, 2.2. Calc. for C₇₄H₁₀₆Br₂N₂O₆: C, 68.9; H, 7.9; N, 2.2.

### Example 3

### Synthesis of PIBDFT-24

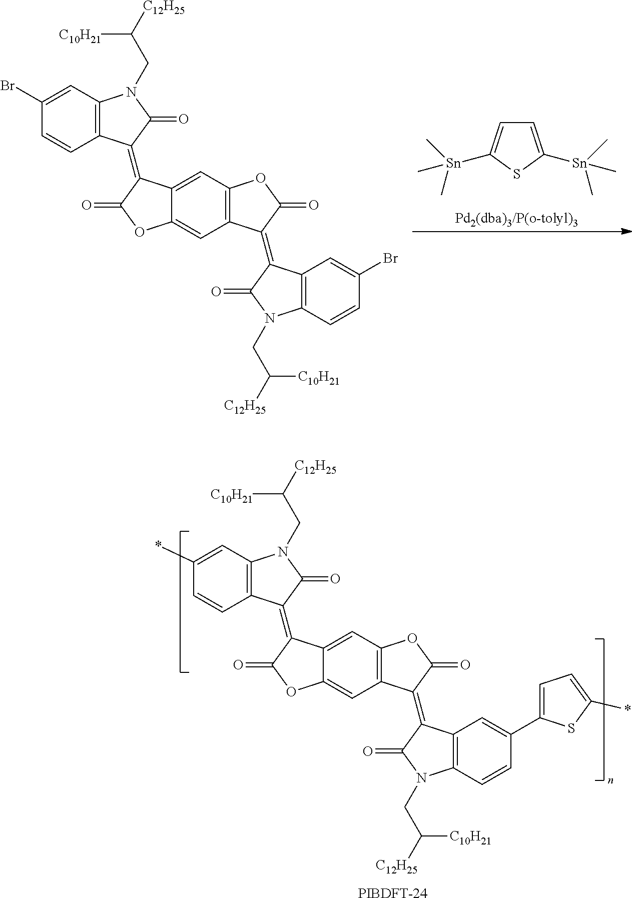

To a 100 cm³ two-necked round bottom flask were added (3E,7E)-3,7-bis(6-bromo-1-(2-decyltetradecyl)-2-oxoindolin-3-ylidene)benzo[1,2-b:4,5-b′]difuran-2,6(3H,7H)-dione (IBDF-24) (0.29 g, 0.227 mmol), 2,5-bis(trimethylstannyl)thiophene (0.093 g, 0.22 mmol) and tri(o-tolyl)phosphine (0.006 g, 0.01816 mmol). The vessel was evacuated and filled with argon three times. Anhydrous chlorobenzene (9 cm³) was added, and a solution of tris(dibenzylideneacetone)dipalladium (3.6 mg, 0.0045 mmol) in 1 cm³ of chlorobenzene was added with a syringe. The reaction mixture was then heated at 130° C. under argon atmosphere for 60 hours. 0.5 cm³ of bromobenzene was added and the reaction mixture was stirred for another 4 hours at 130° C. After cooling down to room temperature, the reaction mixture was added to 150 cm³ of stirring acetone. The polymer was collected by filtration and purified through Soxhlet extraction using acetone and hexane. Finally the polymer was dissolved sequentially with chloroform and chlorobenzene. Yield: 0.158 g (58%) from the chloroform extract and 0.093 g (34%) from the chlorobenzene extract. Found: C, 78.2; H, 9.5; N, 2.6. Calc. for C₇₈H₁₀₈N₂O₆S: C, 78.0; H, 9.1; N, 2.3.

### Example 4

### Synthesis of PIBDFBT-24

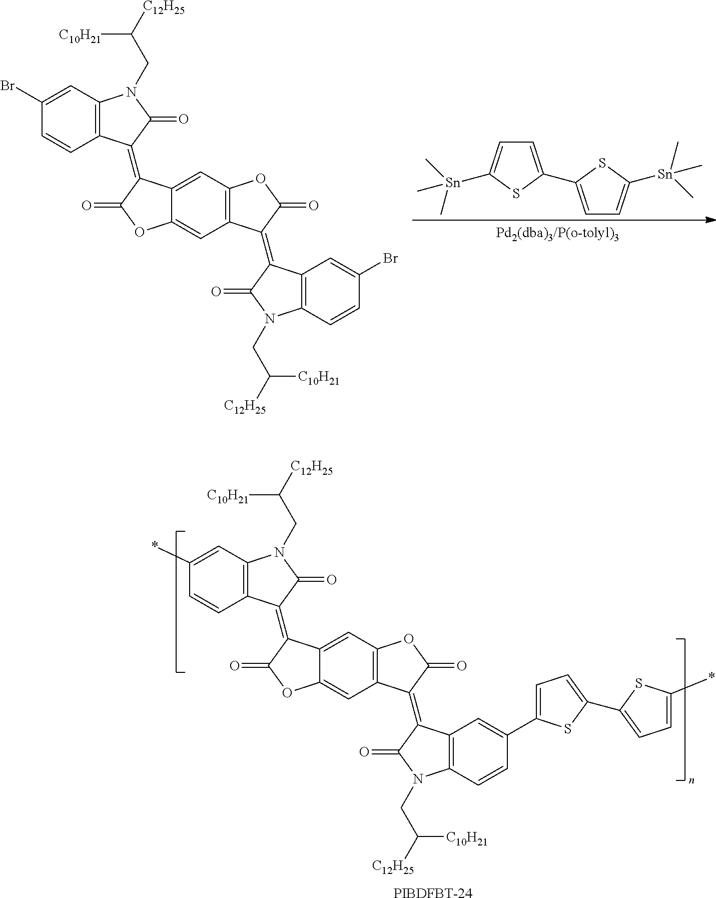

To a 100 cm³ two-neck round bottom flask were added (3E,7E)-3,7-bis(6-bromo-1-(2-decyltetradecyl)-2-oxoindolin-3-ylidene)benzo[1,2-b:4,5-b′]difuran-2,6(3H,7H)-dione (IBDF-24) (0.3 g, 0.23 mmol) and 5,5′-bis(trimethylstannyl)-2,2′-bithiophene (0.115 g, 0.23 mmol). The vessel was evacuated and filled with argon three times. Then toluene (10 cm³) and bis(triphenylphosphine)palladium(II) dichloride (0.005 g, 0.007 mmol) was added. The reaction mixture was then heated at 90° C. for 17 hours and then at 120° C. for 29 hours. Bromobenzene (0.5 cm³) was then added. The mixture was heated at 120° C. for 4 hours and then cooled down to room temperature. The reaction mixture was added to 150 cm³ of stirring methanol. The solid was filtered off and purified by Soxhlet extraction using acetone, hexane, and chloroform. Extraction with tetrachloroethane or chlorobenzene could not dissolve the remaining polymer. Yield of the insoluble fraction: 0.269 g (91%). Found: C, 76.2; H, 9.0; N, 2.4. Calc. for C₈₂H₁₁₀N₂O₆S₂: C, 76.7; H, 8.6; N, 2.2.

### Example 5

### Synthesis of PIBDFV-24

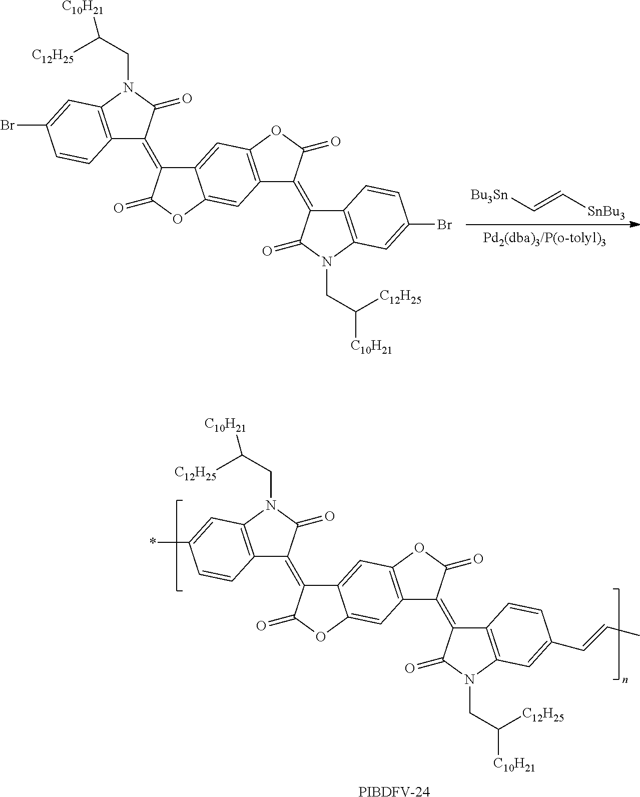

To a 25 mL dry flask were added (3E,7E)-3,7-bis(6-bromo-1-(2-decyltetradecyl)-2-oxoindolin-3-ylidene)benzo[1,2-b:4,5-b′]difuran-2,6(3H,7H)-dione (IBDF-24) (192.1 g, 0.150 mmol), (E)-1,2-bis(tributylstannyl)ethene (0.0909 g, 0.150 mmol) and tri(o-tolyl)phosphine (3.7 mg, 0.012 mmol). After degassing and refilling argon for 3 times, anhydrous chlorobenzene (7 cm³) and tris(dibenzylideneacetone)-dipalladium (Pd₂dba₃) (2.8 mg, 0.003 mmol) were added under an argon atmosphere. The mixture was stirred for 72 hours at 130° C. After cooling down to room temperature, the mixture was poured into methanol (60 cm³) and stirred for 4 hours. The solid was collected by filtration and subject to Soxhlet extraction with acetone and hexane. Finally the polymer was dissolved with chloroform and then chlorobenzene. Yield: 20.0 mg (11.6%) form the chloroform extract and 30.0 mg (17.4%) from the chlorobenzene extract.

### Example 6

### Synthesis of Dimethyl 2-(2-Decyltetradecyl)Malonate

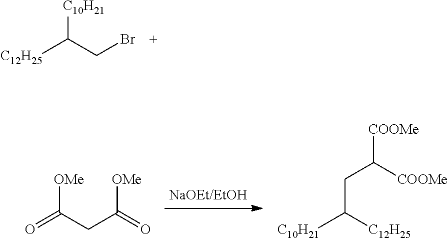

Sodium (1.52 g, 0.066 mmol) was thoroughly reacted with anhydrous ethanol (45 cm³) under argon and then diethyl malonate (7.93 g, 0.06 mol) was added at room temperature. After 30 minutes, 11-(bromomethyl)tricosane (27.87 g, 0.06 mol) was added dropwise. The reaction mixture was stirred under reflux for 16 hours. Then ethanol was evaporated under reduced pressure. DI water was added to dissolve the salt formed. The reaction mixture was extracted three times with diethyl ether. The combined organic phase was dried over the anhydrous Na₂SO₄ and filtered. After removing solvent, the residue was purified by column chromatography on silica gel using hexane:ethyl acetate (15:1, v:v) to give the target product. Yield: 11.7 g (41.5%). ¹H NMR (CDCl₃, 300 MHz, ppm): δ 0.88 (t, 6H), 1.20-1.21 (m, 40H), 2.12 (s, 2H), 3.41 (t, 1H), 4.15-4.26 (m, 6H).

### Example 7

### Synthesis of 4-Decylhexadecanoic Acid

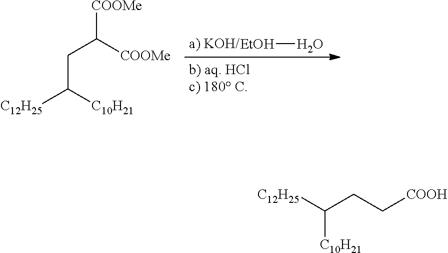

A solution of potassium hydroxide (5.89 g, 0.105 mol) in ethanol (20 cm³) and DI water (10 cm³) was added to dimethyl 2-(2-decyltetradecyl)malonate (10.43 g, 0.021 mol). The reaction mixture was refluxed for 4 hours and then ethanol was distilled off. After addition of more DI water (100 cm³), the mixture was acidified with 12 N HCl (10 cm³). The organic phase was separated using diethyl ether and the combined organic phase was dried over anhydrous NaSO₄ and filtered. After removing solvent, the residue was heated for 30 minutes at 180° C. under a reduced pressure to afford the target compound. Yield: 7.582 g (91.2%). ¹H NMR (CDCl₃, 300 MHz, ppm): δ 0.88 (t, 6H), 1.25-1.26 (m, 40H), 1.56-1.64 (m, 3H), 2.44 (t, 2H).

### Example 8

### Synthesis of 4-decylhexadecan-1-ol

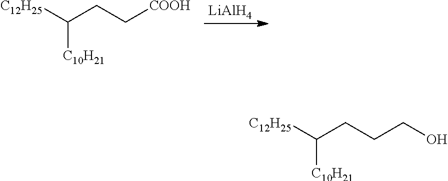

A LiAlH₄ solution (36.2 cm³, 1M in THF, 0.0362 mol) was added dropwise to a suspension of 4-decylhexadecanoic acid (7.18 g, 0.0181 mol) in anhydrous diethyl ether (50 cm³). Then the reaction mixture was refluxed for 6 hours. After cooling down to room temperature, the reaction mixture was added to DI water. The mixture was extracted with diethyl ether three times. The combined organic phase was dried over anhydrous Na₂SO₄ and filtered. After removing solvent, a colorless liquid was obtained. Yield: 5.1 g (74.0%). ¹H NMR (CDCl₃, 300 MHz, ppm): δ 0.88 (t, 6H), 1.24-1.26 (m, 42H), 1.52-1.56 (m, 3H), 3.57-3.64 (m, 2H).

### Example 9

### Synthesis of 11-(3-bromopropyl)tricosane

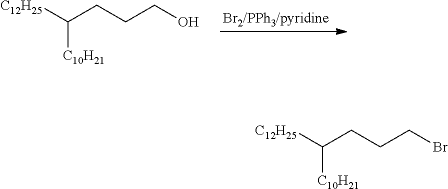

To a solution of triphenylphosphine (3.50 g, 13.33 mmol) in CH₂Cl₂ (25 cm³), bromine (2.13 g, 13.33 mmol) in CH₂Cl₂ (5 cm³) was added slowly at room temperature. After stirring for 1 hour, 4-decylhexadecan-1-ol (5.1 g, 13.33 mmol) and pyridine (1.07 cm³, 13.33 mmol) were added. The mixture was stirred at room temperature for 18 hours. A saturated Na₂SO₃ aqueous solution (25 cm³) was added and stirred for 30 minutes. The organic phase was separated and dried over Na₂SO₄. After removing solvent, the remaining liquid was passed through a silica gel column using hexane as the eluent to give a colourless liquid. Yield: 5.5 g (93.2%). ¹H NMR (CDCl₃, 300 MHz, ppm): δ 0.88 (t, 6H), 1.22-1.28 (m, 42H), 1.54 (m, 1H), 1.83 (br, 2H), 3.39 (t, 2H).

### Example 7

### Synthesis of 6-bromo-1-(4-decylhexadecyl)indoline-2,3-dione

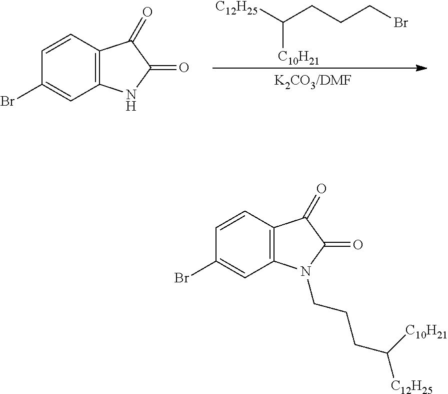

To a mixture of 6-bromoindoline-2,3-dione (0.339 g, 1.5 mmol), K₂CO₃ (0.813 g, 5.88 mmol) and DMF (5 cm³) was added 11-(3-bromopropyl)tricosane (1.002 g, 2.25 mmol). The reaction mixture was stirred at 70° C. under argon for 20 hours. Solvent was then evaporated under reduced pressure and DI water (10 cm³) was added. The mixture was extracted with dichloromethane (3×50 cm³) and the combined organic phase was dried over anhydrous NaSO₄ and filtered. After removing solvent, the residue was passed through a silica gel column using dichloromethane: hexane (1:1, v:v) as the eluent to give the target compound. Yield: 0.60 g (67.7%). ¹H NMR (300 MHz, CDCl₃): δ 0.84-0.89 (m, 6H), 1.15-1.40 (m, 42H), 1.48-1.52 (m, 1H), 1.84 (s, 2H), 3.52-3.59 (q, 2H), 7.01 (s, 1H), 7.27-7.28 (d, J=6.0 Hz, 1H), 7.45-7.47 (d, J=6.0 Hz 1H).

### Example 11

### Synthesis of (3E,7E)-3,7-bis(6-bromo-1-(4-decylhexadecyl)-2-oxoindolin-3-ylidene)benzo[1,2-b:4,5-b′]difuran-2,6(3H,7H)-dione (IBDF-26)

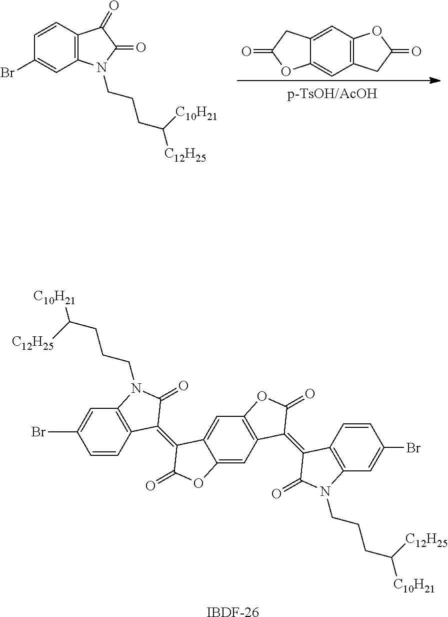

A mixture of benzo[1,2-b:4,5-b′]difuran-2,6(3H,7H)-dione (0.094 g, 0.50 mmol), 6-bromo-1-(4-decylhexadecyl)indoline-2,3-dione (0.591 g, 1.00 mmol), and p-toluenesulfonic acid (0.027 g, 0.14 mmol) in acetic acid (5 cm³) was stirred at 115° C. under argon for 17 hours. The reaction mixture was then cooled down to room temperature and filtered. The solid was washed with acetic acid and methanol and further purified with column chromatography on silica gel using a mixture of dichloromethane:hexane (1:1, v:v) as the eluent to give the target product. Yield: 0.401 g (60.6%). ¹H NMR (300 MHz, CDCl₃): δ 0.88 (m, 12H), 1.14-1.45 (m, 84H), 1.48-1.52 (t, 2H), 1.60-1.66 (s, 4H), 3.52-3.59 (m, 4H), 6.95 (s, 2H), 7.20 (d, J=6.0 Hz, 2H), 8.92 (d, J=6.0 Hz, 2H), 9.11 (s, 2H).

### Example 12

### Synthesis of PIBDFV-26

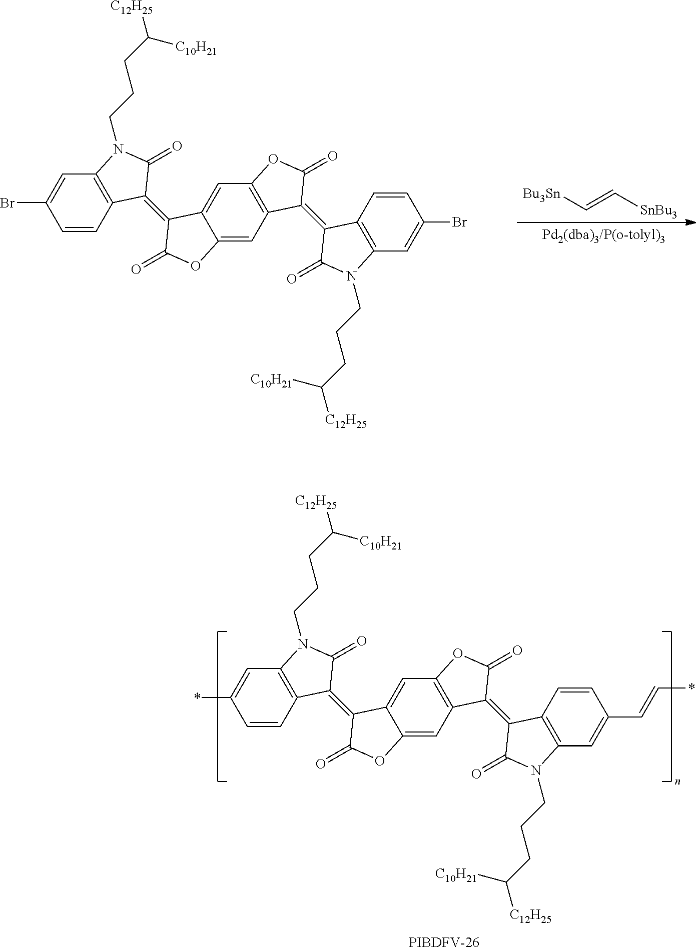

To a 25 cm³ dry flask were added (3E,7E)-3,7-bis(6-bromo-1-(4-decylhexadecyl)-2-oxoindolin-3-ylidene)benzo[1,2-b:4,5-b′]difuran-2,6(3H,7H)-dione (IBDF-26) (191.0 g, 0.1430 mmol), (E)-1,2-bis(tributylstannyl)ethene (0.0867 g, 0.1430 mmol) and tri(o-tolyl)phosphine (3.5 mg, 0.0114 mmol). After degassing and refilling argon for 3 times, anhydrous chlorobenzene (7 cm³) and tris(dibenzylideneacetone)-dipalladium (2.6 mg, 2 mol %, 0.0029 mmol) were added under an argon atmosphere. The mixture was stirred for 72 hours at 130° C. After cooling down to room temperature, the mixture was poured into methanol (60 cm³) and stirred for 4 hours. The solid was collected by filtration and subject to Soxhlet extraction with acetone and hexane for 8 hours. Finally the polymer was dissolved with chloroform and then chlorobenzene. Yield: 23.0 mg (13.4%) in the chloroform fraction and 25.0 mg (14.5%) in the chlorobenzene fraction. GPC (PhCl, 50° C.): Mₙ=78.1 kg mol⁻¹; M_{w}=326.8 kg mol⁻¹.

### Example 13

### Synthesis of heptatriacontan-19-ol

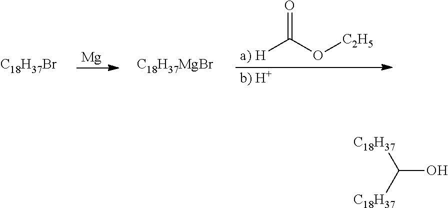

To a 500 cm³ dry 2-necked flask were added magnesium turnings (3.99 g, 0.164 mol). After the flask was degassed and flushed with argon three times, anhydrous ether (70 cm³) was added into the flask through a syringe. A small amount of 1,2-dibromoethane (˜0.3 cm³) was added to initiate the reaction before a solution of 1-bromooctadecane (40.02 g, 0.120 mol) in anhydrous diethyl ether (150 cm³) was added dropwise through a dropping funnel under gentle reflux. After addition of 1-bromooactadecane, the reaction mixture was heated to reflux and maintained for 90 minutes under stirring. The reaction mixture was then cooled down to room temperature and a solution of ethyl formate (4.00 g, 0.054 mol) in anhydrous diethyl ether (50 cm³) was added dropwise. After addition, the reaction mixture was refluxed for 20 hours. The reaction mixture was cooled down to room temperature and poured into methanol containing ice, and acidified with 2N HCl. The solid was filtered off and washed with dichloromethane. Finally the solid was recrystallized using a mixture of THF/methanol. Yield: 23.14 g (79.8%). ¹H NMR (300 MHz, CDCl₃): δ 3.58 (m, 1H), 1.43 (br, 4H), 1.25 (br, 64H), 0.88 (t, J=6.0 Hz, 6H).

### Example 14

### Synthesis of 19-bromoheptatriacontane

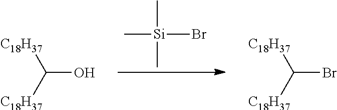

To a 250 cm³ 2-necked flask equipped with a condenser and a dropping funnel was added heptatriacontan-19-ol (22.00 g, 0.041 mol) and the flask was degassed and flushed with argon three times. Anhydrous chloroform (100 cm³) was added and the mixture was heated to 60° C. until the solid was completely dissolved. Then bromotrimethylsilane (25.11 g, 0.164 mol) was added dropwise and the mixture was stirred at 60° C. for 48 hours. After cooling to room temperature, the reaction mixture was poured into chloroform (100 cm³) containing ice. The organic phase was washed three times by DI water. After removing the solvent in vacuo, the solid was recrystallized from hexane three times to give the title product. Yield: 8.85 g (36.0%). ¹H NMR (300 MHz, CDCl₃): δ 3.96-4.06 (m, 1H), 1.72-1.82 (m, 4H), 1.24 (br, 64H), 0.88 (t, J=6.0 Hz, 6H).

### Example 15

### Synthesis of 19-allylheptatriacontane

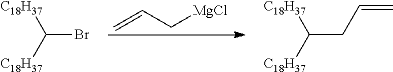

To a 500 cm³ flask was added 19-bromoheptatriacontane (8.8 g, 14.7 mmol) and the flask was degassed and flushed with argon three times. Anhydrous THF (180 cm³) was added to dissolve the solid, followed by dropwise addition of allylmagnesium chloride (15 cm³, 2M in THF, 30 mmol). The mixture was kept under reflux for 20 hours. The reaction mixture was then quenched by an ice/water mixture and acidified by 2N HCl. The aqueous phase was washed with diethyl ether three times. Then the combined organic phase was washed with DI water once. After removing the solvent in vacuo, the solid was stirred in hexane and the solid was filtered off as the product. Yield: 4.8 g (58.2%). ¹H NMR (300 MHz, CDCl₃): δ 5.70-5.84 (m, 1H), 4.95-5.01 (m, 2H), 2.01 (t, J=6.0 Hz, 2H), 1.26-1.43 (br, 65H), 0.88 (t, J=6.0 Hz, 6H).

### Example 16

### Synthesis of 4-octadecyldocosan-1-ol

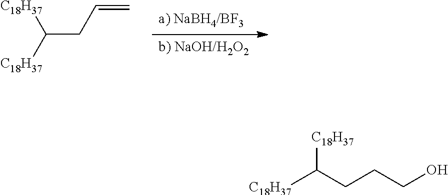

To a 100 cm³ flask were added 19-allylheptatriacontane (4.4 g, 7.9 mmol) and diethylene glycol dimethyl ether (20 cm³). The mixture was heated to 40° C. to dissolve the solid. Sodium borohydride (0.1 g) and then boron trifluoride etherate (0.4 cm³) were added. The mixture was stirred at 40° C. for 2.5 hours before DI water (0.7 cm³) was added slowly. Once the hydrogen generation ceased a solution of NaOH (0.87 g, 0.022 mol) in water (10 cm³) was added. The mixture was cooled down in an ice/water bath and hydrogen peroxide (1.4 cm³, 30% aqueous solution) was added. Then the mixture was stirred at room temperature for 4 hours. The reaction mixture was extracted with diethyl ether, dried over anhydrous Na₂SO₄, and filtered. After removing the solvent, the solid was purified using column chromatography with dichloromethane as the eluent to afford the title product. Yield: 1.9 g (41.6%). ¹H NMR (300 MHz, CDCl₃): δ 3.59-3.63 (m, 2H), 1.43-1.55 (m, 2H), 1.26 (br, 71H), 0.88 (t, J=6.0 Hz, 6H).

### Example 17

### Synthesis of 19-(3-iodopropyl)heptatriacontane

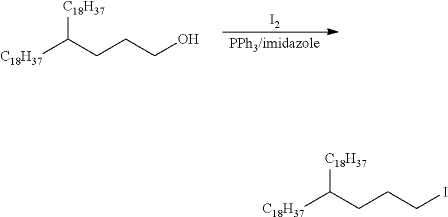

To a solution of 4-octadecyldocosan-1-ol (0.513 g, 0.863 mmol) in dichloromethane (30 cm³) were added PPh₃ (0.282 g, 1.036 mmol) and imidazole (0.077 g, 1.036 mmol). The mixture was cooled with an ice/water bath and iodine (0.263 g, 1.036 mmol) was added. The mixture was then stirred at room temperature for 4 hours before it was quenched with a Na₂SO₃ solution. The organic phase was separated and washed with DI water and dried over anhydrous Na₂SO₄. The product was purified by column chromatography on silica gel using hexane as the eluent. Yield: 0.52 g (71.0%). ¹H NMR (300 MHz, CDCl₃): δ 3.17 (t, J=6.0 Hz, 2H), 1.75-1.84 (m, 2H), 1.26 (br, 71H), 0.88 (t, J=6.0 Hz, 6H).

### Example 18

### Synthesis of 6-bromo-1-(4-octadecyldocosyl)indoline-2,3-dione

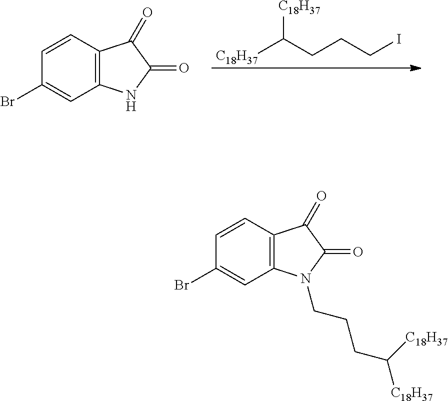

To a 100 cm³ flask were added 19-(3-iodopropyl)heptatriacontane (0.5244 g, 0.761 mmol), 6-bromoindoline-2,3-dione (0.1744 g, 0.761 mmol) and K₂CO₃ (0.2146 g, 1.522 mmol). Then the flask was evacuated and flushed with argon three times before anhydrous THF (9 cm³) and DMF (9 cm³) were added. The mixture was stirred at 50° C. for 18 hours. After the mixture was cooled down to room temperature, solvent was removed in vacuo and the residue was dissolved in dichloromethane (25 cm³). After phase separation using dichloromethane and brine, the combined organic phase was dried over anhydrous Na₂SO₄ and the solvent was removed in vacuo. The residue was purified using column chromatography on silica gel with hexane: dichloromethane (3:2, v:v) as the eluent to give the target product. Yield: 0.46 g (76.7%). ¹H NMR (300 MHz, CDCl₃): δ 7.46 (d, J=6.0 Hz, 1H), 7.29 (d, J=6.0 Hz, 1H), 7.06 (s, 1H), 3.67 (t, J=6.0 Hz, 2H), 1.66 (br, 2H), 1.26 (br, 71H), 0.88 (t, J=6.0 Hz, 6H).

### Example 19

### Synthesis of (3E,7E)-3,7-bis(6-bromo-1-(4-octadecyldocosyl)-2-oxoindolin-3-ylidene)benzo[1,2-b:4,5-b′]difuran-2,6(3H,7H)-dione (IBDF-40)

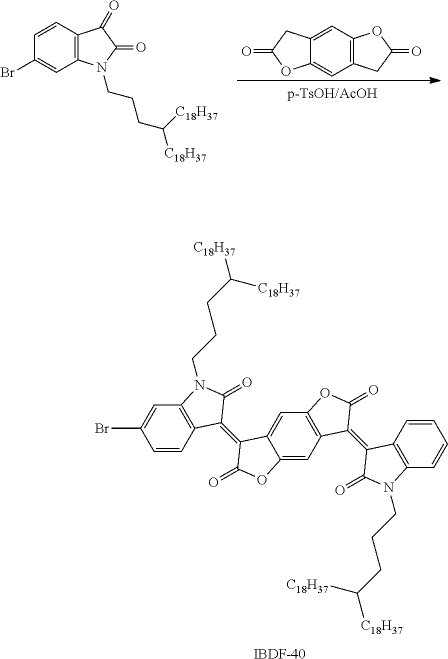

To a 25 cm³ flask were added 6-bromo-1-(4-octadecyldocosyl)indoline-2,3-dione (0.420 g, 0.534 mmol), benzo[1,2-b:4,5-b′]difuran-2,6(3H,7H)-dione (0.051 g, 0.267 mmol), and p-toluenesulfonic acid (p-TsOH) (0.015 g, 0.076 mmol) and the flask was evacuated and flushed with argon three times. Then acetic acid (4 cm³) was added and the mixture was heated to 115° C. and maintained at this temperature for 18 hours under stirring. After the mixture was cooled down to room temperature, the precipitate was separated from the solvent through filtration. The solid was washed with methanol and acetic acid, and further purified with column chromatography on silica gel with hexane: dichloromethane (2:1, v:v) to give the target product. Yield: 0.265 g (57.6%). ¹H NMR (300 MHz, CDCl₃): δ 9.12 (s, 2H), 8.95 (d, J=4.5 Hz, 2H), 7.23 (d, 2H), 6.96 (s, 2H), 3.74 (t, J=7.5 Hz, 4H), 1.68 (br, 4H), 1.24 (br, 142H), 0.87 (t, J=6.0 Hz, 12H).

### Example 20

### Synthesis of PIBDFBT-40

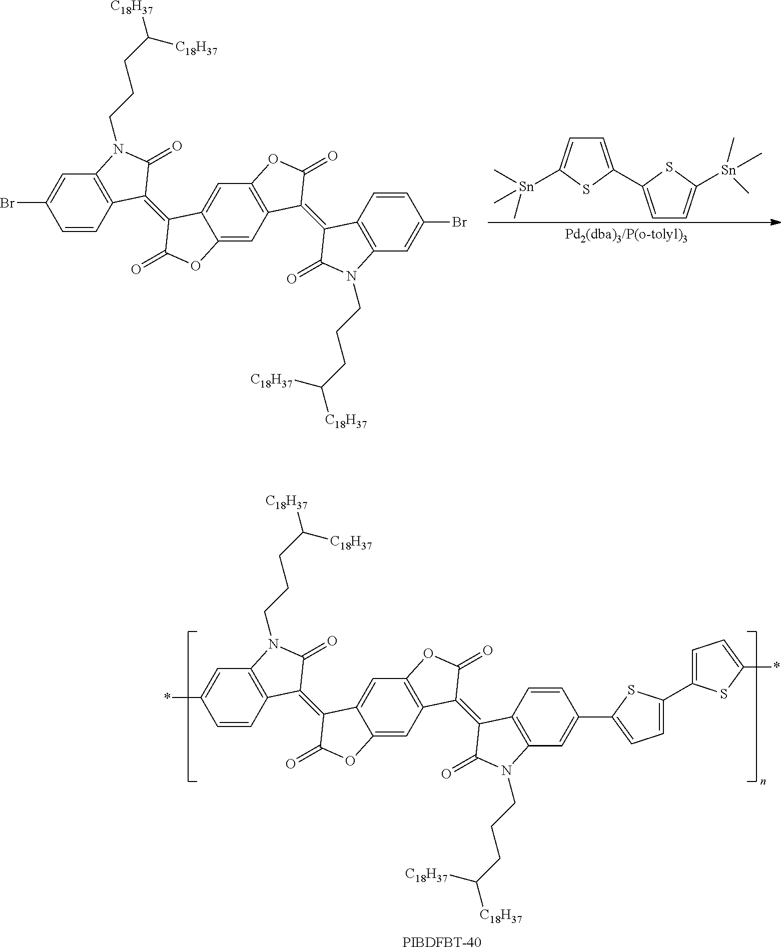

To a 25 cm³ dry flask were added (3E,7E)-3,7-bis(6-bromo-1-(4-octadecyldocosyl)-2-oxoindolin-3-ylidene)benzo[1,2-b:4,5-b′]difuran-2,6(3H,7H)-dione (IBDF-40) (98.2 mg, 56.8 μmol), 5,5′-bis(trimethylstannyl)bithiophene (27.9 mg, 56.8 μmol), and tri(o-tolyl)phosphine (1.4 mg, 4.56 μmol). The flask was evacuated and flushed with argon three times. Then anhydrous chlorobenzene (4 cm³) and tris(dibenzylideneacetone)-dipalladium (1.0 mg, 1.14 μmol) were added. The mixture was heated to 130° C. and stirred at this temperature for 72 hours. After cooling down to room temperature, the reaction mixture was added to methanol under stirring. The solid was filtered off and extracted with a Soxhlet apparatus sequentially with acetone and hexane. Finally the polymer was dissolved with chloroform to obtain the product after drying in vacuo. Yield: 82.0 mg (83.2%). GPC (PhCl, 50° C.): Mₙ=92.4 kg mol⁻¹; M_{w}=248.1 kg mol⁻¹.

### Example 21

### Synthesis of PIBDFV-40

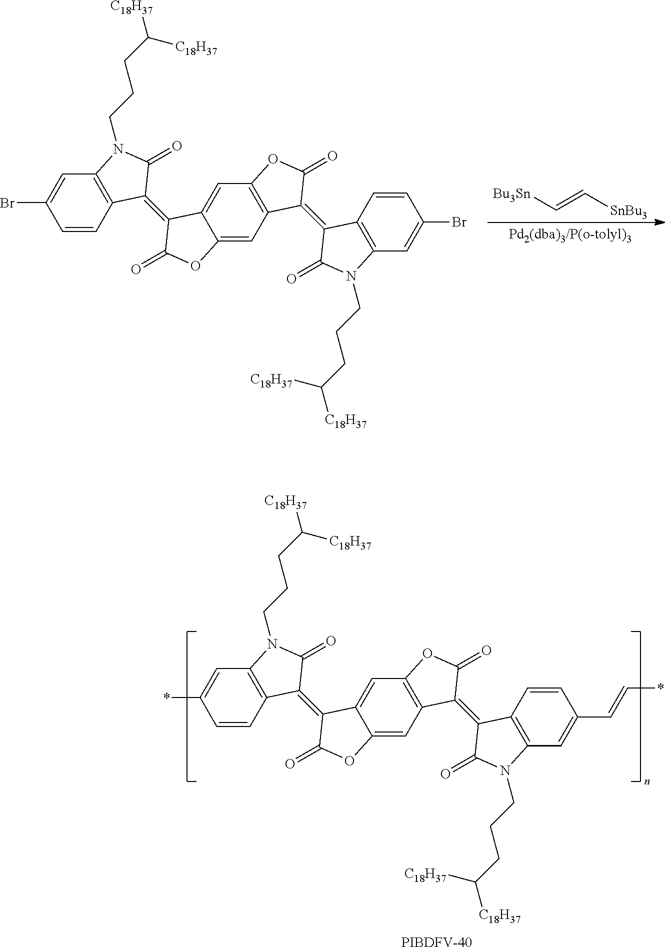

To a 25 cm³ dry flask was added (3E,7E)-3,7-bis(6-bromo-1-(4-octadecyldocosyl)-2-oxoindolin-3-ylidene)benzo[1,2-b:4,5-b′]difuran-2,6(3H,7H)-dione (IBDF-40) (108.7 mg, 62.92 μmol), (E)-1,2-bis(tributylstannyl)ethene (38.1 mg, 62.92 μmol) and tri(o-tolyl)phosphine (1.5 mg, 5.03 μmol). After degassing and refilling argon for 3 times, tris(dibenzylideneacetone)-dipalladium (1.2 mg, 1.26 μmol) and dry chlorobenzene (4 cm³) were added under argon. The mixture was stirred for 72 hours at 130° C. After cooling down to room temperature, the mixture was poured into methanol (60 cm³) and stirred for 4 hours. The solid was collected by filtration and subject to Soxhlet extraction with acetone and hexane. Finally the polymer was dissolved with chloroform to give a black film after removing solvent. Yield: 69.8 mg (69.6%). GPC (PhCl, 50° C.): Mₙ=50.2 kg mol⁻¹; M_{w}=127.9 kg mol⁻¹.

### Example 22

### Fabrication and Characterization of OTFT Devices

A bottom-contact, bottom-gate OTFT configuration was used. Heavily p-doped Si wafer functions as the gate electrode and a thermally grown SiO₂ layer (˜200 nm) with a capacitance of ˜17 nF/cm² on top of the Si layer was used as the insulating dielectric. Gold source and drain electrode pairs were pre-deposited on the SiO₂ layer with the common photolithography method. The substrate was cleaned with DI water, acetone, and isopropanol in an ultrasonic bath, followed by O₂ plasma. Subsequently, the substrate was immerged in a dodecyltrichlorosilane (DTS) solution in toluene (10 mg/cm³) at 70° C. for 20 minutes. After washing with toluene, the substrate was dried under a nitrogen flow. A polymer solution in chloroform (10 mg/cm³) was spin coated on the substrate at 3000 rpm for 60 seconds to give a film, which was subject to thermal annealing at different temperature for 15 minutes in a glove box. The devices were encapsulated with a 500 nm-thick PMMA (M_{w}=120,000 g mol⁻¹) layer by spin coating a PMMA solution in butyl acetate (8 wt %) at 3000 rpm for 50 seconds, and dried at 80° C. for 30 minutes in a glove box under nitrogen. OTFT devices have a channel length (L) of 30 μm and a channel width (W) of 1000 μm. The devices were characterized in air using an Agilent 4155C Semiconductor Analyzer. The carrier mobility in the saturated regime, μₛₐₜ, was calculated according to the equation of I_{DS}=Cᵢμₛₐₜ(W/2 L)(V_{G}−V_{T})², where I_{DS} is the drain current, C, is the capacitance per unit area of the gate dielectric, W and L are, respectively, the semiconductor channel width and length, and V_{G} and V_{T} are, respectively, the gate voltage and threshold voltage. V_{T} of the devices was determine from extrapolation of the linear fit of the (I_{DS})^{1/2} versus V_{G} curve in the saturation regime at I_{DS}=0.

OTFT characteristics of the exemplary polymers are summarized in Table 1 for electron enhancement mode and in Table 2 for hole enhancement mode, with μₑ denoting the electron mobility, μₕ denoting the hole mobility, and Iₒₙ/I_{off} denoting the current on-to-off ratio. Output and transfer curves of typical devices using the exemplary polymers are shown in FIG. 5 through FIG. 10.

TABLE 1
Electron enhancement mode

| | Annealing | | |
|---|---|---|---|
| | temperature | μₑ | |
| Polymer | [° C.] | [cm²V⁻¹s⁻¹] | Iₒₙ/I_{off} |
| PIBDFT-24 | 150 | 2.4-3.0 × 10⁻³ | ~10³ |
| | 200 | 4.2-5.4 × 10⁻³ | ~10³-10⁴ |
| PIBDFV-24 | 150 | 0.09-0.10 | ~10⁶-10⁷ |
| | 200 | 0.11-0.14 | ~10⁵ |
| PIBDFV-26 | 150 | 0.11-0.14 | ~10⁶-10⁷ |
| | 200 | 0.06-0.10 | ~10⁴-10⁵ |
| PIBDFV-40 | 150 | 0.09-0.13 | ~10⁵-10⁶ |
| | 200 | 0.12-0.16 | ~10⁵-10⁶ |
| PIBDFBT-40 | 150 | 0.57-0.70 | ~10²-10⁵ |
| | 200 | 0.29-0.66 | ~10²-10⁵ |

TABLE 2
Hole enhancement mode

| | Annealing | | |
|---|---|---|---|
| | temperature | μₕ | |
| Polymer | [° C.] | [cm²V⁻¹s⁻¹] | Iₒₙ/I_{off} |
| PIBDFBT-40 | 150 | 0.14-0.23 | ~10²-10³ |
| | 200 | 0.09-0.23 | ~10²-10³ |

1. A monomer, oligomer or polymer comprising a fused-ring moiety (I): 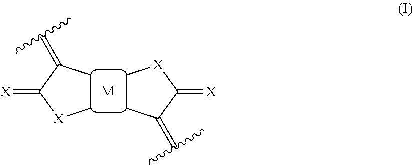
X is independently oxygen (O), sulphur (S), or NR (R is independently hydrogen or optionally substituted hydrocarbon with 1 to 60 carbon atoms such as, for example, alkyl, substituted alkyl, alkenyl, substituted alkenyl, alkynyl, substituted alkynyl, aryl, and substituted aryl, or any other suitable group); 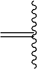
indicates the linkage with another moiety (or moieties);
M is a conjugated moiety.

2. The monomer, oligomer or polymer according to claim 1, wherein M is selected from a group of structures: 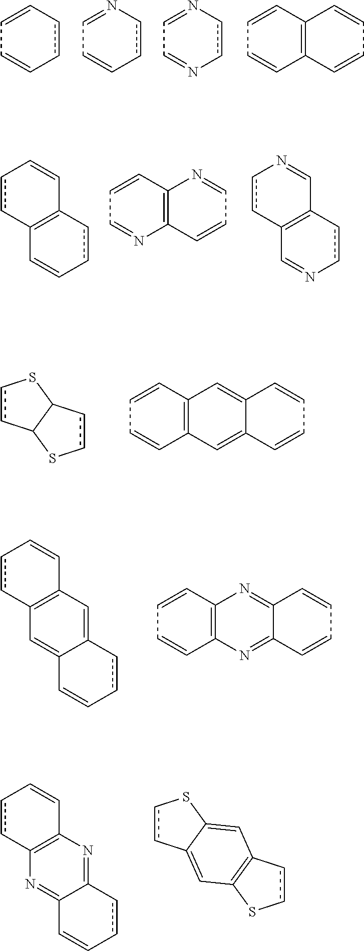
Wherein:
--- indicates the fusion of M with the adjacent five-membered rings and the fusion can be in any suitable direction;
each structure can be substituted, where is applicable, with one or more suitable groups independently selected from optionally substituted hydrocarbon with about 1 to about 60 carbon atoms (such as, for example, alkyl, substituted alkyl, alkenyl, substituted alkenyl, alkynyl, substituted alkynyl, aryl, substituted aryl, alkoxy, and substituted alkoxy), cyano (CN), nitro, and halogen, or any other suitable group.

3. The monomer, oligomer or polymer according to claim 1, wherein (I) is selected from the following structures: 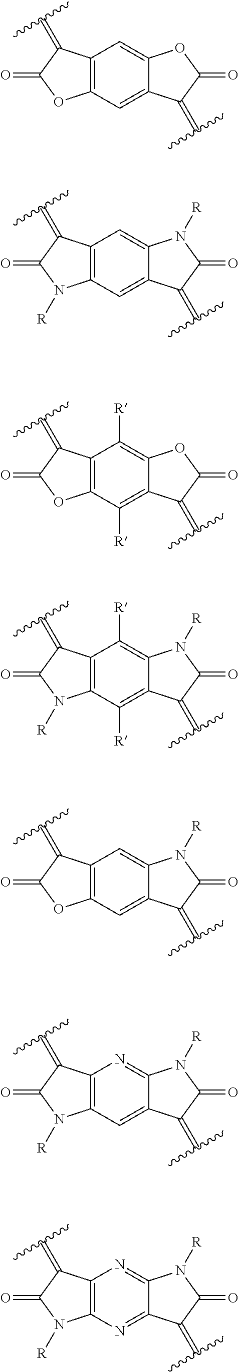 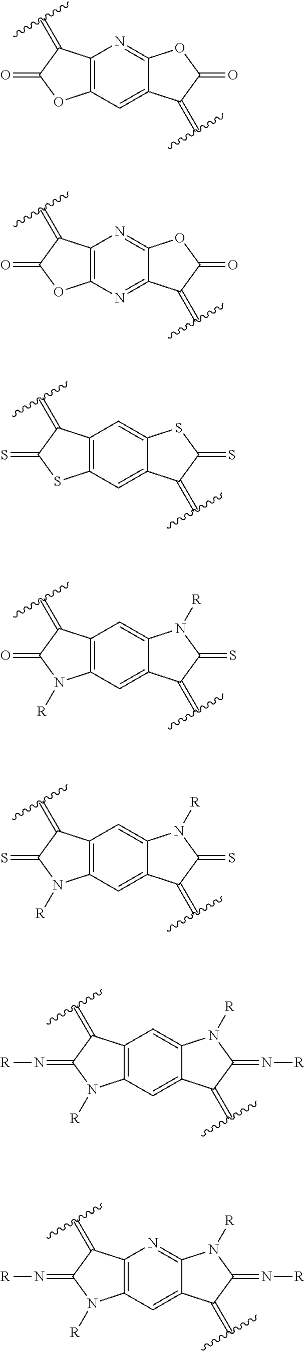 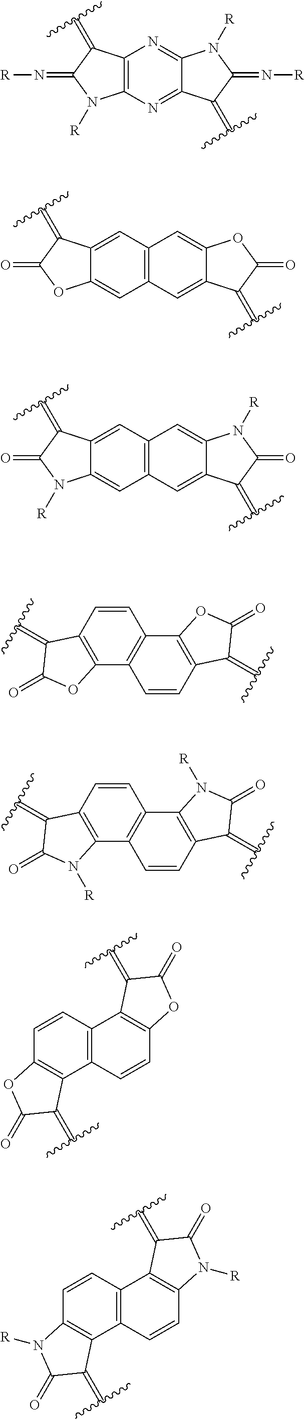
where R is independently hydrogen or optionally substituted hydrocarbon with 1 to 60 carbon atoms such as, for example, alkyl, substituted alkyl, alkenyl, substituted alkenyl, alkynyl, substituted alkynyl, aryl, and substituted aryl, or any other suitable group;
R′ is independently hydrogen or optionally substituted hydrocarbon with 1 to 60 carbon atoms such as, for example, alkyl, substituted alkyl, alkenyl, substituted alkenyl, alkynyl, substituted alkynyl, aryl, and substituted aryl, or any other suitable group;
each structure can be further substituted, where is applicable, with one or more suitable groups independently selected from optionally substituted hydrocarbon with 1 to 60 carbon atoms (such as, for example, alkyl, substituted alkyl, alkenyl, substituted alkenyl, alkynyl, substituted alkynyl, aryl, substituted aryl, alkoxy, and substituted alkoxy), cyano (CN), nitro, and halogen, or any other suitable group.

4. A monomer, oligomer or polymer according to claim 1 with the following structure PI and PII: 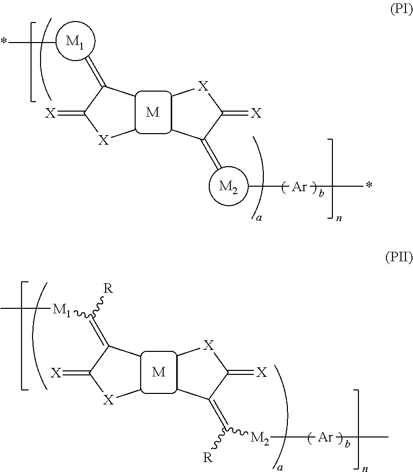
wherein
X, M and R are defined as above;
a is an integer from 1 to 20;
b or c is an integer from 0 (zero) to 20;
The unit Ar and the unit M₁-(I)-M₂ can be connected in a random or alternating manner, i.e., (PI) or (PII) can be a random copolymer, an alternating copolymer, or a block copolymer;
n is a number from 1 to 1,000,000; [symbol]
indicates the linkage can be a cis- or trans-structure;
the terminal “*” can be hydrogen or any other suitable group or moiety;
Ar is independently a π-conjugated moiety.

5. The monomer, oligomer or polymer according to claim 4, wherein Ar is independently selected from the following structures or a combination of the following structures: 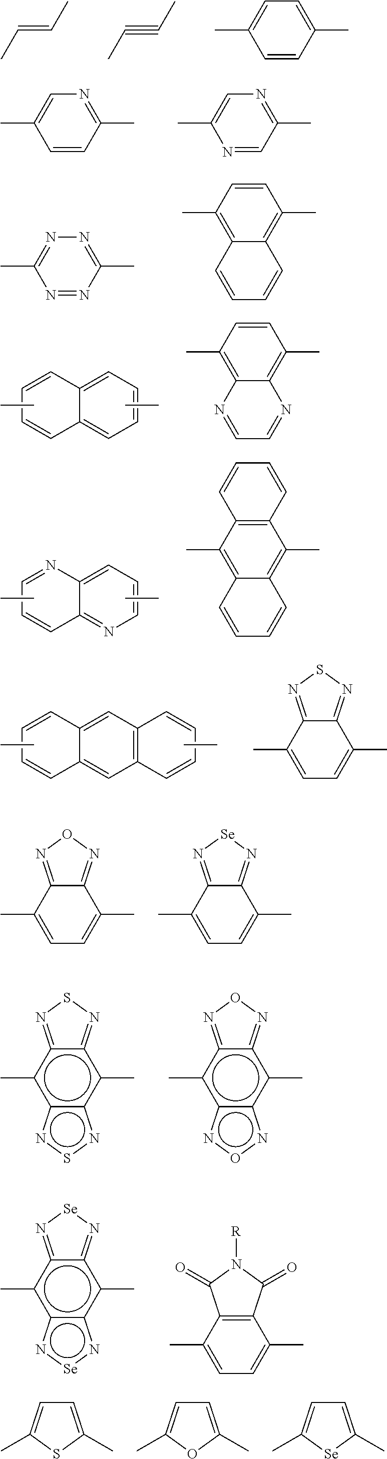 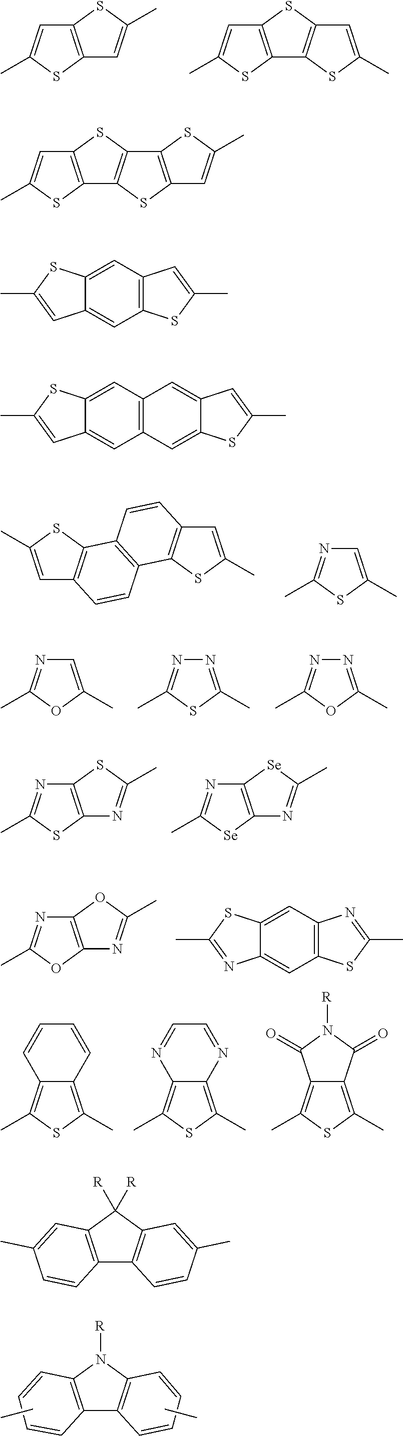 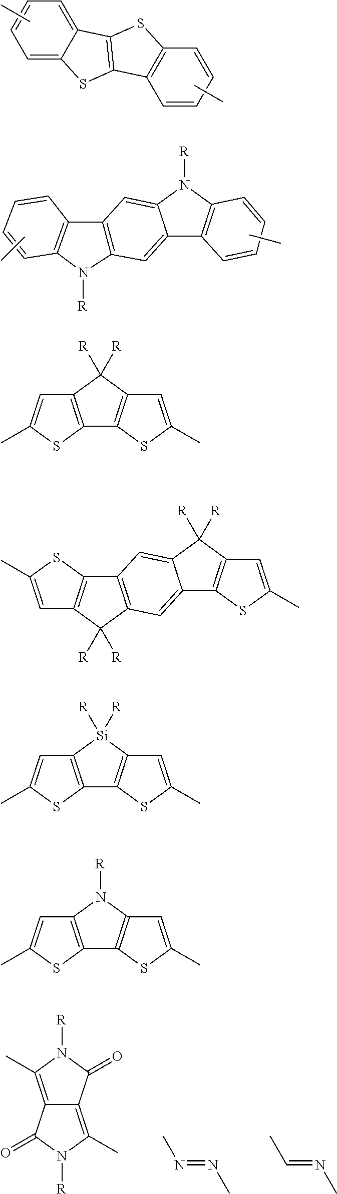 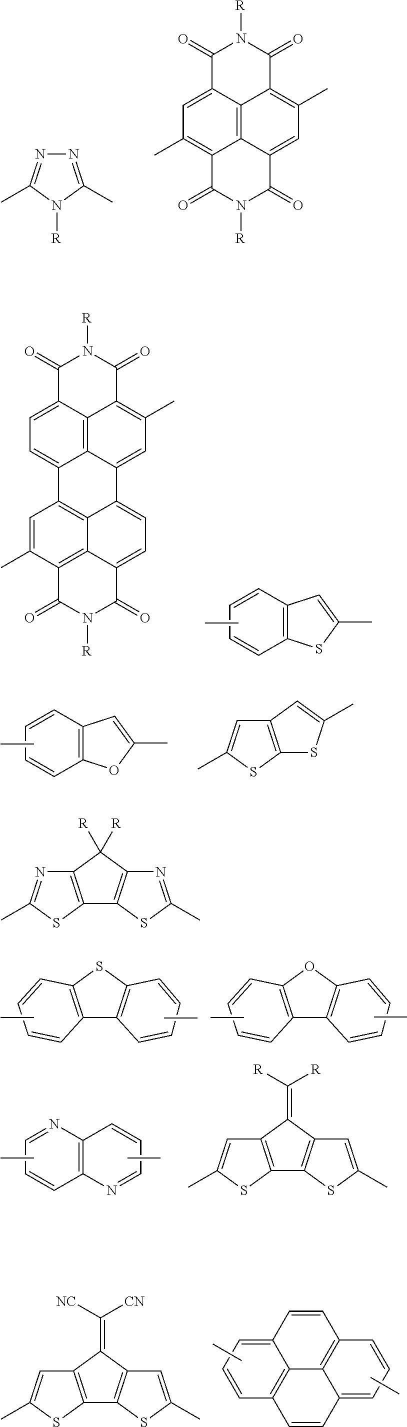 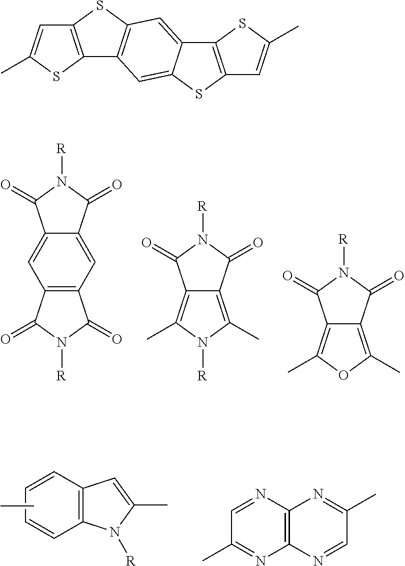
wherein R is independently hydrogen or optionally hydrocarbon of alkyl, substituted alkyl, alkenyl, substituted alkenyl, alkynyl, substituted alkynyl, aryl, or substituted aryl with 1 to 60 carbon atoms;
each structure can be substituted, where is applicable, with one or more suitable groups independently selected from optionally substituted hydrocarbon with 1 to 60 carbon atoms (such as, for example, alkyl, substituted alkyl, alkenyl, substituted alkenyl, alkynyl, substituted alkynyl, aryl, substituted aryl, alkoxy, and substituted alkoxy), cyano (CN), nitro, and halogen, or any other suitable group.

6. The monomer, oligomer or polymer according to claim 4, wherein Ar are optionally substituted with one or more suitable groups independently selected from optionally substituted hydrocarbon with 1 to 60 carbon atoms, nitro, and halogen.

7. The monomer, oligomer or polymer according to claim 4, which has one of the following structures. 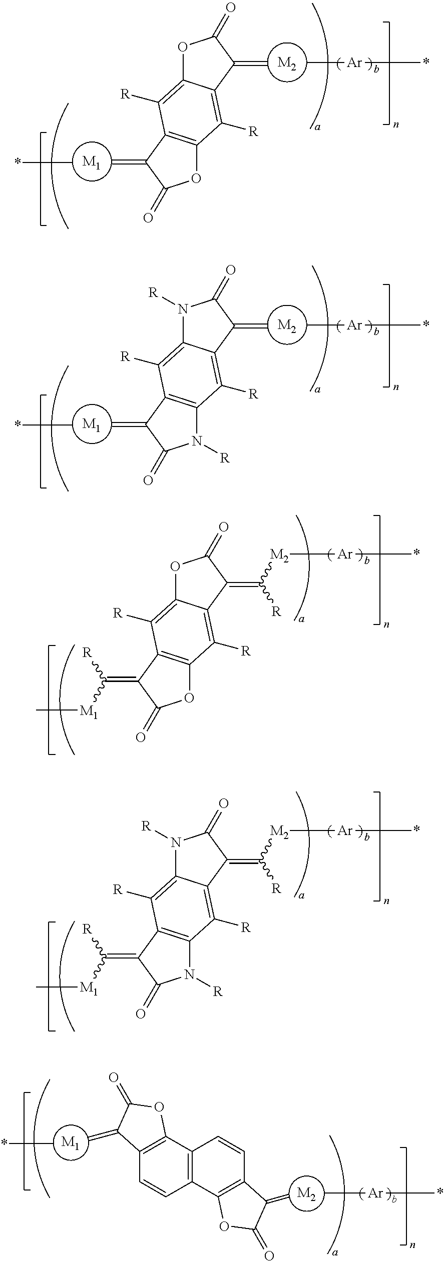 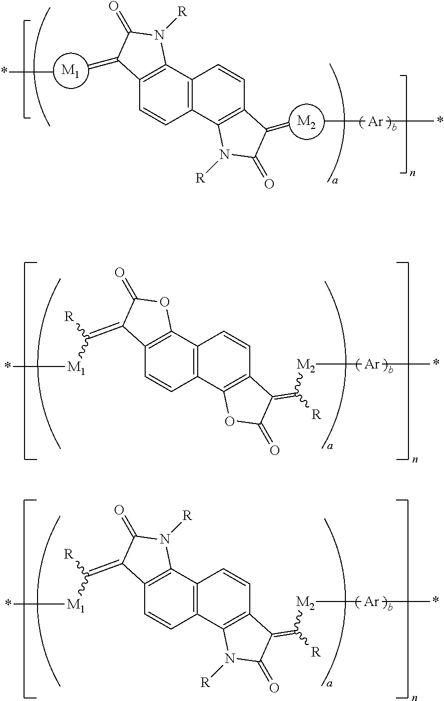
wherein:
M₁, M₂, Ar, R, a, b, and n are defined as above;
each structure can be substituted, where is applicable, with one or more suitable groups independently selected from optionally substituted hydrocarbon with 1 to 60 carbon atoms (such as, for example, alkyl, substituted alkyl, alkenyl, substituted alkenyl, alkynyl, substituted alkynyl, aryl, substituted aryl, alkoxy, and substituted alkoxy), cyano (CN), nitro, and halogen, or any other suitable group;
n is a number from 1 to 1,000,000; [symbol]
indicates the linkage can be a cis- or trans-structure;
the terminal “*” can be hydrogen or any other suitable group or moiety.

8. The monomer, oligomer or polymer according to claim 4 is selected from the structures (1) through (230): 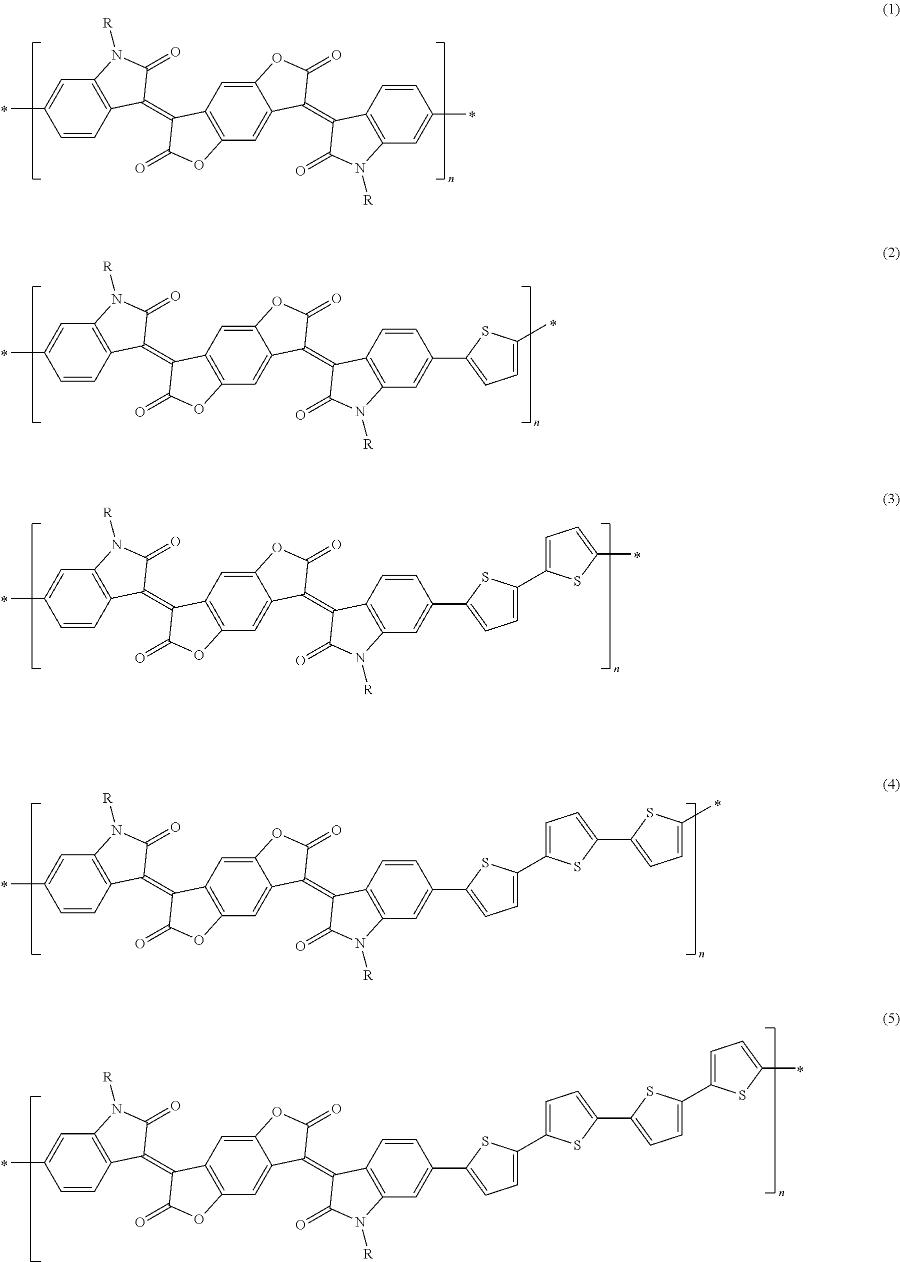 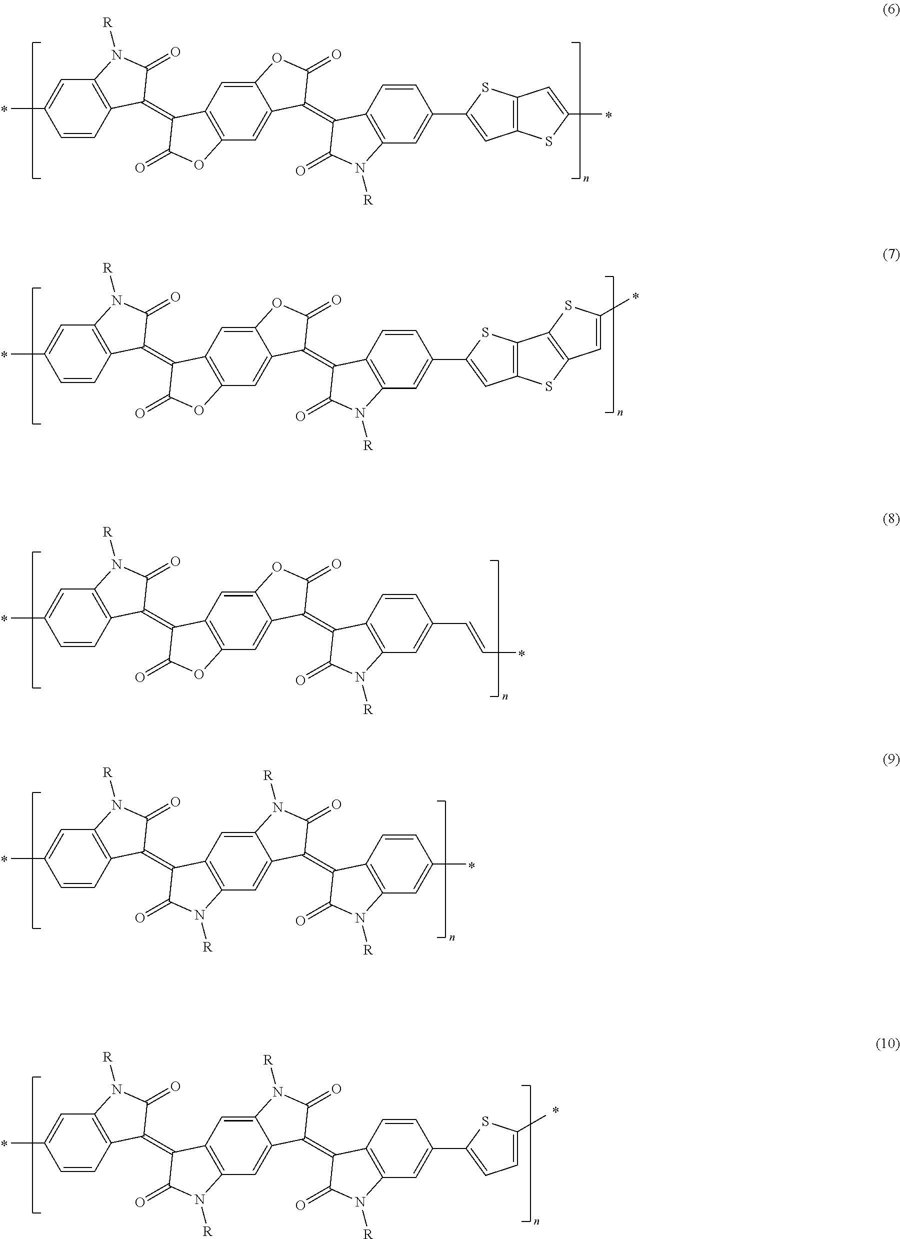 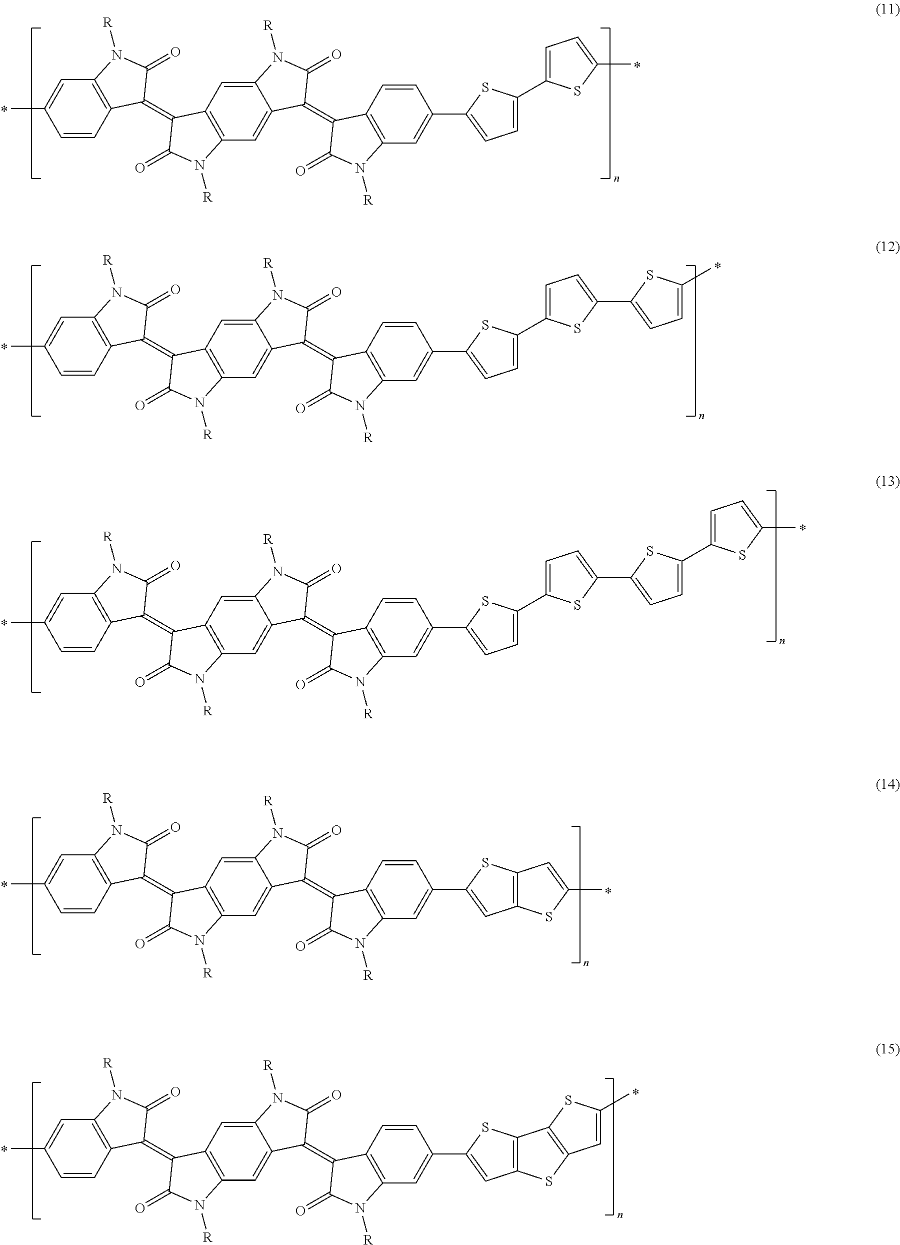 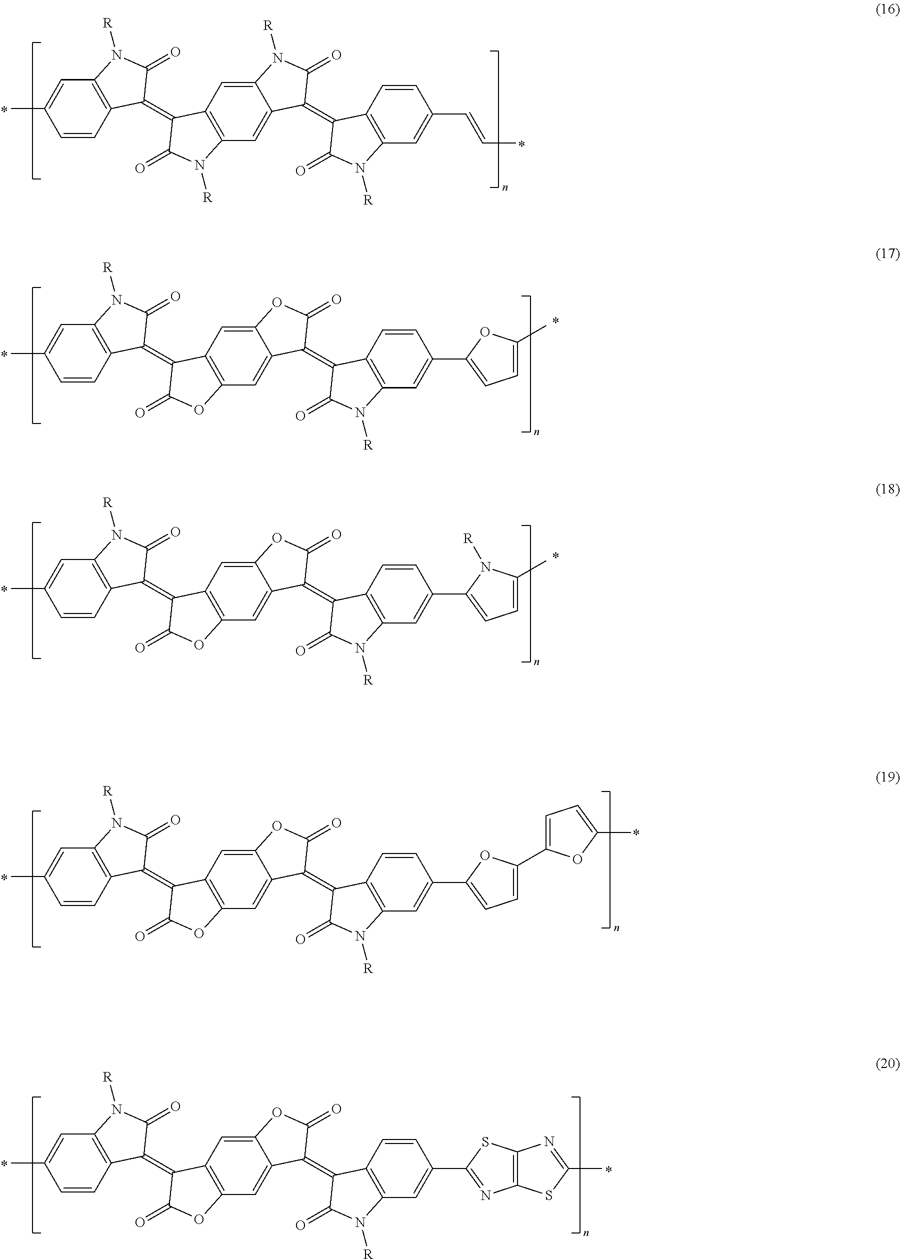 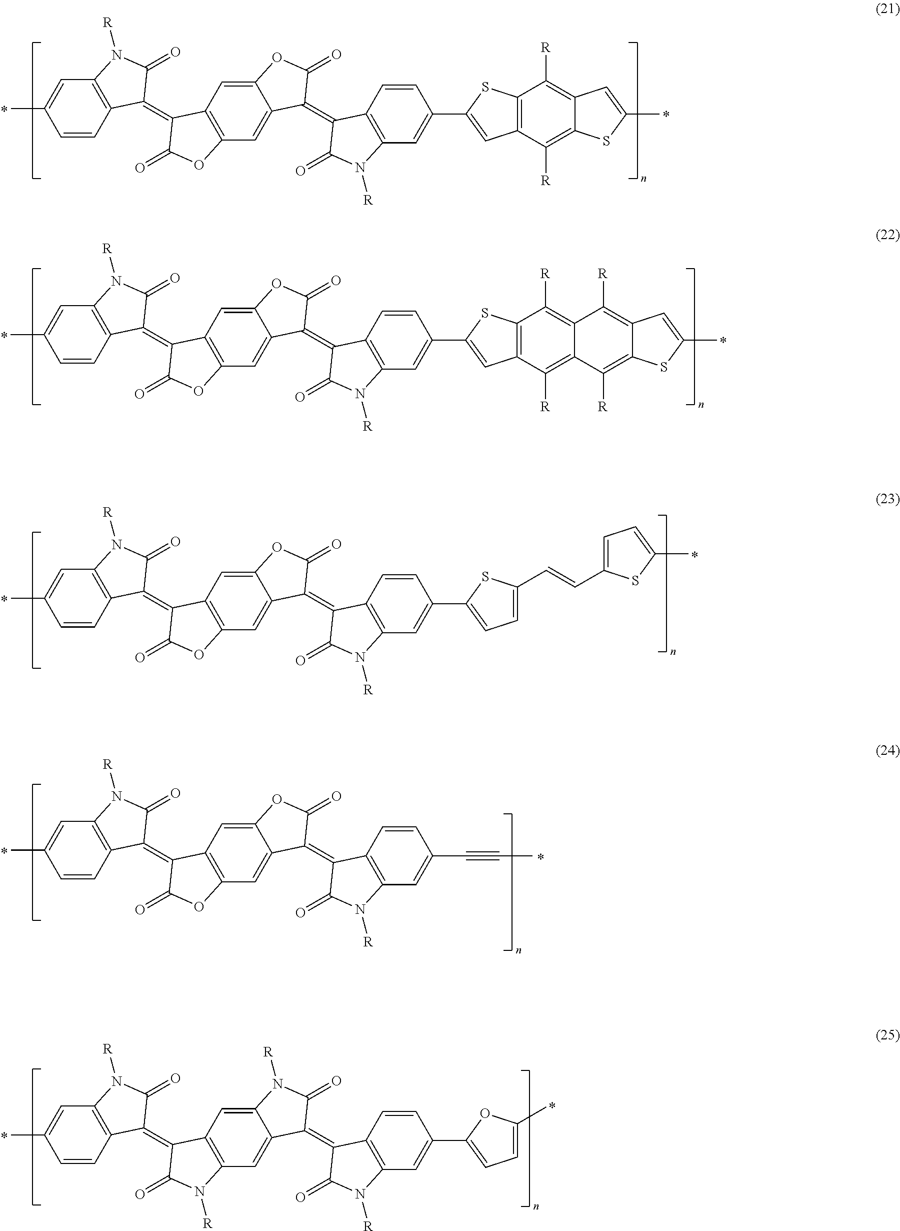 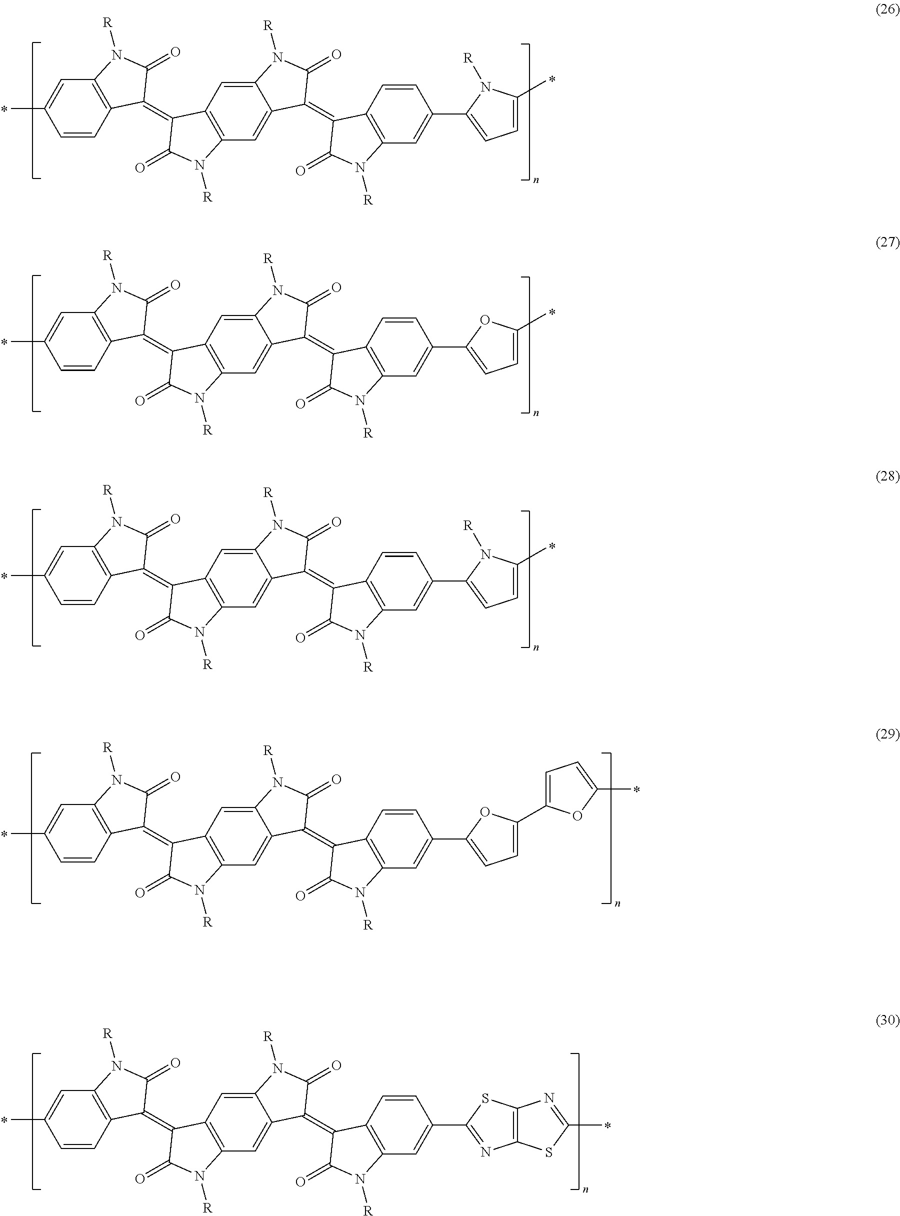 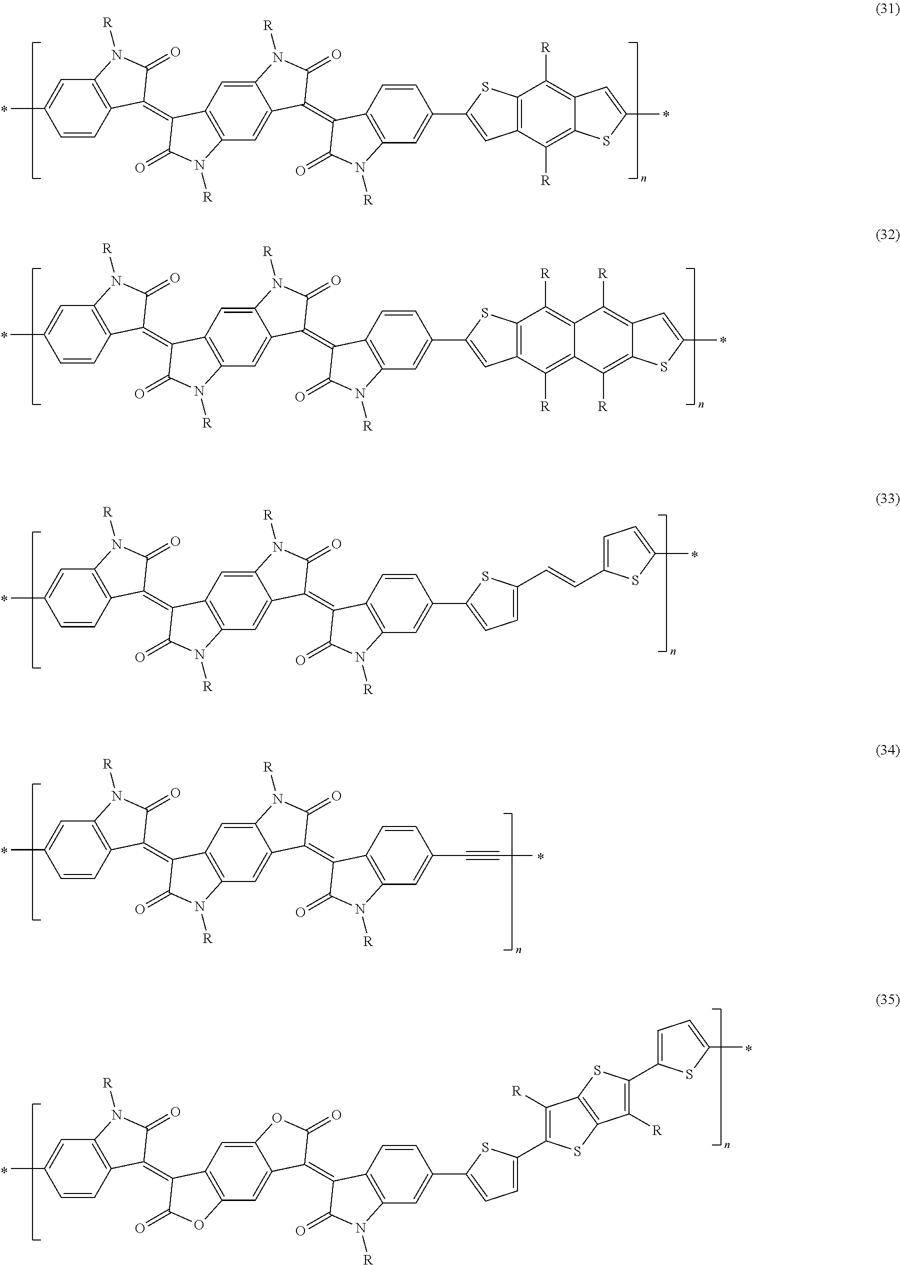 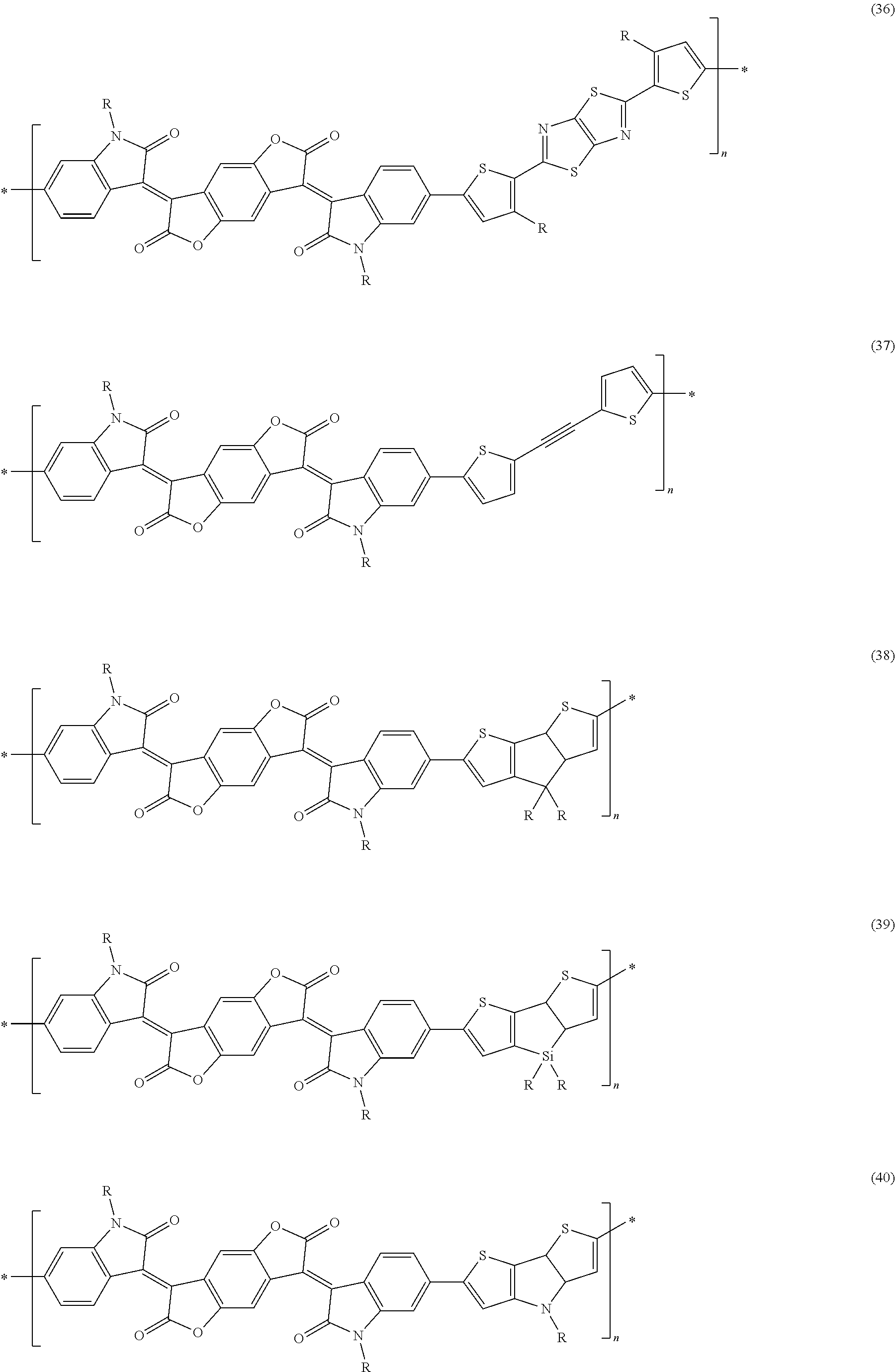 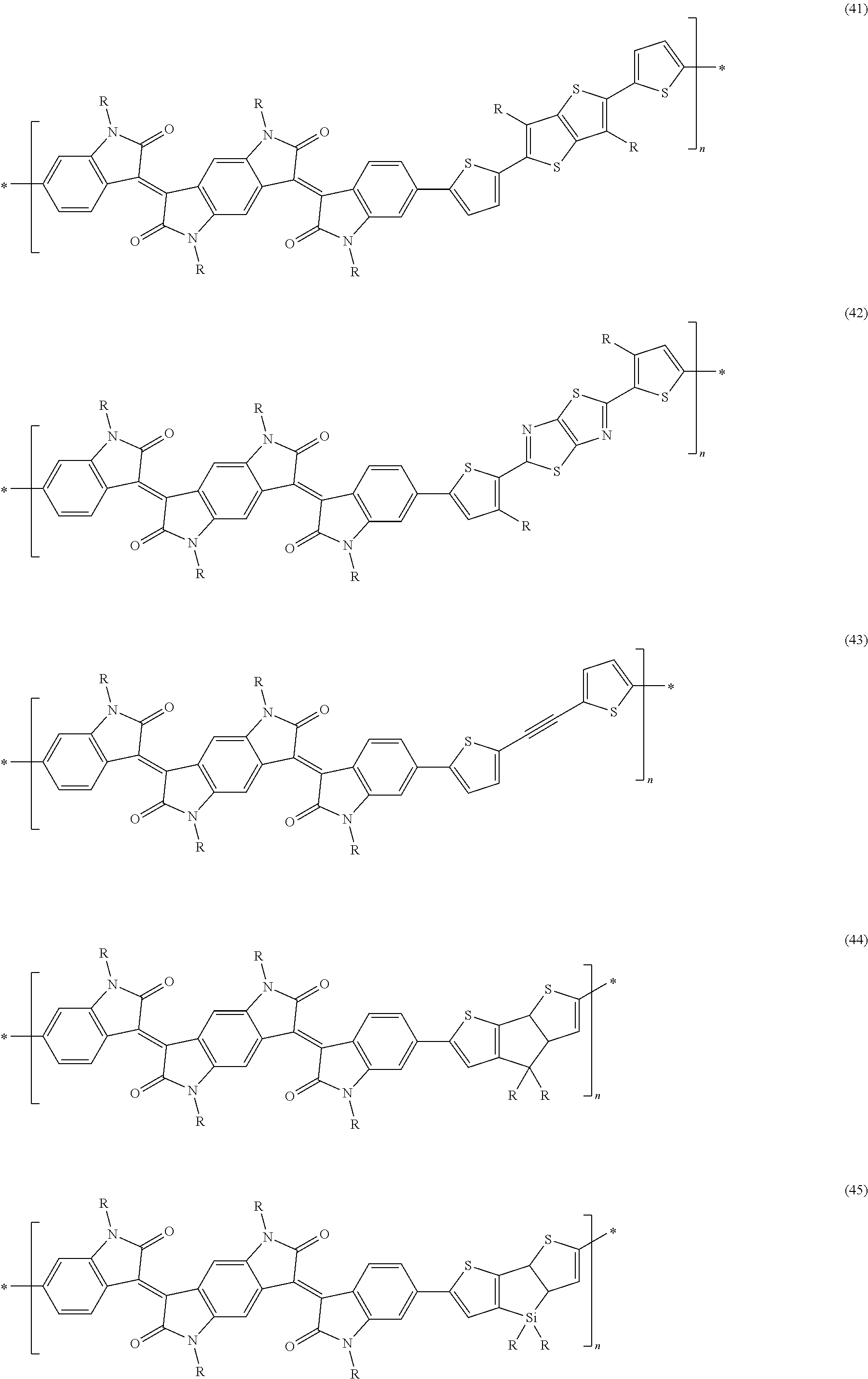 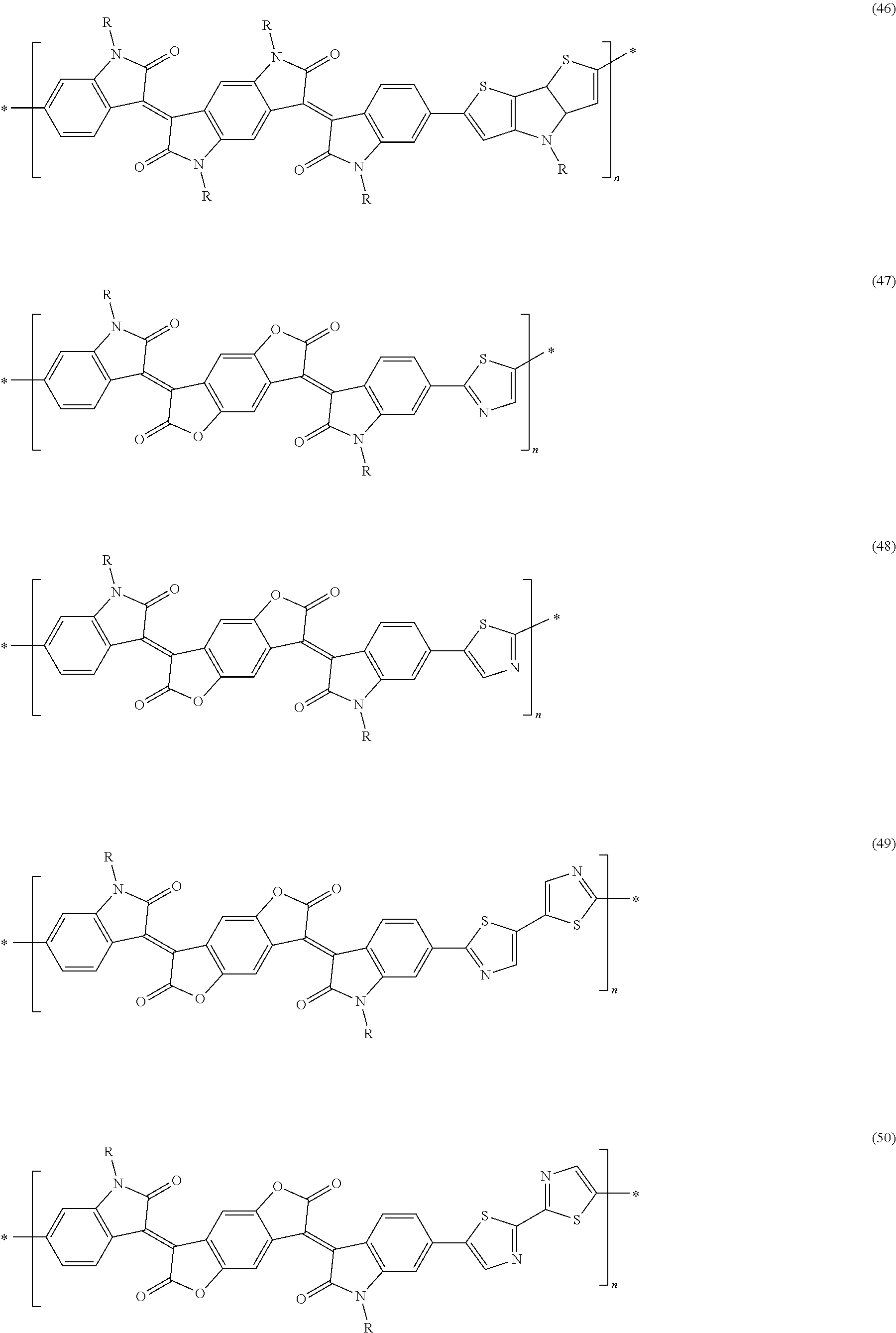 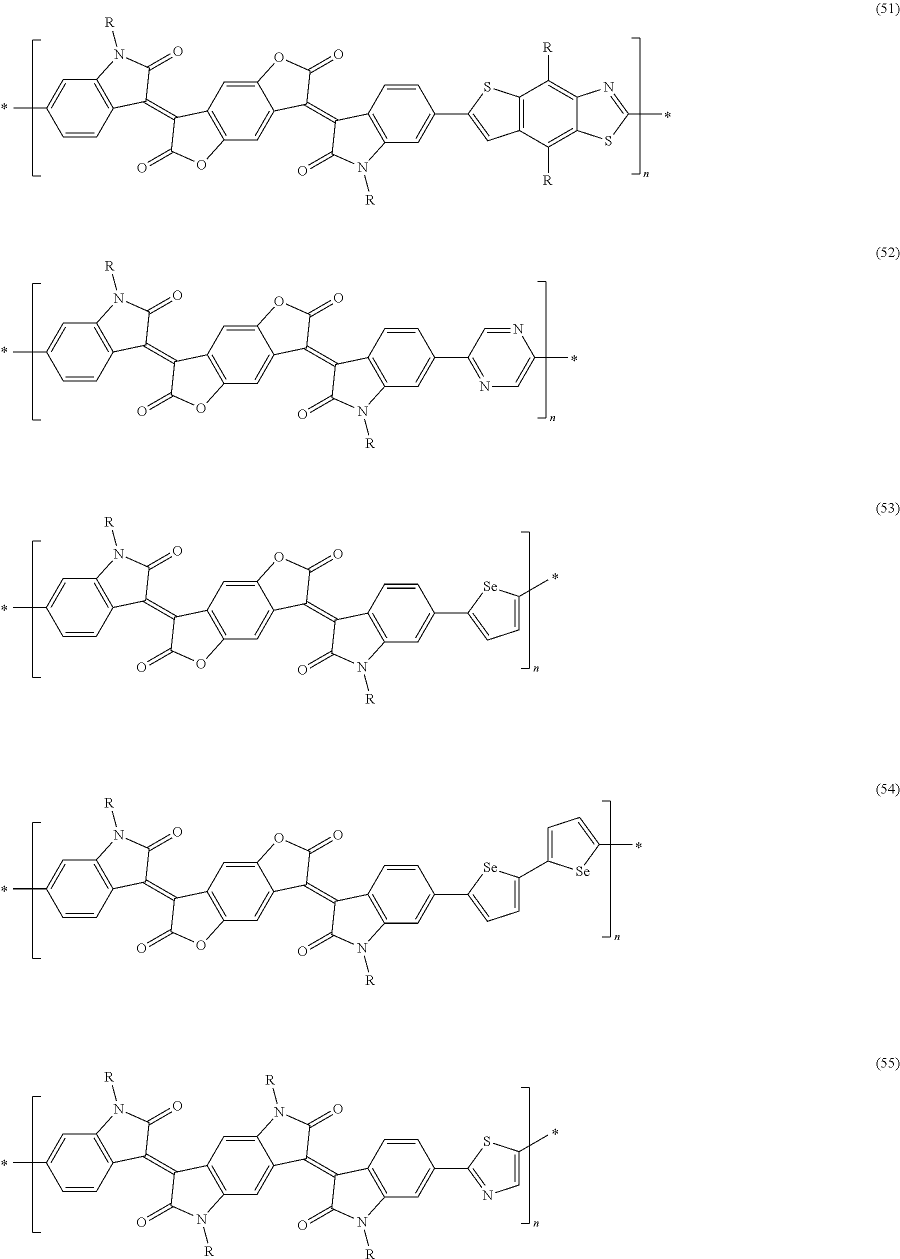 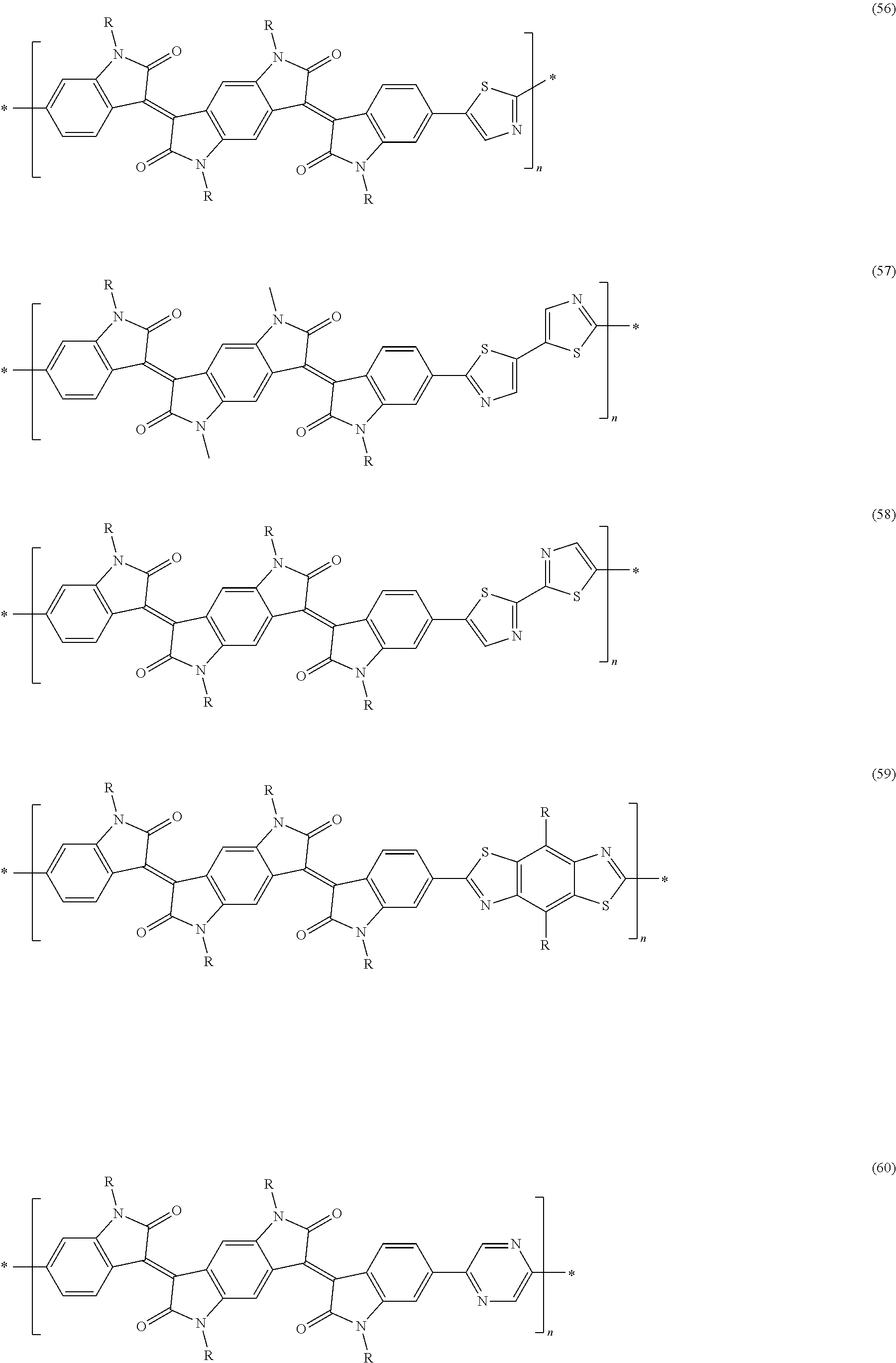 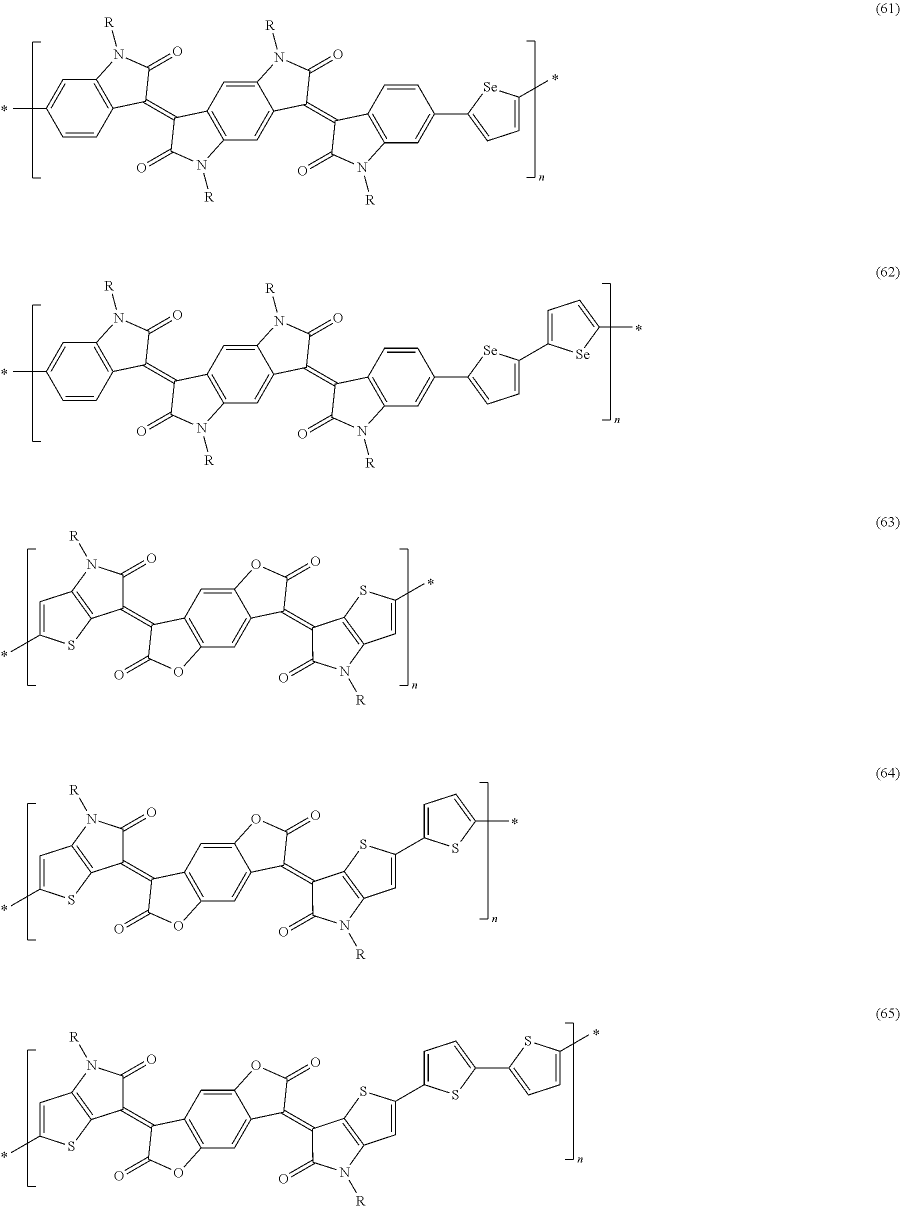 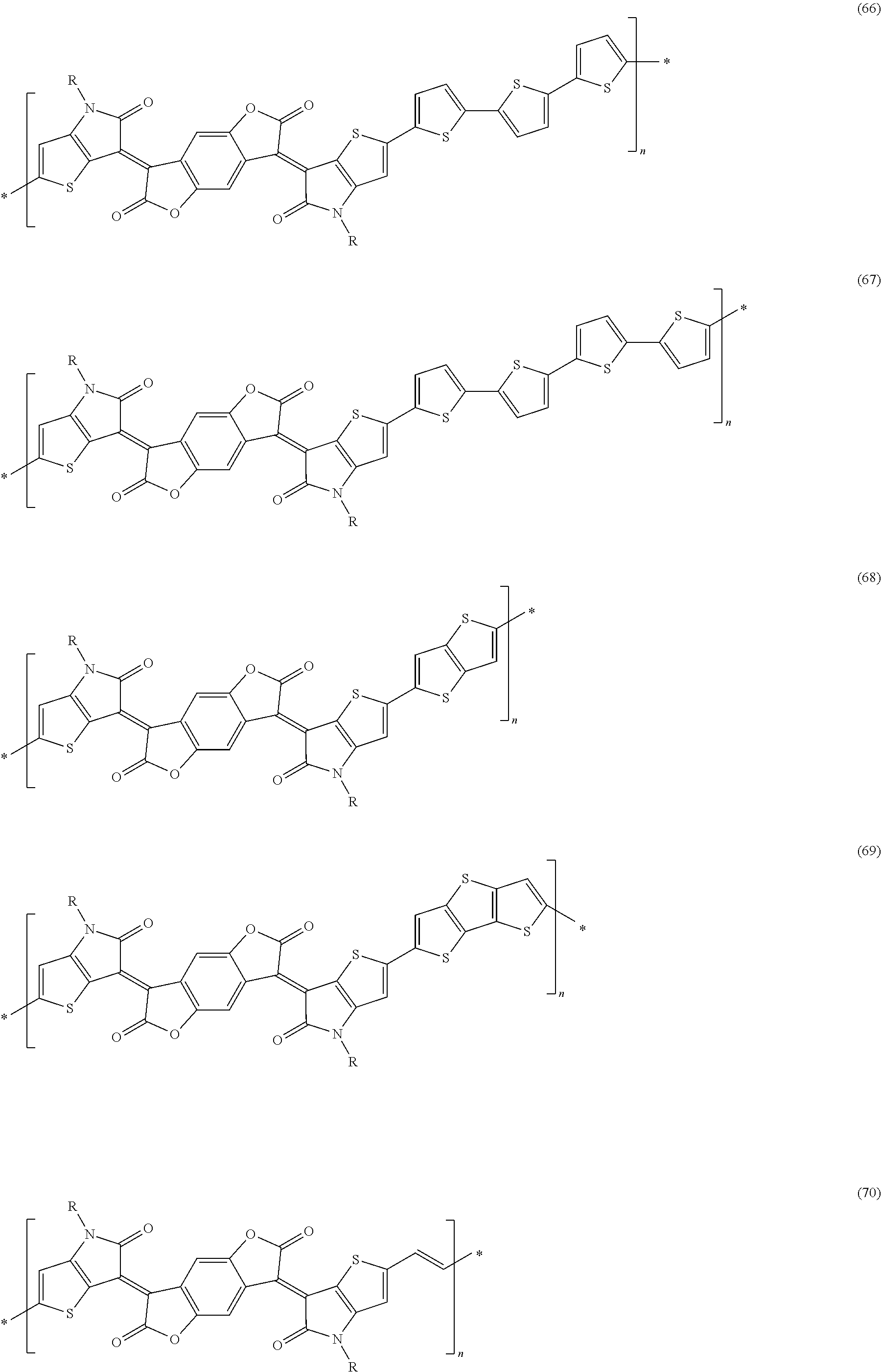 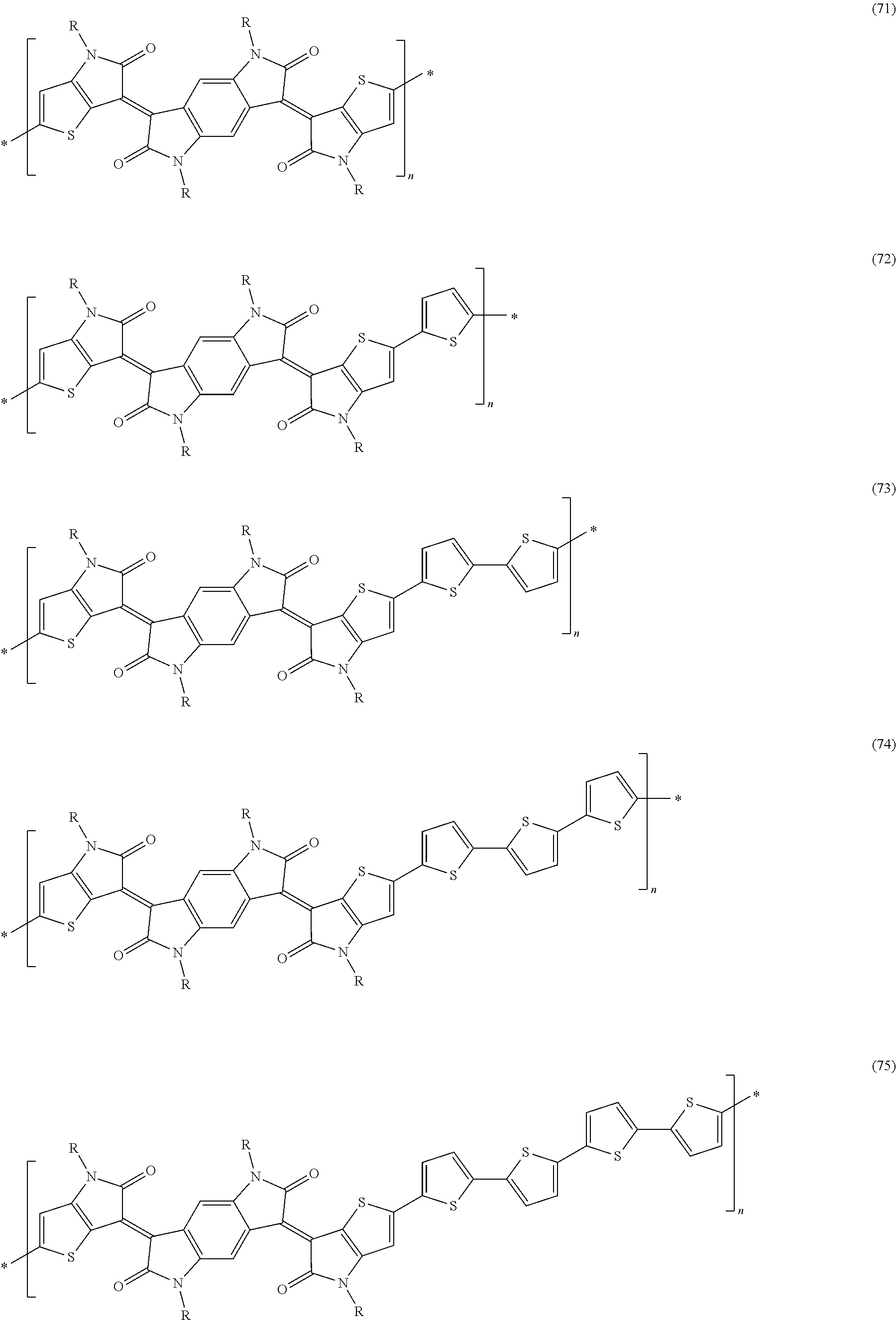 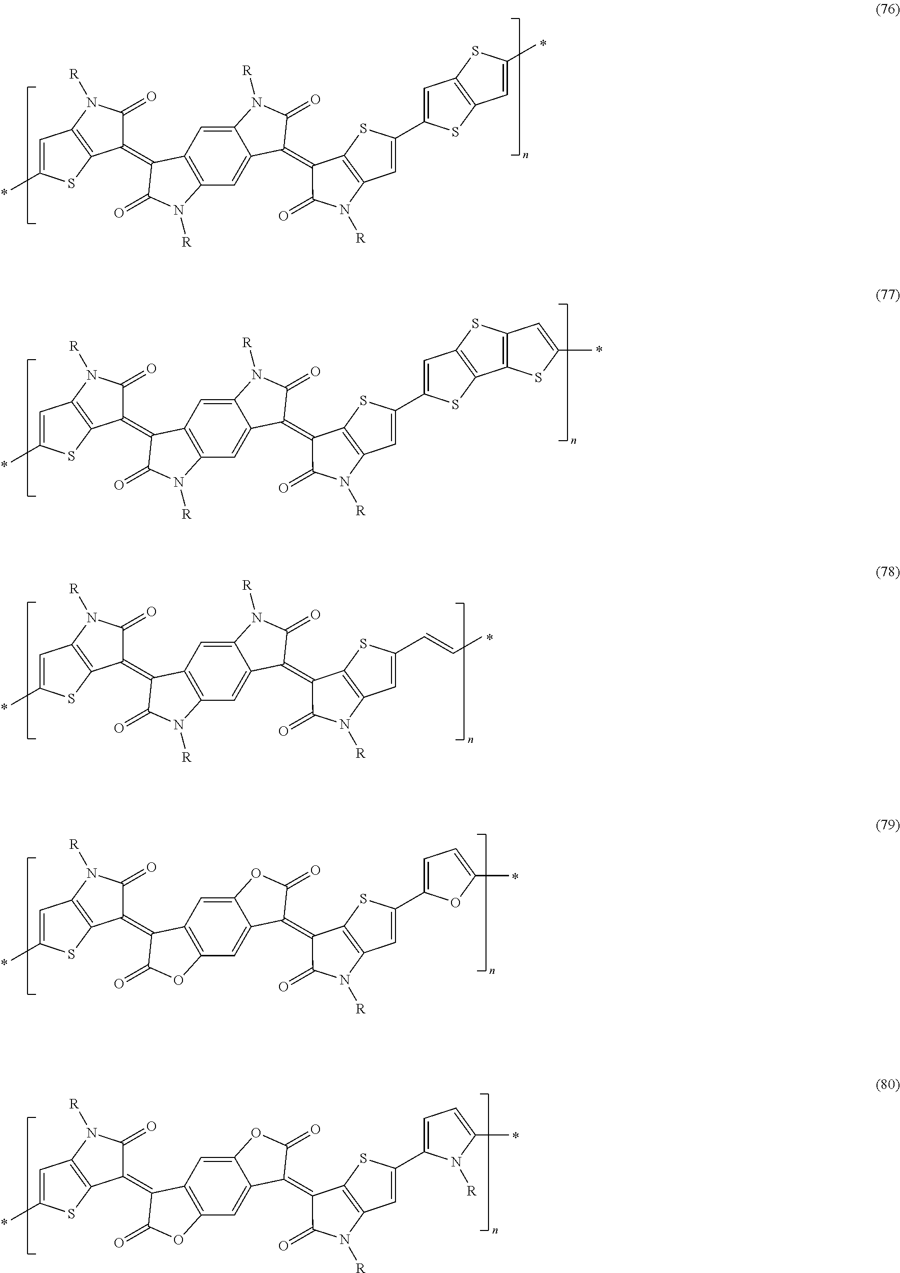 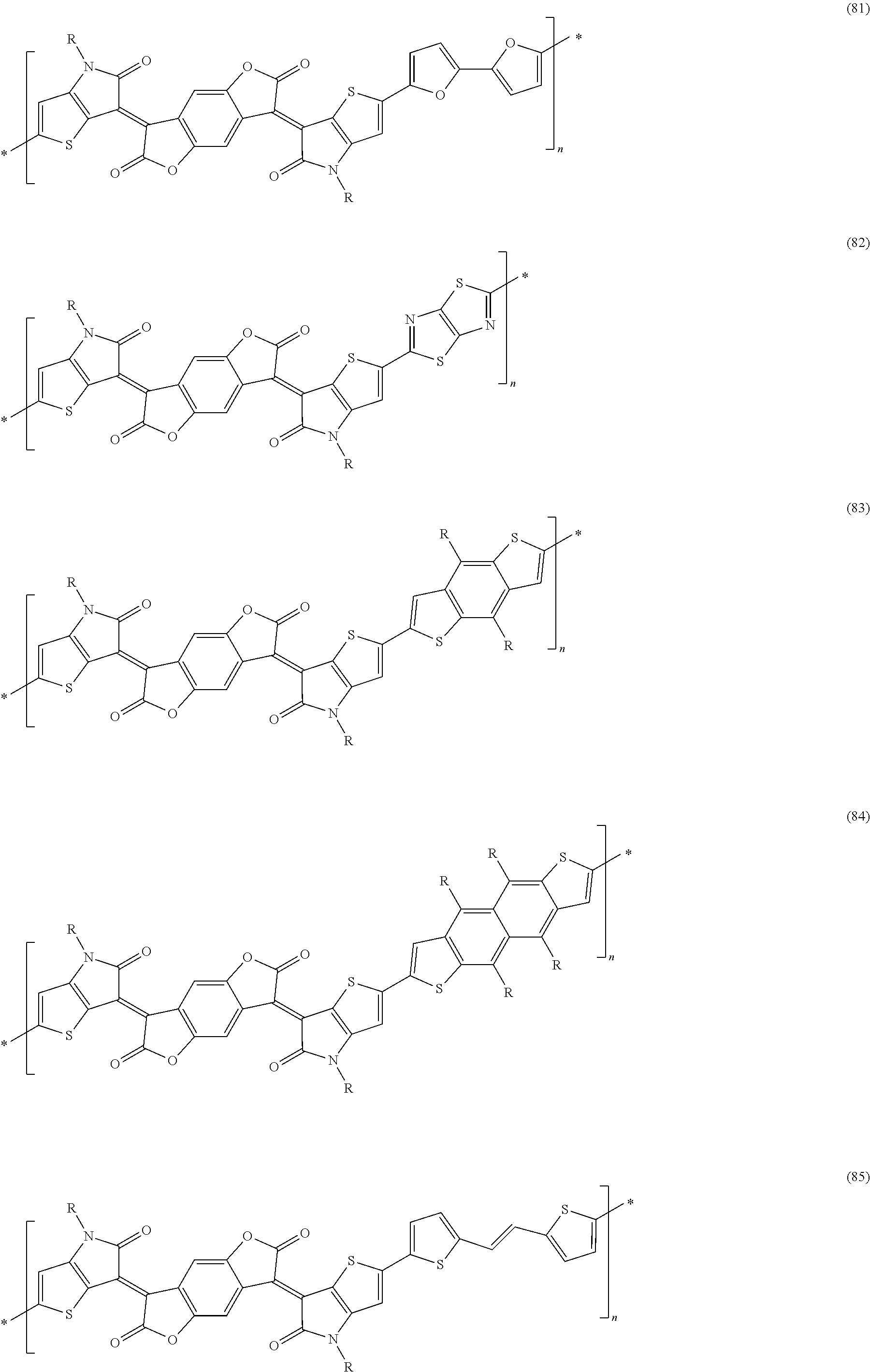 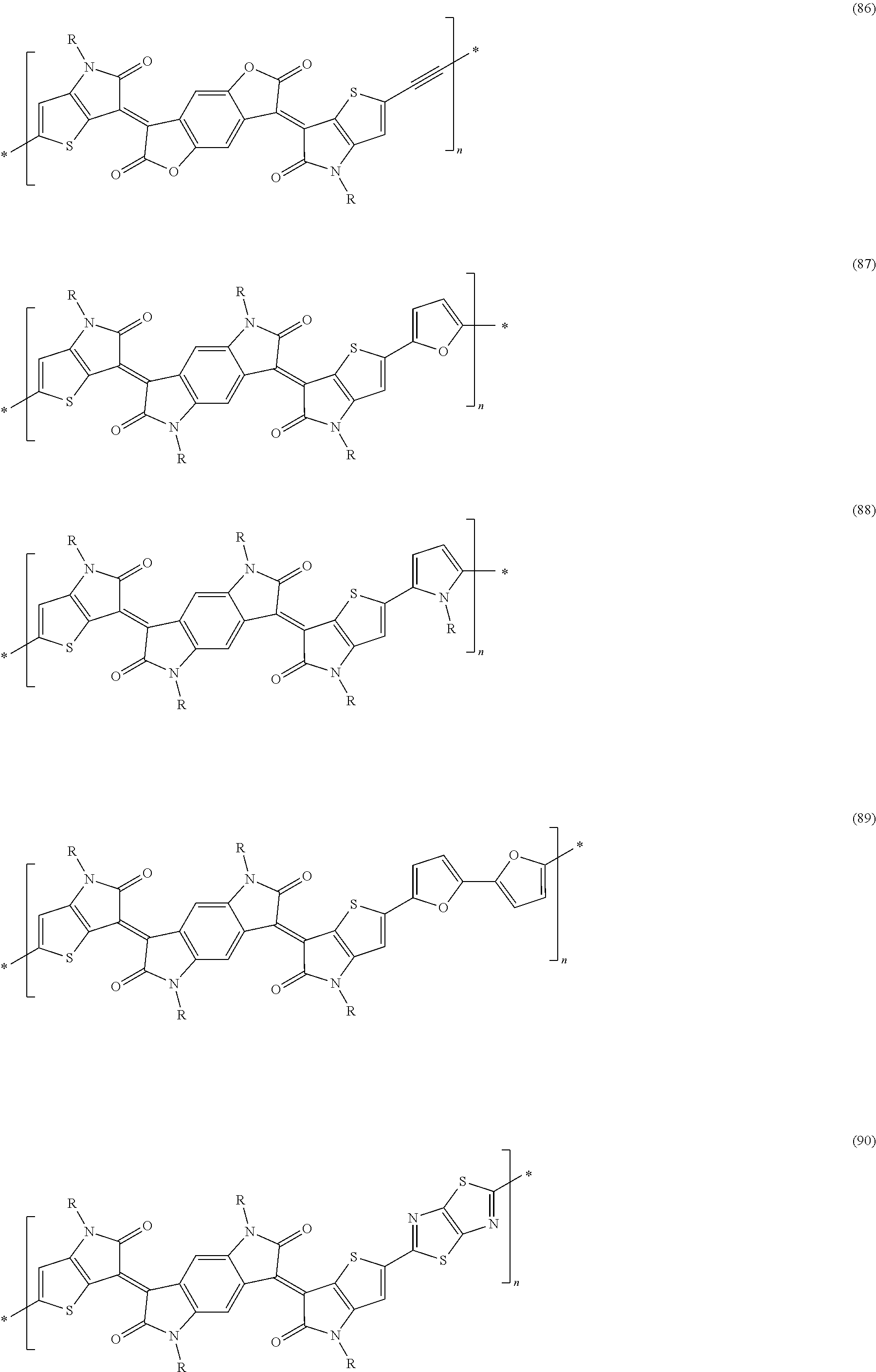 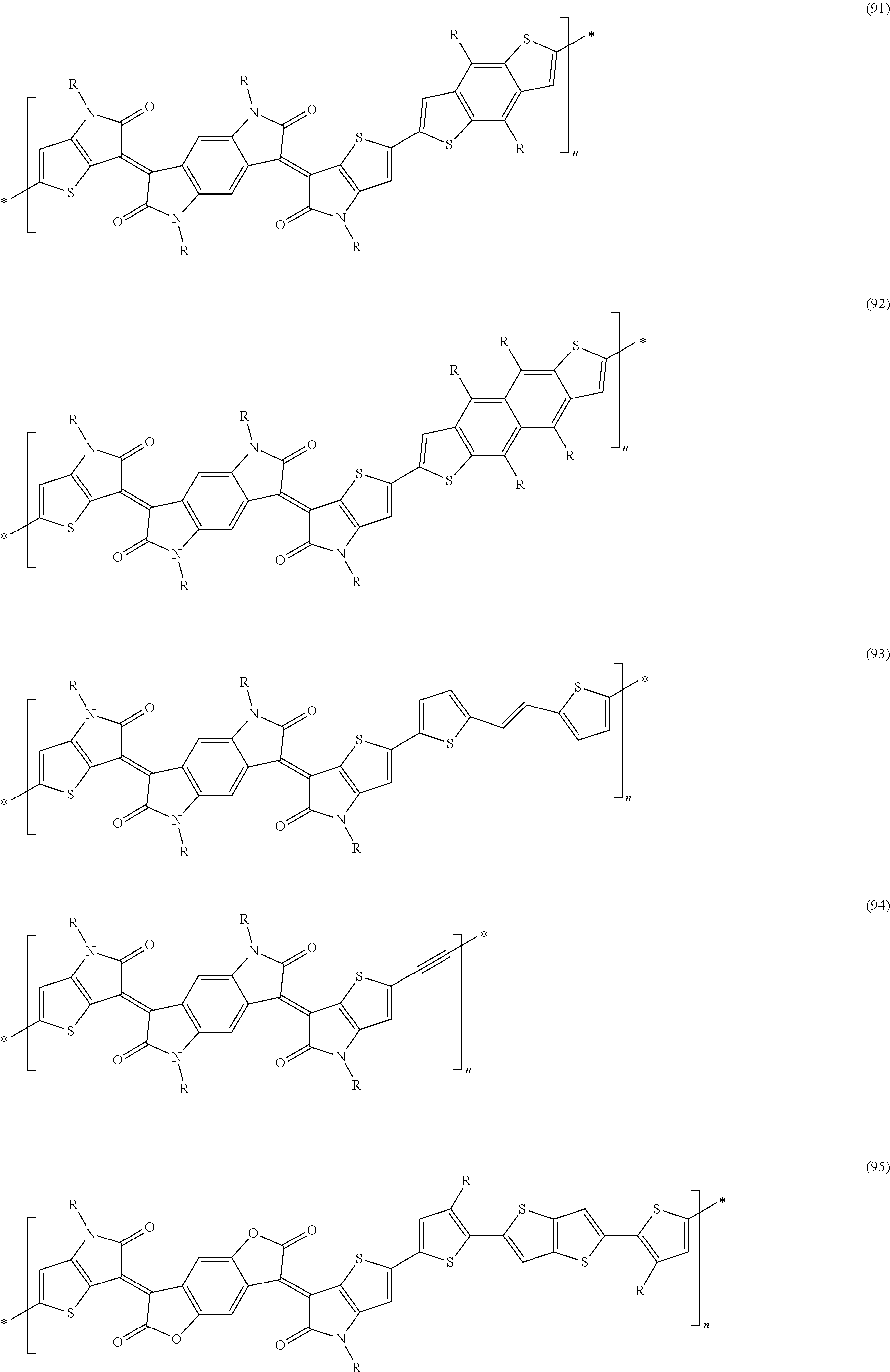 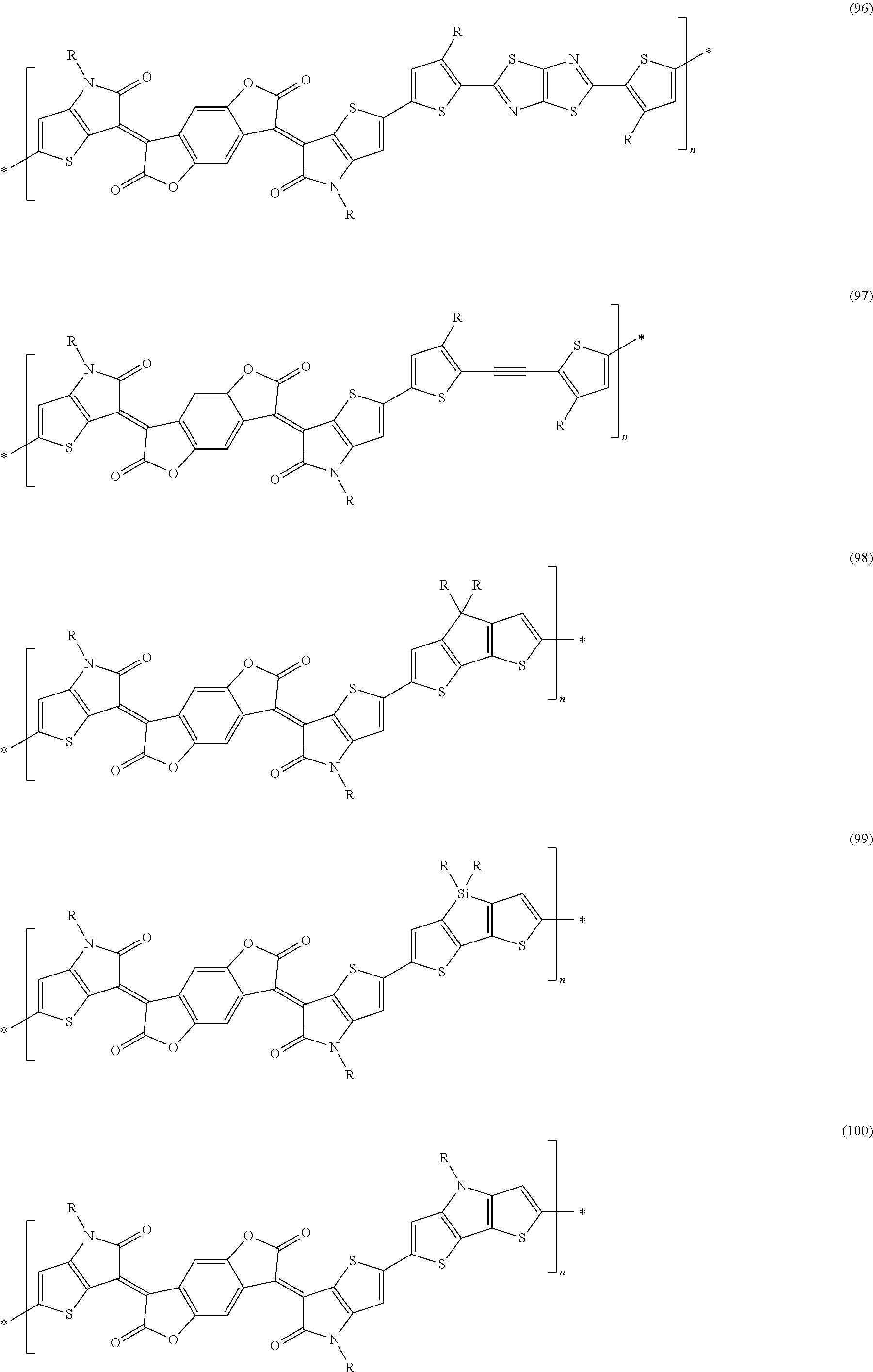 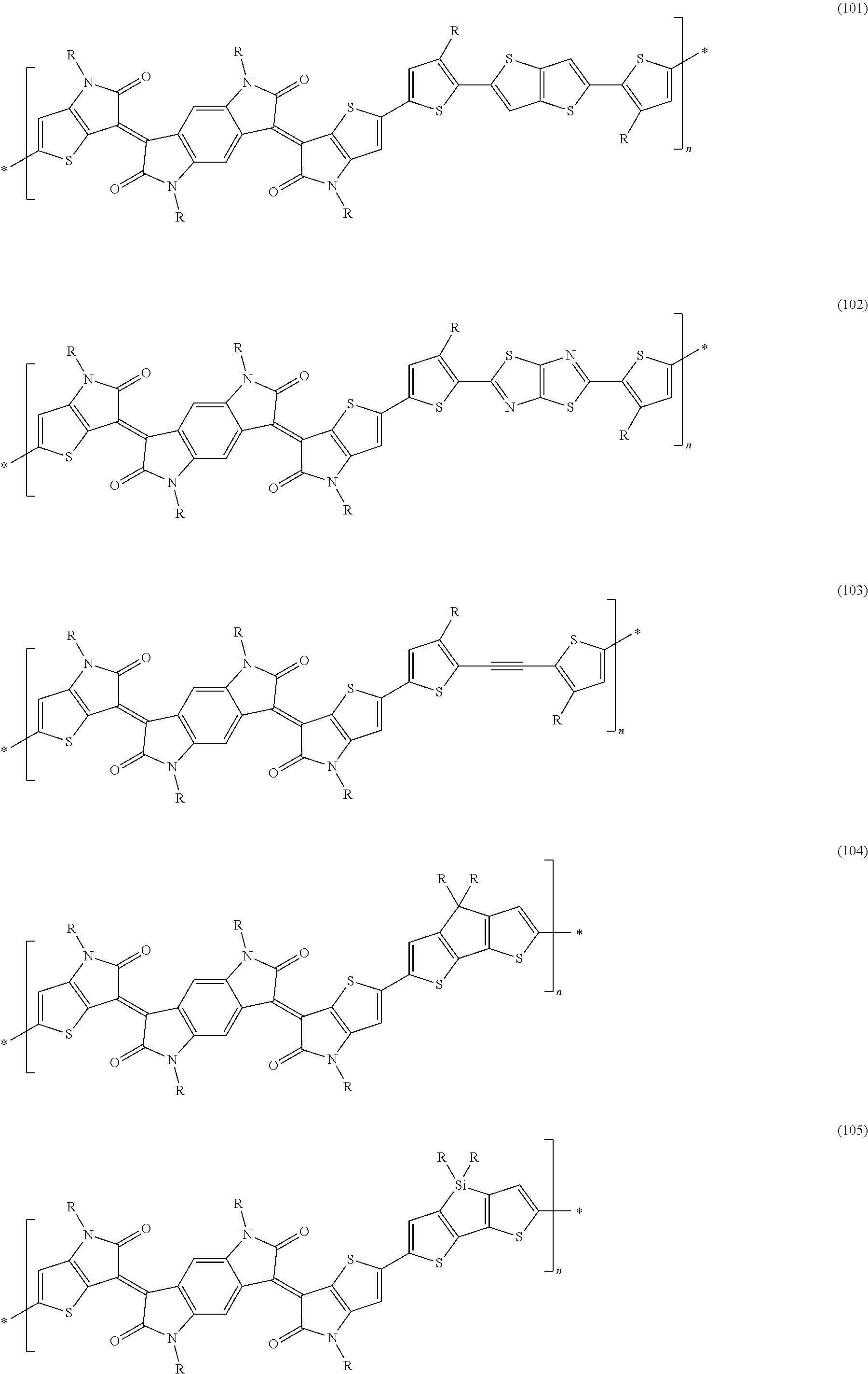 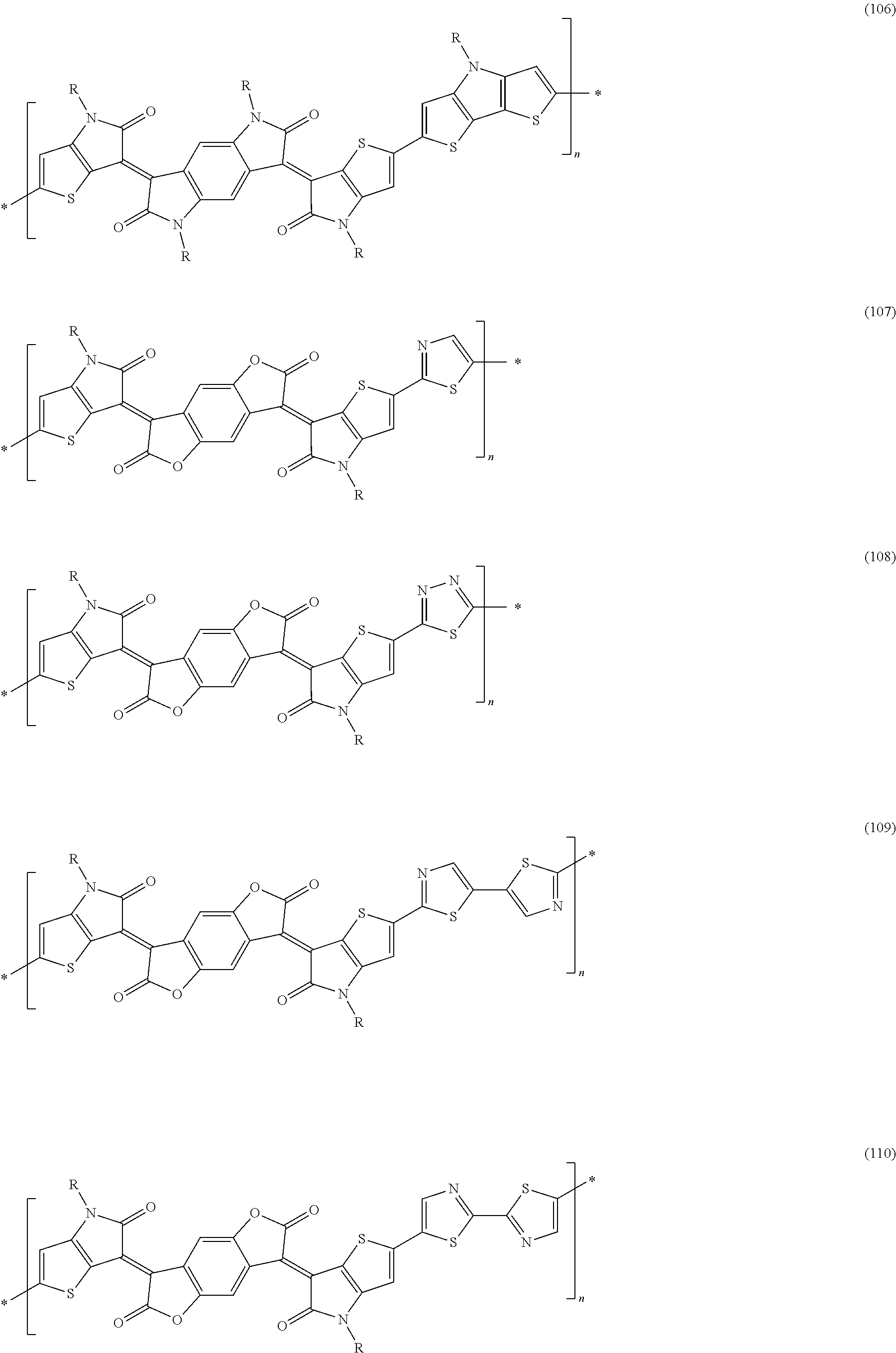 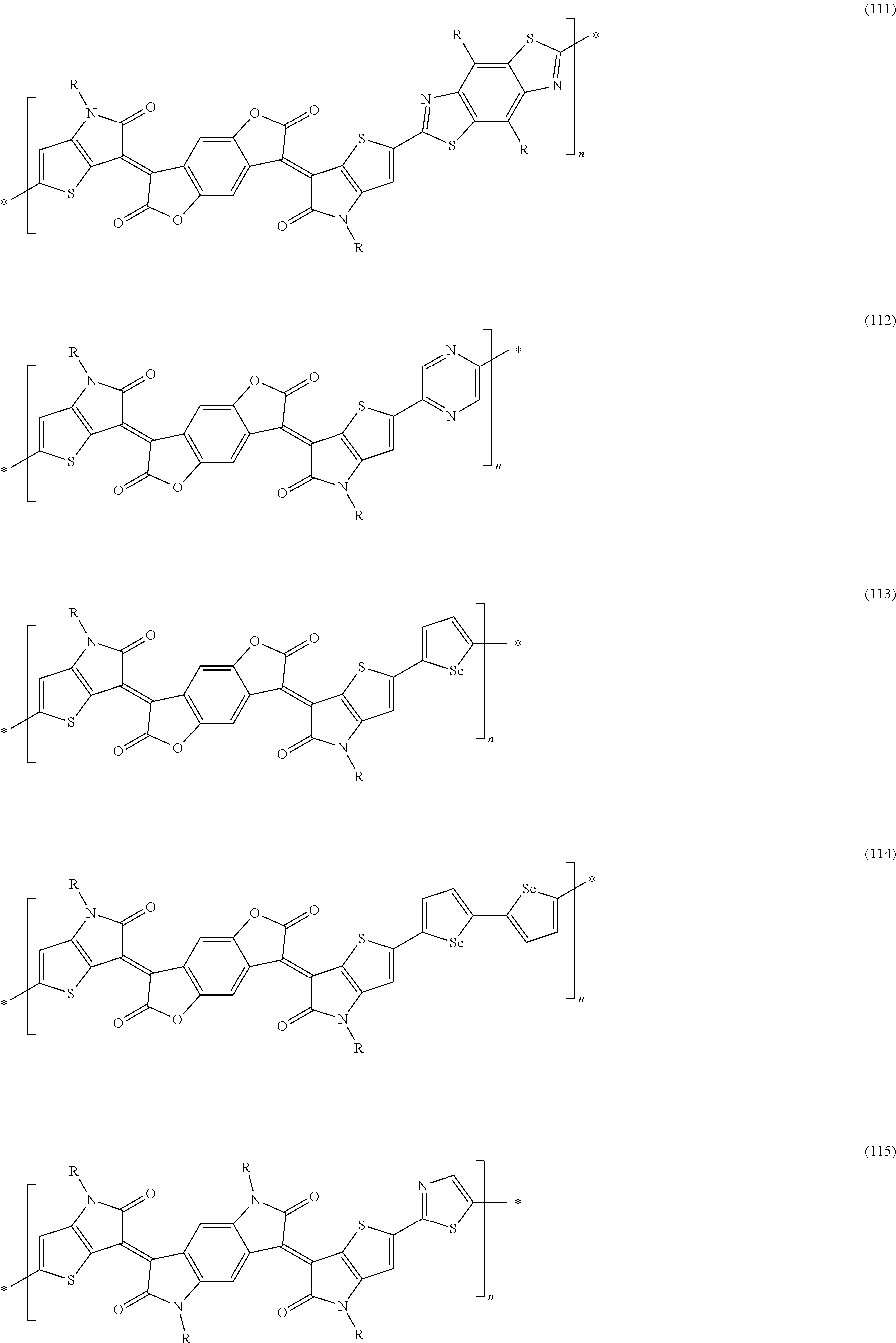 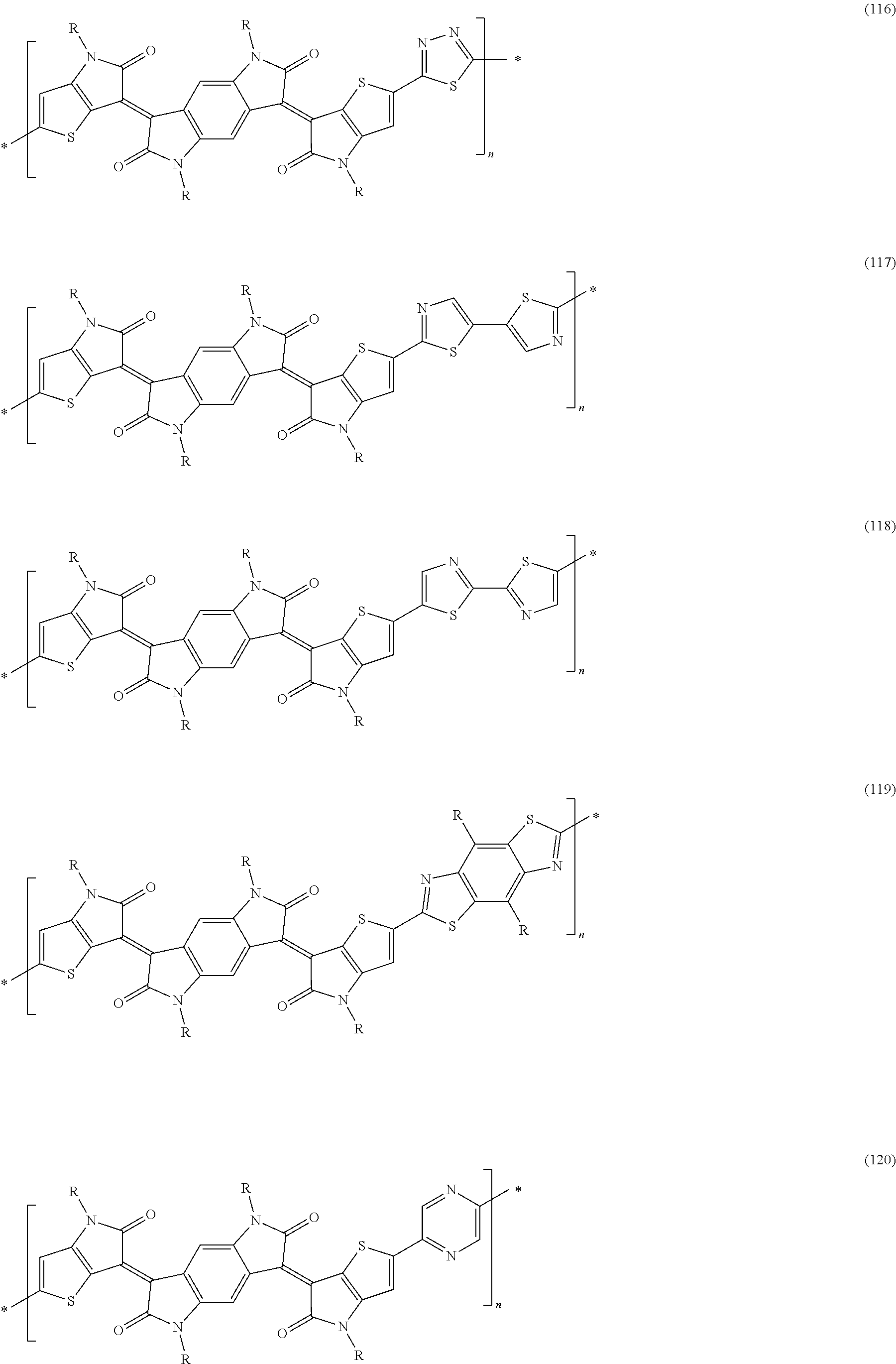 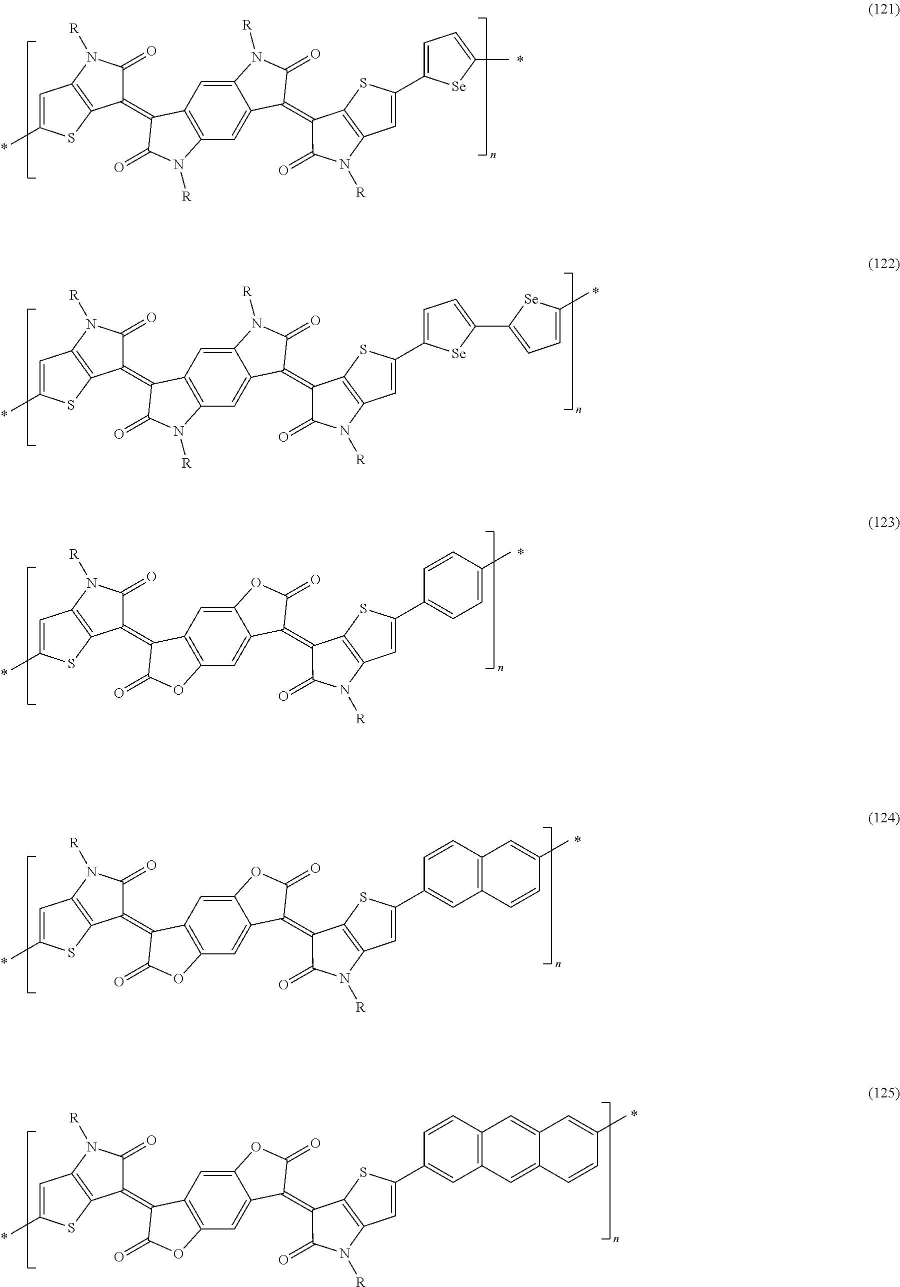 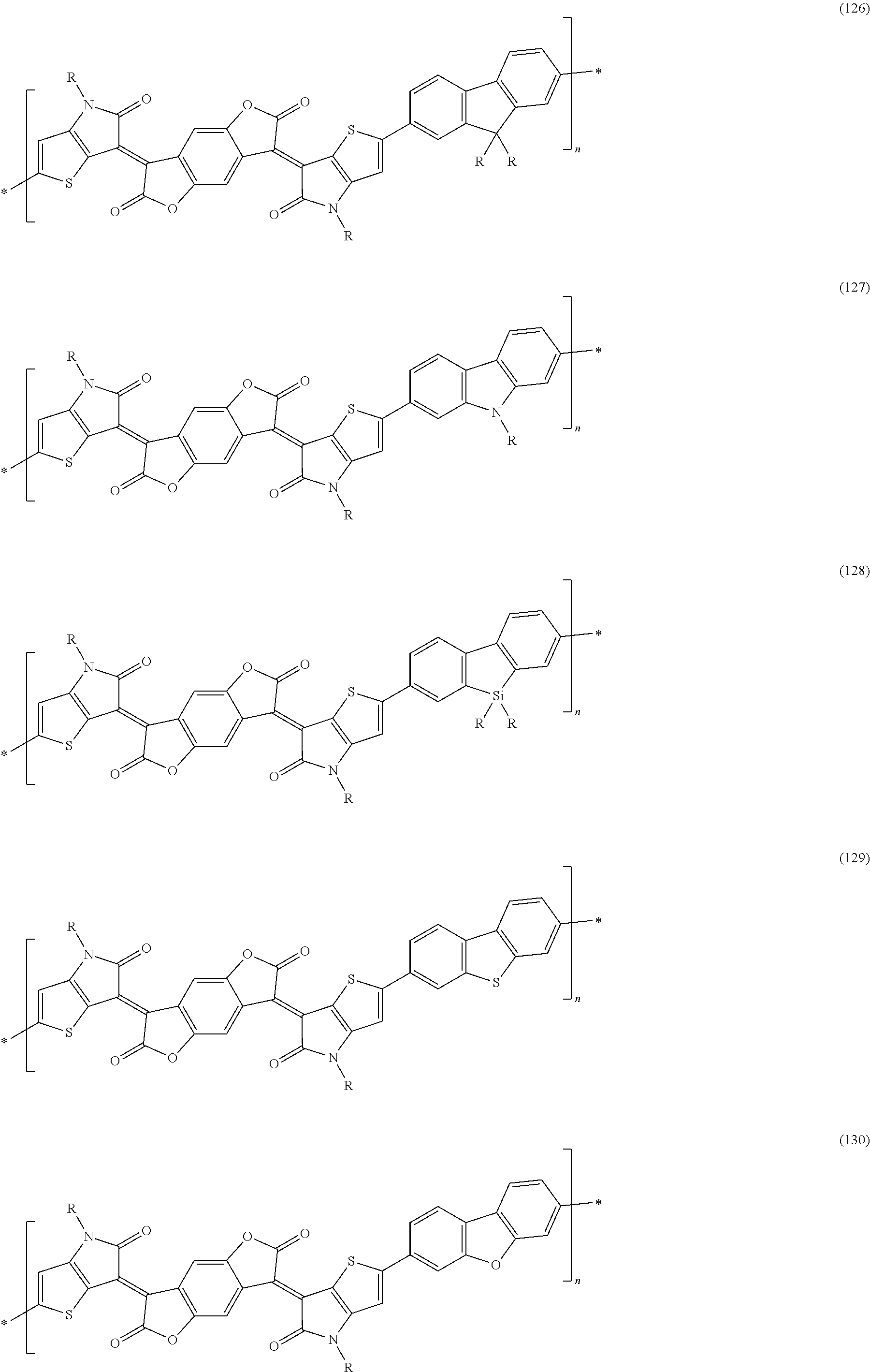 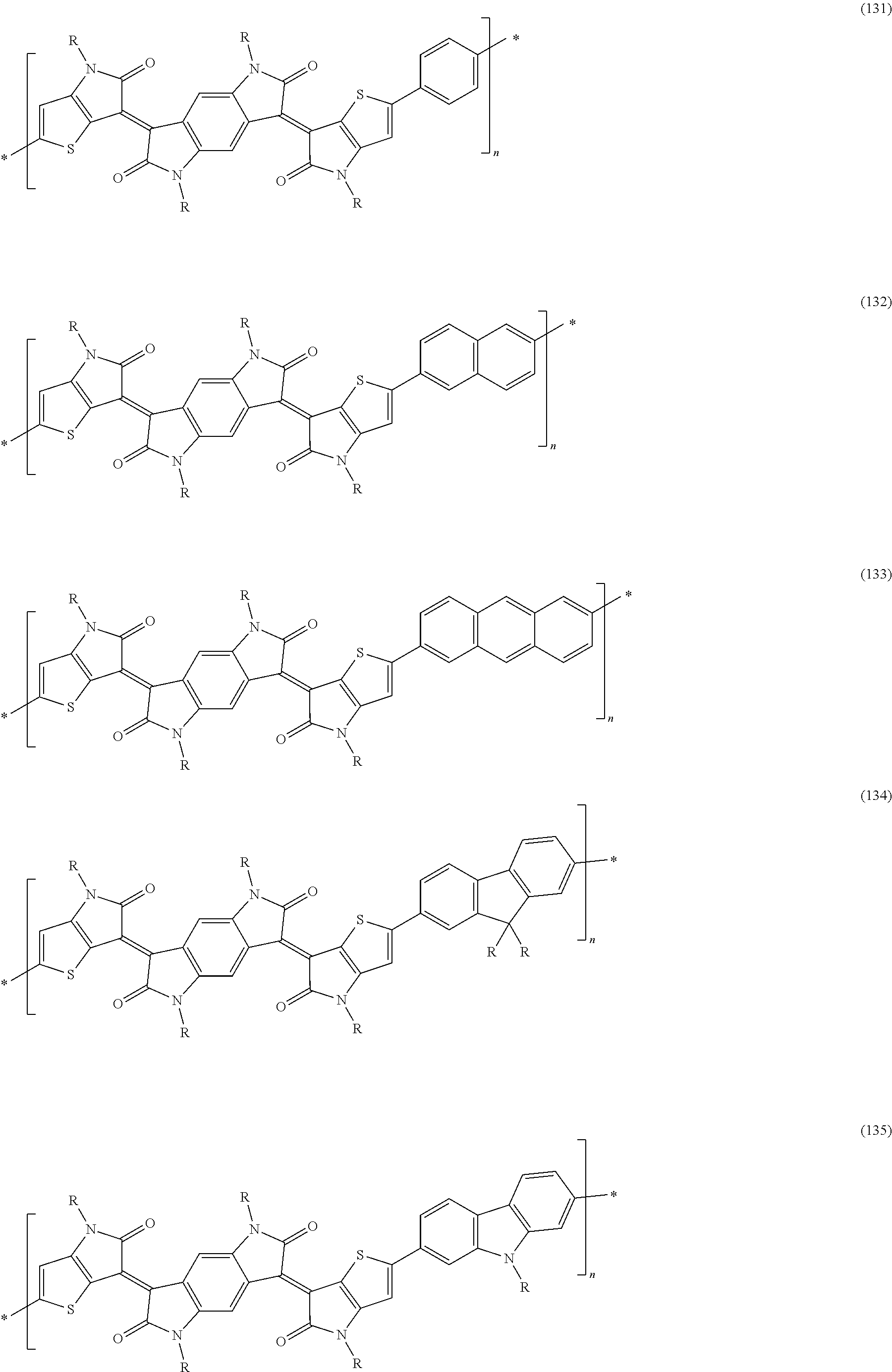 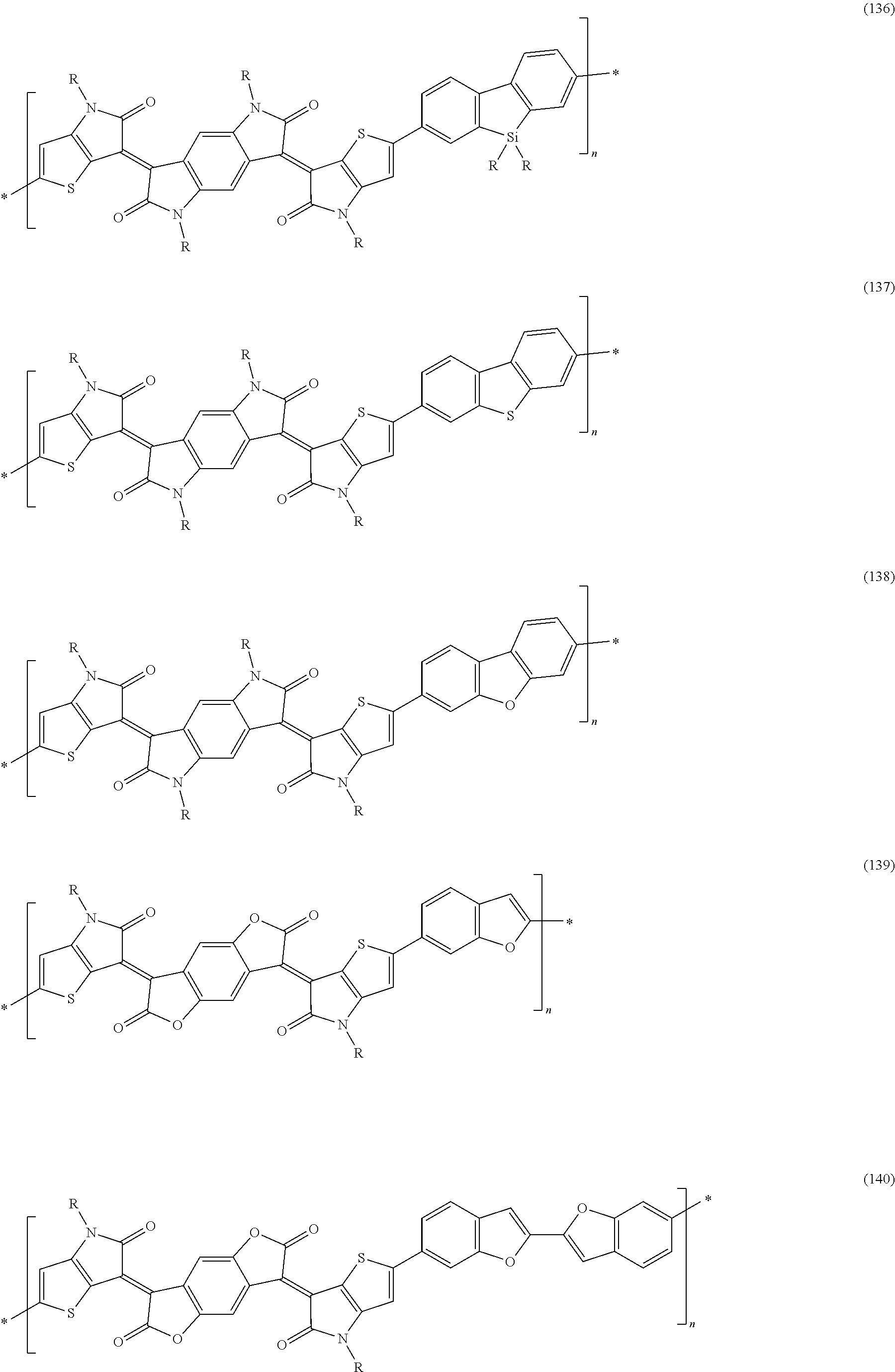 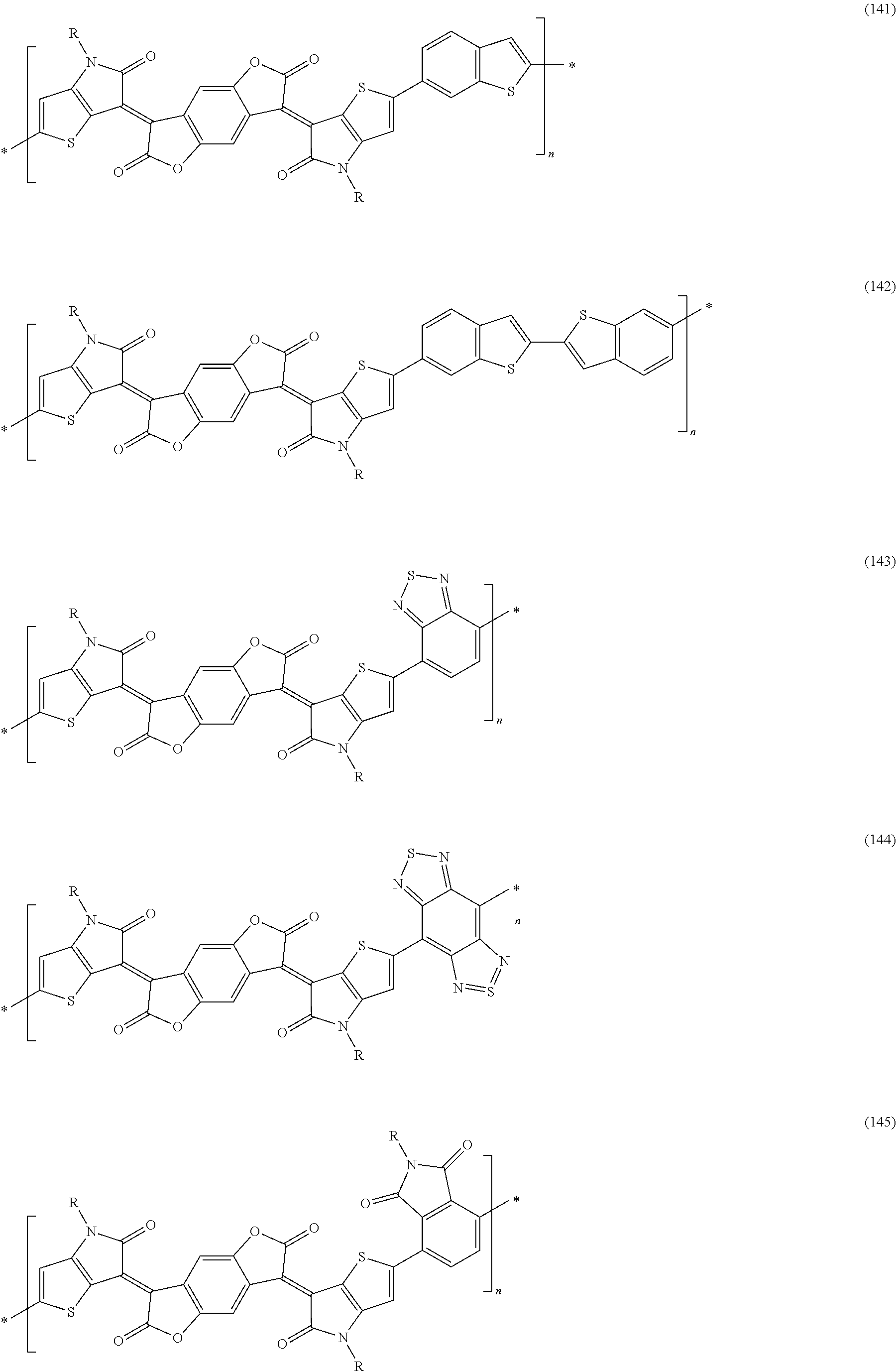 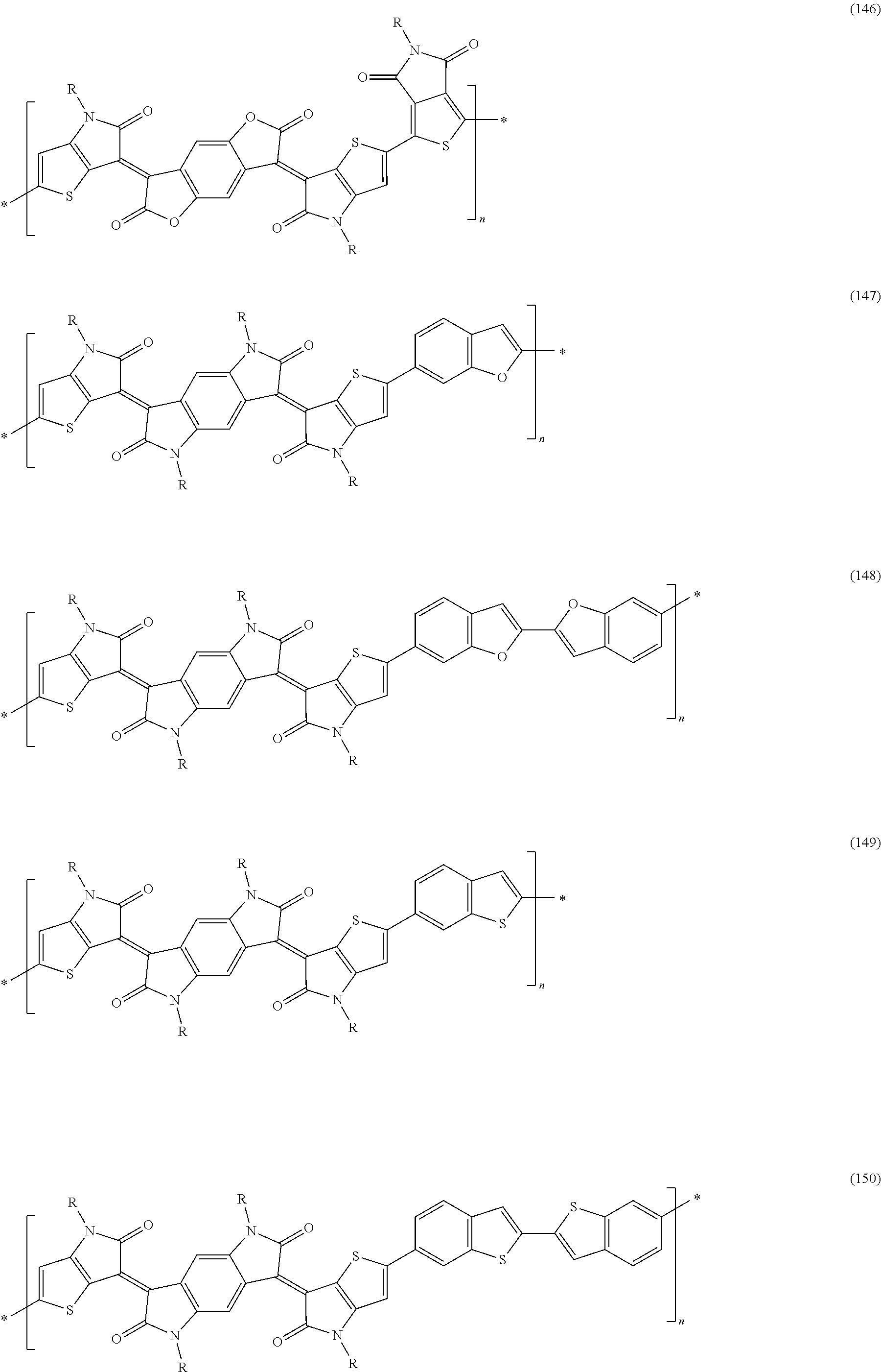 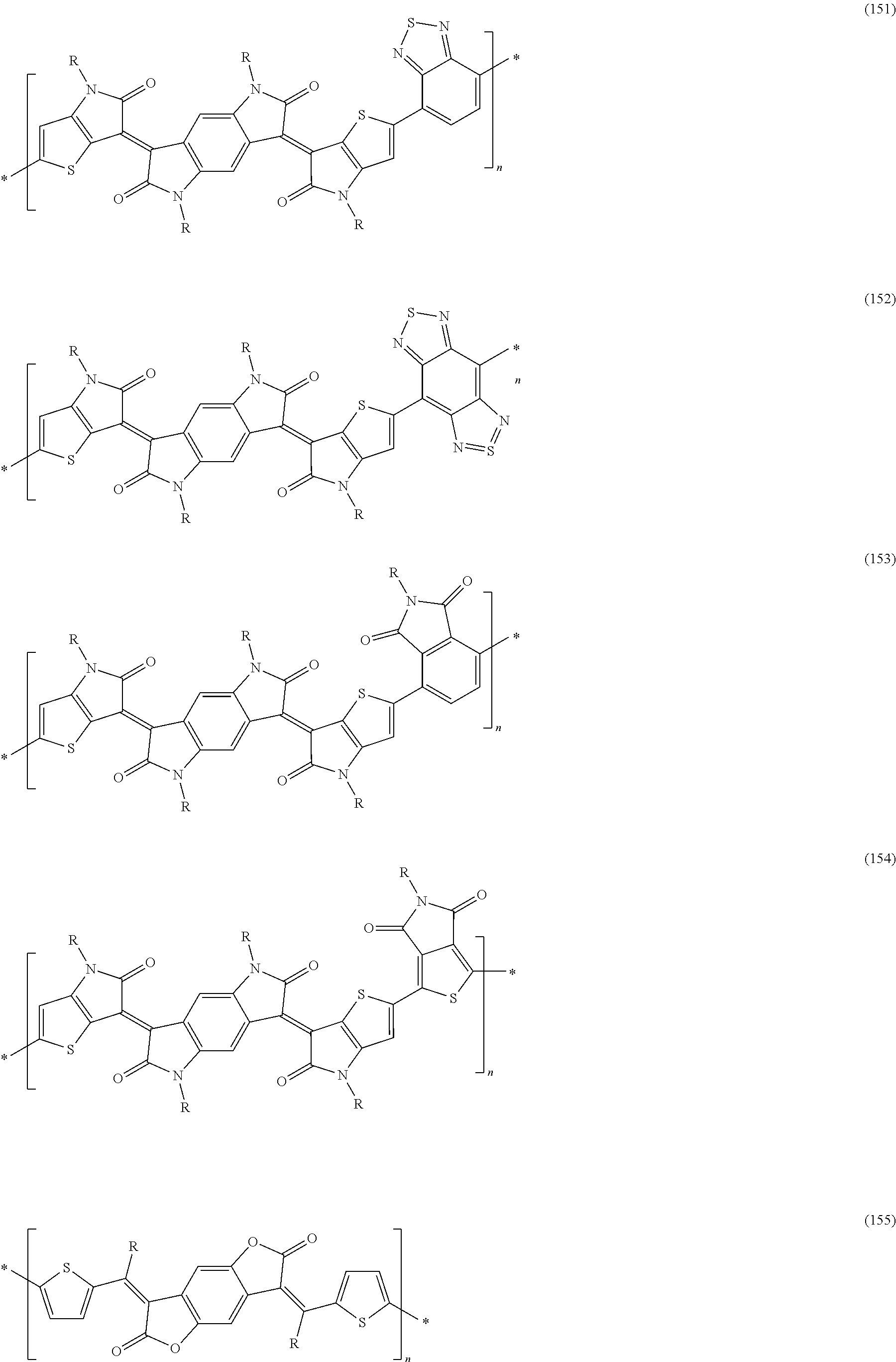 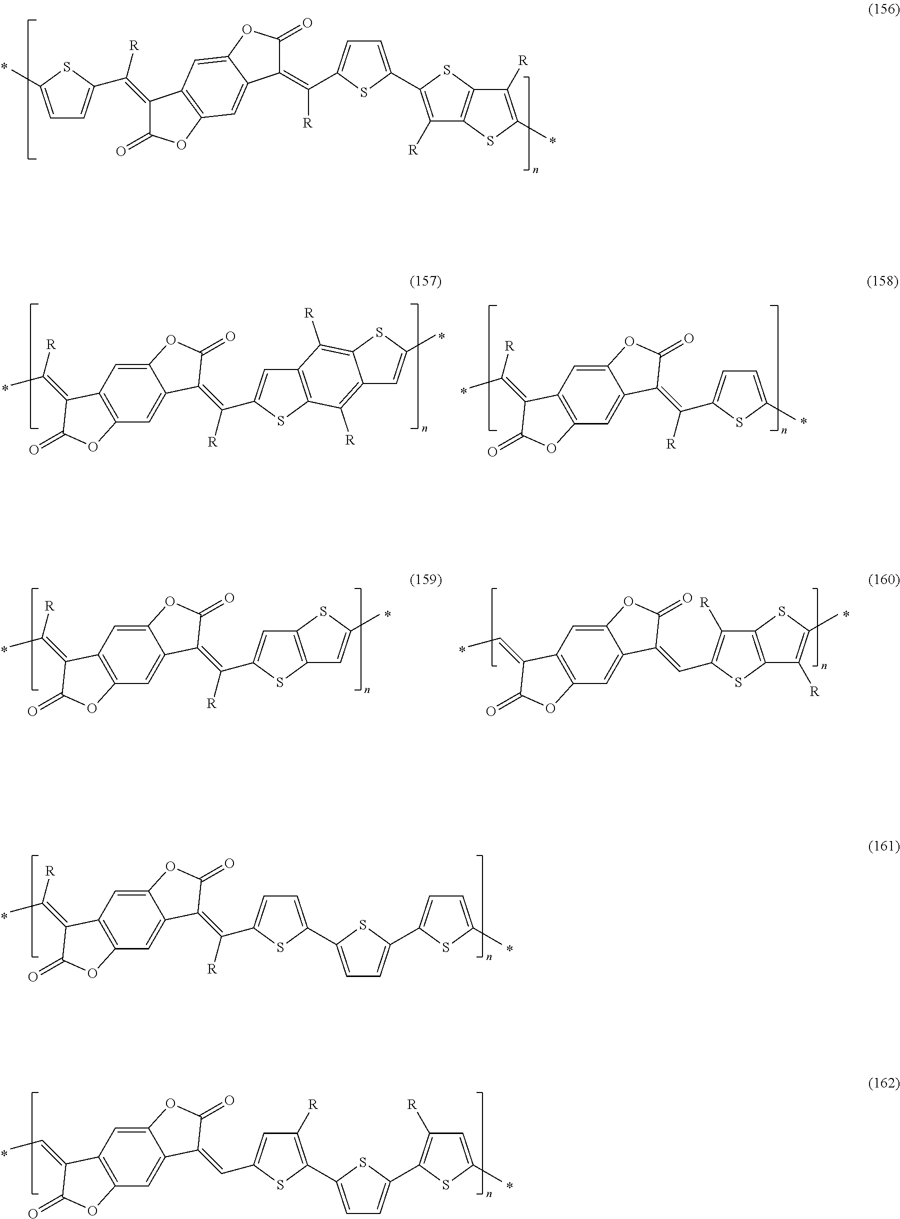 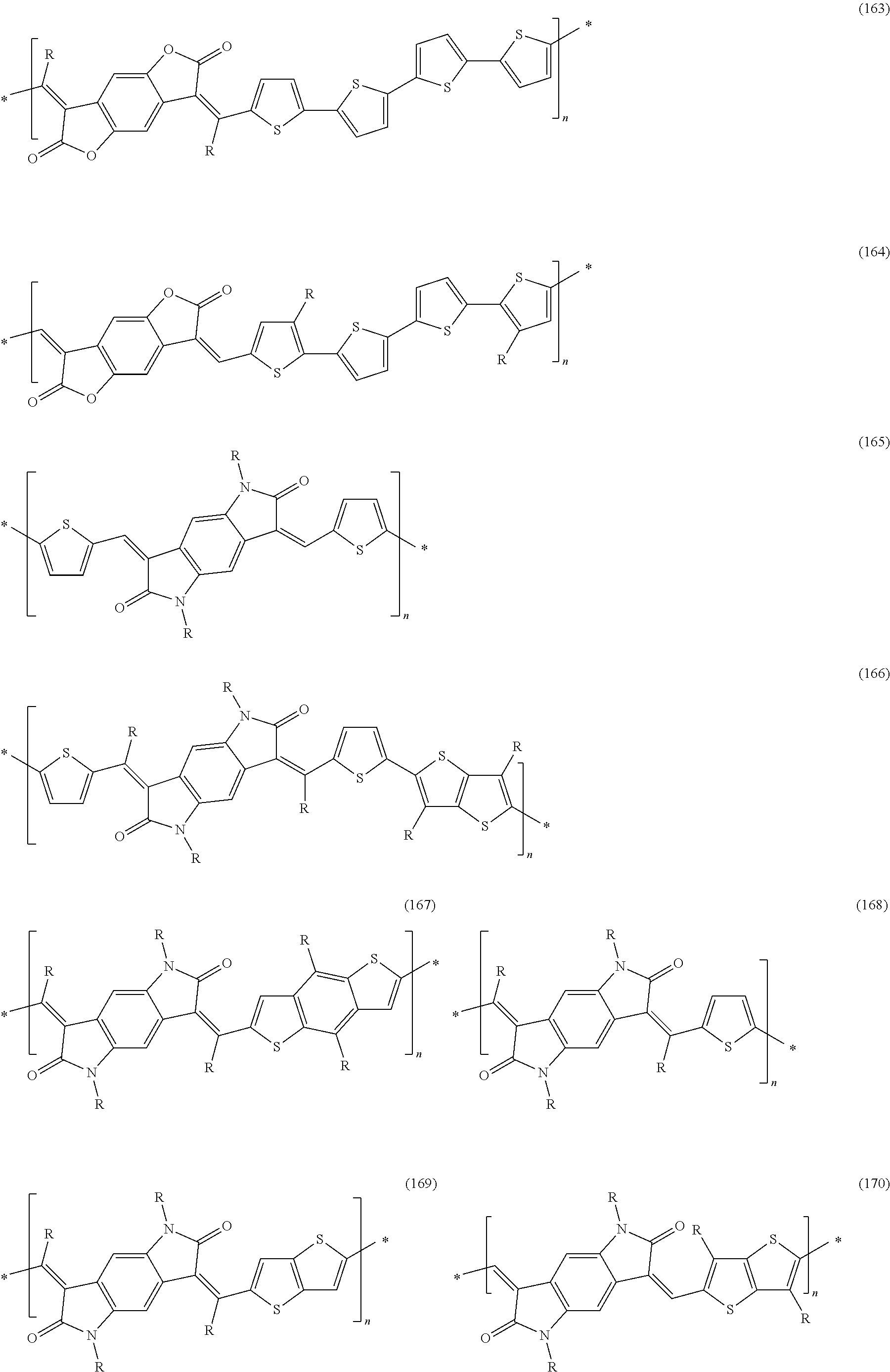 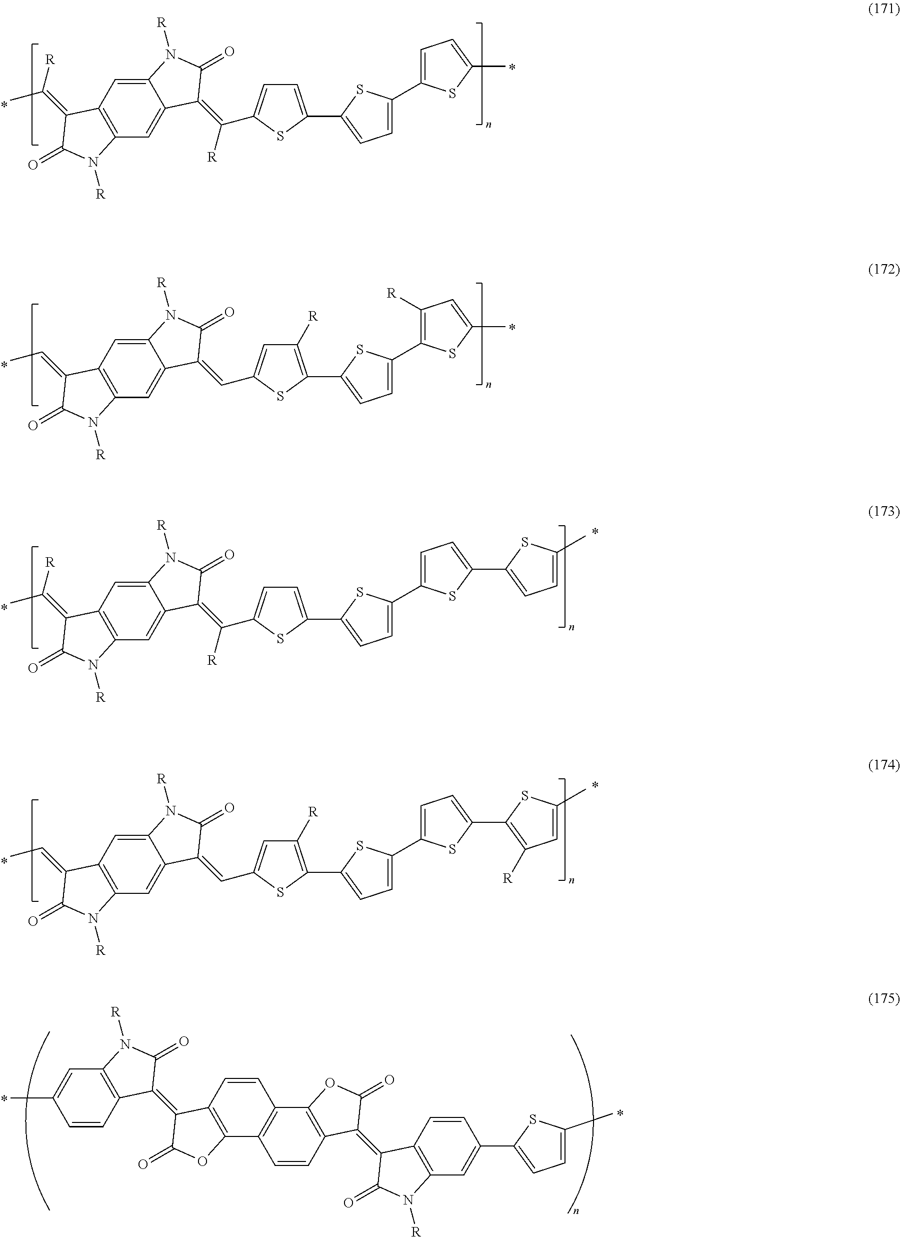 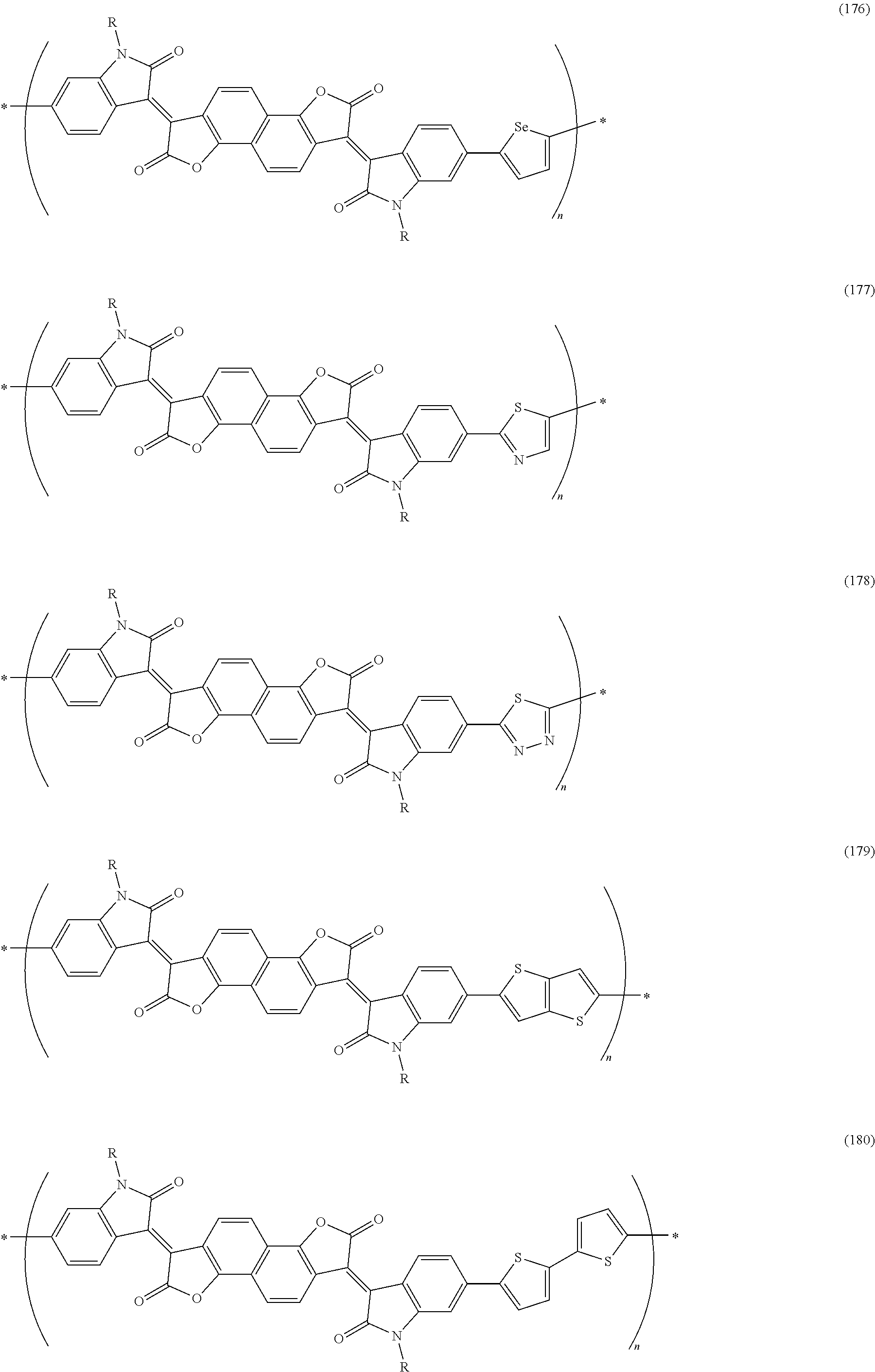 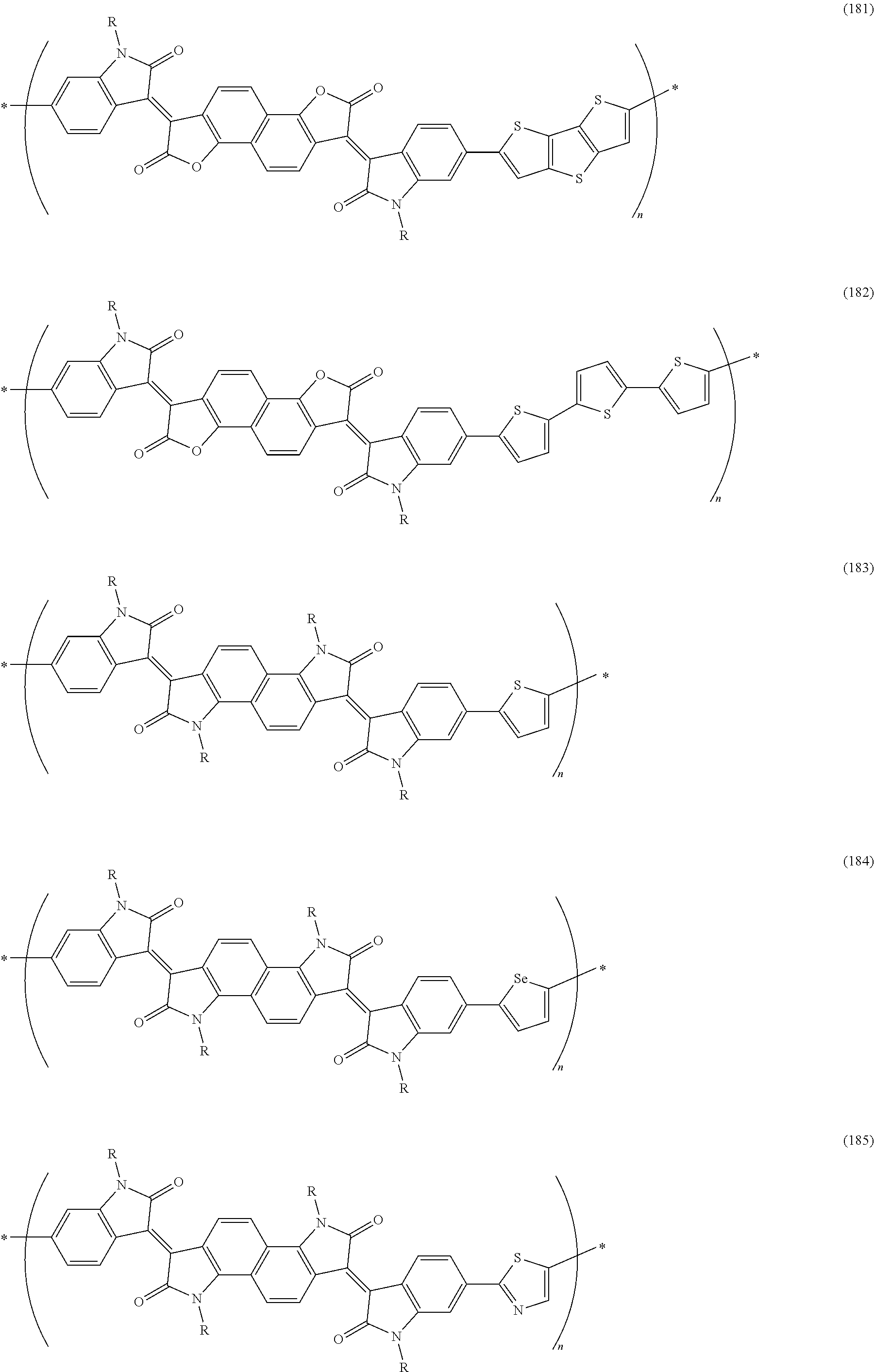 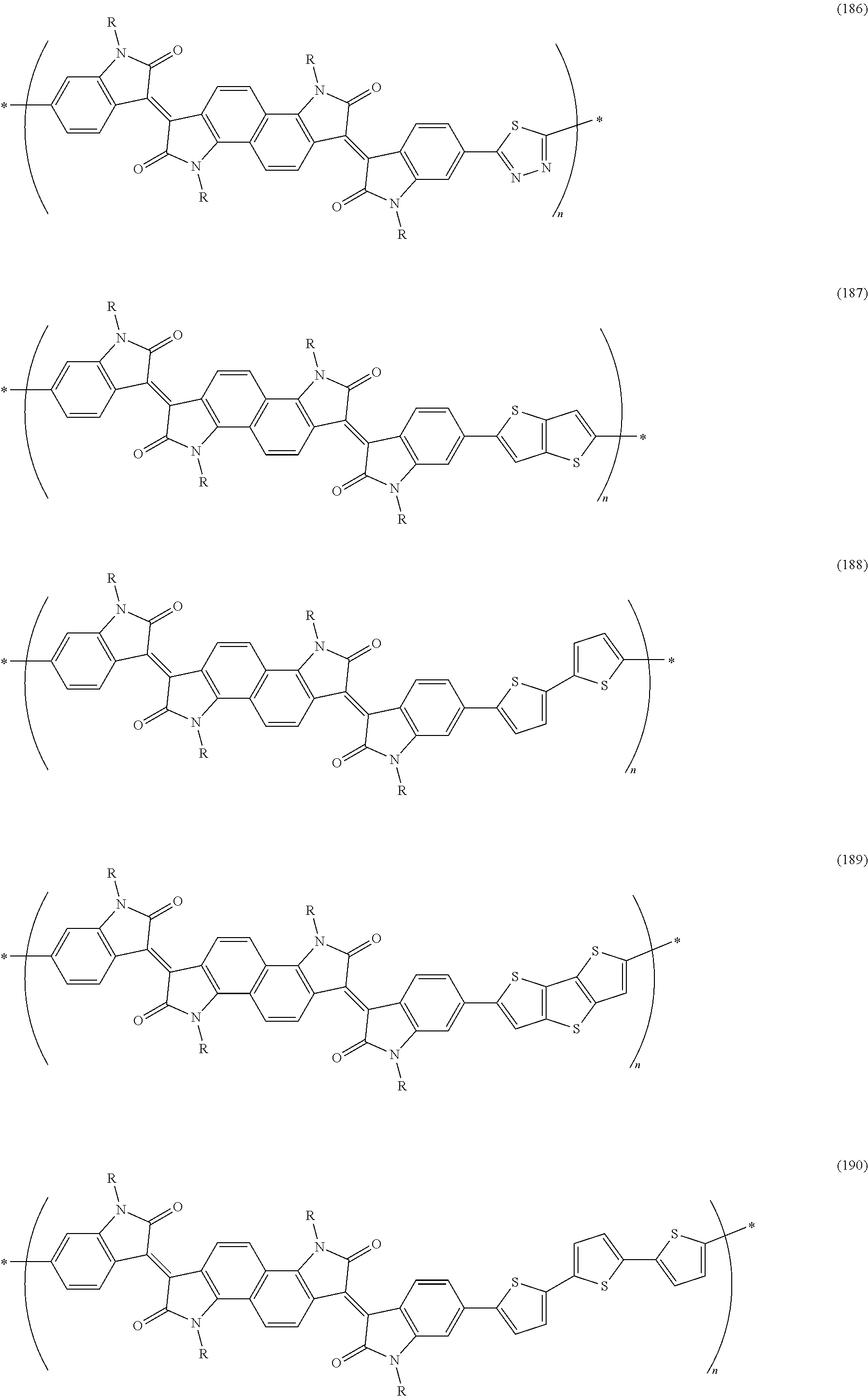 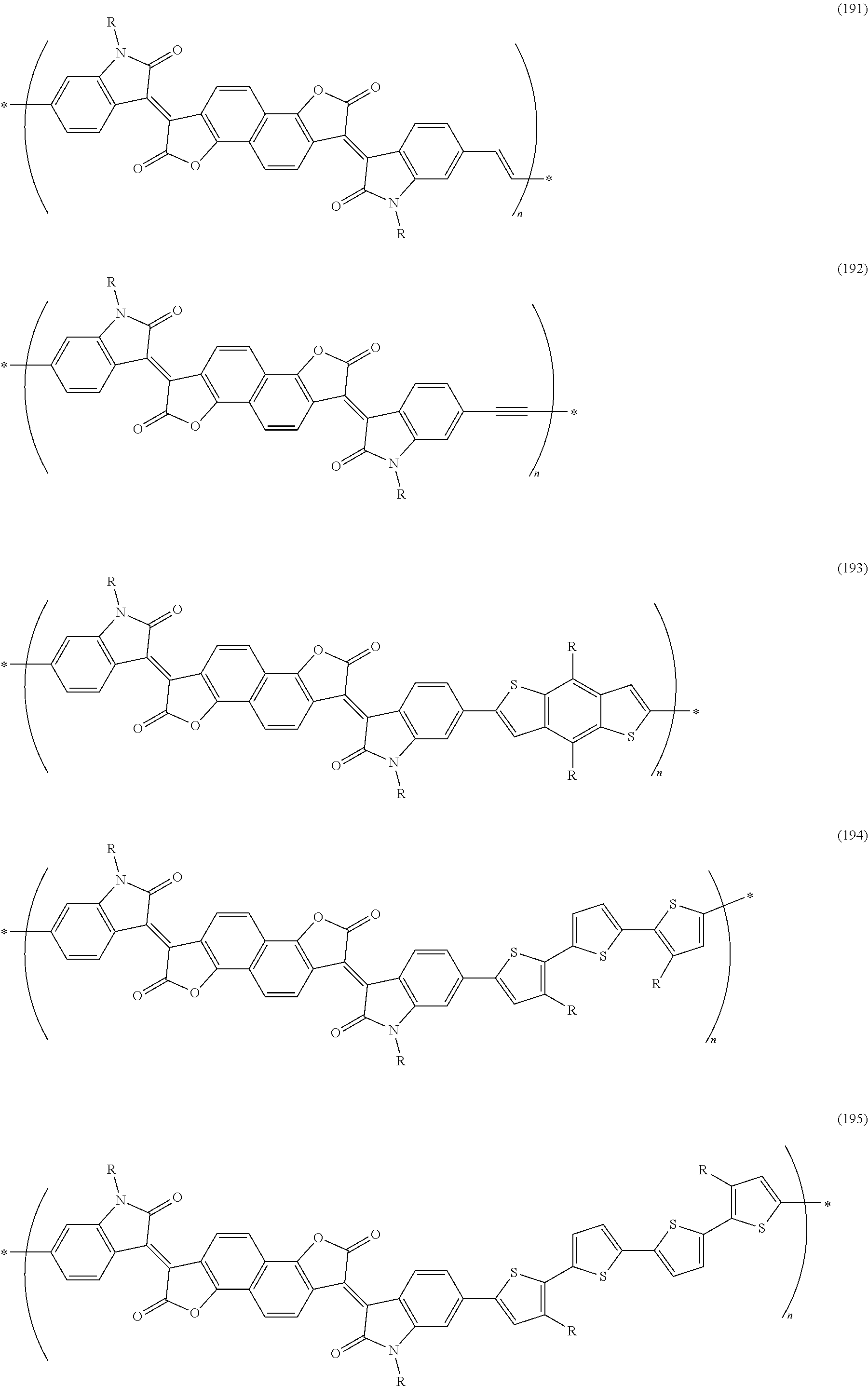 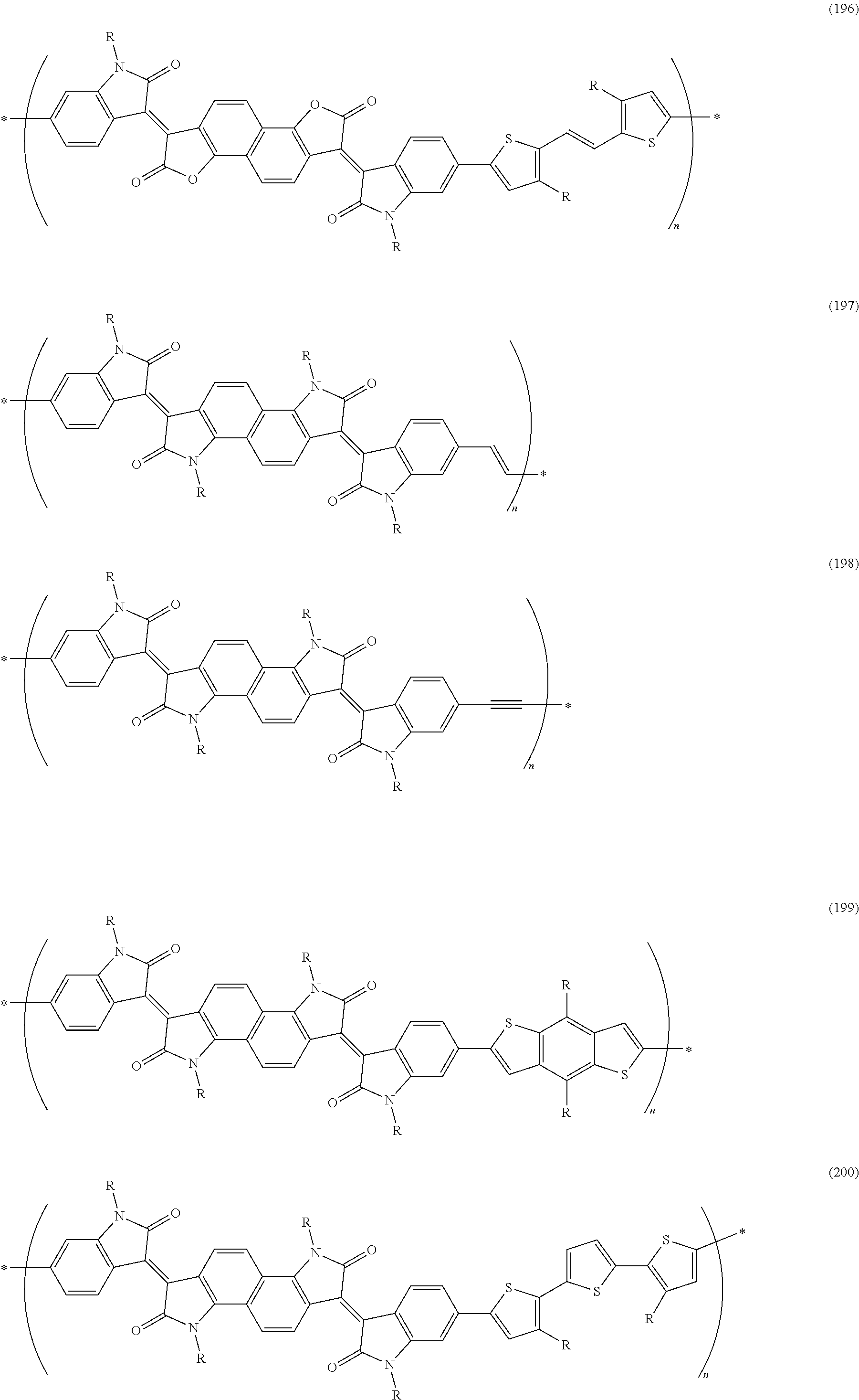 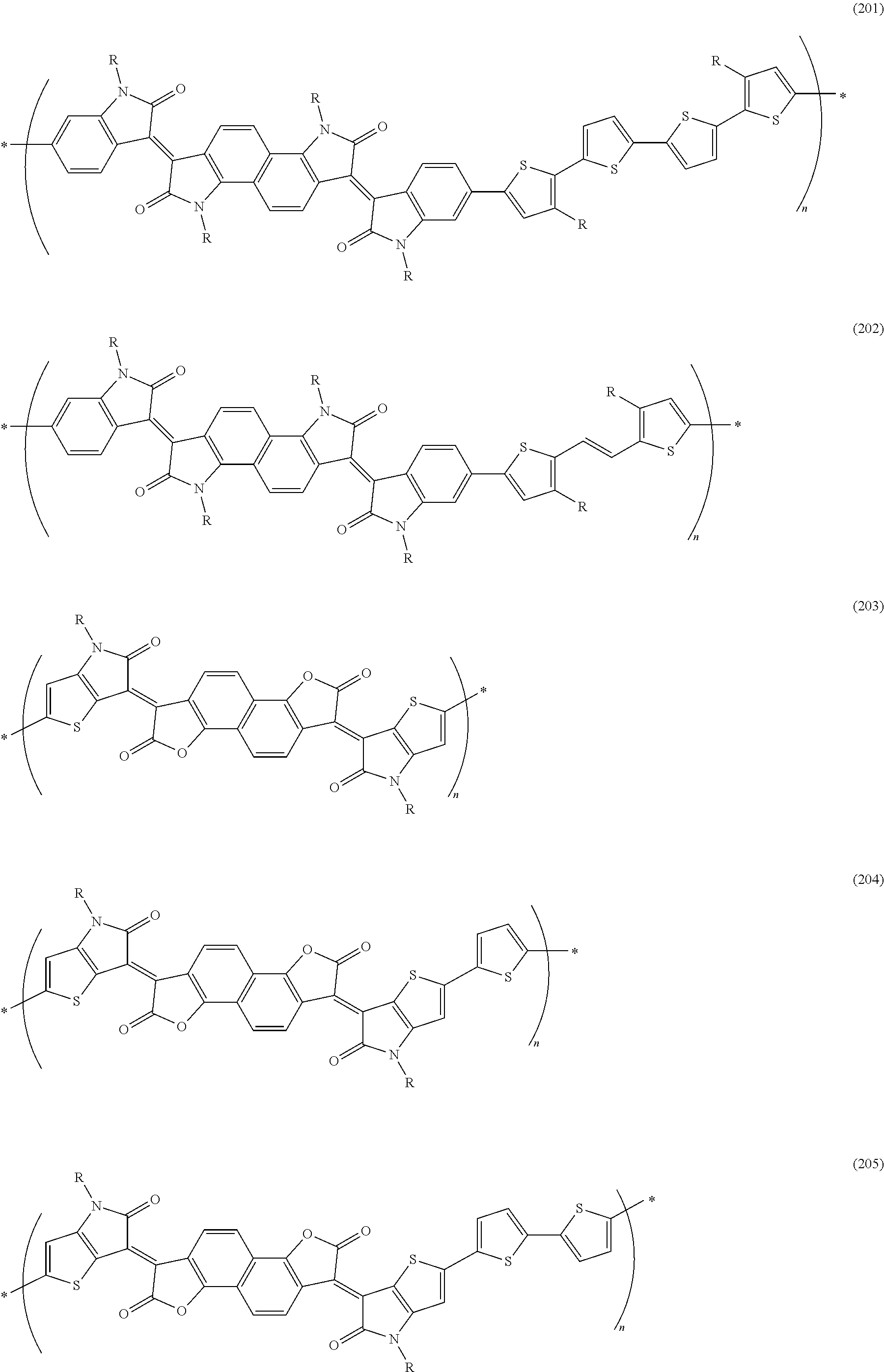 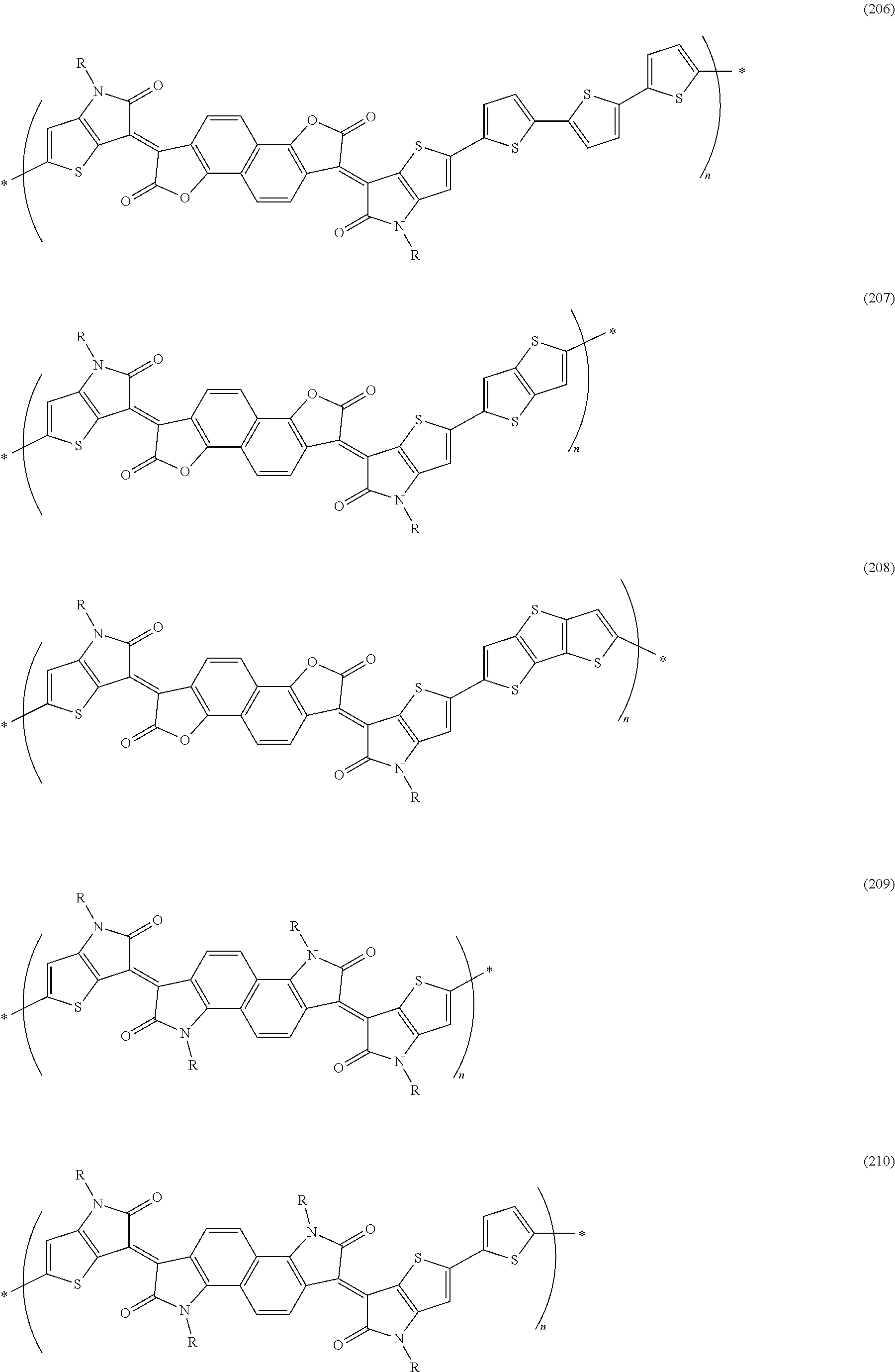 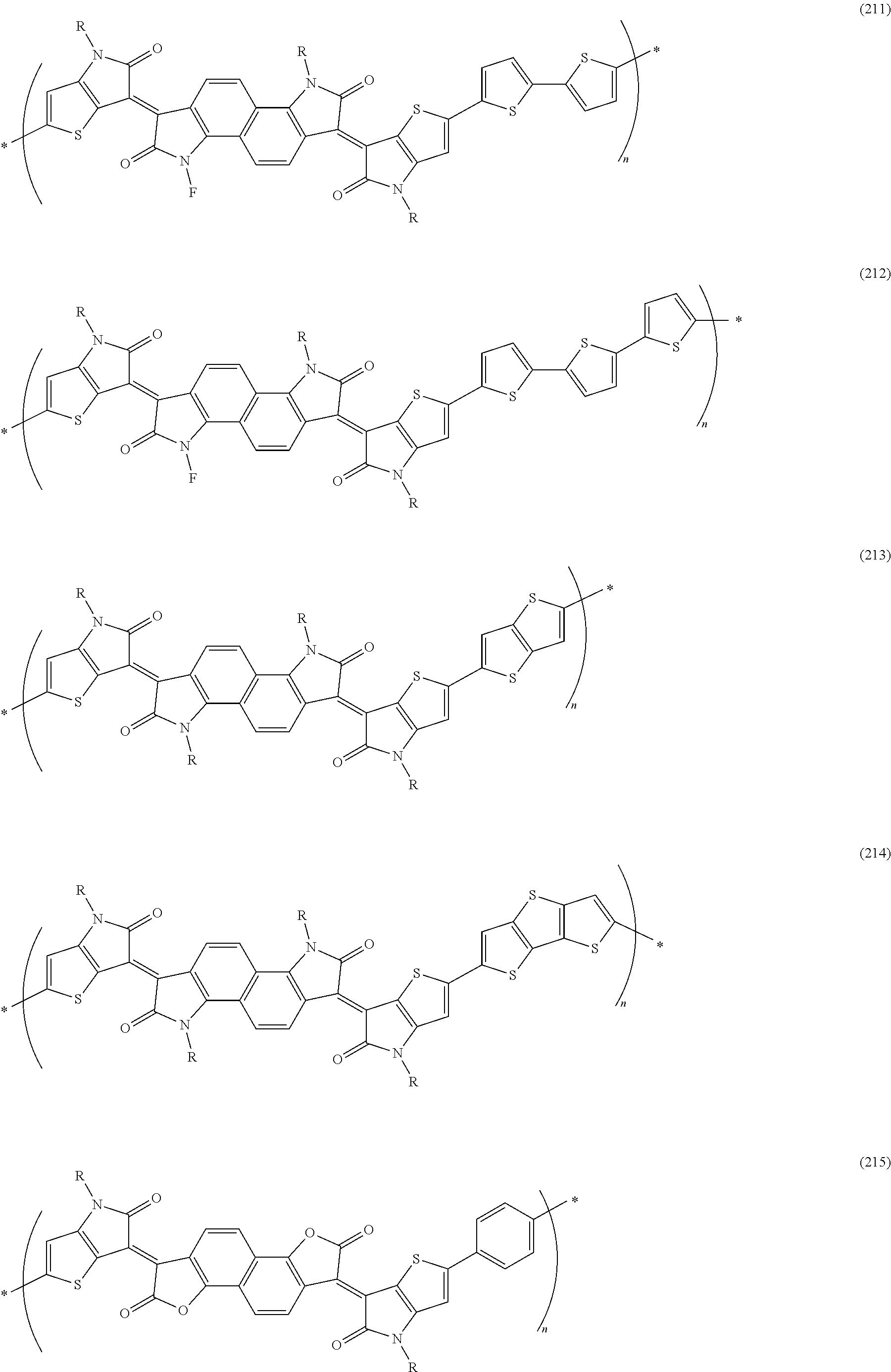 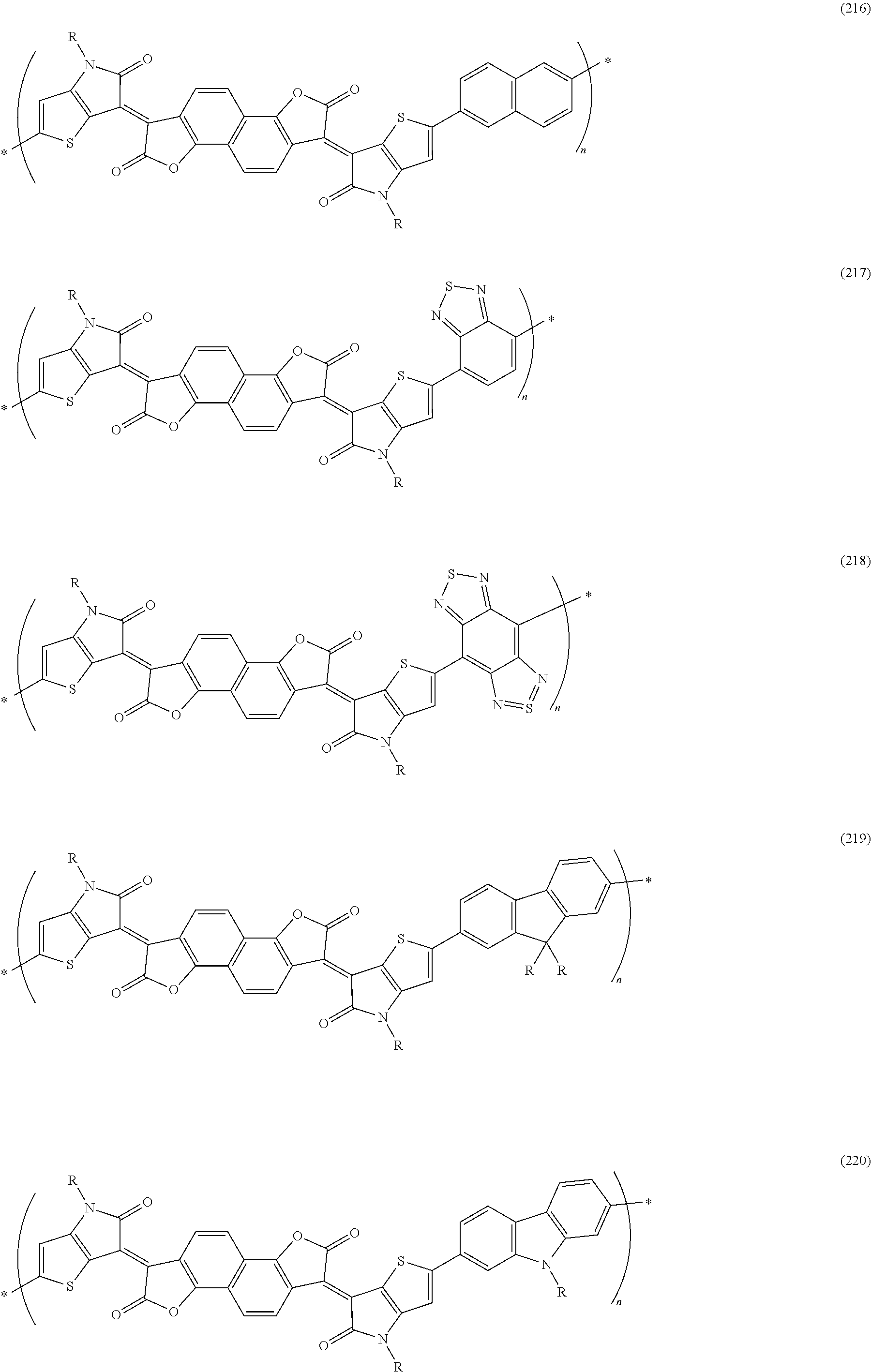 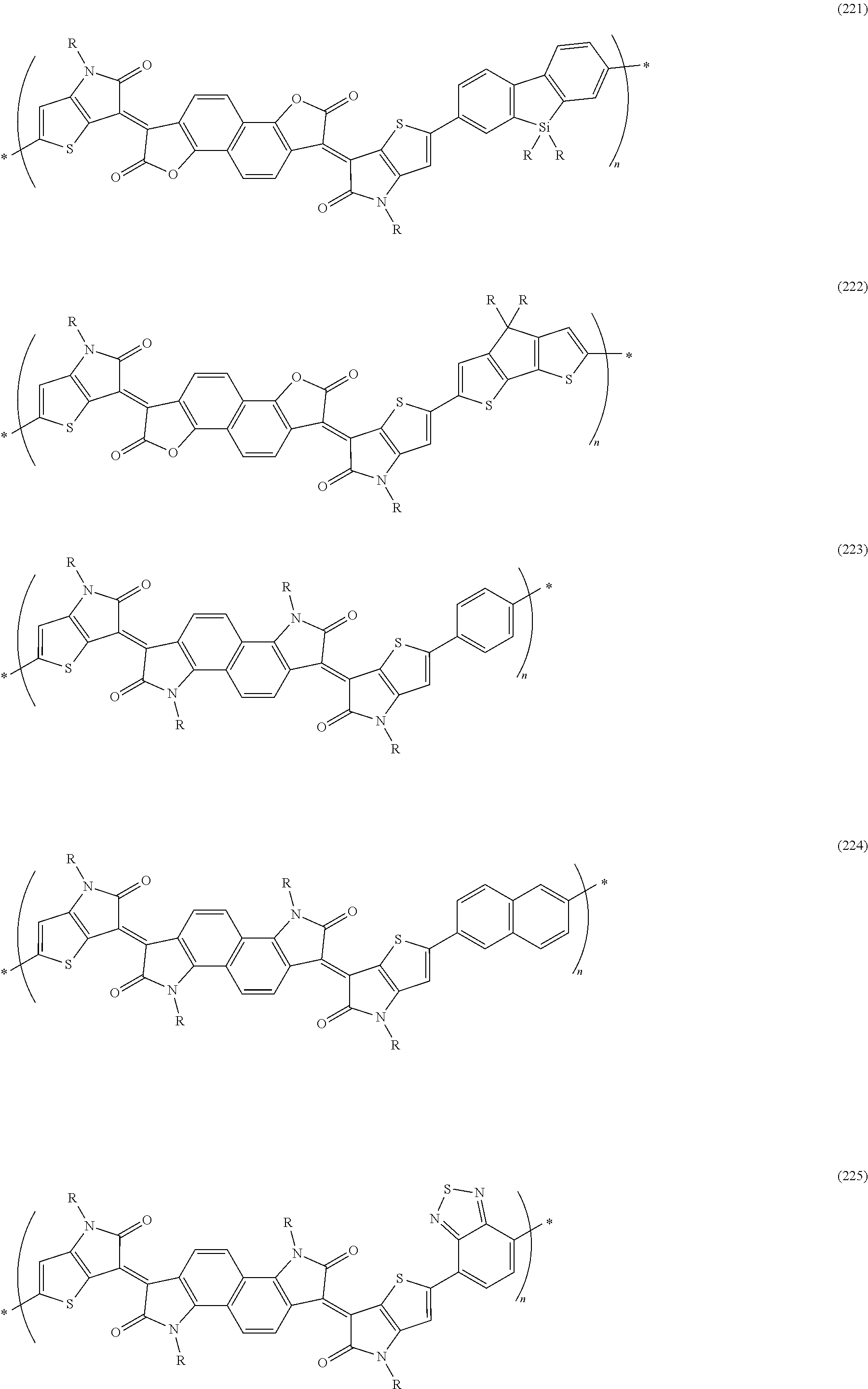 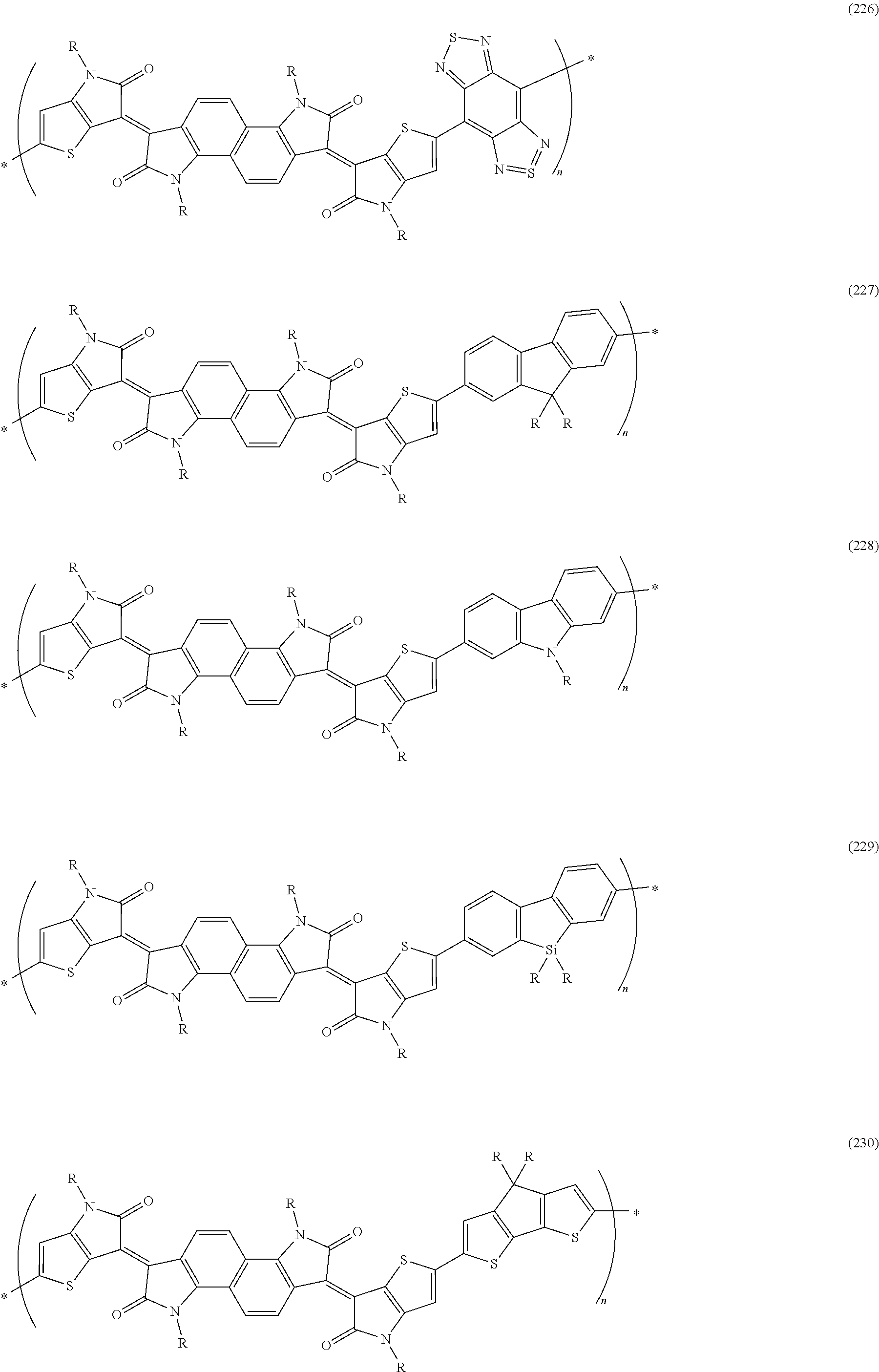
wherein:
R is independently selected from hydrogen, optionally substituted hydrocarbon with 1 to 60 carbon atoms (such as, for example, alkyl, substituted alkyl, alkenyl, substituted alkenyl, alkynyl, substituted alkynyl, aryl, and substituted aryl), or any other suitable group;
each structure can be substituted, where is applicable, with one or more suitable groups independently selected from optionally substituted hydrocarbon with 1 to 60 carbon atoms (such as, for example, alkyl, substituted alkyl, alkenyl, substituted alkenyl, alkynyl, substituted alkynyl, aryl, substituted aryl, alkoxy, and substituted alkoxy), cyano (CN), nitro, and halogen, or any other suitable group;
n is a number from 1 to 1,000,000;
the terminal “*” can be hydrogen or any other suitable group or moiety.

9. An electronic device comprising a monomer, oligomer or polymer according to claim 1.

10. An electronic device according to claim 9, said electronic device being selected from the group consisting of organic field effect transistors (OFET), thin film transistors (TFT), integrated circuits (IC), logic circuits, capacitors, radio frequency identification (RFID) tags, devices or components, organic light emitting diodes (OLED), organic light emitting transistors (OLET), flat panel displays, backlights of displays, organic photovoltaic devices (OPV), organic solar cells (O-SC), photodiodes, laser diodes, photoconductors, organic photodetectors (OPD), electrophotographic devices, electrophotographic recording devices, organic memory devices, sensor devices, charge injection layers, charge transport layers or interlayers in polymer light emitting diodes (PLEDs), Schottky diodes, planarising layers, antistatic films, polymer electrolyte membranes (PEM), conducting substrates, conducting patterns, electrode materials in batteries, alignment layers, biosensors, biochips, security markings, security devices, and components or devices for detecting and discriminating DNA sequences.

11. The electronic device according to claim 9 is an organic thin film transistor.

12. The electronic device according to claim 9 is an organic photovoltaic cell or organic photodiode.

13. A mixture or a blend comprising one or more compounds of claim 1 and one or more compounds or polymers having semiconducting, charge transport, hole transport, electron transport, hole blocking, electron blocking, electrically conducting, photoconducting or light emitting properties.

14. Formulation comprising the compound of claim 1 and an organic solvent.

15. The use of the compound of claim 1 as charge transport, semiconducting, electrically conducting, photoconducting or light emitting material in optical, electrooptical, electronic, electroluminescent or photoluminescent components or devices.

16. Charge transport, semiconducting, electrically conducting, photoconducting or light emitting material comprising the compound of claim 1.
